(19) United States
(12) Patent Application Publication
(10) Pub. No.: US 2020/0131144 A1
(43) Pub. Date: Apr. 30, 2020

(54) AMINE OR (THIO)AMIDE CONTAINING LXR MODULATORS

(71) Applicant: Phenex-FXR GmbH, Heidelberg (DE)

(72) Inventors: Christian Gege, Ehingen (DE); Manfred Birkel, Seeheim-Jugenheim (DE); Eva Hambruch, Mannheim (DE); Ulrich Deuschle, Speyer (DE); Claus Kremoser, Heidelberg (DE)

(73) Assignee: Phenex-FXR GmbH, Heidelberg (DE)

(21) Appl. No.: 16/605,649

(22) PCT Filed: Jul. 18, 2018
(86) PCT No.: PCT/EP2018/069515

§ 371 Date: Oct. 16, 2019

(30) Foreign Application Priority Data
Jul. 18, 2017 (EP) 17001230.6

(51) Int. Cl. C07D 307/52; C07D 417/12; C07D 405/12; C07D 471/04; C07D 413/12; C07D 487/04; A61P 1/16

(52) U.S. Cl. CPC C07D 307/52; C07D 417/12; C07D 405/12; A61P 1/16; C07D 413/12; C07D 487/04; C07D 471/04

(57) ABSTRACT

The present invention relates to derivatives of formula (I) which bind to the liver X receptor (LXRα and/or LXRβ) and act preferably as inverse agonists of LXR.

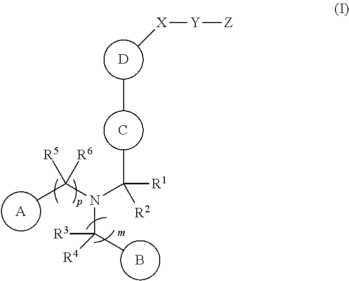

The present invention relates to novel compounds which are Liver X Receptor (LXR) modulators and to pharmaceutical compositions containing same. The present invention further relates to the use of said compounds in the prophylaxis and/or treatment of diseases which are associated with the modulation of the Liver X Receptor.

### BACKGROUND

The Liver X Receptors, LXRα (NR1H3) and LXRβ (NR1H2) are members of the nuclear receptor protein superfamily. Both receptors form heterodimeric complexes with Retinoid X Receptor (RXRα, β or γ) and bind to LXR response elements (e.g. DR4-type elements) located in the promoter regions of LXR responsive genes. Both receptors are transcription factors that are physiologically regulated by binding ligands such as oxysterols or intermediates of the cholesterol biosynthetic pathways, such as desmosterol. In the absence of a ligand, the LXR-RXR heterodimer is believed to remain bound to the DR4-type element in complex with co-repressors, such as NCOR¹, resulting in repression of the corresponding target genes. Upon binding of an agonist ligand, either an endogenous one such as the oxysterols or steroid intermediates mentioned before or a synthetic, pharmacological ligand, the conformation of the heterodimeric complex is changed, leading to the release of corepressor proteins and to the recruitment of coactivator proteins such as NCOA1 (SRC1), resulting in transcriptional stimulation of the respective target genes. While LXRβ is expressed in most tissues, LXRα is expressed more selectively in cells of the liver, the intestine, adipose tissue and macrophages. The relative expression of LXRα and LXRβ at the mRNA or the protein level may vary between different tissues in the same species or between different species in a given tissue. The LXR's control reverse cholesterol transport, i.e. the mobilization of tissue-bound peripheral cholesterol into HDL and from there into bile and feces, through the transcriptional control of target genes such as ABCA1 and ABCG1 in macrophages and ABCG5 and ABCG8 in liver and intestine. This explains the anti-atherogenic activity of LXR agonists in dietary LDLR-KO mouse models. The LXRs, however, do also control the transcription of genes involved in lipogenesis (e.g. SREBF1, SCD, FASN, ACACA) which accounts for the liver steatosis observed following prolonged treatment with LXR agonists.

The liver steatosis liability is considered a main barrier for the development of non-selective LXR agonists for atherosclerosis treatment.

Non-alcoholic fatty liver disease (NAFLD) is regarded as a manifestation of metabolic syndrome in the liver and NAFLD has reached epidemic prevalences worldwide (Marchesini et al., Curr. Opin. Lipidol. 2005; 16:421). The pathologies of NAFLD range from benign and reversible steatosis to steatohepatitis (nonalcoholic steatohepatitis, NASH) that can develop towards fibrosis, cirrhosis and potentially further towards hepatocellular carcinogenesis. Classically, a two-step model has been employed to describe the progression of NAFLD into NASH, with hepatic steatosis as an initiating first step sensitizing towards secondary signals (exogenous or endogenous) that lead to inflammation and hepatic damage (Day et al., Gastroenterology 1998; 114:842).

Notably, LXR expression was shown to correlate with the degree of fat deposition, as well as with hepatic inflammation and fibrosis in NAFLD patients (Ahn et al., Dig. Dis. Sci. 2014; 59:2975). Furthermore, serum and liver desmosterol levels are increased in patients with NASH but not in people with simple liver steatosis. Desmosterol has been characterized as a potent endogenous LXR agonist (Yang et al., J. Biol. Chem. 2006; 281:27816). NAFLD/NASH patients might therefore benefit from blocking the increased LXR activity observed in the livers of these patients through small molecule antagonists or inverse agonists that shut off LXRs' activity. While doing so it needs to be taken care that such LXR antagonists or inverse agonists do not interfere with LXRs in peripheral tissues or macrophages to avoid disruption of the anti-atherosclerotic reverse cholesterol transport governed by LXR in these tissues or cells.

Certain publications (e.g. Peet et al., Cell 1998; 93:693 and Schultz et al., Genes Dev. 2000; 14:2831) have highlighted the role of LXRα, in particular, for the stimulation of lipidogenesis and hence establishment of NAFLD in the liver. They indicate that it is mainly LXRα being responsible for the hepatic steatosis, hence an LXRα-specific antagonist or inverse agonist might suffice or be desirable to treat just hepatic steatosis. These data, however, were generated only by comparing LXRα, LXRβ or double knockout with wild-type mice with regards to their susceptibility to develop steatosis on a high fat diet. They do not account for a major difference in the relative expression levels of LXRα and LXRβ in the human as opposed to the murine liver. Whereas LXRα is the predominant LXR subtype in the rodent liver, LXRβ is expressed to about the same if not higher levels in the human liver compared to LXRα. This was exemplified by testing an LXRβ selective agonist in human phase I clinical studies (Kirchgessner et al., Cell Metab. 2016; 24:223) which resulted in the induction of strong hepatic steatosis although it was shown to not activate human LXRα.

Hence it can be assumed that it should be desirable to have no strong preference of an LXR modulator designed to treat NAFLD or NASH for a particular LXR subtype. A certain degree of LXRsubtype selectivity might be allowed if the pharmacokinetic profile of such a compound clearly ensures sufficient liver exposure and resident time to cover both LXRs in clinical use.

In summary, the treatment of diseases such as NAFLD or NASH would need LXR modulators that block LXRs in a hepato-selective fashion and this could be achieved through hepatotropic pharmacokinetic and tissue distribution properties that have to be built into such LXR modulators.

### PRIOR ART

Zuercher et al. describes with the tertiary sulfonamide (GSK2033) the first potent, cell-active LXR antagonists (J. Med. Chem. 2010; 53:3412; D3 in search report). Later, this compound was reported to display a significant degree of promiscuity, targeting a number of other nuclear receptors (Griffett & Burris, Biochem. Biophys. Res. Commun. 2016; 479:424). All potent examples have a MeSO₂-group and also the SO₂-group of the sulfonamide seems necessary for potency. A replacement of the sulfon from the sulfonamide moiety with a carbonyl or a methylene spacer as in (A1) and (A2) reduced LXR affinity dramatically (pIC₅₀<5.0) not mentioned are the matched pairs of (A1) and (A2) with a MeSO₂-group. It is stated, that GSK2033 showed rapid clearance (Cl_{Int}>1.0 mL/min/mg prot) in rat and human liver microsome assays and that this rapid hepatic metabolism of GSK2033 precludes its use in vivo. As such GSK2033 is an useful chemical probe for LXR in cellular studies only.

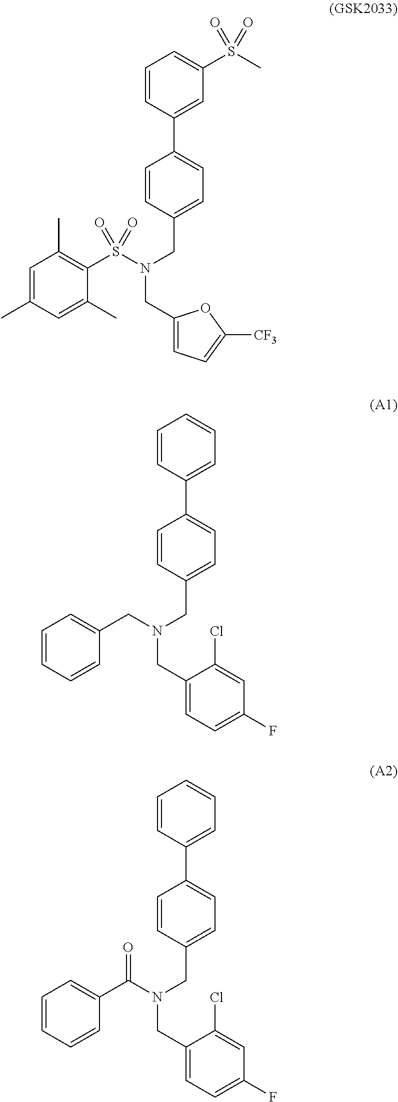

WO2014/085453 (D2 in search report) describes the preparation of small molecule LXR inverse agonists of structure (A) in addition to structure GSK2033 above,

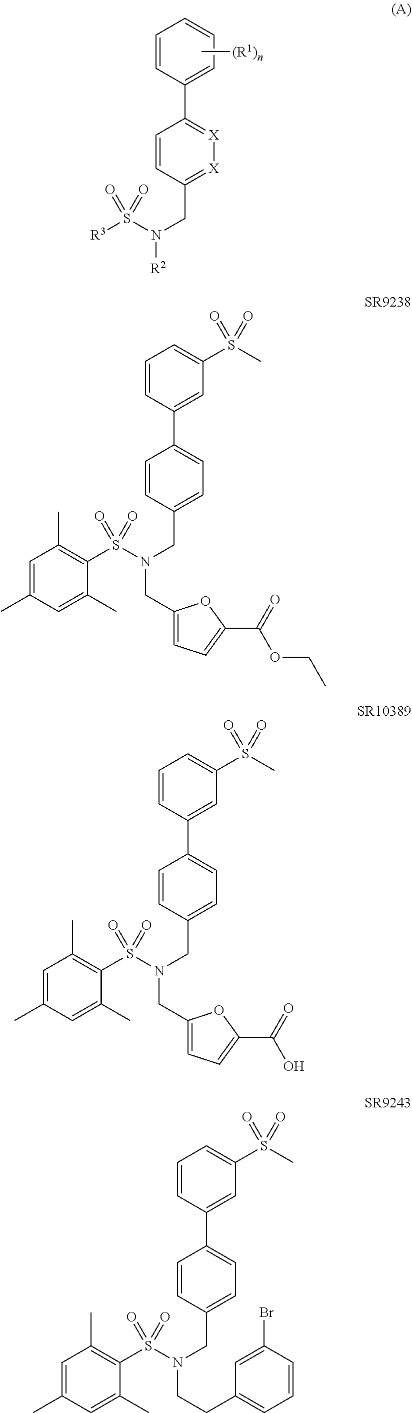

wherein
R¹ is selected from the group consisting of (halo)alkyl, cycloalkyl, (halo)alkoxy, halo, CN, NO₂, OR, SO_{q}R, CO₂R, CONR₂, OCONR₂, NRCONR₂, —SO₂alkyl, —SO₂NR-alkyl, —SO₂-aryl, —SO₂NR-aryl, heterocyclyl, heterocyclyl-alkyl or N- and C-bonded tetrazoyl;
R is selected from H, (halo)alkyl, cycloalkyl, cycloalkyl-alkyl, (hetero)aryl, (hetero)aryl-alkyl, heterocyclyl or heterocyclyl-alkyl;
n is selected from 1 to 3 and q is selected from 0 is 2;
X is selected from N or CH;
R² is selected from alkyl, alkenyl, alkynyl, cycloalkyl, alkyl-C(═O)O-alkyl, aryl-alkyl-C(═O)O-alkyl, aryl-alkyl-O—C(═O)-alkyl, (hetero)aryl, (hetero)aryl-alkyl, heterocyclyl or heterocyclyl-alkyl, wherein all R² residues are substituted with 0 to 3 J-groups;
R³ is selected from alkyl, (hetero)aryl or (hetero)aryl-alkyl, wherein all R³ residues are substituted with 0 to 3 J-groups; and
J is selected from (halo)alkyl, cycloalkyl, heterocyclyl, (hetero)aryl, haloalkyoxy, halo, CN, NO₂, OR, SO_{q}R, CO₂R, CONR₂, O—CO₂R, OCONR₂, NRCONR₂ or NRCO₂R.

The following compounds from this application, in particular, are further described in some publications, mainly from the same group of inventors/authors: SR9238 is described as a liver-selective LXR inverse agonist that suppresses hepatic steatosis upon parenteral administration (Griffett et al., ACS Chem. Biol. 2013; 8:559). After ester saponification of SR9238 the LXR inactive acid derivative SR10389 is formed. This compound then has systemic exposure. In addition, it was described, that SR9238 suppresses fibrosis in a model of NASH again after parenteral administration (Griffett et al., Mol. Metab. 2015; 4:35). With a related SR9243 the effects on aerobic glycolysis (Warburg effect) and lipogenesis were described (Flaveny et al., Cancer Cell 2015; 28:42) and the NASH-supressing data obtained with SR9238 was confirmed by Huang et al. (BioMed Res. Int. 2018; 8071093) using SR9243.

Remarkably, all these derivatives have a methyl sulfone group in the biphenyl portion and the SAR shown in WO2014/085453 suggests, that a replacement or orientation of the MeSO₂-group by other moieties (e.g. —CN, —CONH₂, N-linked tetrazoyl) is inferior for LXR potency. For all compounds shown, no oral bioavailability was reported.

As shown in the experimental section, we confirmed that neutral sulfonamide GSK2033 and SR9238 are not orally bioavailable and hepatoselective. In addition, when the ester in SR9238 is cleaved, the formed acid SR10389 is inactive on LXR.

WO2010/039977 describes heteroaryl antagonists of the prostaglandin D2 receptor with general Formula (B),

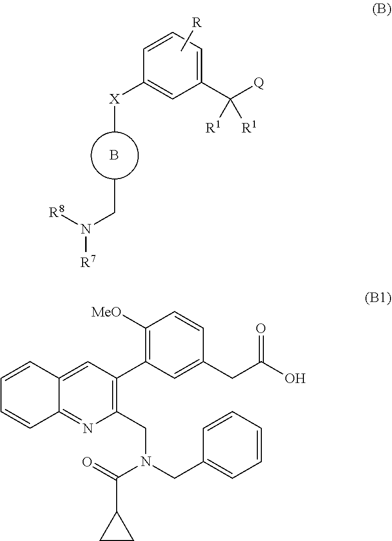

wherein
X is a bond, —O—, —S—, —S(═O)—, —S(O)₂—, —NR¹³—, —CH₂— or —C(O)—;
Q is —C(═O)-Q¹, tertrazolyl or a carboxylic acid bioisostere,
with Q¹ is —OH, —OR, —NHSO₂R, —NR₂, —NH—OH or —NH—CN;
   each R¹ is independently selected from H, F, —CH₃ and —CH₂CH₃;
   ring B is a substituted or unsubstituted heteroaryl;
   R⁷ is selected from a broad range and can be —C(═O)R¹¹,
with R¹¹ is again from a very broad range and can be an optionally substituted cycloalkyl, heterocycloalkyl, aryl or heteroaryl;
   R⁸ is from a very broad range and can be —C₁-C₄-alkylene-R¹⁴,
with R¹⁴ is again from a very broad range and can be an optionally substituted aryl or heteroaryl;

The closest example to the present invention is compound (B1).

WO2002/055484 describes the preparation of small molecules of structure (C), which can be used to increase the amount of low-density lipoprotein (LDL) receptor and are useful as blood lipid depressants for the treatment of hyperlipidemia, atherosclerosis or diabetes mellitus.

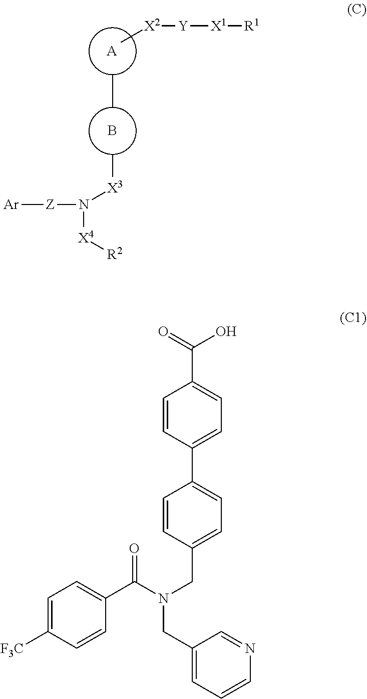

Claimed are structures of Formula (C), wherein

A and B represents independently an optionally substituted 5- or 6-membered aromatic ring;
R¹, R² and R³ is independently selected from H, an optionally substituted hydrocarbon group or an optionally substituted heterocycle;
X¹, X², X³ and X⁴ is independently selected from a bond or an optionally substituted divalent hydrocarbon group;
Y is selected from —NR³CO—, —CONR³—, —NR³—, —SO₂—, —SO₂R³— or —R³—CH₂—;
Z is selected from —CONH—, —CSNH—, —CO— or —SO₂—; and
Ar is selected from an optionally substituted cyclic hydrocarbon group or an optionally substituted heterocycle.

In all carboxamide examples (Z is CO) the X²—Y—X¹—R¹-moiety is in para-position and (C1) is the only example, where the X²—Y—X¹—R¹-moiety contains a carboxylic acid.

WO2006/009876 describes compounds of Formula (D) for modulating the activity of protein tyrosine phosphatases,

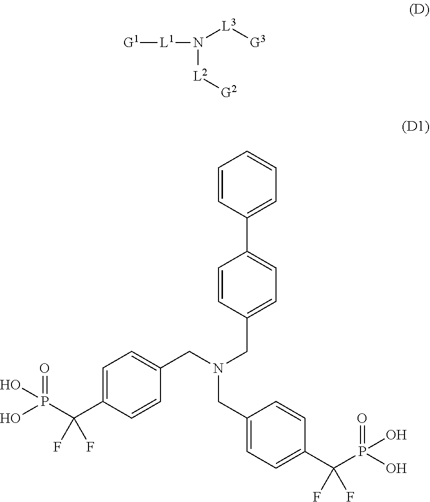

wherein
L¹, L², L³ is independently selected from a bond or an optionally substituted group selected from alkylene, alkenylene, alkynylene, cycloalkylene, oxocycloalkylene, amidocycloalkylene, heterocyclylene, heteroarylene, C═O, sulfonyl, alkylsulfonyl, alkenylsulfonyl, alkynylsulfonyl, amide, carboxamido, alkylamide, alkylcarboxamido and alkoxyoxo;
G¹, G², G³ is independently selected from alkyl, alkenyl, alkynyl, aryl, alkaryl, arylalkyl, alkarylalkyl, alkenylaryl, alkylsulfonyl, alkenylsulfonyl, alkynylsulfonyl, amido, alkylamino, alkylaminoaryl, arylamino, aminoalkyl, aminoaryl, alkoxy, alkoxyaryl, aryloxy, alkylamido, alkylcarboxamido, arylcarboxamido, alkoxyoxo, biaryl, alkoxyoxoaryl, amidocycloalkyl, carboxyalkylaryl, carboxyaryl, carboxyamidoaryl, carboxamido, cyanoalkyl, cyanoalkenyl, cyanobiaryl, cycloalkyl, cycloalkyloxo, cycloalkylaminoaryl, haloalkyl, haloalkylaryl, haloaryl, heterocyclyl, heteroaryl, hydroxyalkylaryl and sulfonyl; wherein each residue is optionally substituted with 1 to 3 substituents selected from H, alkyl, alkenyl, alkynyl, aryl, arylalkyl, alkoxy, alkoxyoxo, alkylthia, amino, amido, arylamino, aryloxy, alkylamino, alkylsulfonyl, alkylcarboxyalkylphosphonato, arylcarboxamido, carboxy, carboxyoxo, carboxyalkyl, carboxyalkyloxa, carboxyalkenyl, carboxyamido, carboxyhydroxyalkyl, cycloalkyl, am ido, cyano, cyanoalkenyl, cyanoaryl, amidoalkyl, amidoalkenyl, halo, haloalkyl, haloalkylsulfonyl, heterocyclyl, heteroaryl, heteroarylalkyl, heteroarylalkoxy, hydroxy, hydroxyalkyl, hydroxyamino, hydroxyimino, heteroarylalkyloxa, nitro, phosphonato, phosphonatoalkyl and phosphonatohaloalkyl.

From the huge range of possible substituents compound (D1) is closest to the scope of the present invention. Most examples have a sulfonamide moiety (L¹ is SO₂) instead a carboxamide or tertiary amine in that position.

WO2006/063697 describes compounds of Formula (E) with a direct attached carboxylic acid in meta-position of the biphenyl for inhibiting the activity of phosphotyrosine phosphatase 1B (PTP1B),

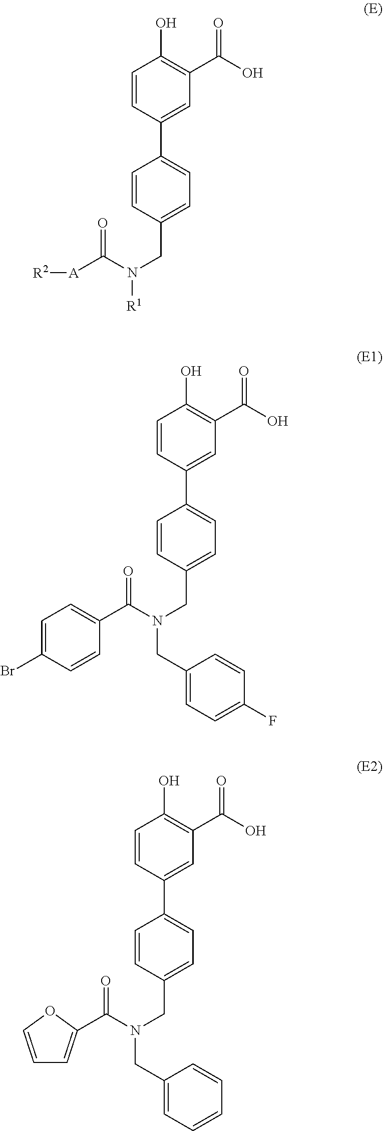

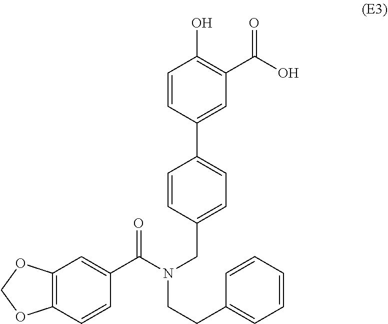

wherein
R¹ is selected from a very broad range of substituents and can be —(C₁-C₆)-alkyl-aryl or —(C₁-C₆)-alkyl-cycloalkyl, wherein alkyl, cycloalkyl and aryl can be optionally substituted;
R² is selected from a cycloalkyl or heterocycle, both of them can be optionally substituted;
A is selected from a bond, O, NH or S.

Representative examples are (E1) to (E3).

An additional example for a direct attached carboxylic acid in meta-position of the bihetroaryl moiety is compound (F), which is used as a flexible polydendate ligand (Charbonniëre et al. Tetrahedron Lett. 2001; 42:659).

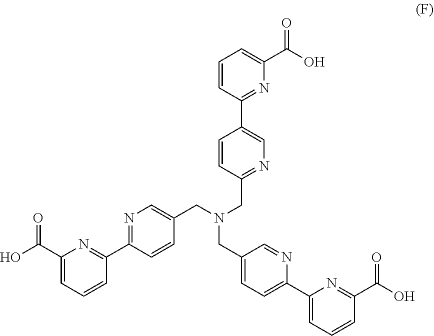 [SMILES: O=C(O)c1cccc(-c2ccc(CN(Cc3ccc(-c4cccc(C(=O)O)n4)nc3)Cc3ccc(-c4cccc(C(=O)O)n4)nc3)nc2)n1]

WO2005/030702 (U.S. Pat. No. 7,534,894) describes compounds as inhibitors of PAI-1 with general Formula (G). An acid or acid isoster is attached to the biphenyl moiety via a linker element,

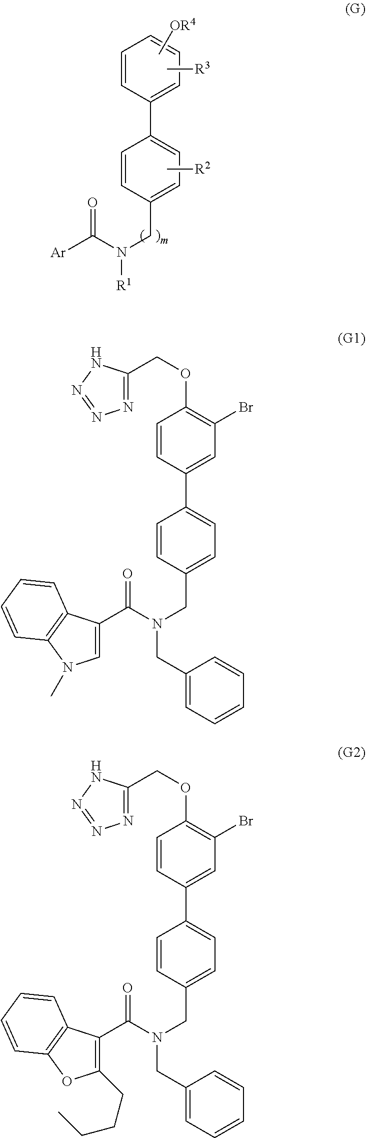

wherein
Ar is selected from phenyl, naphthyl, furanyl, thiophenyl, benzofuranyl, benzothiophenyl, indolyl, pyrazolyl, oxazolyl, fluorenyl, phenylcycloalkyl or dihydroindenyl;
R¹ is hydrogen, C₁-C₆-alkyl or —(CH₂)ᵣ-phenyl;
R² and R³ are independently hydrogen, C₁-C₆-alkyl, —(CH₂)ₚ-phenyl, halogen and C₁-C₃-perfluoroalkyl;
R⁴ is —CHR₅CO₂H, —CH₂-tetrazole or an acid mimic;
R⁵ is hydrogen or benzyl;
n is selected from 0 or 1, r is selected from 0 to 6 and p is selected from 0 to 3;
wherein Ar, alkyl, phenyl and benzyl groups are optionally substituted.

No structures with a meta-linked carboxylic acid or isoster are exemplified. The closest derivatives with that moiety in para-position are (G1) and (G2).

An example for a sulfonylacetic acid moiety is described by Faucher et al. (J. Med. Chem. 2004; 47:18), however the carboxamide moiety of compound (H) is in an orientation, which is outside the scope of the present invention.

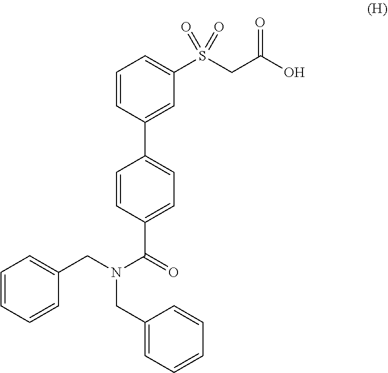 [SMILES: O=C(O)CS(=O)(=O)c1cccc(-c2ccc(C(=O)N(Cc3ccccc3)Cc3ccccc3)cc2)c1]

WO2005/102388 (US2008/0132574) describes compounds of general Formula (J) for the treatment of a BLT2-mediated disease

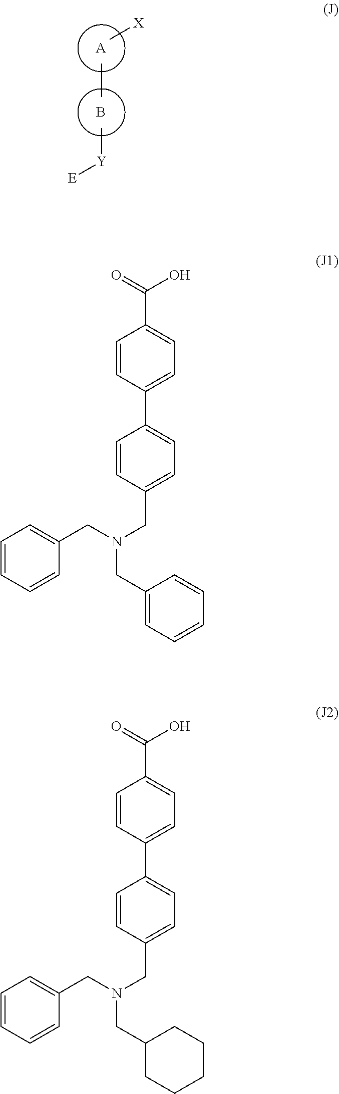

wherein
X represents an acidic group;
Y represents a bond or a spacer (1 to 3 atoms);
E represents an amino group, which may be substituted; and
A and B each represent a optionally substituted ring.

Compound (J1) and (J2) are the closest biphenyl derivatives however the acidic group is directly attached to the aryl.

The ortho-substituted direct carboxamide (K) is commercially available according SciFinder (CAS: 2027377-21-3).

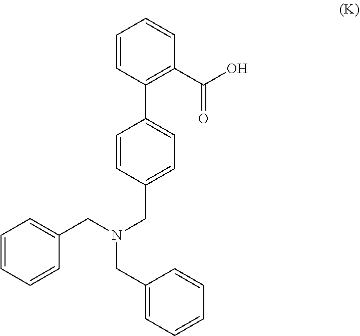 [SMILES: O=C(O)c1ccccc1-c1ccc(CN(Cc2ccccc2)Cc2ccccc2)cc1]

WO2017/006261 (D1 in search report) describes pyridin-3-yl acetic acid derivatives of general Formula (L) as inhibitors of human immunodeficiency virus replication

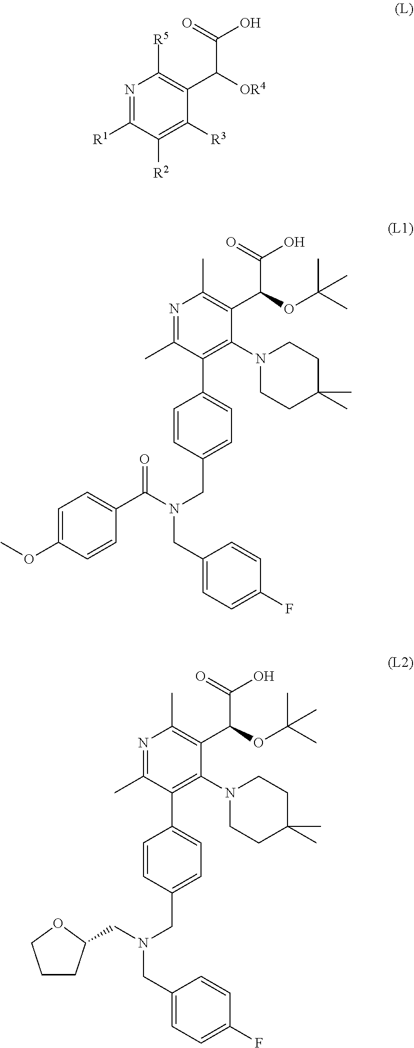

wherein
R¹ selected from hydrogen or alkyl;
R² is selected from ((R⁶O)CR⁹R¹⁰)phenyl, ((R⁶S)CR⁹R¹⁰)phenyl or (((R⁶)(R⁷)N)CR⁹R¹⁰)phenyl;
R³ is selected from azetidinyl, pyrrolidinyl, piperidinyl, piperazinyl, morpholinyl, homo-piperidinyl, homopiperazinyl, or homomorpholinyl and is substituted with 0-3 substituents selected from cyano, halo, alkyl, haloalkyl, alkoxy or haloalkoxy;
R⁴ is selected from alkyl or haloalkyl;
R⁵ is alkyl;
R⁶ is selected from alkyl, cycloalkyl, (cycloalkyl)alkyl, (R⁸)C₁₋₃-alkyl, or (Ar¹)C₀₋₃-alkyl;
R⁷ is selected from hydrogen, alkyl, (furanyl)alkyl, alkoxy, alkylcarbonyl, cycloalkylcarbonyl, (phenoxy)methylcarbonyl, alkoxycarbonyl, benzyloxycarbonyl, (R⁸)carbonyl, (Ar²)carbonyl, alkylsulfonyl, phenyl sulfonyl or mesitylenesulfonyl;
R⁹ and R¹⁰ is independently selected from hydrogen or alkyl;
Ar¹ is a monocyclic heteroaryl or phenyl substituted with 0-3 substituents selected from halo, alkyl, haloalkyl, alkoxy, haloalkoxy, carboxy and alkoxycarbonyl;
Ar² is selected from phenyl, furanyl, or thienyl, and is substituted with 0-3 substituents selected from halo, alkyl, haloalkyl, alkoxy and haloalkoxy.

Compound (L1) and (L2) are the closest derivatives to the present invention the —R³-group has to be present in all compounds.

WO2003/082802 (D4 in search report) describes LXR agonists of general Formula (M):

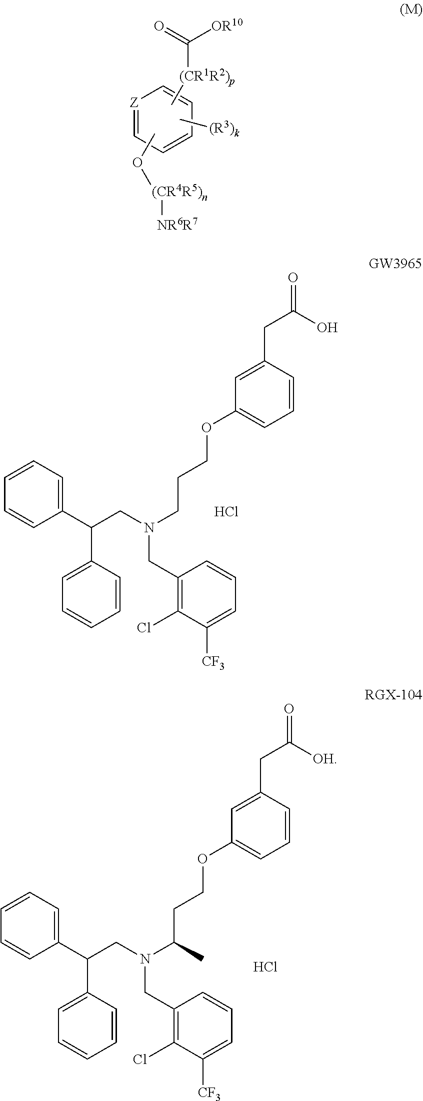

In all examples the acid containing (hetero)aryl moiety is linked via an oxygen atom to the rest of the molecule. Most interesting examples are GW3965 (Collins et al. J. Med. Chem. 2002; 45:1963) and clinical candidate RGX-104 from Rgenix.

### SUMMARY OF THE INVENTION

The present invention relates to compounds according to Formula (I)

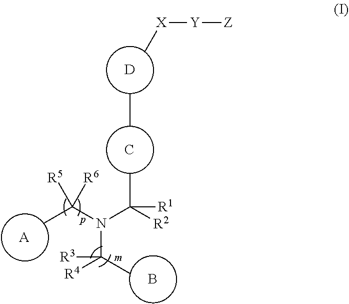

an enantiomer, diastereomer, tautomer, N-oxide, solvate, prodrug and pharmaceutically acceptable salt thereof,
wherein A, B, C, D, X, Y, Z, R¹ to R⁶, m and p are defined as in claim 1.

We surprisingly found, that potent, orally bioavailable LXR modulators with hepatoselective properties can be obtained, when a carboxylic acid or a carboxylic acid isoster (see e.g. Ballatore et al., ChemMedChem 2013; 8:385, Lassalas et al., J. Med. Chem. 2016; 59:3183) is tethered covalently to the methylsulfon moiety of (GSK2033) or the methylsulfon moiety of (GSK2033) is replaced by another carboxylic acid- or carboxylic acid isoster-containing moiety. The compounds of the present invention have a similar or better LXR inverse agonistic, antagonistic or agonistic activity compared to the known LXR-modulators without an acidic moiety. Furthermore, the compounds of the present invention exhibit an advantageous liver/blood-ratio after oral administration so that disruption of the anti-atherosclerotic reverse cholesterol transport governed by LXR in peripheral macrophages can be avoided. The incorporation of an acidic moiety (or a bioisoster thereof) can improve additional parameters, e.g. microsomal stability, solubility and lipophilicity, in a beneficial way, in addition.

Thus, the present invention further relates to a pharmaceutical composition comprising a compound according to Formula (I) and at least one pharmaceutically acceptable carrier or excipient.

The present invention is further directed to compounds according to Formula (I) for use in the prophylaxis and/or treatment of diseases mediated by LXRs.

Accordingly, the present invention relates to the prophylaxis and/or treatment of non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis and hepatitis C virus infection.

### DETAILED DESCRIPTION OF THE INVENTION

The desired properties of an LXR modulator in conjunction with hepatoselectivity, can be yielded with compounds that follow the structural pattern represented by Formula (I)

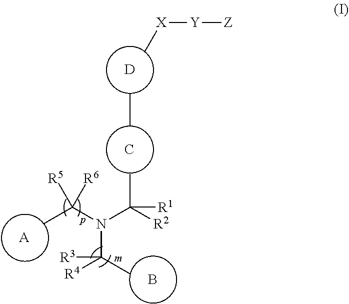

an enantiomer, diastereomer, tautomer, N-oxide, solvate, prodrug and pharmaceutically acceptable salt thereof, wherein
R¹, R² are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;
or R¹ and R² together are a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, halo-C₁₋₄-alkyl, O—C₁₋₄-alkyl, O-halo-C₁₋₄-alkyl;
or R¹ and an adjacent residue from ring C form a 5- to 8-membered saturated or partially unsaturated cycloalkyl or a 5- to 8-membered saturated or partially unsaturated heterocycloalkyl containing 1 to 4 heteroatoms independently selected from N, O and S, wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
R³, R⁴ are independently selected from H and C₁₋₄-alkyl; wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl, O-halo-C₁₋₄-alkyl;
or R³ and R⁴ together are a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, halo-C₁₋₄-alkyl, O—C₁₋₄-alkyl, O-halo-C₁₋₄-alkyl;
or R³ and an adjacent residue from ring B form a 5- to 8-membered partially unsaturated cycloalkyl or a 5- to 8-membered partially unsaturated heterocycloalkyl containing 1 to 4 heteroatoms independently selected from N, O and S, wherein the cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
R⁵, R⁶ are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;
or R⁵ and R⁶ together are oxo, thioxo, a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, 0-C₁₋₄-alkyl, O-halo-C₁₋₄-alkyl;
or R⁵ and an adjacent residue from ring A form a 5- to 8-membered saturated or partially unsaturated cycloalkyl or a 5- to 8-membered saturated or partially unsaturated heterocycloalkyl containing 1 to 4 heteroatoms independently selected from N, O and S, wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;

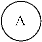

is selected from the group consisting of 4- to 10-membered cycloalkyl, 4- to 10-membered heterocycloalkyl containing 1 to 4 heteroatoms independently selected from N, O and S, 6- to 14-membered aryl and 5- to 14-membered heteroaryl containing 1 to 4 heteroatoms independently selected from N, O and S, wherein cycloalkyl, heterocycloalkyl, aryl and heteroaryl are unsubstituted or substituted with 1 to 6 substituents independently selected from the group consisting of halogen, CN, NO₂, oxo, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, Cₘ-alkylene-NR⁵¹S(O)₂R⁵¹, Cₘ-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², Cₘ-alkylene-NR⁵¹—COR⁵¹, Cₘ-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;

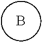

is selected from the group consisting of 6- or 10-membered aryl and 5- to 10-membered heteroaryl containing 1 to 4 heteroatoms independently selected from N, O and S, wherein the 6-membered aryl and 5- or 6-membered heteroaryl are substituted with 1 to 4 substituents independently selected from the group consisting of halogen, CN, NO₂, OXO, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆ alkylene-O—CONR⁶¹R⁶², C₀₋₆-alkylene-NR⁶¹—CO₂R⁶¹ and C₀₋₆-alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; and wherein the 10-membered aryl or 7- to 10-membered heteroaryl are unsubstituted or substituted with 1 to 4 substituents independently selected from the group consisting of halogen, CN, NO₂, OXO, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;

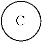

is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S, wherein cycloalkyl, heterocycloalkyl, aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is optionally substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; wherein the residue —CR¹R²— on ring C is linked at least with one 1,4-orientation regarding the connection towards ring D;

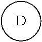

is selected from the group consisting of 6-membered aryl and 5- to 6-membered heteroaryl containing 1 to 4 heteroatoms independently selected from N, O and S, wherein aryl and heteroaryl are unsubstituted or substituted with 1 to 4 substituents independently selected from the group consisting of halogen, CN, NO₂, OXO, C₁₋₄-alkyl, C₀₋₆-alkylene-OR¹, C₀₋₆-alkylene-(3- to 6-membered cycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹ and C₀₋₆-alkylene-NR⁸¹R⁸², wherein alkyl, alkylene and cycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents on the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; wherein the residue X—Y—Z on ring D is linked in 1,3-orientation regarding the connection towards ring C;
X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹;
Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S, wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂;
Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, —P(═)H(OH), —B(OH)₂,

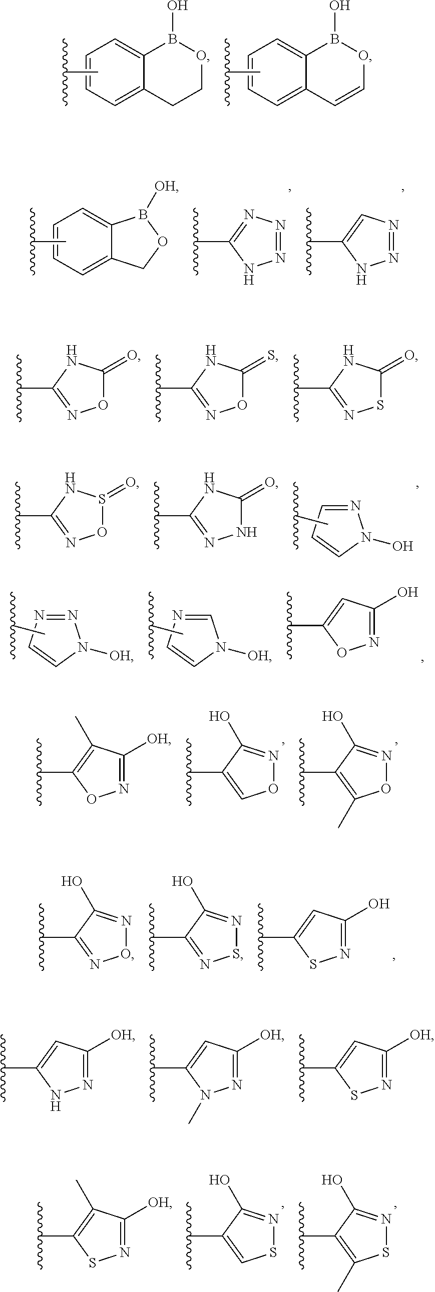

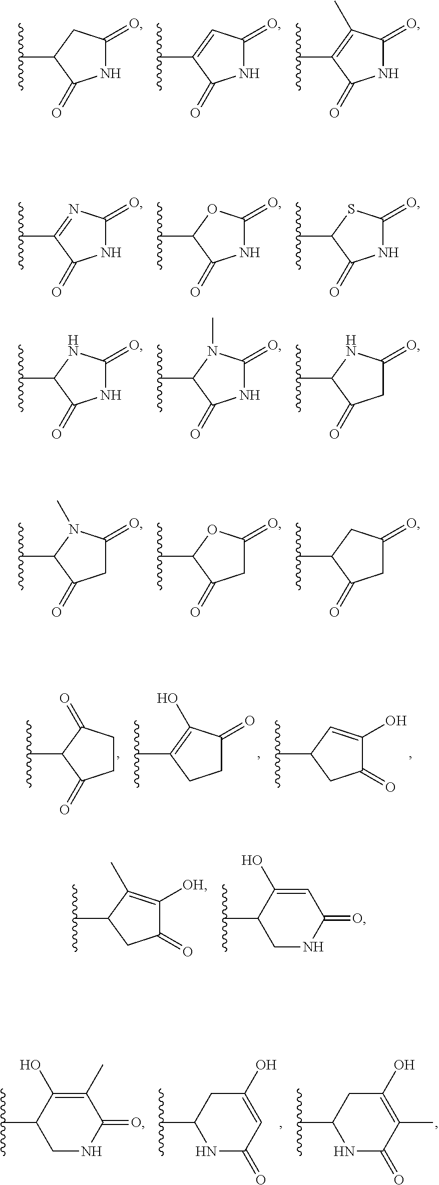

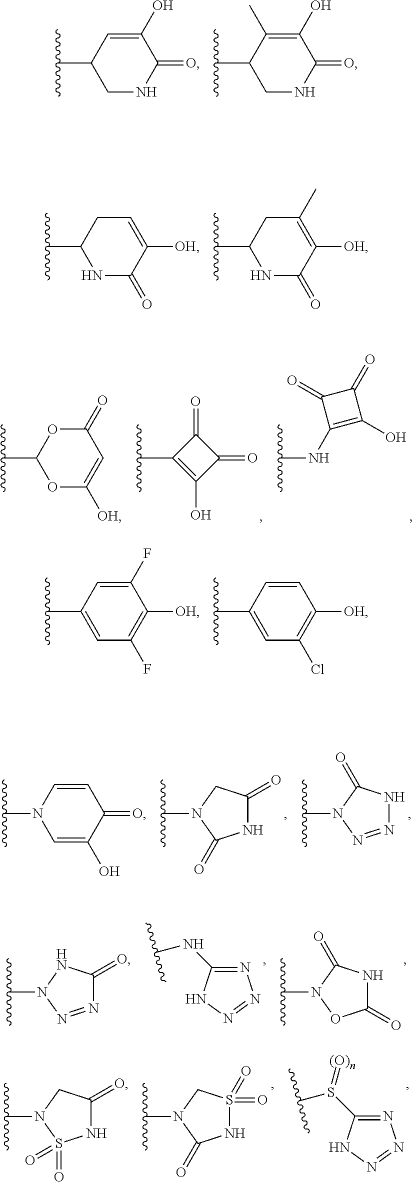

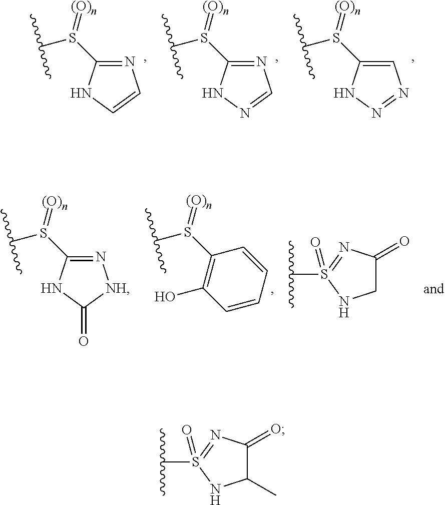

R¹¹ is selected from H, CN, NO₂, C₁₋₄-alkyl, C(═O)—C₁₋₄alkyl, C(═O)—O—C₁₋₄-alkyl, halo-C₁₋₄-alkyl, C(═O)-halo-C₁₋₄-alkyl and C(═O)—O-halo-C₁₋₄-alkyl;
R⁵¹, R⁵², R⁶¹, R⁶², R⁷¹, R⁷², R⁸¹, R⁸² are independently selected from H and C₁₋₄-alkyl,
wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁14-alkyl;
or R⁵¹ and R⁵², R⁶¹ and R⁶², R⁷¹ and R⁷², respectively, when taken together with the nitrogen to which they are attached complete a 3- to 6-membered ring containing carbon atoms and optionally containing 1 or 2 heteroatoms independently selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₄-alkyl;
R⁹⁰ is independently selected from C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
R⁹¹, R⁹² are independently selected from H and C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
or R⁹¹ and R⁹² when taken together with the nitrogen to which they are attached complete a 3- to 6-membered ring containing carbon atoms and optionally containing 1 or 2 heteroatoms selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
n is selected from 0 to 2; m and p is independently selected from 1 and 2.

In a preferred embodiment in combination with any of the above or below embodiments R¹ and R² are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;

or R¹ and R² together are a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, O-halo-C₁₋₄-alkyl;
or R¹ and an adjacent residue from ring C form a 5- to 8-membered saturated or partially unsaturated cycloalkyl or a 5- to 8-membered saturated or partially unsaturated heterocycloalkyl containing 1 to 4 heteroatoms independently selected from N, O and S, wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl.

In a more preferred embodiment in combination with any of the above or below embodiments R¹ and R² are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl.

In a most preferred embodiment in combination with any of the above or below embodiments R¹ and R² are both H.

In a preferred embodiment in combination with any of the above or below embodiments R³ and R⁴ are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl, O-halo-C₁₋₄-alkyl;

or R³ and R⁴ together are a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, O-halo-C₁₋₄-alkyl;
or R³ and an adjacent residue from ring B form a 5- to 8-membered partially unsaturated cycloalkyl or a 5- to 8-membered partially unsaturated heterocycloalkyl containing 1 to 4 heteroatoms independently selected from N, O and S, wherein the cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl.

In a more preferred embodiment in combination with any of the above or below embodiments R³ and R⁴ are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl, O-halo-C₁₋₄-alkyl.

In a even more preferred embodiment in combination with any of the above or below embodiments R³ and R⁴ are independently selected from H and Me.

In a most preferred embodiment in combination with any of the above or below embodiments R³ and R⁴ are both H.

In a preferred embodiment in combination with any of the above or below embodiments R⁵ and R⁶ are independently selected from H and C₁₋₄-alkyl, wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;

or R⁵ and R⁶ together are oxo, thioxo, a 3- to 6-membered cycloalkyl or a 3- to 6-membered heterocycloalkyl containing 1 to 4 heteroatoms independently selected from N, O and S, wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, O-halo-C₁₋₄-alkyl;
or R⁵ and an adjacent residue from ring A form a 5- to 8-membered saturated or partially unsaturated cycloalkyl or a 5- to 8-membered saturated or partially unsaturated heterocycloalkyl containing 1 to 4 heteroatoms independently selected from N, O and S, wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl.

In a more preferred embodiment in combination with any of the above or below embodiments R⁵ and R⁶ are independently selected from H and C₁₋₄-alkyl, wherein alkyl is unsubstituted or substituted with 1 to 3 substituents independently selected from halogen, N, OH, oxo, C₁₋₄-alkyl, halo-C₁₋₄-alkyl, O—C₁₋₄-alkyl and O-halo-C₁₋₄-alkyl; or R⁵ and R⁶ together are oxo.

In a most preferred embodiment in combination with any of the above or below embodiments R⁵ and R⁶ are independently selected from H and Me.

In a similar most preferred embodiment in combination with any of the above or below embodiments R⁵ and R⁶ are together oxo.

In a preferred embodiment in combination with any of the above or below embodiments m and p is independently selected from 1 and 2.

In a more preferred embodiment in combination with any of the above or below embodiments p is 1 and m is selected from 1 and 2.

In a most preferred embodiment in combination with any of the above or below embodiments both m and p are 1.

In a preferred embodiment in combination with any of the above or below embodiments m and p is 1, R¹, R², R³ and R⁴ are independently selected from H or Me, R⁵ and R⁶ are independently selected from H or Me or R⁵ and R⁶ together are oxo.

In a preferred embodiment in combination with any of the above or below embodiments R⁵¹, R⁵², R⁶¹, R⁶², R⁷¹, R⁷², R⁸¹, R⁸² are independently selected from H, Me and Et;

or R⁵¹ and R⁵², R⁶¹ and R⁶², R⁷¹ and R⁷², respectively, when taken together with the nitrogen to which they are attached complete a ring system independently selected from azetidine, piperidine and morpholine.

In a more preferred embodiment in combination with any of the above or below embodiments R⁵¹, R⁵², R⁶¹, R⁶², R⁷¹, R⁷², R⁸¹, R⁸² are independently selected from H and Me.

In a preferred embodiment in combination with any of the above or below embodiments R⁹⁰ is Me and Et.

In a more preferred embodiment in combination with any of the above or below embodiments R⁹⁰ is Me.

In a preferred embodiment in combination with any of the above or below embodiments R⁹¹, R⁹² are independently selected from H, Me and Et.

In a more preferred embodiment in combination with any of the above or below embodiments R⁹¹, R⁹² is independently selected from H and Me.

In another preferred embodiment in combination with any of the above or below embodiments

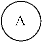

is selected from the group consisting of 4- to 10-membered cycloalkyl, 4- to 10-membered heterocycloalkyl containing 1 to 4 heteroatoms independently selected from N, O and S, 6- to 14-membered aryl and 5- to 14-membered heteroaryl containing 1 to 4 heteroatoms independently selected from N, O and S, wherein cycloalkyl, heterocycloalkyl, aryl and heteroaryl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents on the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; and wherein optionally two adjacent substituents on the cycloalkyl or heterocycloalkyl moiety form a 5- to 6-membered unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

Within a first alternative, in a more preferred embodiment in combination with any of the above or below embodiments

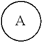

is selected from the group consisting of 6- to 14-membered aryl and 5- to 14-membered heteroaryl containing 1 to 4 heteroatoms independently selected from N, O and S, wherein aryl and heteroaryl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C_{0O6}-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents on the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; or

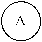

is selected from the group consisting of 4- to 10-membered cycloalkyl and 4- to 10-membered heterocycloalkyl containing 1 to 4 heteroatoms independently selected from N, O and S, wherein cycloalkyl and heterocycloalkyl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein two adjacent substituents on the cycloalkyl or heterocycloalkyl moiety form a 5- to 6-membered unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

Within this first alternative, in a more preferred embodiment in combination with any of the above or below embodiments

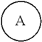

is selected from phenyl, pyridyl, imidazopyrimidinyl, imidazopyridinyl, imidazopyridazinyl, triazolopyridinyl, pyrazolopyridazinyl, pyrazolopyrimidinyl, naphthyl, benzo[b]thiophenyl, 1,2,3,4-tetrahydronaphthyl, chromanyl, isochromanyl, quinoline, isoquinoline, quinolin-2(1H)-onyl, isoquinolin-2(1H)-onyl, naphthyridinyl, pyridopyrimidinyl, cinnolinyl, phthalazinyl, anthracenyl, acridinyl and 1,2,3,4-tetrahydroanthracenyl, wherein said moiety is unsubstituted or substituted with 1 to 4 substituents independently selected from F, Cl, Br, CN, NO₂, OH, oxo, Me, Et, cyclopropyl, CHF₂, OF₃, OMe, OEt, OCHF₂ and OCF₃.

Within this first alternative, in an even more preferred embodiment in combination with any of the above or below embodiments

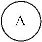

is selected from phenyl, pyridyl, naphthyl, benzo[b]thiophenyl, 1,2,3,4-tetrahydronaphthyl, chromanyl, isochromanyl, quinoline, isoquinoline, quinolin-2(1H)-onyl, isoquinolin-2(1H)-onyl, naphthyridinyl, cinnolinyl, phthalazinyl, anthracenyl, acridinyl and 1,2,3,4-tetrahydroanthracenyl, wherein said moiety is unsubstituted or substituted with 1 to 4 substituents independently selected from F, Cl, Br, CN, NO₂, OH, oxo, Me, Et, CHF₂, OF₃, OMe, OEt, OCHF₂ and OCF₃.

Within this first alternative, in a most preferred embodiment in combination with any of the above or below embodiments

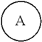

is selected from

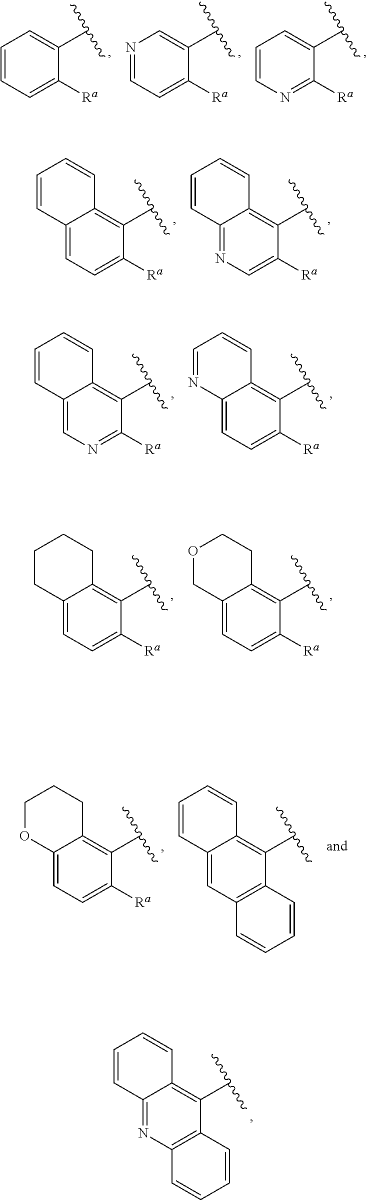

wherein R^{a} is selected from Cl, CN, Me, Et, CHF₂, CF₃, OMe, OCHF₂ and OCF₃; and

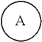

is unsubstituted or substituted with 1 to 3 substituents independently selected from F, Cl, Br, CN, NO₂, OH, oxo, Me, Et, CHF₂, OF₃, OMe, OEt, OCHF₂ and OCF₃.

Within this first alternative, in an even most preferred embodiment in combination with any of the above or below embodiments

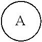

is selected from

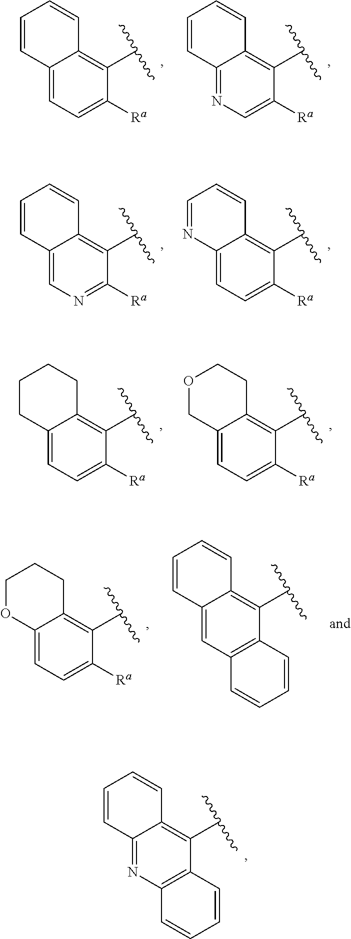

wherein R^{a} is selected from Cl, CN, Me, Et, CHF₂, CF₃, OMe, OCHF₂ and OCF₃; and

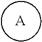

is unsubstituted or substituted with 1 to 3 substituents independently selected from F, Cl, Br, CN, NO₂, OH, oxo, Me, Et, CHF₂, CF₃, OMe, OEt, OCHF₂ and OCF₃.

Within this first alternative, in a similar preferred embodiment in combination with any of the above or below embodiments

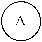

is selected from

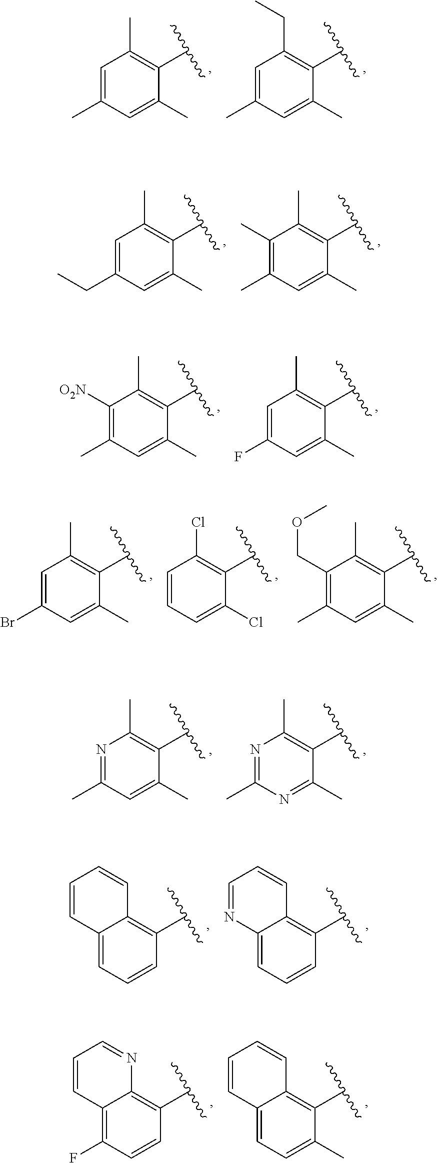

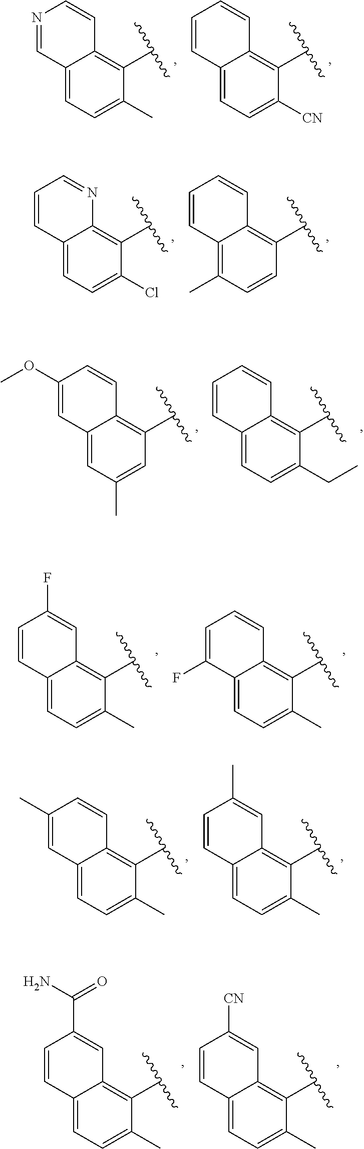

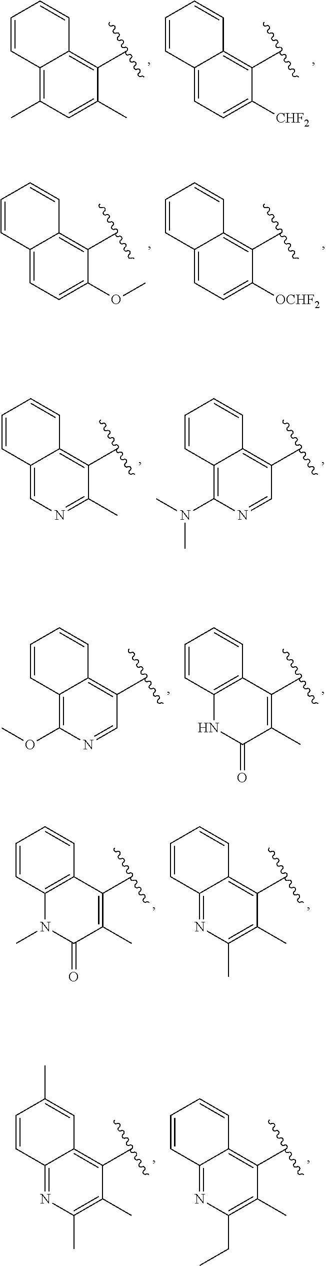

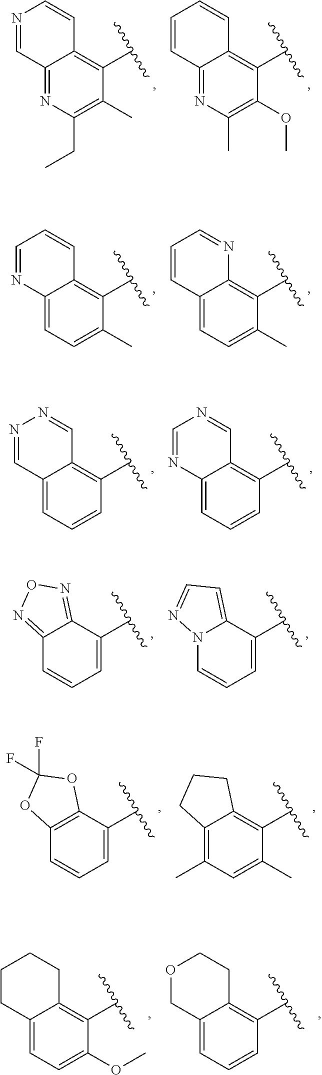

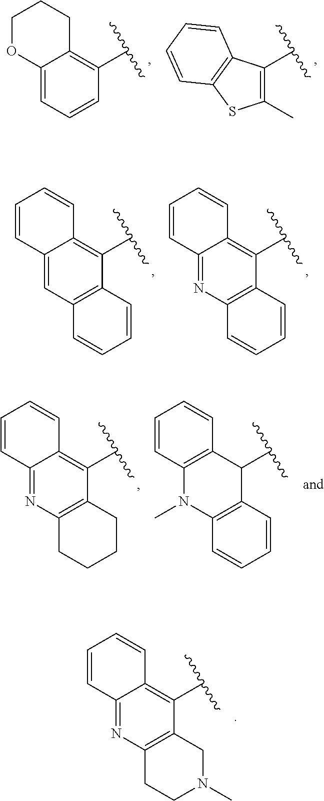

Within this first alternative, in a similar more preferred embodiment in combination with any of the above or below embodiments

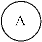

is selected from

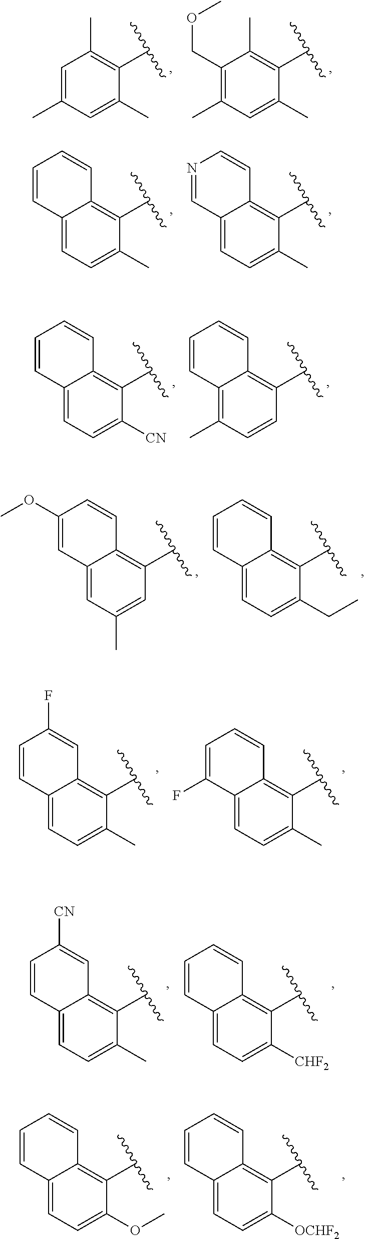

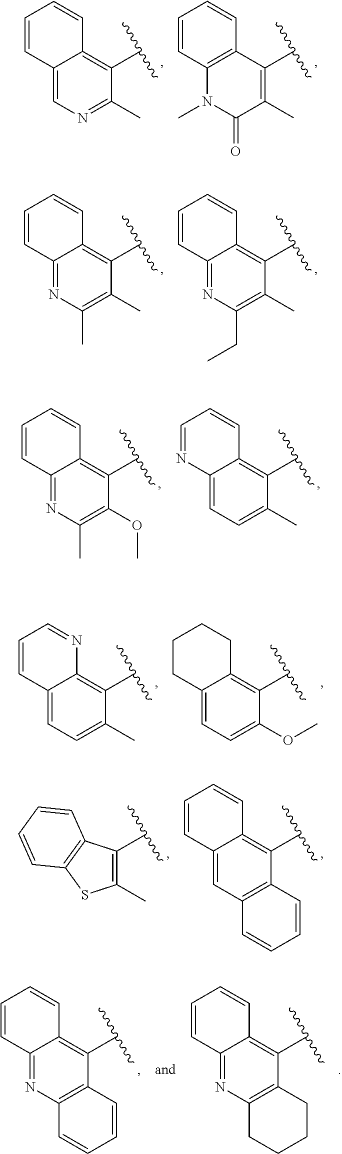

Within this first alternative, in a similar most preferred embodiment in combination with any of the above or below embodiments

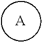

is selected from

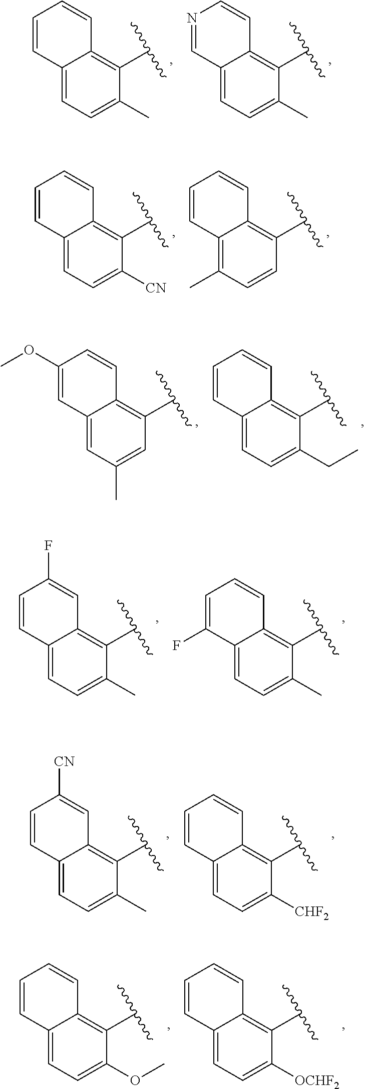

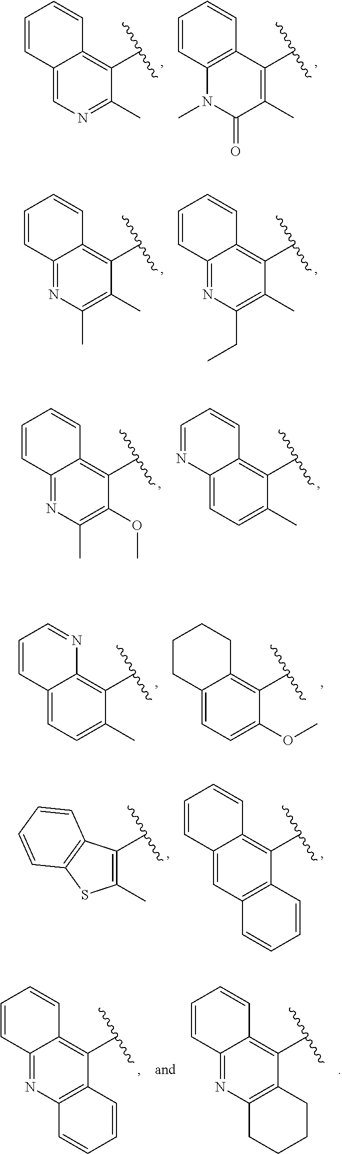

Within a second first alternative, a preferred embodiment in combination with any of the above or below embodiments

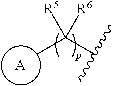

is selected from

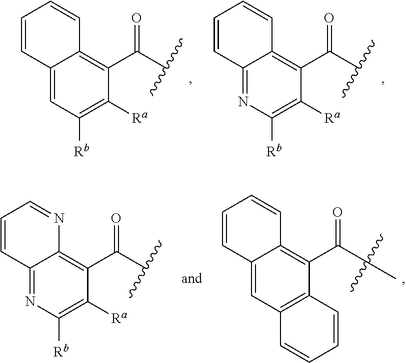

wherein R^{a} and R^{b} is independently selected from H, Cl, CN, Me, Et, cyclopropyl, CHF₂, CF₃, OH, OMe, OCHF₂ and OCF₃; and

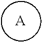

may be further substituted with 1 to 3 additional substituents independently selected from F, Cl, Br, CN, OH, Me, Et, CHF₂, CF₃, OMe, OEt, OCHF₂ and OCF₃.

Within this second alternative, in a more preferred embodiment in combination with any of the above or below embodiments

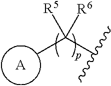

is selected from

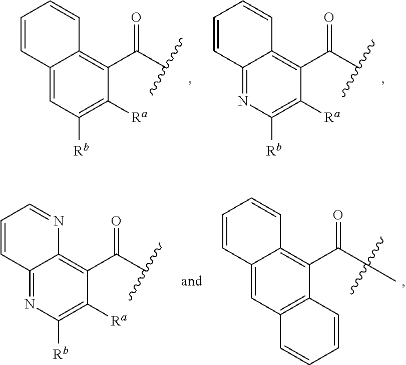

wherein R^{a} is H, and R^{b} is selected from H, Cl, CN, Me, Et, cyclopropyl, CHF₂, OF₃, OMe, OCHF₂ and OCF₃; and

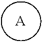

may be further substituted with 1 to 3 additional substituents independently selected from F, Cl, Br, CN, OH, Me, Et, CHF₂, CF₃, OMe, OEt, OCHF₂ and OCF₃.

Within this second alternative, in an even more preferred embodiment in combination with any of the above or below embodiments

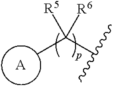

is selected from

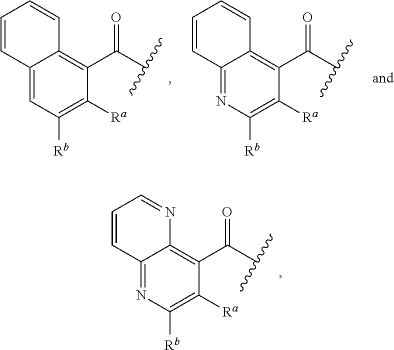

wherein R^{a} is H, and R^{b} is selected from H, Cl, CN, Me, Et, cyclopropyl, CHF₂, CF₃, OMe, OCHF₂ and OCF₃; and

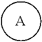

may be further substituted with 1 to 3 additional substituents independently selected from F, Cl, Br, CN, OH, Me, Et, CHF₂, CF₃, OMe, OEt, OCHF₂ and OCF₃.

Within this second alternative, in a most preferred embodiment in combination with any of the above or below embodiments

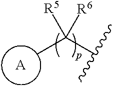

is selected from

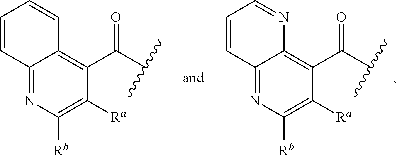

wherein R^{a} is H, and R^{b} is selected from Me, Et, cyclopropyl, CHF₂, CF₃, OMe, OCHF₂ and OCF₃; and

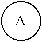

may be further substituted with 1 to 3 additional substituents independently selected from F, CN, Me, Et, CHF₂, CF₃, OMe, OEt, OCHF₂ and OCF₃.

In an equally preferred embodiment of the second alternative in combination with any of the above or below embodiments

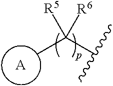

is selected from

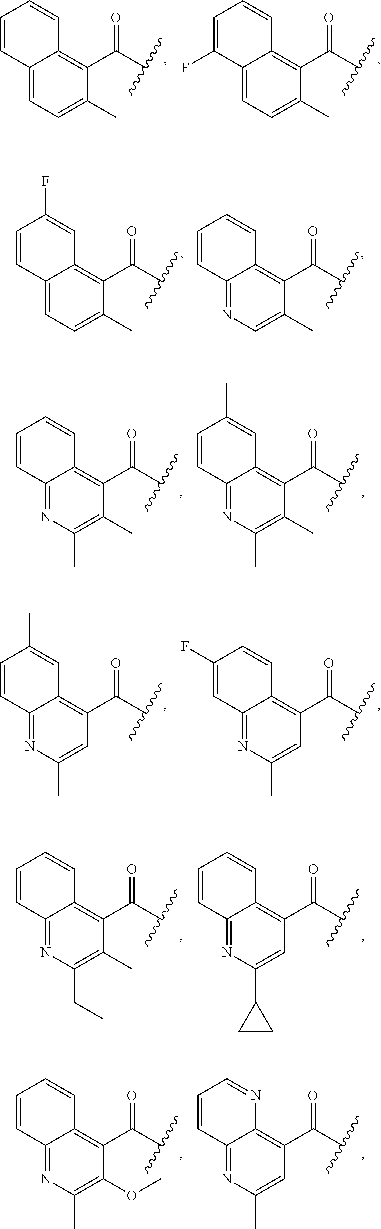

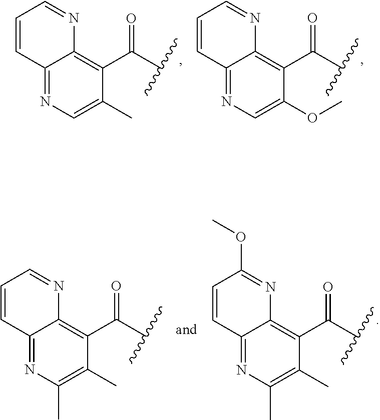

In an equally most preferred embodiment of the second alternative in combination with any of the above or below embodiments

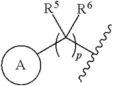

is selected from

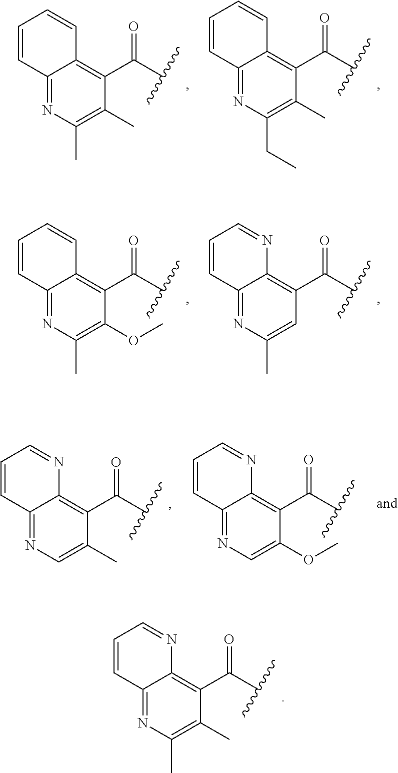

In a further preferred embodiment in combination with any of the above or below embodiments

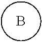

is selected from the group consisting of 6- or 10-membered aryl and 5- to 10-membered heteroaryl containing 1 to 4 heteroatoms independently selected from N, O and S, wherein the 6-membered aryl and 5- or 6-membered heteroaryl are substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and
wherein optionally two adjacent substituents in the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; and
wherein the 10-membered aryl or 7- to 10-membered heteroaryl are unsubstituted or substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶⁹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

In a more preferred embodiment in combination with any of the above or below embodiments

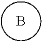

is selected from the group consisting of 6-membered aryl and 5- to 6-membered heteroaryl containing 1 to 4 heteroatoms independently selected from N, O and S, wherein the 6-membered aryl and 5- or 6-membered heteroaryl are substituted with 1 to 4 substituents independently selected from the group consisting of halogen, CN, NO₂, OXO, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl.

In a more preferred embodiment in combination with any of the above or below embodiments

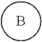

is selected from furanyl, thiophenyl, thiazolyl, pyrrolyl, phenyl and pyridyl, wherein the aryl moiety is substituted with 1 to 2 substituents independently selected from the group consisting of halogen, CN, CO₂—C₁₋₄-alkyl, CONH₂, CONHC₁₋₄-alkyl, CON(C₁₋₄-alkyl)₂, C₁₋₄-alkyl, halo-C₁₋₄-alkyl, O—C₁₋₄-alkyl and O-halo-C₁₋₄-alkyl.

In an even more preferred embodiment in combination with any of the above or below embodiments

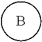

is selected from

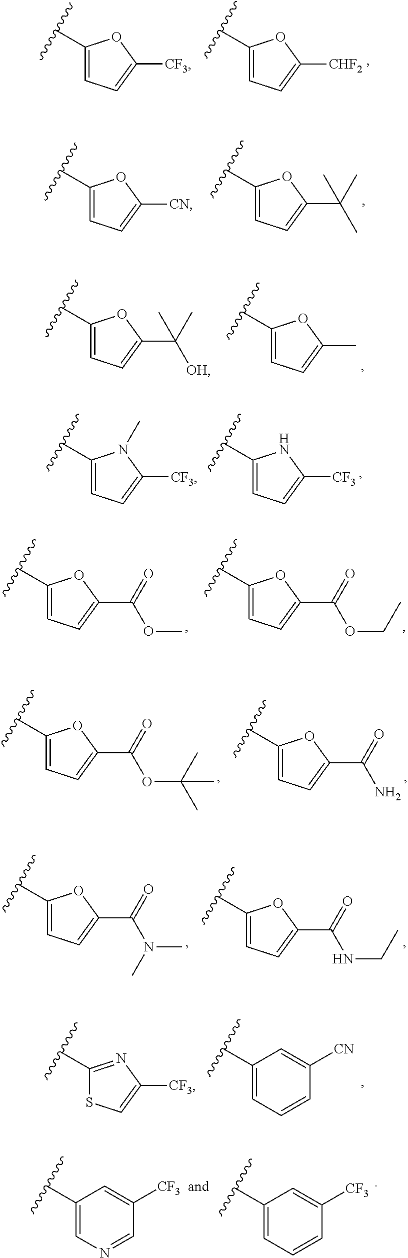

In an even more preferred embodiment in combination with any of the above or below embodiments

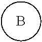

is selected from

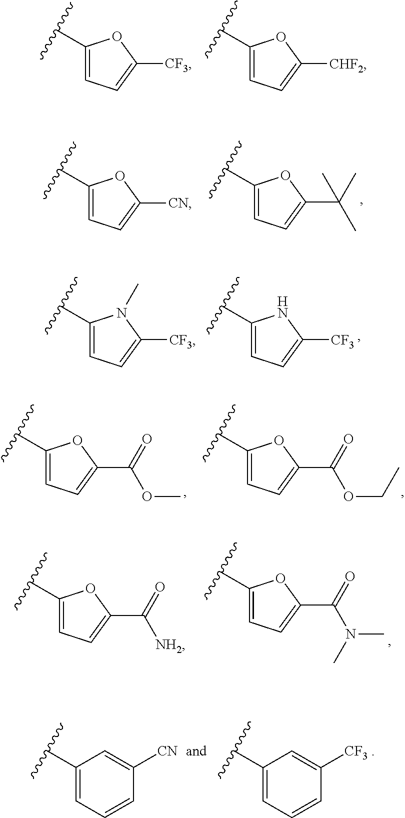

In a most preferred embodiment in combination with any of the above or below embodiments

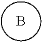

is selected from

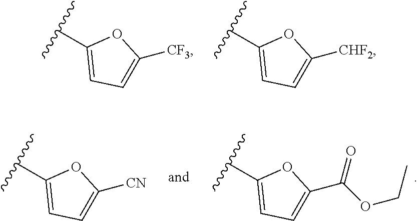 [SMILES: *c1ccc(C(F)(F)F)o1 ; *c1ccc(C(F)F)o1 ; *c1ccc(C#N)o1 ; *c1ccc(C(=O)OCC)o1]

In a further preferred embodiment in combination with any of the above or below embodiments

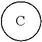

is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S, wherein cycloalkyl, heterocycloalkyl, aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is optionally substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; wherein the residue —CR¹R²— on ring C is linked at least with one 1,4-orientation regarding the connection towards ring D.

Within a first alternative, in a more preferred embodiment in combination with any of the above or below embodiments

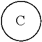

is selected from the group consisting of 6-membered aryl and 5- to 6-membered heteroaryl containing 1 to 4 heteroatoms independently selected from N, O and S, wherein aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein the residue —CR¹R²— on ring C is linked at least with one 1,4-orientation regarding the connection towards ring D.

Within this first alternative, in an even more preferred embodiment in combination with any of the above or below embodiments

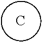

is selected from the group consisting of phenyl, thiophenyl, pyridinyl, pyrimidinyl, pyridazinyl and pyrazinyl, wherein phenyl, thiophenyl, pyridinyl, pyrimidinyl, pyridazinyl and pyrazinyl is unsubstituted or substituted with 1 to 2 substituents independently selected from the group consisting of F, Cl, Br, CN, C₁₋₄-alkyl, fluoro-C₁₋₄-alkyl, OH, oxo, OC₁₋₄-alkyl, O-fluoro-C₁₋₄-alkyl, CONH₂, NH₂, NHC₁₋₄-alkyl and N(C₁₋₄-alkyl)₂; and wherein the residue —CR¹R²— on ring C is linked at least with one 1,4-orientation regarding the connection towards ring D.

Within this first alternative, in an even more preferred embodiment in combination with any of the above or below embodiments

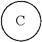

is selected from the group consisting of phenyl, thiophenyl and pyridinyl, wherein phenyl, thiophenyl and pyridinyl is unsubstituted or substituted with 1 to 2 substituents independently selected from the group consisting of F, Cl, Br, CN, C₁₋₄-alkyl, fluoro-C₁₋₄-alkyl, OH, oxo, OC₁₋₄-alkyl, O-fluoro-C₁₋₄-alkyl, CONH₂, NH₂, NHC₁₋₄-alkyl and N(C₁₋₄-alkyl)₂; and wherein the residue —CR¹R²— on ring C is linked at least with one 1,4-orientation regarding the connection towards ring D.

Within this first alternative, in a most preferred embodiment in combination with any of the above or below embodiments

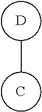

is selected from

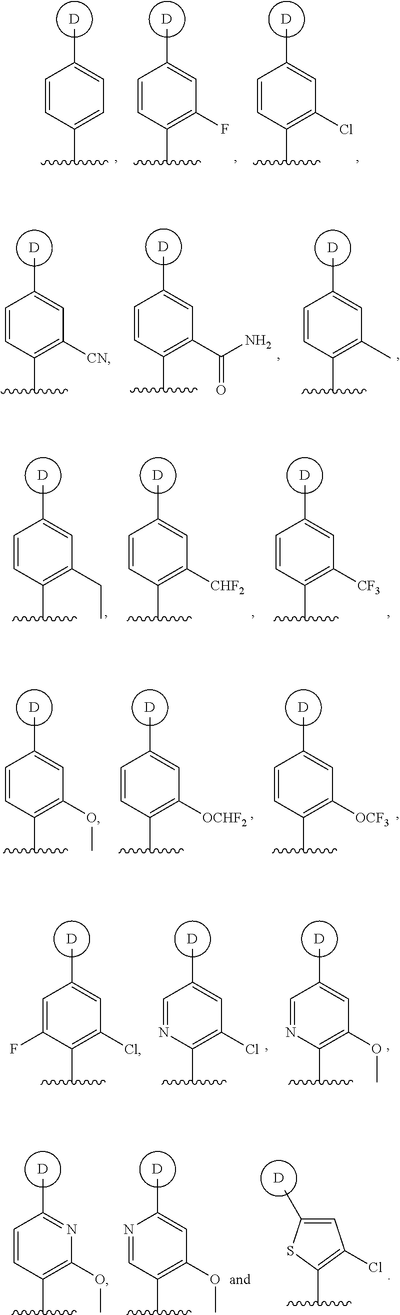

Within a second alternative, in a more preferred embodiment in combination with any of the above or below embodiments

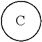

is phenyl, wherein phenyl is unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², CO₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷², wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein the residue —CR¹R²-on ring C is linked in para-orientation regarding the connection towards ring D.

Within this second alternative, in an even more preferred embodiment in combination with any of the above or below embodiments

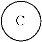

is phenyl, wherein phenyl is unsubstituted or substituted with 1 to 2 substituents independently selected from the group consisting of F, Cl, Br, CN, C₁₋₄-alkyl, fluoro-C₁₋₄-alkyl, OH, OC₁₋₄-alkyl and O-fluoro-C₁₋₄-alkyl; and wherein the residue —CR¹R²— on ring C is linked in para-orientation regarding the connection towards ring D.

Within this second alternative, a most preferred embodiment in combination with any of the above or below embodiments

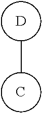

is selected from

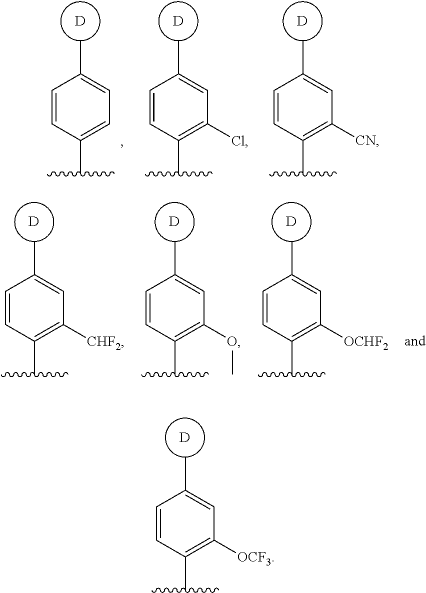

In a further preferred embodiment in combination with any of the above or below embodiments

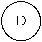

is selected from the group consisting of 6-membered aryl and 5- to 6-membered heteroaryl containing 1 to 4 heteroatoms independently selected from N, O and S, wherein aryl and heteroaryl are unsubstituted or substituted with 1 to 4 substituents independently selected from the group consisting of halogen, CN, NO₂, oxo, C₁₋₄-alkyl, C₀₋₆-alkylene-OR⁸¹, C₀₋₆-alkylene-(3- to 6-membered cycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR¹—CONR⁸¹R⁸², C₀₋₆-alkylene-O—CONR⁸¹R⁸², C₀₋₆alkylene-NR⁸¹—CO₂R⁸¹ and C₀₋₆-alkylene-NR⁸¹R⁸², wherein alkyl, alkylene and cycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein optionally two adjacent substituents on the aryl or heteroaryl moiety form a 5- to 8-membered partially unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; and wherein the residue X—Y—Z on ring D is linked in 1,3-orientation regarding the connection towards ring C.

In a more preferred embodiment in combination with any of the above or below embodiments

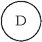

is selected from the group consisting of 6-membered aryl and 5- to 6-membered heteroaryl containing 1 to 4 heteroatoms independently selected from N, O and S, wherein aryl and heteroaryl are unsubstituted or substituted with 1 to 4 substituents independently selected from the group consisting of halogen, CN, NO₂, oxo, C₁₋₄-alkyl, C₀₋₆-alkylene-OR⁸¹, C₀₋₆-alkylene-(3- to 6-membered cycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹ and C₀₋₆-alkylene-NR⁸¹R⁸², wherein alkyl, alkylene and cycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and wherein the residue X—Y—Z on ring D is linked in 1,3-orientation regarding the connection towards ring C.

In an even more preferred embodiment in combination with any of the above or below embodiments

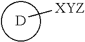

is selected from

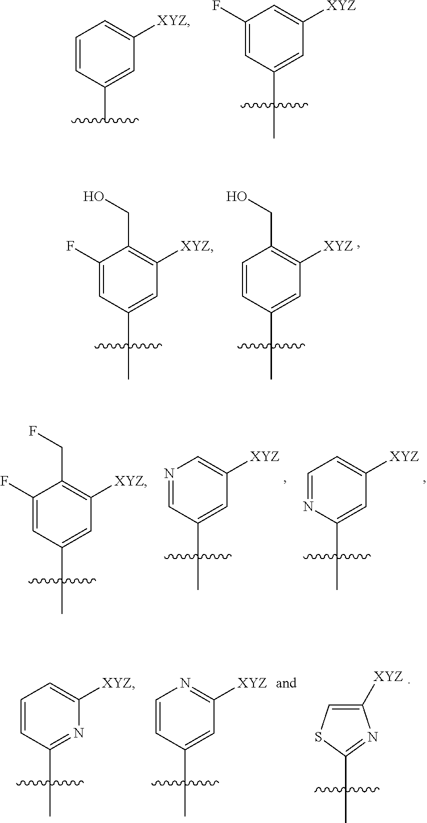

In a most preferred embodiment in combination with any of the above or below embodiments

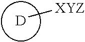

is selected from

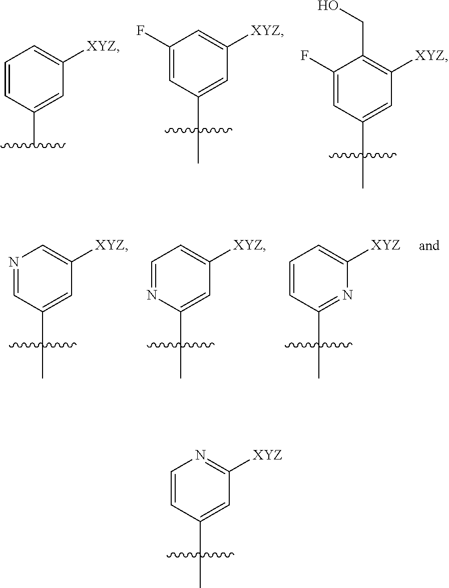

and in an even most preferred embodiment in combination with any of the above or below embodiments

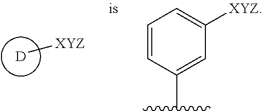

In a further preferred embodiment in combination with any of the above or below embodiments X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹—; wherein
R¹¹ is selected from H, CN, NO₂, C₁₋₄-alkyl, C(═O)—C₁₋₄-alkyl, C(═O)—O—C₁₋₄-alkyl, halo-C₁₋₄-alkyl, C(═O)-halo-C₁₋₄-alkyl and C(═O)—O-halo-C₁₋₄-alkyl; and
R⁹¹ is independently selected from H and C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl; and n is selected from 0 to 2.

In a more preferred embodiment in combination with any of the above or below embodiments X is selected from a bond, —S(═O)₂— and —O—.

In a most preferred embodiment in combination with any of the above or below embodiments X is a bond.

In a further preferred embodiment in combination with any of the above or below embodiments Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S, wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂.

In a more preferred embodiment in combination with any of the above or below embodiments Y is selected from C₁₋₃-alkylene, 3- to 6-membered cycloalkylene or 3- to 6-membered heterocycloalkylene containing 1 heteroatom selected from N, O and S, wherein alkylene, cycloalkylene or heterocycloalkylene is unsubstituted or substituted with 1 to 6 substituents independently selected from halogen, CN, C₁₋₄-alkyl, halo-C₁₋₄-alkyl, OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂.

In an even more preferred embodiment in combination with any of the above or below embodiments Y is selected from

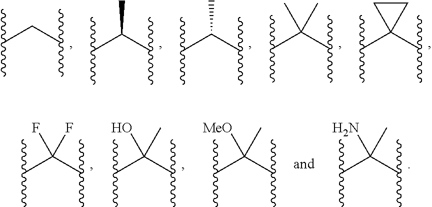

In a most preferred embodiment in combination with any of the above or below embodiments Y is selected from

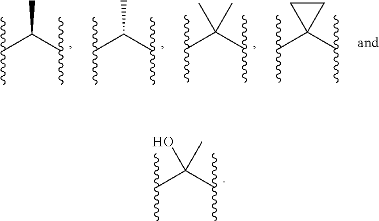

In a further preferred embodiment in combination with any of the above or below embodiments Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, —P(═O)H(OH), —B(OH)₂,

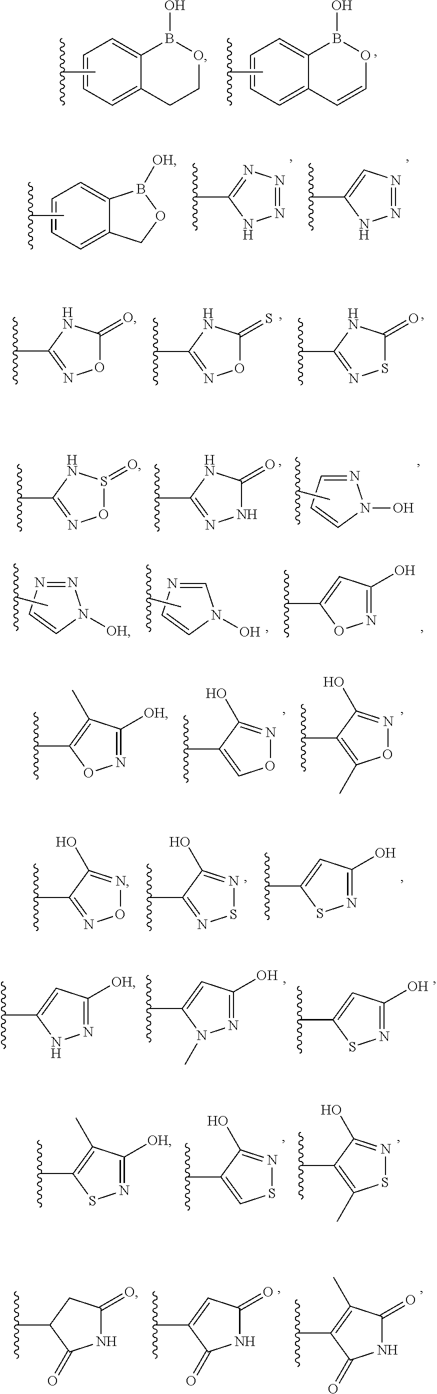

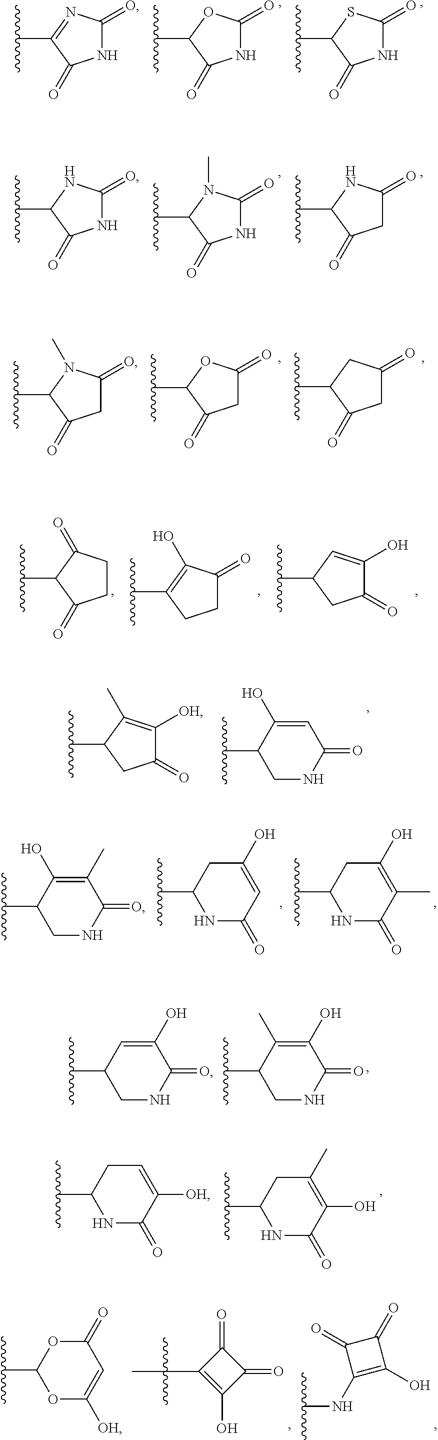

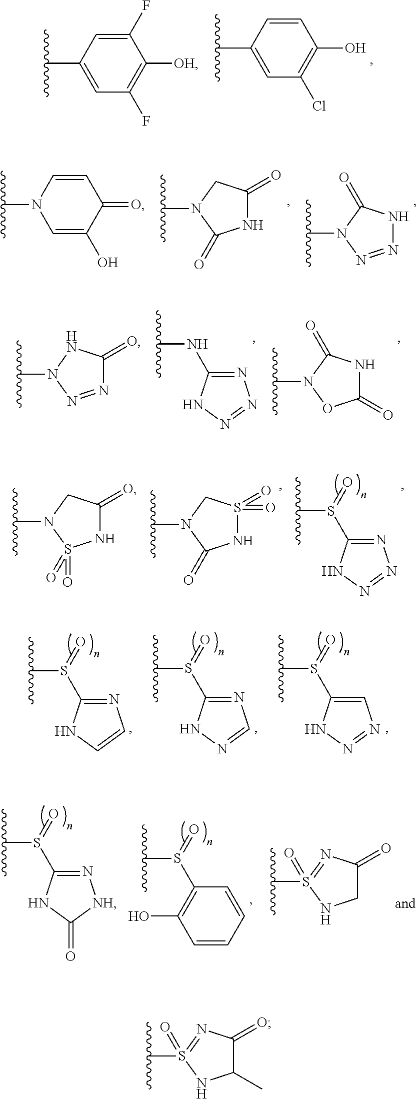

wherein
R⁹⁰ is independently selected from C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
R⁹¹, R⁹² are independently selected from H and C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl; or R⁹¹ and R⁹² when taken together with the nitrogen to which they are attached complete a 3- to 6-membered ring containing carbon atoms and optionally containing 1 or 2 heteroatoms selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl; and n is selected from 0 to 2; or a prodrug and pharmaceutically acceptable salt thereof.

In a more preferred embodiment in combination with any of the above or below embodiments Z is selected from —CO₂H, —CONHO—C₁₋₄-alkyl, —CON(C₁₋₄-alkyl)OH, —CONHOH, —CONHSO₂—C₁₋₄-alkyl, —CONHSO₂—N(C₁₋₄-alkyl)₂,

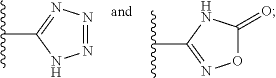 [SMILES: *c1nnn[nH]1 ; *c1noc(=O)[nH]1]

or a prodrug and pharmaceutically acceptable salt thereof.

In an even more preferred embodiment in combination with any of the above or below embodiments Z is —CO₂H; or a prodrug and pharmaceutically acceptable salt thereof.

In a most preferred embodiment in combination with any of the above or below embodiments Z is —CO₂H.

In a further preferred embodiment in combination with any of the above or below embodiments

X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹—;
Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S, wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂;
Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, —P(═O)H(OH), —B(OH)₂,

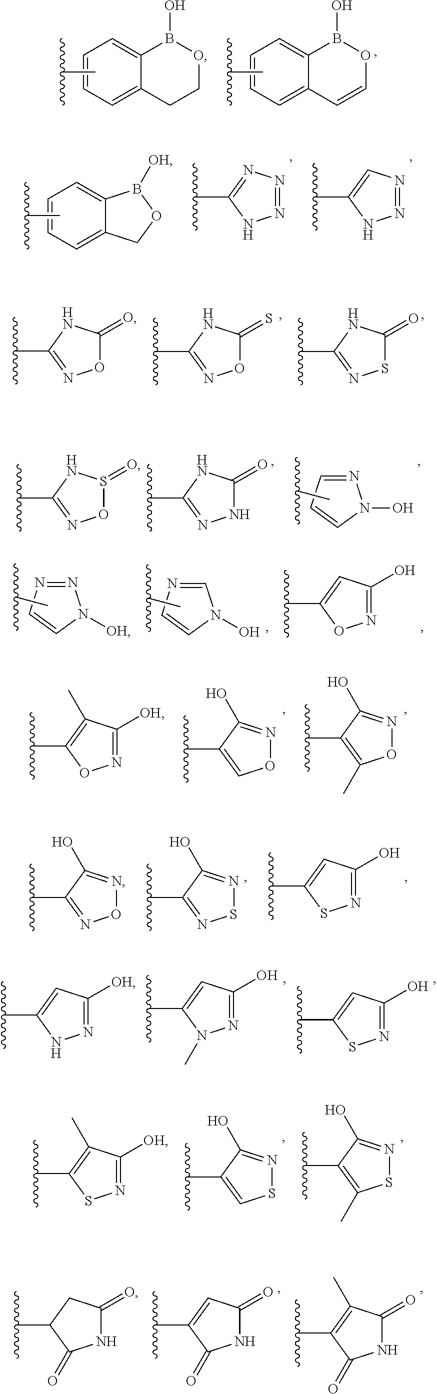

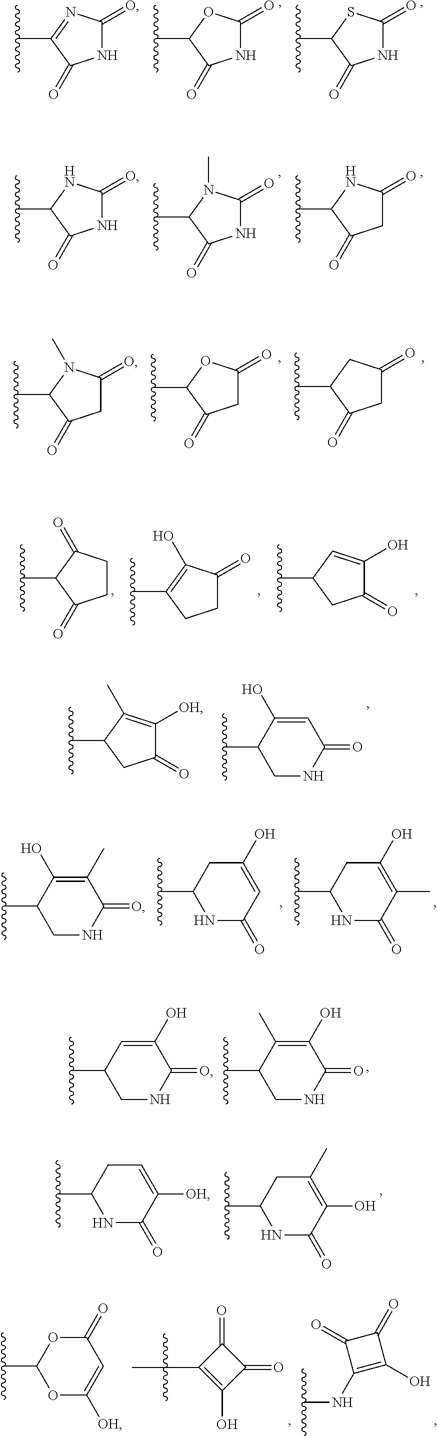

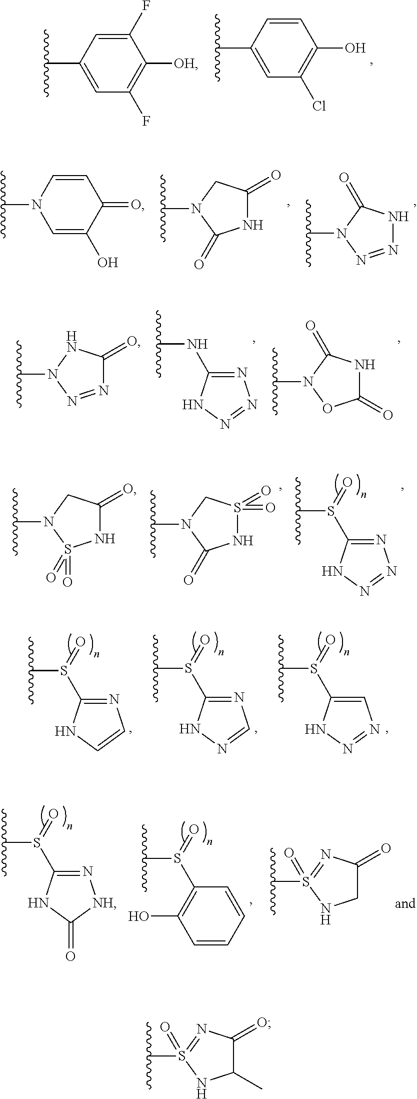

R¹¹ is selected from H, CN, NO₂, C₁₋₄-alkyl, C(═O)—C₁₋₄-alkyl, C(═H)—O—C₁₋₄-alkyl, halo-C₁₋₄-alkyl, C(═O)-halo-C₁₋₄-alkyl and C(═O)—O-halo-C₁₋₄-alkyl;
R⁹⁰ is independently selected from C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
R⁹¹, R⁹² are independently selected from H and C₁₋₄-alkyl, wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl; or R⁹¹ and R⁹² when taken together with the nitrogen to which they are attached complete a 3- to 6-membered ring containing carbon atoms and optionally containing 1 or 2 heteroatoms selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl; and n is selected from 0 to 2; or a prodrug and pharmaceutically acceptable salt thereof.

In a more preferred embodiment in combination with any of the above or below embodiments X is selected from a bond, C₀₋₆-alkylene-S(═O)ₙ, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-(═O)₂NR⁹¹—C₀₋₆-alkylene-S(═NR¹¹)(═O)—NR⁹¹— and C₀₋₆-alkylene-S(═NR¹¹)—NR⁹¹—;

Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S; wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂;
Z is selected from —CO₂H, —CONHO—C₁₋₄-alkyl, —CON(C₁₋₄-alkyl)OH, —CONHOH, —CONHSO₂—C₁₋₄-alkyl, —CONHSO₂—N(C₁₋₄-alkyl)₂,

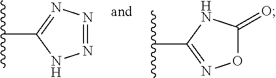 [SMILES: *c1nnn[nH]1 ; *c1noc(=O)[nH]1]

or a prodrug and pharmaceutically acceptable salt thereof.

In a more preferred embodiment in combination with any of the above or below embodiments

X is selected from a bond, O and S(═O)₂;
Y is selected from C₁₋₃-alkylene, 3- to 6-membered cycloalkylene and 3- to 6-membered heterocycloalkylene containing 1 to 4 heteroatoms independently selected from N, O and S, wherein alkylene, cycloalkylene or heterocycloalkylene is unsubstituted or substituted with 1 to 2 substituents independently selected from fluoro, CN, C₁₋₄-alkyl, halo-C₁₋₄-alkyl, OH, NH₂, oxo, O—C₁₋₄-alkyl and O-halo-C₁₋₄-alkyl; and
Z is selected from —CO₂H, —CONHO—C₁₋₄-alkyl, —CON(C₁₋₄-alkyl)OH, —CONHOH, —CONHSO₂—C₁₋₄-alkyl, —CONHSO₂—N(C₁₋₄-alkyl)₂,

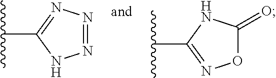 [SMILES: *c1nnn[nH]1 ; *c1noc(=O)[nH]1]

or a prodrug and pharmaceutically acceptable salt thereof.

In an even more preferred embodiment in combination with any of the above or below embodiments XYZ is selected from

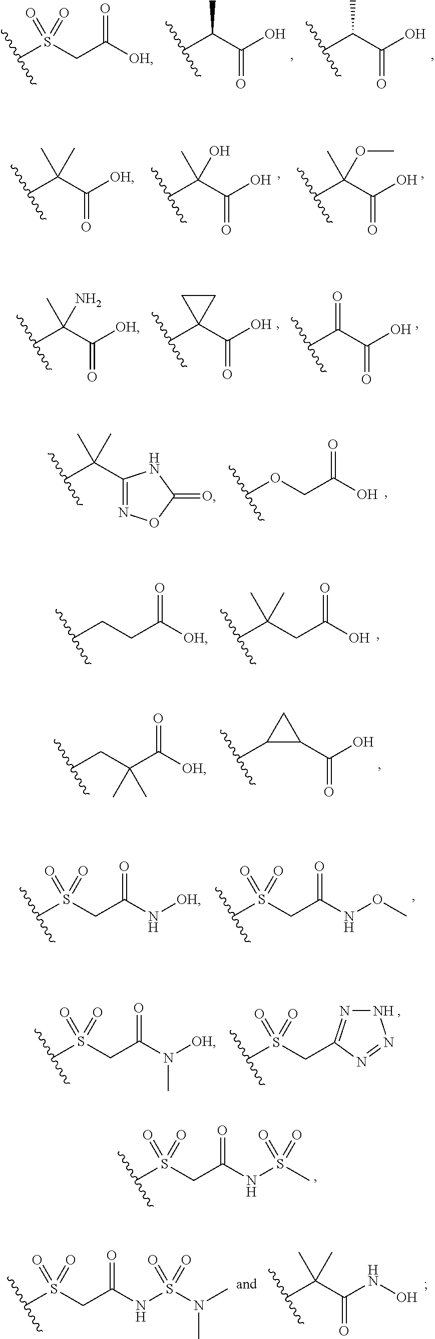

or a prodrug and pharmaceutically acceptable salt thereof.

In a most preferred embodiment in combination with any of the above or below embodiments XYZ is selected from

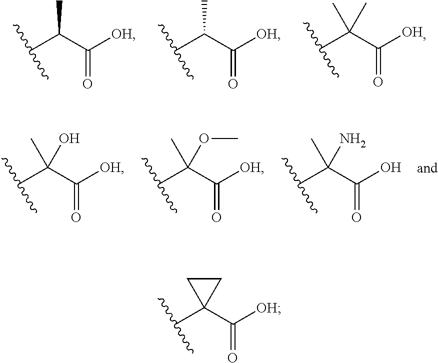

or a prodrug and pharmaceutically acceptable salt thereof.

In an even most preferred embodiment in combination with any of the above or below embodiments XYZ is selected from

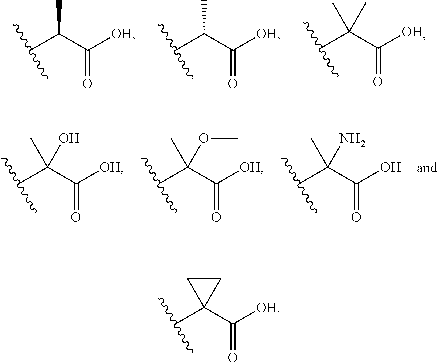

In a further preferred embodiment in combination with any of the above or below embodiments

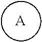

is selected from

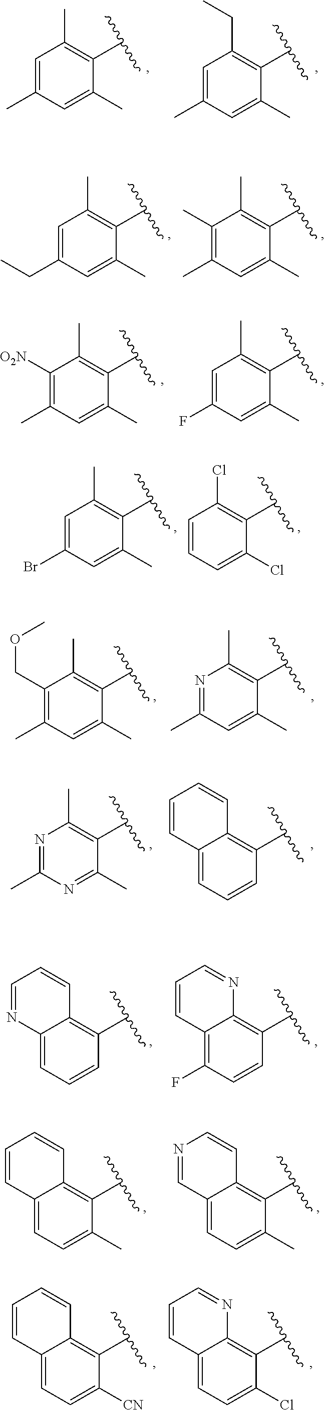

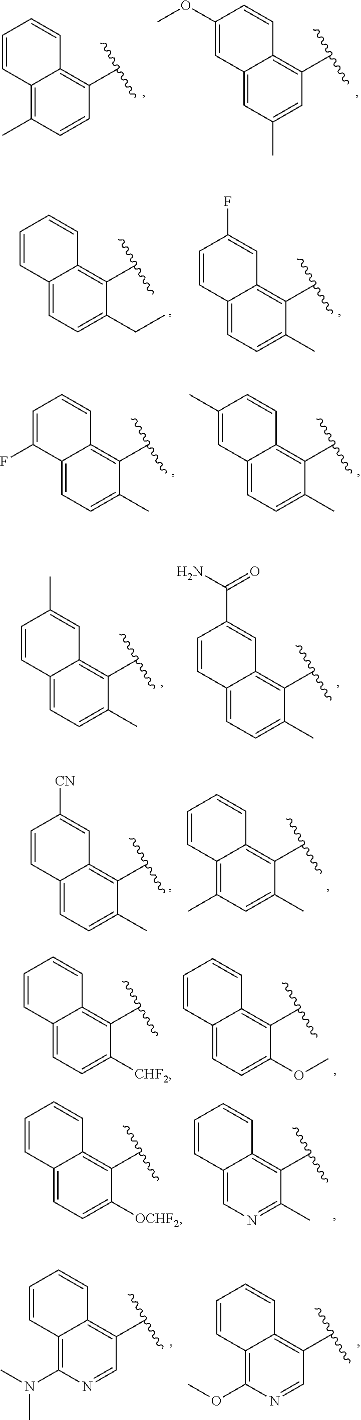

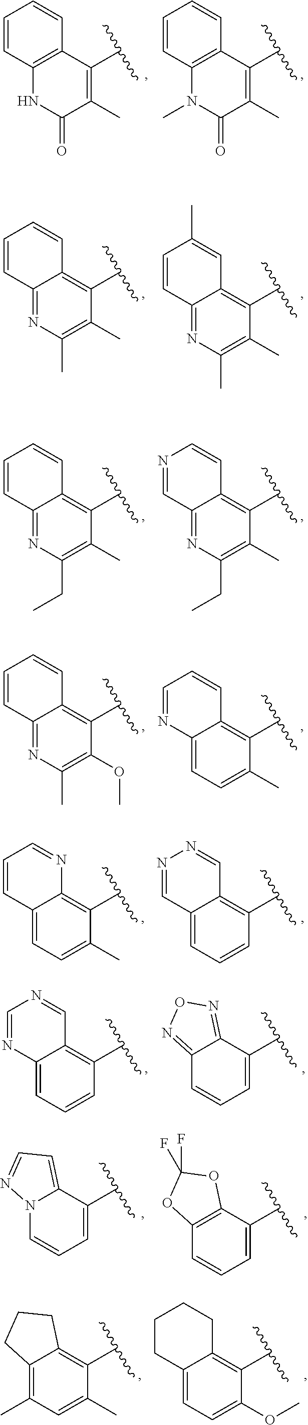

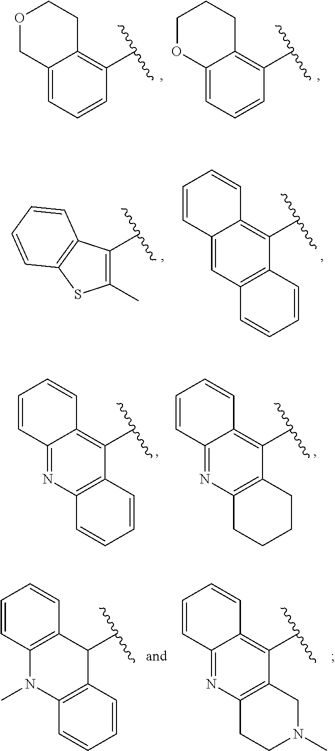

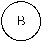

is selected from

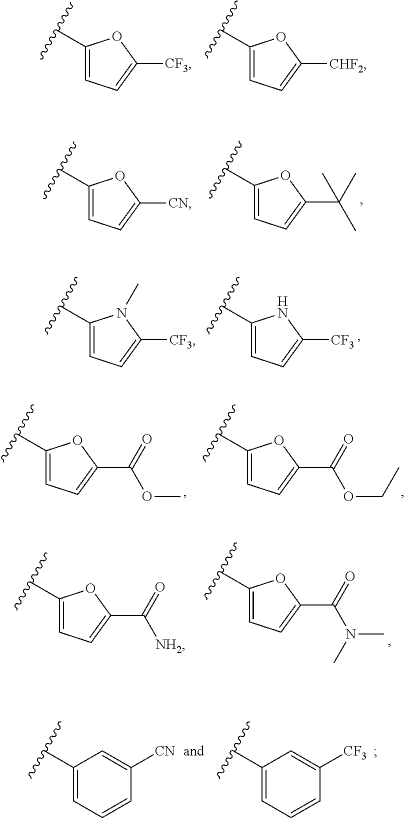

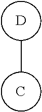

is selected from

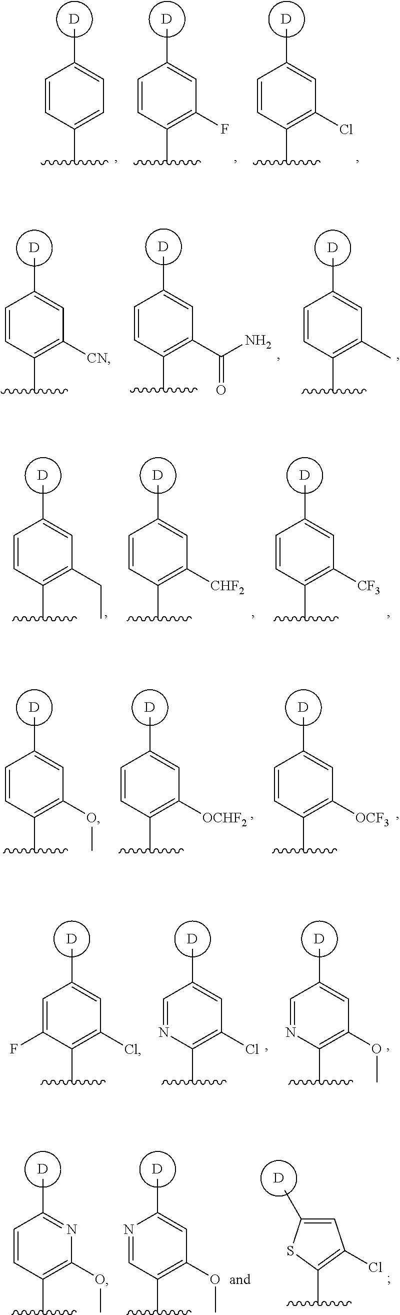

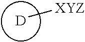

is selected from

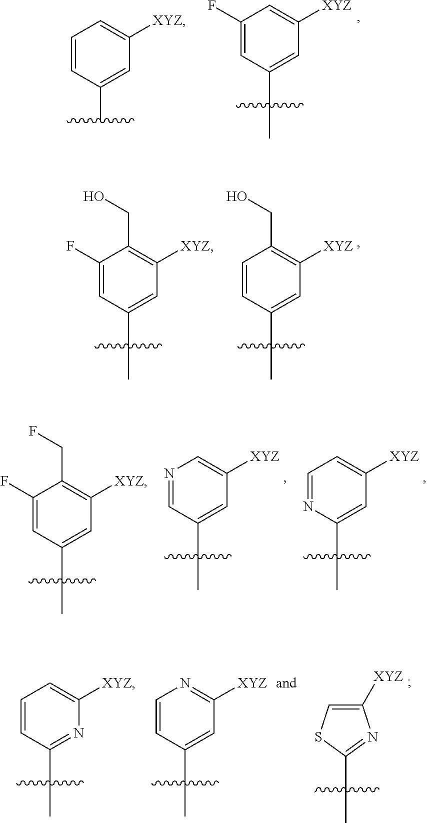

XYZ is selected from

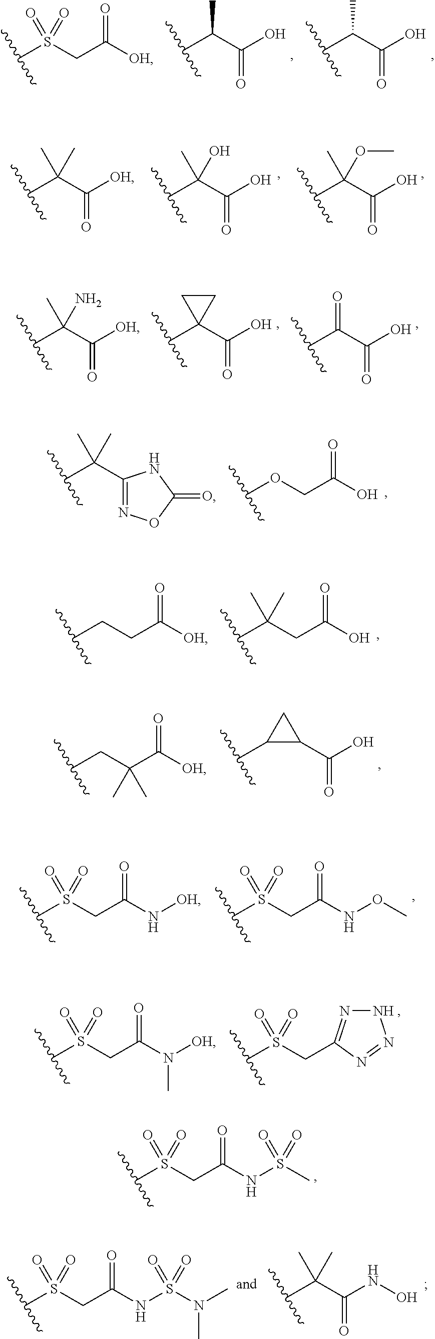

R¹, R², R³ and R⁴ are independently selected from H and Me; R⁵ and R⁶ are independently selected from H and Me or R⁵ and R⁶ together are oxo; m and p is 1.

In a more preferred embodiment in combination with any of the above or below embodiments

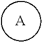

is selected from

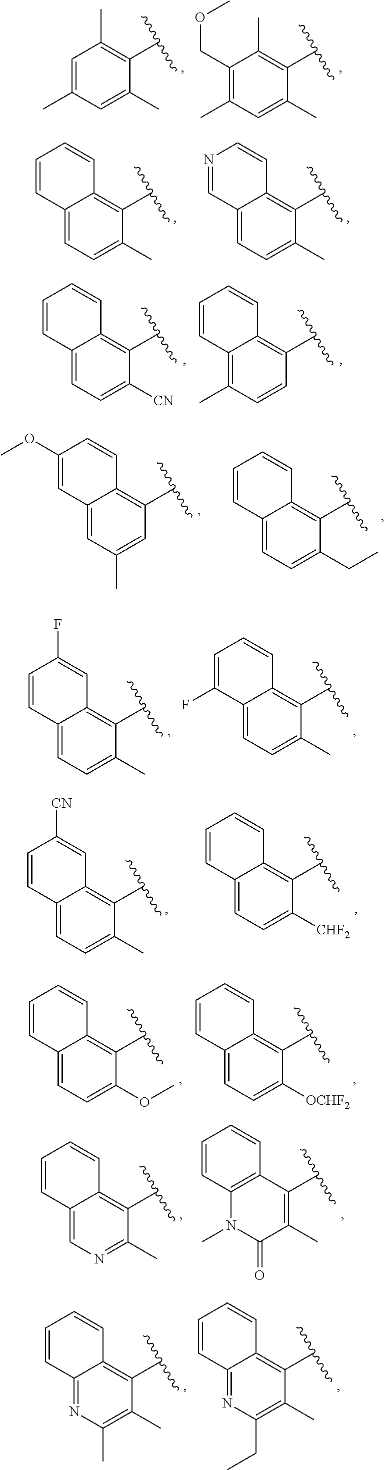

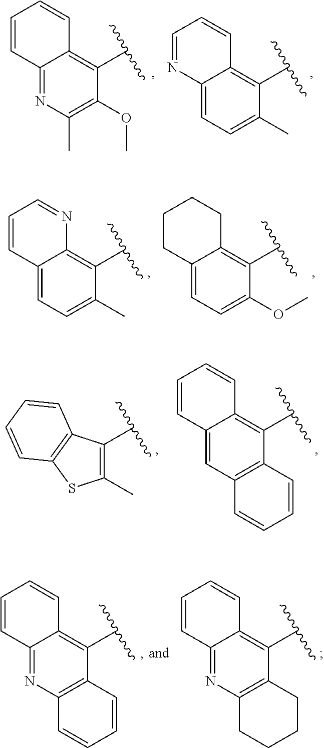

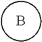

is selected from

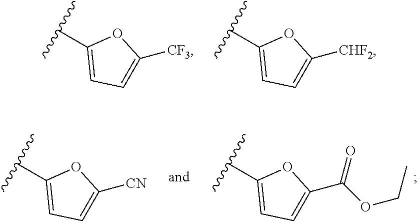 [SMILES: *c1ccc(C(F)(F)F)o1 ; *c1ccc(C(F)F)o1 ; *c1ccc(C#N)o1 ; *c1ccc(C(=O)OCC)o1]

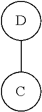

is selected from

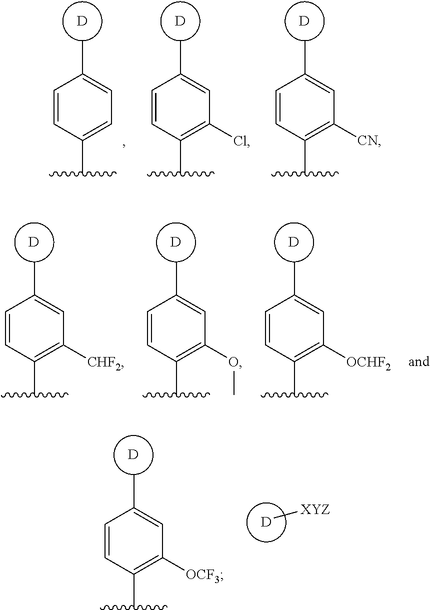

is selected from

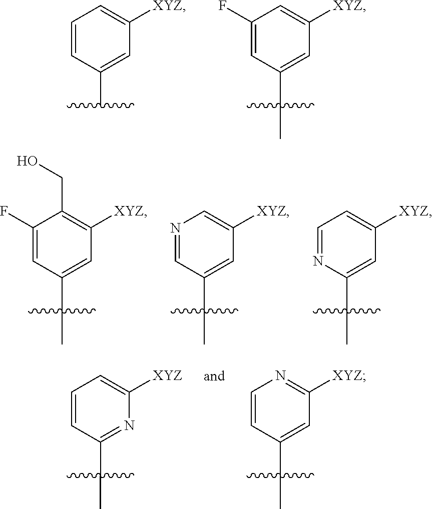

XYZ is selected from

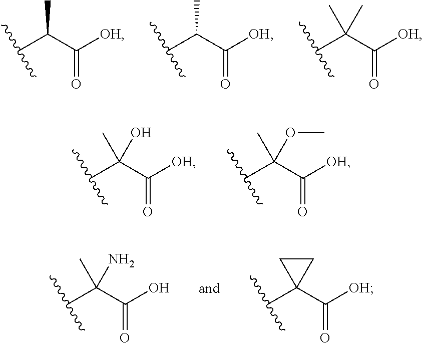

R¹, R², R³ and R⁴ are H; R⁵ and R⁶ are independently H or R⁵ and R⁶ together are oxo; m and p is 1.

In an additional preferred embodiment in combination with any of the above or below embodiments

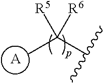

is selected from

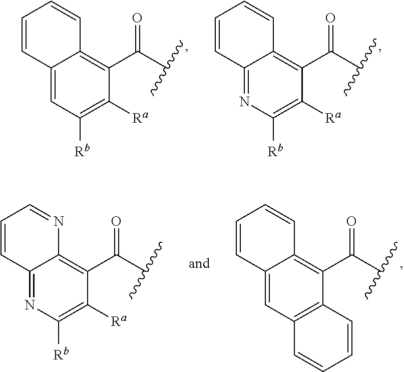

wherein R^{a} and R^{b} is independently selected from H, Cl, CN, Me, Et, cyclopropyl, CHF₂, CF₃, OH, OMe, OCHF₂ and OCF₃; and \textcircled{A} may be further substituted with 1 to 3 additional substituents independently selected from F, Cl, Br, CN, OH, Me, Et, CHF₂, CF₃, OMe, OEt, OCHF₂ and OCF₃;

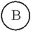

is selected from

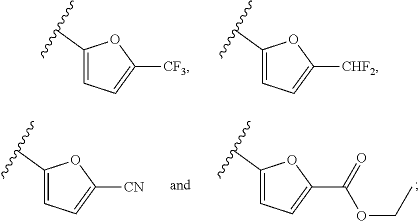 [SMILES: *c1ccc(C(F)(F)F)o1 ; *c1ccc(C(F)F)o1 ; *c1ccc(C#N)o1 ; *c1ccc(C(=O)OCC)o1]

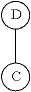

is selected from

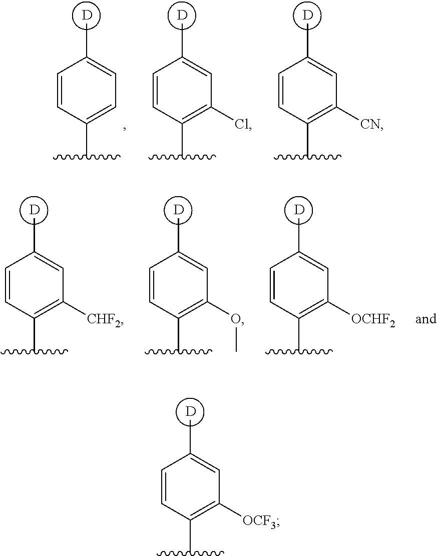

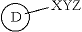

is selected from

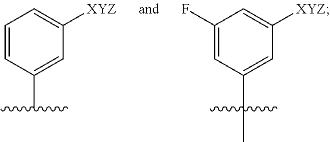

XYZ is selected from

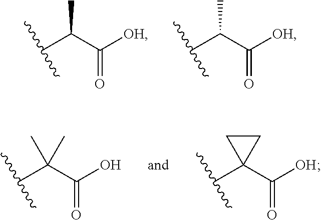

R¹, R², R³ and R⁴ are H; m is 1.

In an additional more preferred embodiment in combination with any of the above or below embodiments

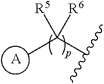

is selected from

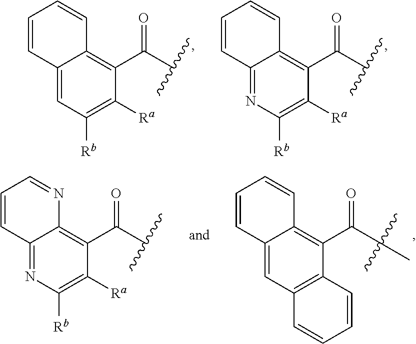

wherein R^{a} is H, and R^{b} is selected from H, Cl, CN, Me, Et, cyclopropyl, CHF₂, CF₃, OMe, OCHF₂ and OCF₃; and

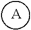

may be further substituted with 1 to 3 additional substituents independently selected from F, Cl, Br, CN, OH, Me, Et, CHF₂, CF₃, OMe, OEt, OCHF₂ and OCF₃;

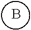

is selected from

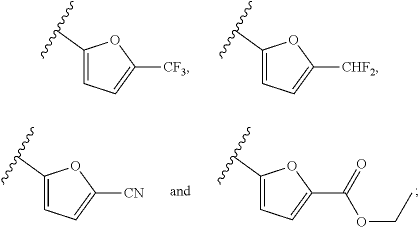 [SMILES: *c1ccc(C(F)(F)F)o1 ; *c1ccc(C(F)F)o1 ; *c1ccc(C#N)o1 ; *c1ccc(C(=O)OCC)o1]

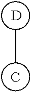

is selected from and

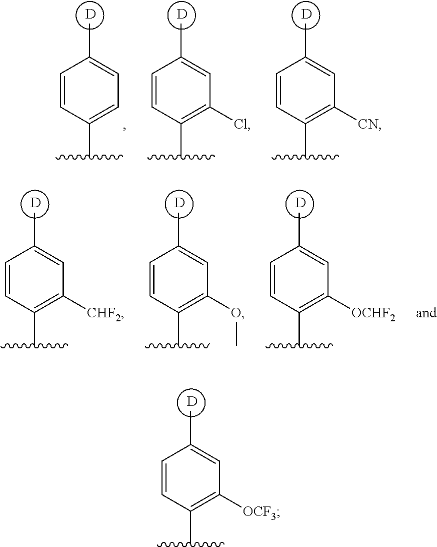

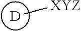

is selected from

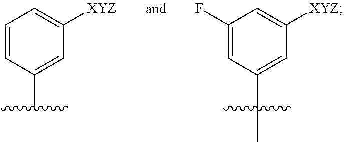

XYZ is selected from

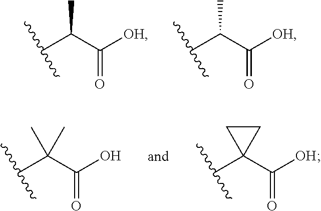

R¹, R², R³ and R⁴ are H; m is 1.

In an additional most preferred embodiment in combination with any of the above or below embodiments

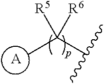

is selected from

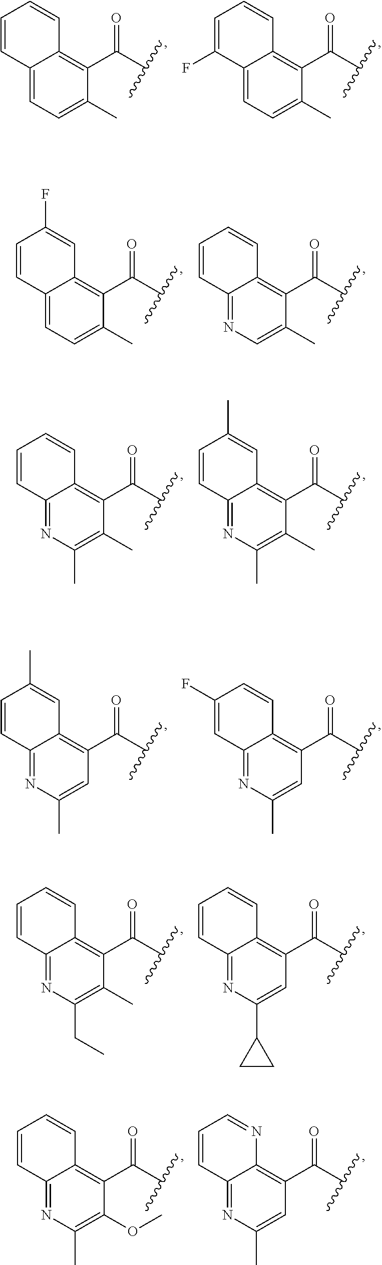

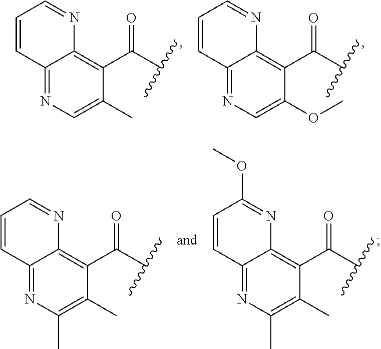

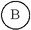

is selected from

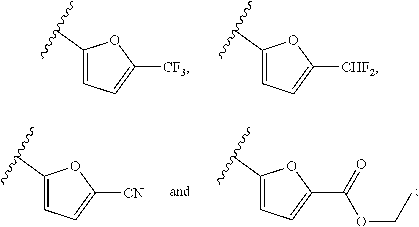 [SMILES: *c1ccc(C(F)(F)F)o1 ; *c1ccc(C(F)F)o1 ; *c1ccc(C#N)o1 ; *c1ccc(C(=O)OCC)o1]

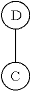

is selected from

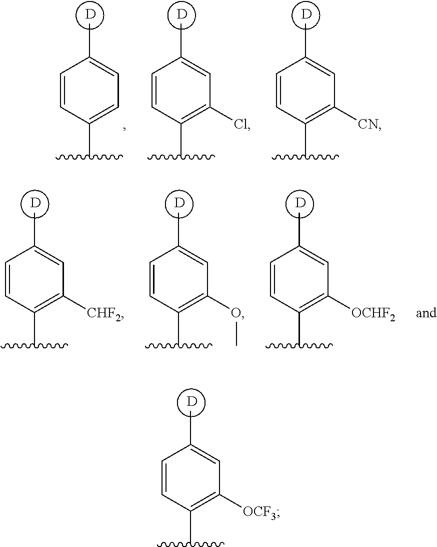

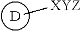

is selected from

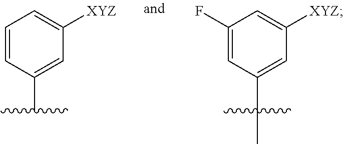

XYZ is selected from

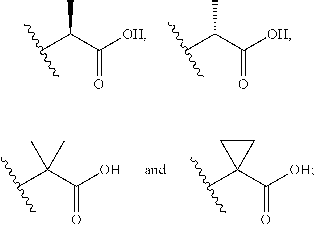

R¹, R², R³ and R⁴ are H; m is 1.

In a most preferred embodiment, the compound is selected from

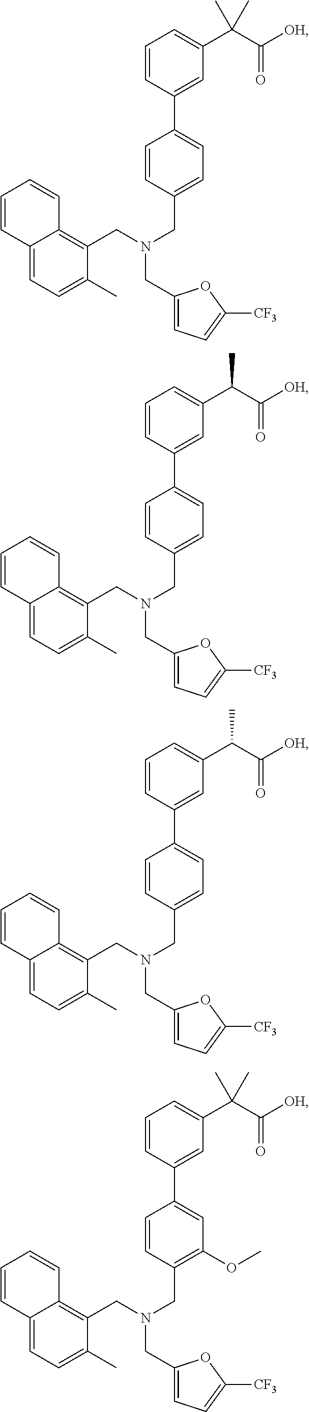

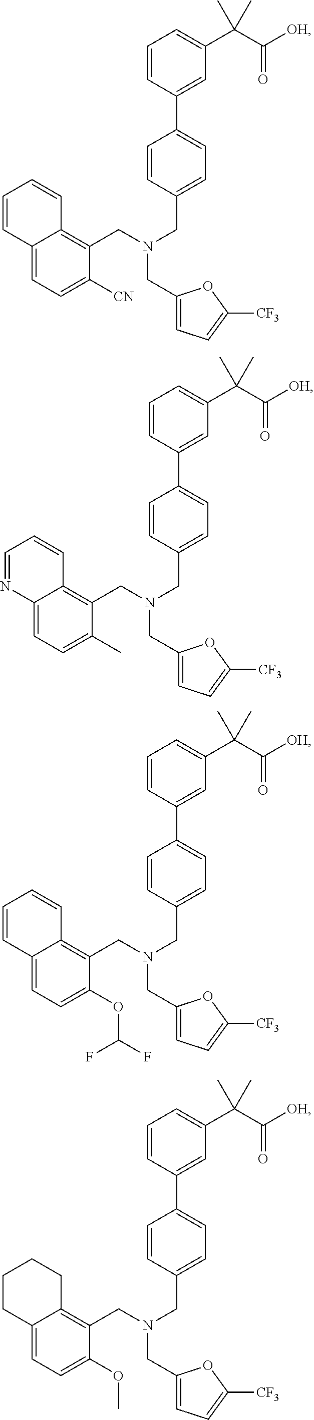

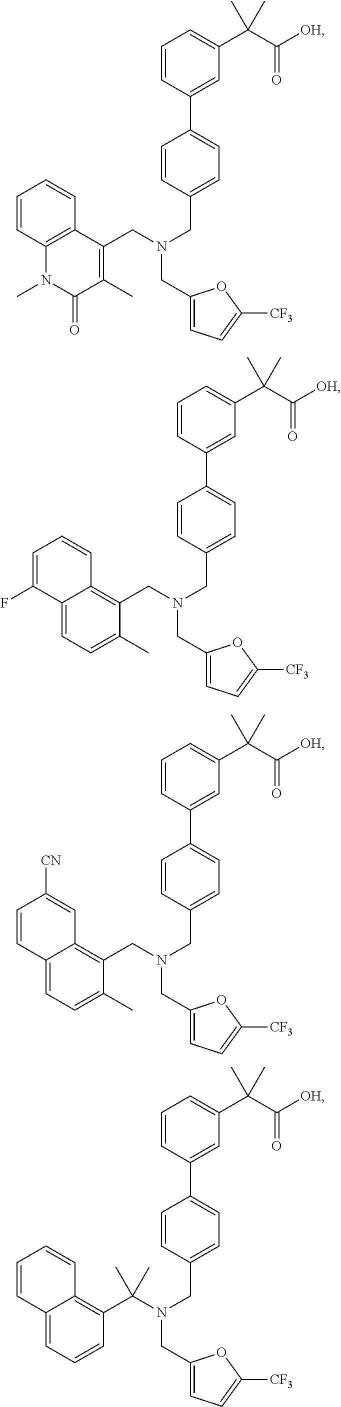

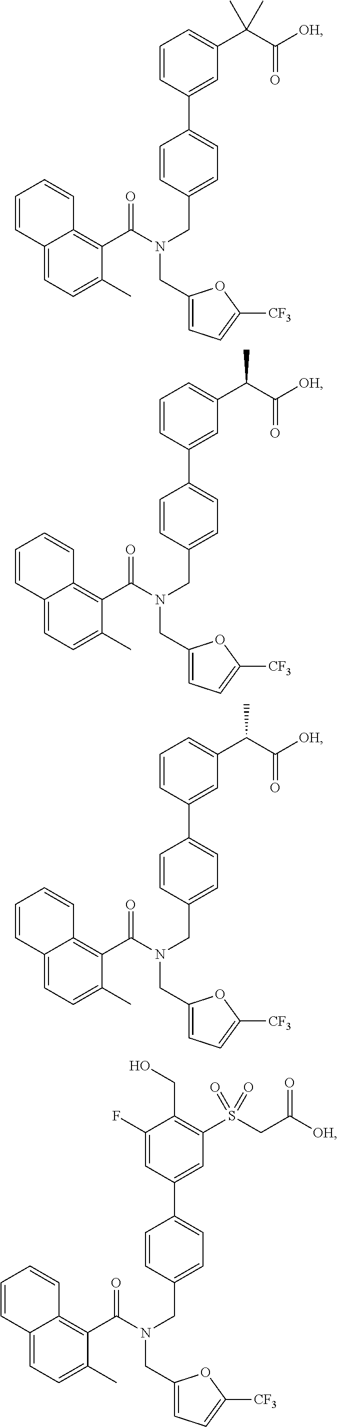

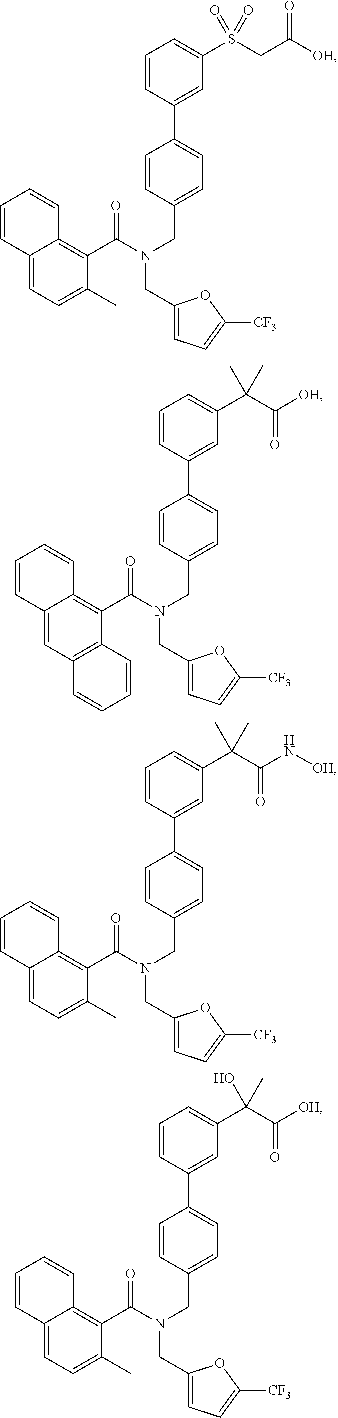

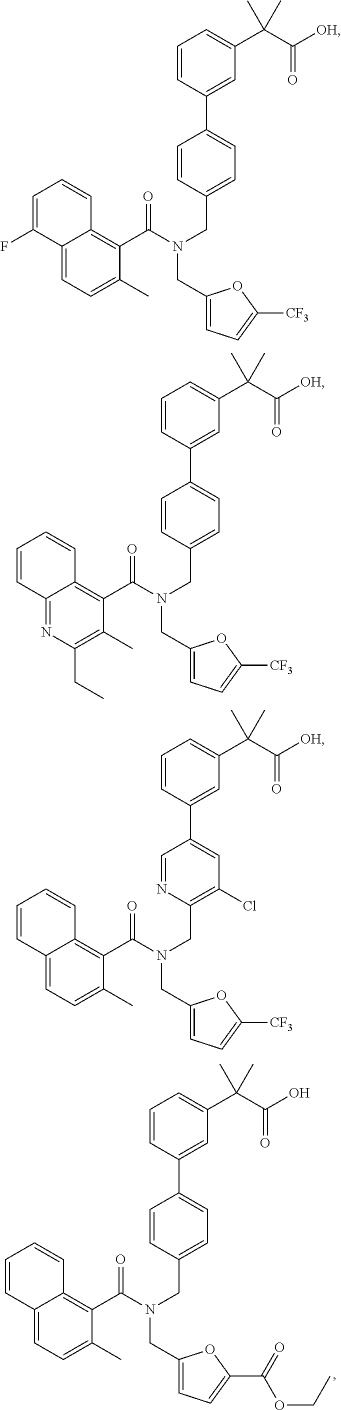

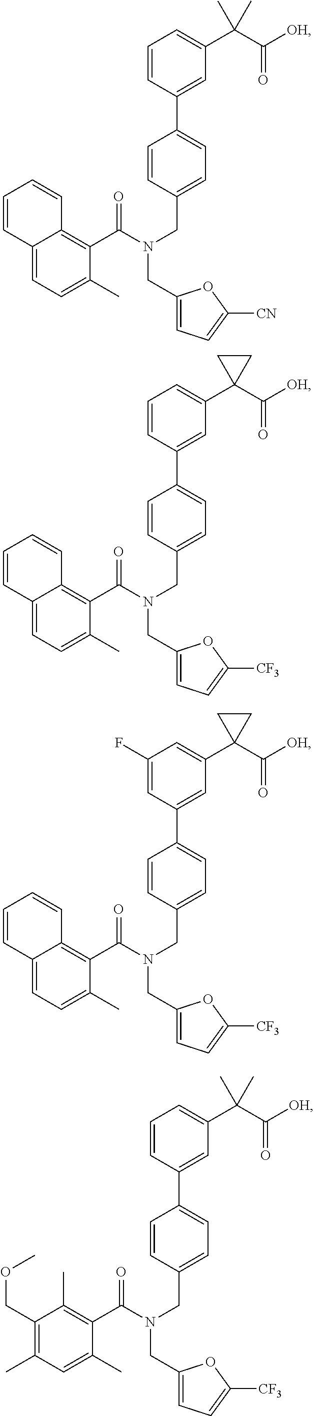

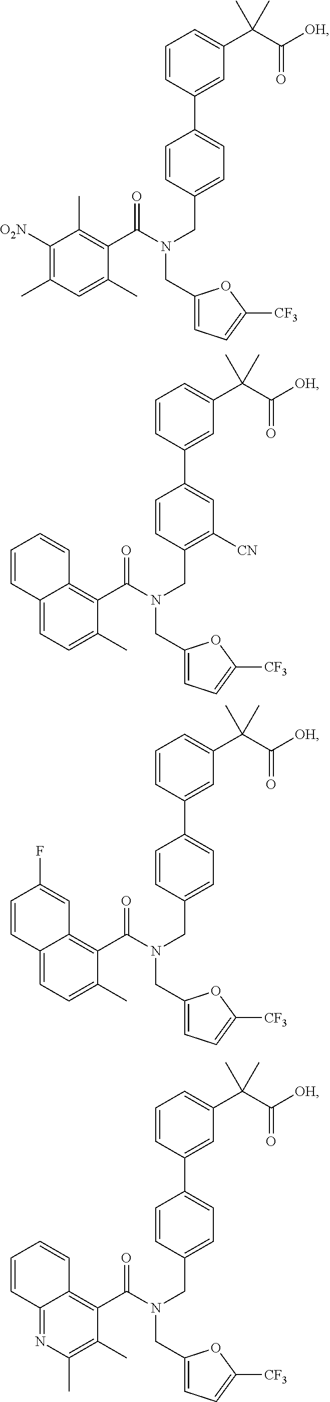

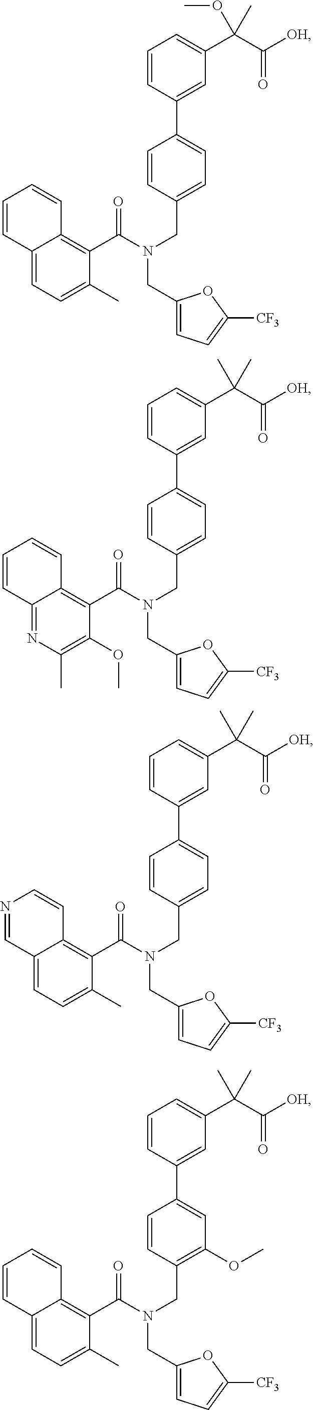

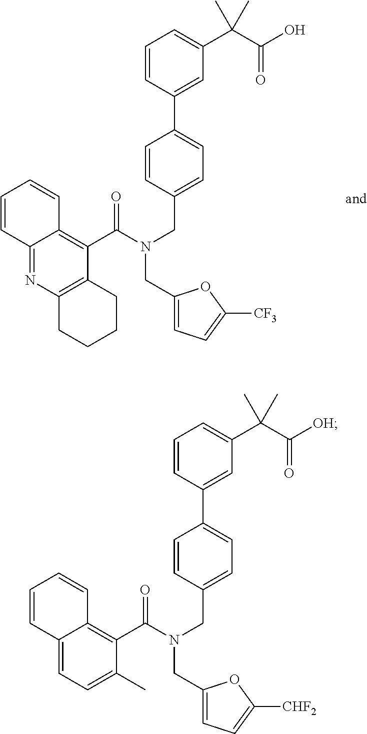 [SMILES: CC(C)(C(=O)O)c1cccc(-c2ccc(CN(Cc3ccc(C(F)(F)F)o3)C(=O)c3c4c(nc5ccccc35)CCCC4)cc2)c1 ; Cc1ccc2ccccc2c1C(=O)N(Cc1ccc(-c2cccc(C(C)(C)C(=O)O)c2)cc1)Cc1ccc(C(F)F)o1]

an enantiomer, diastereomer, tautomer, N-oxide, solvate, prodrug and pharmaceutically acceptable salt thereof.

In a similar most preferred embodiment the compound is selected from

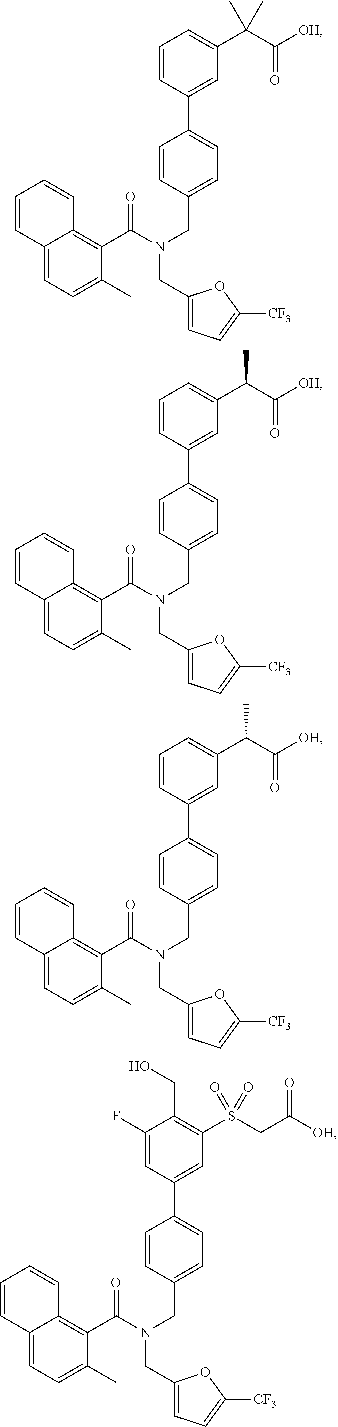

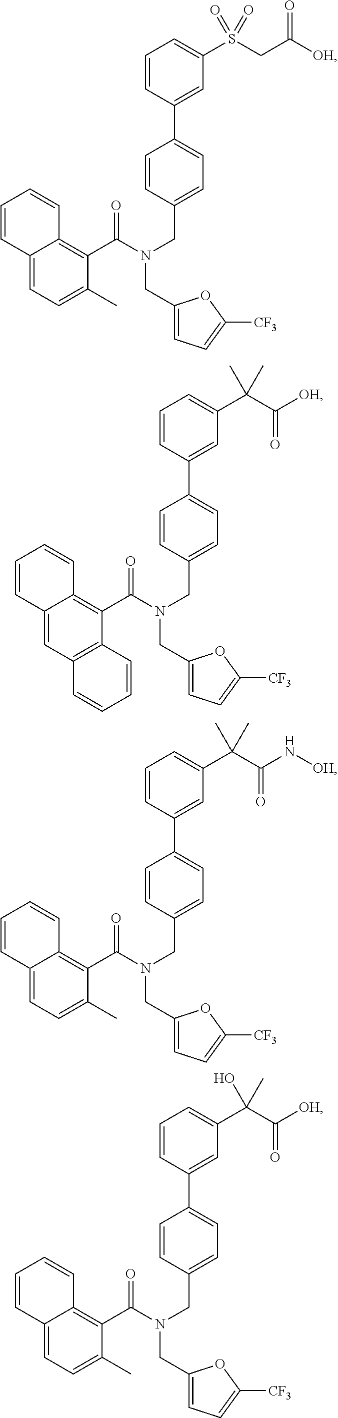

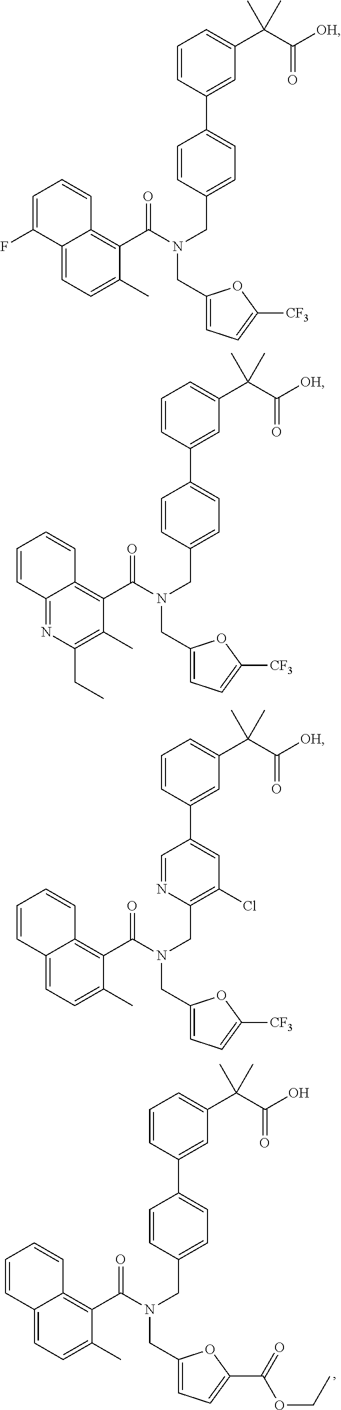

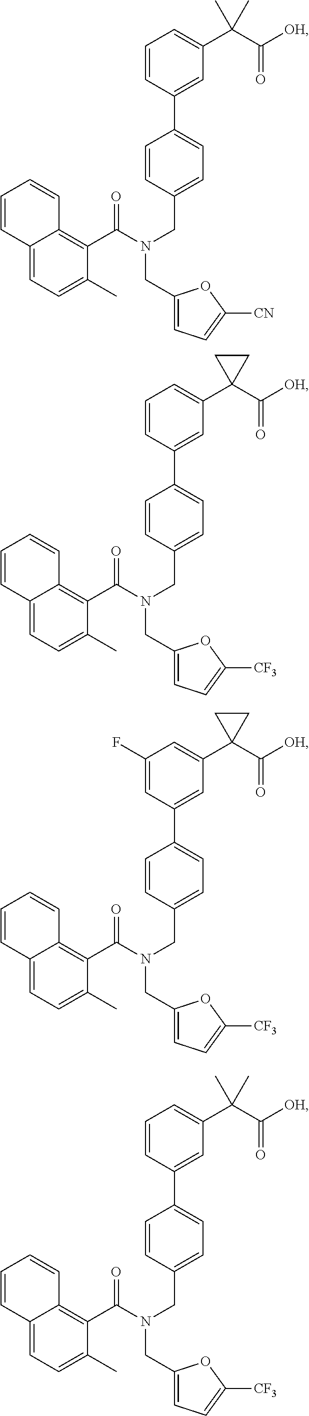

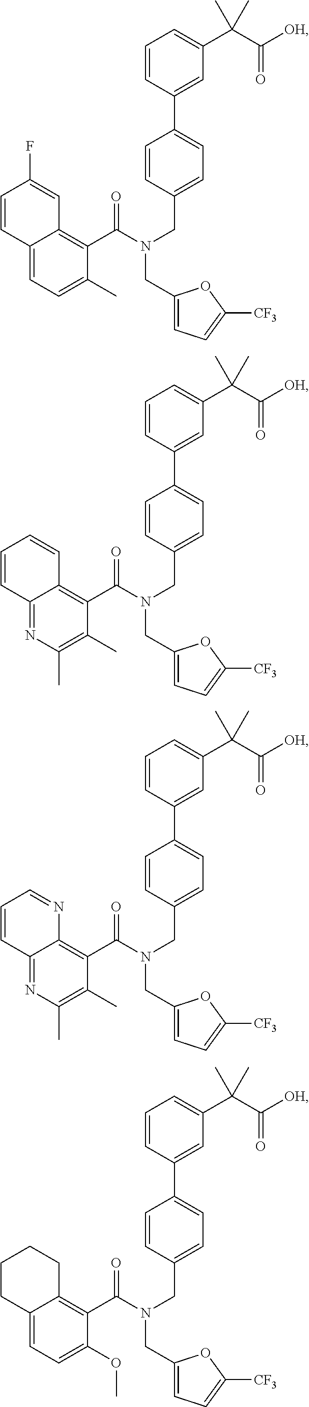

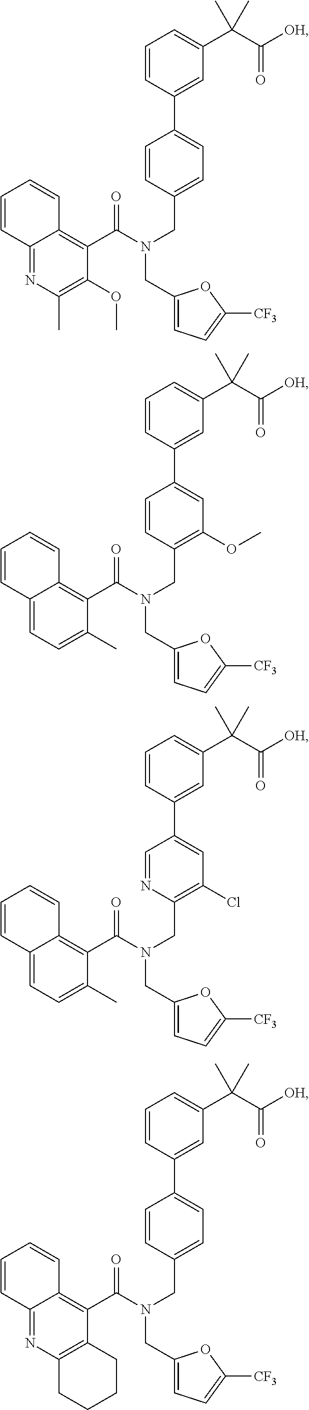

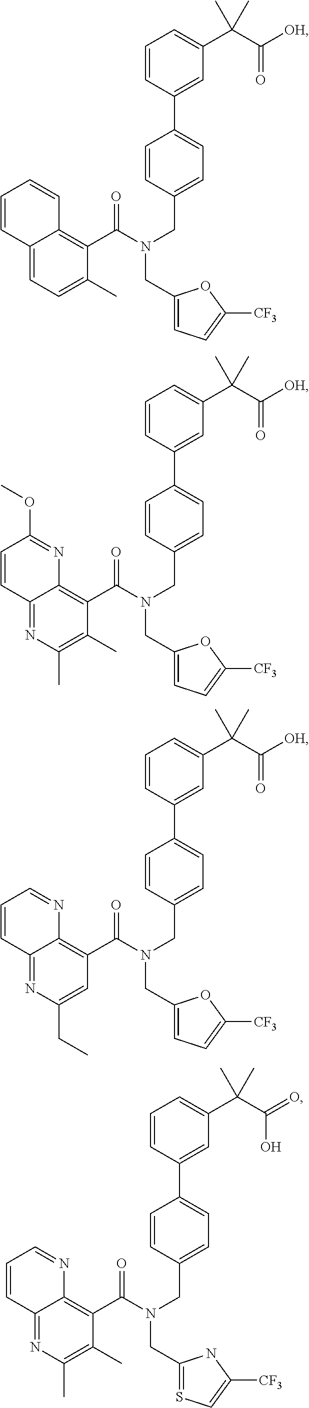

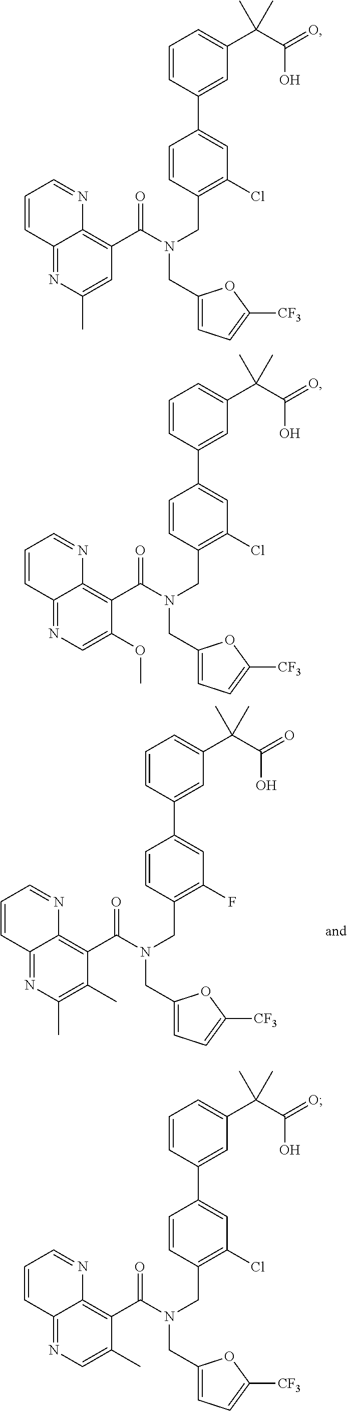

an enantiomer, diastereomer, tautomer, N-oxide, solvate, prodrug and pharmaceutically acceptable salt thereof.

Finally, in an upmost preferred embodiment, the compound is selected from

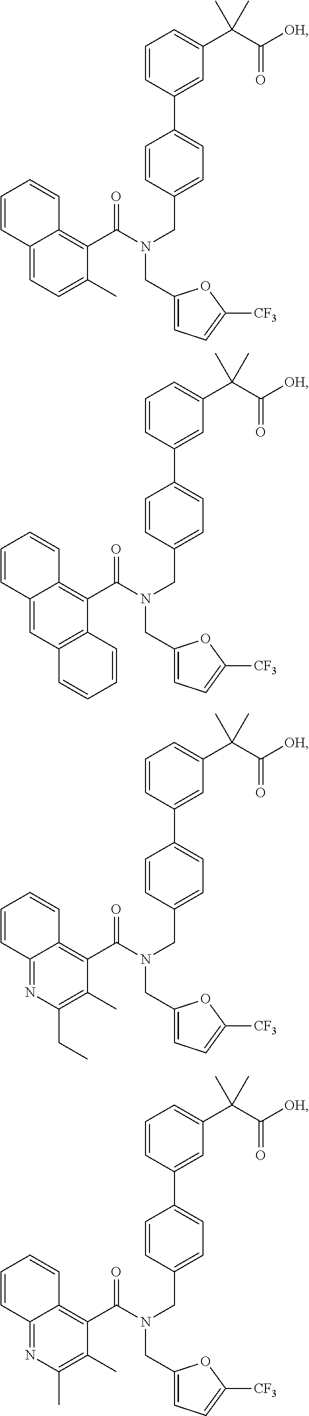

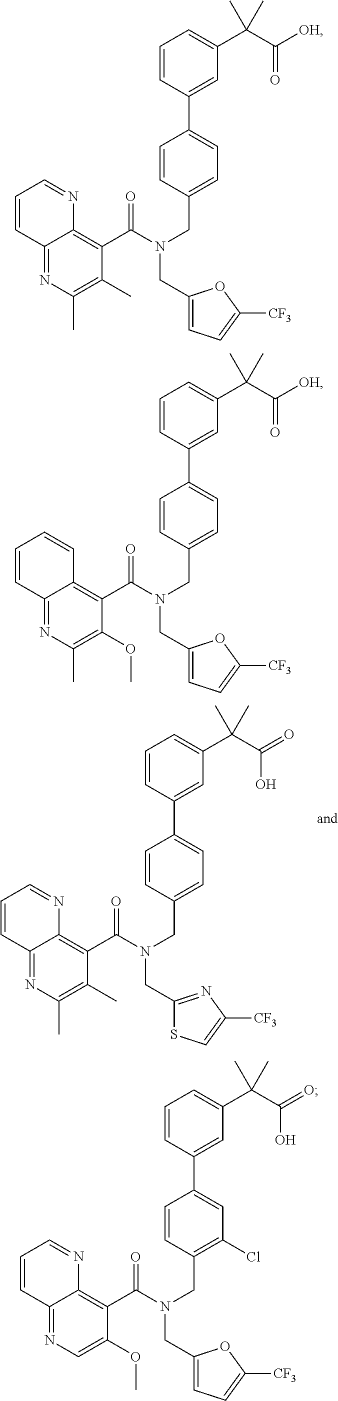

an enantiomer, diastereomer, tautomer, N-oxide, solvate, prodrug and pharmaceutically acceptable salt thereof.

The invention also provides the compound of the invention for use as a medicament.

Also provided is the compound of the present invention for use in the prophylaxis and/or treatment of diseases mediated by LXRs.

Also provided is the compound of the invention for use in treating a LXR mediated disease selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

The invention further relates to a method for preventing and/or treating diseases mediated by LXRs, the method comprising administering a compound of the present invention in an effective amount of to a subject in need thereof.

More specifically, the invention relates to a method for preventing and treating diseases selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

Moreover, the invention also relates to the use of a compound according to the present invention in the preparation of a medicament for the prophylaxix and/or treatment of a LXR mediated disease.

More specifically, the invention relates to the use of a compound according to the present invention in the preparation of a medicament for the prophylaxix and/or treatment of a LXR mediated disease, wherein the disease is selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

Also provided is a pharmaceutical composition comprising the compound of the invention and a pharmaceutically acceptable carrier or excipient.

In the context of the present invention “C₁₋₄-alkyl” means a saturated alkyl chain having 1 to 4 carbon atoms which may be straight chained or branched. Examples thereof include methyl, ethyl, propyl, isopropyl, n-butyl, isobutyl, and tert-butyl.

The term “halo-C₁₋₄-alkyl” means that one or more hydrogen atoms in the alkyl chain are replaced by a halogen. A preferred example thereof is CF₃.

A “C₀₋₆-alkylene” means that the respective group is divalent and connects the attached residue with the remaining part of the molecule. Moreover, in the context of the present invention, “C₀-alkylene” is meant to represent a bond, whereas C₁-alkylene means a methylene linker, C₂-alkylene means a ethylene linker or a methyl-substituted methylene linker and so on. In the context of the present invention, a C₀₋₆-alkylene preferably represents a bond, a methylene, a ethylene group or a propylene group.

Similarly, a “C₂₋₆-alkenylene” and a “C₂₋₆-alkinylene” means a divalent alkenyl or alkynyl group which connects two parts of the molecule.

A 3- to 10-membered cycloalkyl group means a saturated or partially unsaturated mono-, bi-, spiro- or multicyclic ring system comprising 3 to 10 carbon atoms. Examples include cyclopropyl, cyclobutyl, cyclopentyl, cyclohexyl, cyclohexenyl, bicyclo[2.2.2]octyl, bicyclo[3.2.1]octanyl, spiro[3.3]heptyl, bicyclo[2.2.1]heptyl, adamantyl and pentacyclo[4.2.0.0^{2.5}.0³⁸.0^{4,7}]octyl. Consequently, a 3- to 6-membered cycloalkyl group means a saturated or partially unsaturated mono- bi-, or spirocyclic ring system comprising 3 to 6 carbon atoms whereas a 5- to 8-membered cycloalkyl group means a saturated or partially unsaturated mono-, bi-, or spirocyclic ring system comprising 5 to 8 carbon atoms.

A 3- to 10-membered heterocycloalkyl group means a saturated or partially unsaturated 3 to 10 membered carbon mono-, bi-, spiro- or multicyclic ring wherein 1, 2, 3 or 4 carbon atoms are replaced by 1, 2, 3 or 4 heteroatoms, respectively, wherein the heteroatoms are independently selected from N, O, S, SO and SO₂. Examples thereof include epoxidyl, oxetanyl, pyrrolidinyl, tetrahydrofuranyl, piperidinyl, piperazinyl tetrahydropyranyl, 1,4-dioxanyl, morpholinyl, 4-quinuclidinyl, 1,4-dihydropyridinyl and 6-azabicyclo[3.2.1]octanyl. The heterocycloalkyl group can be connected with the remaining part of the molecule via a carbon, nitrogen (e.g. in morpholine or piperidine) or sulfur atom. An example for a S-linked heterocycloalkyl is the cyclic sulfonimidamide

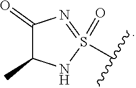 [SMILES: *S1(=O)=NC(=O)[C@H](C)N1]

A 5- to 14-membered mono-, bi- or tricyclic heteroaromatic ring system (within the application also referred to as heteroaryl) means an aromatic ring system containing up to 6 heteroatoms independently selected from N, O, S, SO and SO₂. Examples of monocyclic heteroaromatic rings include pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzofuranyl, benzoxazolyl, indolyl, indolizinyl 1,5-naphthyridinyl, 1,7-naphthyridinyl and pyrazolo[1,5-a]pyrimidinyl. Examples of tricyclic heteroaromatic rings include acridinyl, benzo[b][1,5]naphthyridinyl and pyrido[3,2-b][1,5]naphthyridinyl.

The nitrogen or sulphur atom of the heteroaryl system may also be optionally oxidized to the corresponding N-oxide, S-oxide or S,S-dioxide.

If not stated otherwise, the heteroaryl system can be connected via a carbon or nitrogen atom. Examples for N-linked heterocycles are

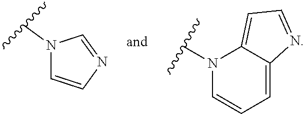 [SMILES: *n1ccnc1 ; *n1cccc2nccc1-2]

A 6- to 14-membered mono-, bi- or tricyclic aromatic ring system (within the application also referred to as aryl) means an aromatic carbon cycle such as phenyl, naphthyl, anthracenyl or phenanthrenyl.

The term “N-oxide” denotes compounds, where the nitrogen in the heteroaromatic system (preferably pyridinyl) is oxidized. Such compounds can be obtained in a known manner by reacting a compound of the present invention (such as in a pyridinyl group) with H₂O₂ or a peracid in an inert solvent.

Halogen is selected from fluorine, chlorine, bromine and iodine, more preferably fluorine or chlorine and most preferably fluorine.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, 13C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds of Formula (I) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the compounds of the present invention are partly subject to tautomerism. For example, if a heteroaromatic group containing a nitrogen atom in the ring is substituted with a hydroxy group on the carbon atom adjacent to the nitrogen atom, the following tautomerism can appear:

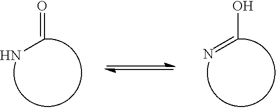

A cycloalkyl or heterocycloalkyl group can be connected straight or spirocyclic, e.g. when cyclohexane is substituted with the heterocycloalkyl group oxetane, the following structures are possible:

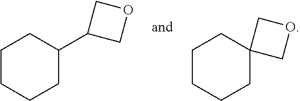 [SMILES: C1CCC(C2COC2)CC1 ; C1CCC2(CC1)COC2]

The term “1,4-orientation” means that on a ring the substituents have at least one possibility, where are 4 atoms between the two substituents attached to the ring system:

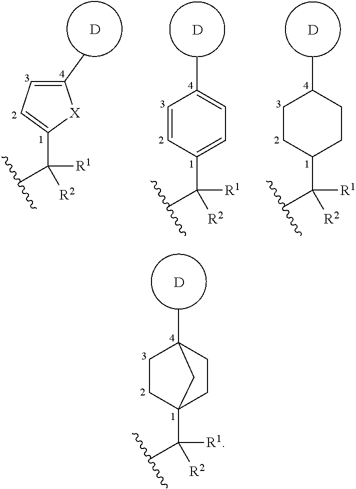

The term “1,3-orientation” means that on a ring the substituents have at least one possibility, where 3 atoms are between the two substituents attached to the ring system, e.g.

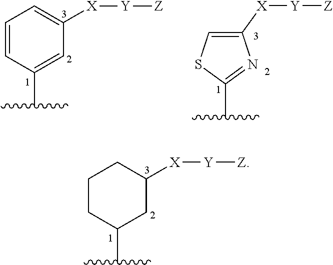

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

The compounds of the present invention can be in the form of a prodrug compound. “Prodrug compound” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods. Other examples of the prodrug are compounds (referred to as “ester prodrug” in the application, wherein the carboxylate in a compound of the present invention is, for example, converted into an alkyl-, aryl-, arylalkylene-, amino-, choline-, acyloxyalkyl-, 1-((alkoxycarbonyl)oxy)-2-alkyl, or linolenoyl- ester. Exemplary structures for prodrugs of carboxylic acids are

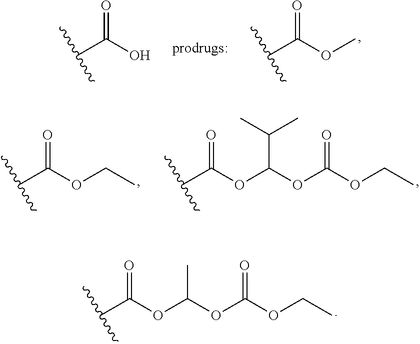

A ester prodrug can also be formed, when a carboxylic acid forms a lactone with a hydroxy group from the molecule. An exemplary example is

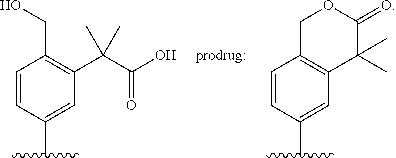 [SMILES: *c1ccc(CO)c(C(C)(C)C(=O)O)c1 ; *c1ccc2c(c1)C(C)(C)C(=O)OC2]

The term “—CO₂H or an ester thereof” means that the carboxylic acid and the alkyl esters are intented, e.g.

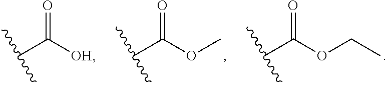

Metabolites of compounds of the present invention are also within the scope of the present invention.

Where tautomerism, like e.g. keto-enol tautomerism, of compounds of the present invention or their prodrugs may occur, the individual forms, like e.g. the keto and enol form, are each within the scope of the invention as well as their mixtures in any ratio. Same applies for stereoisomers, like e.g. enantiomers, cis/trans isomers, conformers and the like.

If desired, isomers can be separated by methods well known in the art, e.g. by liquid chromatography. Same applies for enantiomers by using e.g. chiral stationary phases.

Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials. Another way to obtain pure enantiomers from racemic mixtures would use enantioselective crystallization with chiral counterions.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present on these groups and can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Further the compounds of the present invention may be present in the form of solvates, such as those which include as solvate water, or pharmaceutically acceptable solvates, such as alcohols, in particular ethanol.

Furthermore, the present invention provides pharmaceutical compositions comprising at least one compound of the present invention, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier.

The pharmaceutical composition of the present invention may additionally comprise one or more other compounds as active ingredients like a prodrug compound or other nuclear receptor modulators.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient.

They may be conveniently presented in unit dosage form and prepared by any of the methods well-known in the art of pharmacy.

The compounds of the present invention act as LXR modulators.

Ligands to nuclear receptors including LXR ligands can either act as agonists, antagonists or inverse agonists. An agonist in this context means a small molecule ligand that binds to the receptor and stimulates its transcriptional activity as determined by e.g. an increase of mRNAs or proteins that are transcribed under control of an LXR response element.

Transcriptional activity can also be determined in biochemical or cellular in vitro assays that employ just the ligand binding domain of LXRa or LXRP but use the interaction with a cofactor (i.e. a corepressor or a coactivator), potentially in conjunction with a generic DNA-binding element such as the Gal4 domain, to monitor agonistic, antagonistic or inverse agonistic activity.

Whereas an agonist by this definition stimulates LXR- or LXR-Gal4- driven transcriptional activity, an antagonist is defined as a small molecule that binds to LXRs and thereby inhibits transcriptional activation that would otherwise occur through an endogenous LXR ligand.

An inverse agonist differs from an antagonist in that it not only binds to LXRs and inhibits transcriptional activity but in that it actively shuts down transcription directed by LXR, even in the absence of an endogenous agonist. Whereas it is difficult to differentiate between LXR antagonistic and inverse agonistic activity in vivo, given that there are always some levels of endogenous LXR agonist present, biochemical or cellular reporter assays can more clearly distinguish between the two activities. At a molecular level an inverse agonist does not allow for the recruitment of a coactivator protein or active parts thereof whereas it should lead to an active recruitment of corepressor proteins are active parts thereof. An LXR antagonist in this context would be defined as an LXR ligand that neither leads to coactivator nor to corepressor recruitment but acts just through displacing LXR agonists. Therefore, the use of assays such as the Gal4-mammalian-two-hybrid assay is mandatory in order to differentiate between coactivator or corepressor-recruiting LXR compounds (Kremoser et al., Drug Discov. Today 2007; 12:860; Gronemeyer et al., Nat. Rev. Drug Discov. 2004; 3:950).

Since the boundaries between LXR agonists, LXR antagonists and LXR inverse agonists are not sharp but fluent, the term “LXR modulator” was coined to encompass all compounds which are not clean LXR agonists but show a certain degree of corepressor recruitment in conjunction with a reduced LXR transcriptional activity. LXR modulators therefore encompass LXR antagonists and LXR inverse agonists and it should be noted that even a weak LXR agonist can act as an LXR antagonist if it prevents a full agonist from full transcriptional activation.

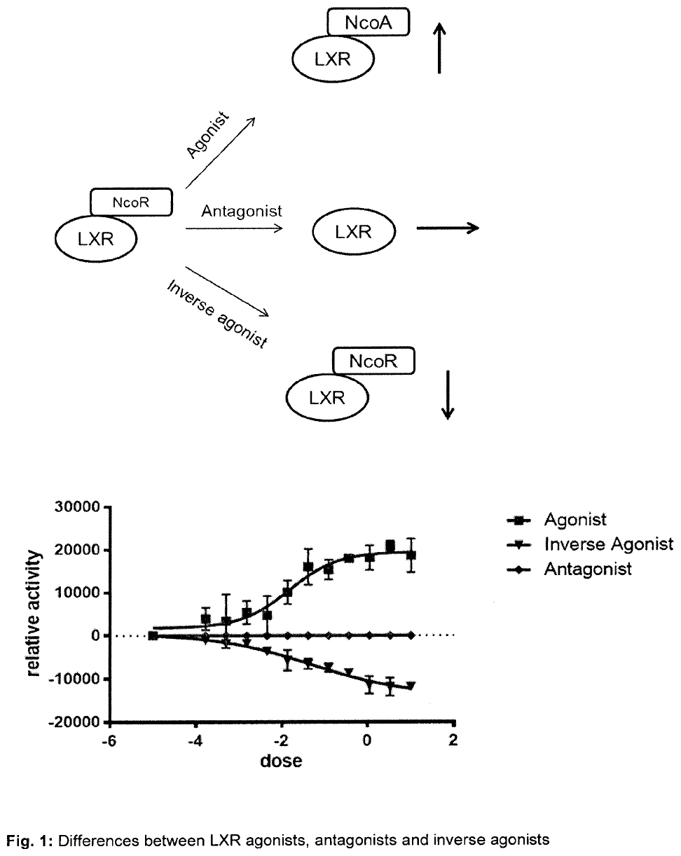

FIG. 1 shall illustrate the differences between LXR agonists, antagonists and inverse agonists here differentiated by their different capabilities to recruit coactivators or corepressors.

The compounds are useful for the prophylaxis and/or treatment of diseases which are mediated by LXRs. Preferred diseases are all disorders associated with steatosis, i.e. tissue fat accumulation. Such diseases encompass the full spectrum of non-alcoholic fatty liver disease including non-alcoholic steatohepatitis, liver inflammation and liver fibrosis, furthermore insulin resistance, metabolic syndrome and cardiac steatosis. An LXR modulator based medicine might also be useful for the treatment of hepatitis C virus infection or its complications and for the prevention of unwanted side-effects of long-term glucocorticoid treatment in diseases such as rheumatoid arthritis, inflammatory bowel disease and asthma.

A different set of applications for LXR modulators might be in the treatment of cancer. LXR antagonists or inverse agonists might useful to counteract the so-called Warburg effect which is associated with a transition from normal differentiated cells towards cancer cells (see Liberti et al., Trends Biochem. Sci. 2016; 41:211; Ward & Thompson, Cancer Cell 2012; 21:297-308). Furthermore, LXR is known to modulate various components of the innate and adaptive immune system. Oxysterols, which are known as endogenous LXR agonists were identified as mediators of an LXR-dependent immunosuppressive effect found in the tumor microenvironment (Traversari et al., Eur. J. Immunol. 2014; 44:1896). Therefore, it is reasonable to assume that LXR antagonists or inverse agonists might be capable of stimulating the immune system and antigen-presenting cells, in particular, to elicit an anti-tumor immune response. The latter effects of LXR antagonists or inverse agonists might be used for a treatment of late stage cancer, in general, and in particular for those types of cancerous solid tumors that show a poor immune response and highly elevated signs of Warburg metabolism.

In more detail, anti-cancer activity of the LXR inverse agonist SR9243 was shown to be mediated by interfering with the Warburg effect and lipogenesis in different tumor cells in vitro and SW620 colon tumor cells in athymic mice in vivo (see Flaveny et al. Cancer Cell. 2015; 28:42; Steffensen, Cancer Cell 2015; 28:3).

LXR modulators (preferably LXR inverse agonists) may counteract the diabetogenic effects of glucocorticoids without compromising the anti-inflammatory effects of glucocorticoids and could therefore be used to prevent unwanted side-effects of long-term glucocorticoid treatment in diseases such as rheumatoid arthritis, inflammatory bowel disease and asthma (Patel et al. Endocrinology 2017:158:1034).

LXR modulators (preferably LXR inverse agonists) may be useful for the treatment of hepatitis C virus mediated liver steatosis (see García-Mediavilla et al. Lab. Invest. 2012; 92:1191).

LXR modulators (preferably LXR inverse agonists) may be useful for the treatment of viral myocarditis (see Papageorgiou et al. Cardiovasc. Res. 2015; 107:78).

LXR modulators (preferably LXR inverse agonists) may be useful for the treatment of insulin resistance (see Zheng et al. PLoS One 2014; 9:e101269).

LXR modulators (preferably LXR inverse agonists) may be useful for the treatment of familial hypercholesterolemia (see Zhou et al. J. Biol. Chem. 2008; 283:2129).

LXR modulators (preferably LXR inverse agonists) may be useful for the treatment of hypercholesterolemia in nephrotic syndrome (see Liu & Vazizi in Nephrol. Dial. Transplant. 2014; 29:538).

### EXPERIMENTAL SECTION

The compounds of the present invention can be prepared by a combination of methods known in the art including the procedures described in Schemes I and II below.

In case when R⁵ and R⁶ is not together an oxygen or sulfur atom, the compounds of the present invention can be prepared as outlined in Scheme I: Protected amine derivative I-a is alkylated with halogen compound I-b using an appropriate base (e.g. NaH, LiHMDS or Cs₂CO₃) in a suitable solvent (e.g. dry DMF). Then the protecting group (PG) is cleaved to afford secondary amine I-c. This amine can be alkylated again with halogen compound I-d using an appropriate base (e.g. NaH or Cs₂CO₃) in a suitable solvent (e.g. dry DMF) to afford tertiary amine I-e. Optionally, when appropriate, the derivatives I-e can also be assembled using aldehyde/ketone I-j and reduction agent (e.g. NaBH(OAc)₃, NaBH₄ or Ti(i-PrO)₄) and optinally catalytic amounts of acid (e.g. AcOH). Coupling of halogen derivative I-e with boronic acid or boronic ester building block under Suzuki conditions affords, after optional manipulation of the X—Y—Z-moiety (e.g. oxidation, hydrogenation and/or saponification), target molecule I-h. Optionally, the boronic ester intermediate can be formed first and then halogen derivative I-g is coupled under Suzuki conditions and treated as described before. Even in situ generation of boronic ester with B₂Pin₂ under Suzuki conditions can be applied. As outlined in the Examples an alternate order of the synthetic steps can be applied.

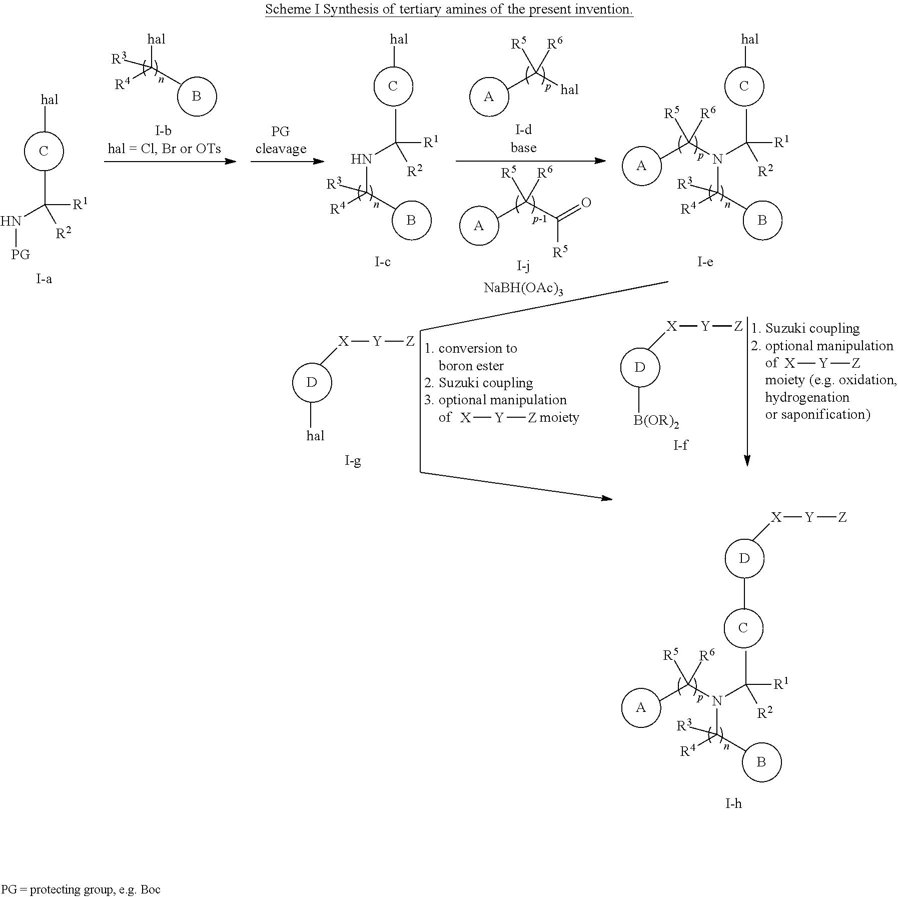

In case when one R⁵/R⁶-pair is together an oxygen or sulfur atom, the compounds of the present invention can be prepared as outlined in Scheme II: Protected amine derivative I-a is alkylated with halogen compound I-b using an appropriate base (e.g. NaH, LiHMDS or Cs₂CO₃) in a suitable solvent (e.g. dry DMF). Then the protecting group (PG) is cleaved to afford secondary amine I-c. This amine can be reacted with (thio)acid chloride II-d and an appropriate base (e.g. NEt₃) to afford (thio)amide II-e. Alternatively amide coupling (e.g. with HATU or EDCl) using an acid derivative can be applied. Similar as outlined in Scheme I, the target compound II-h can be prepared. As outlined in the Examples an alternate order of the synthetic steps can be applied.

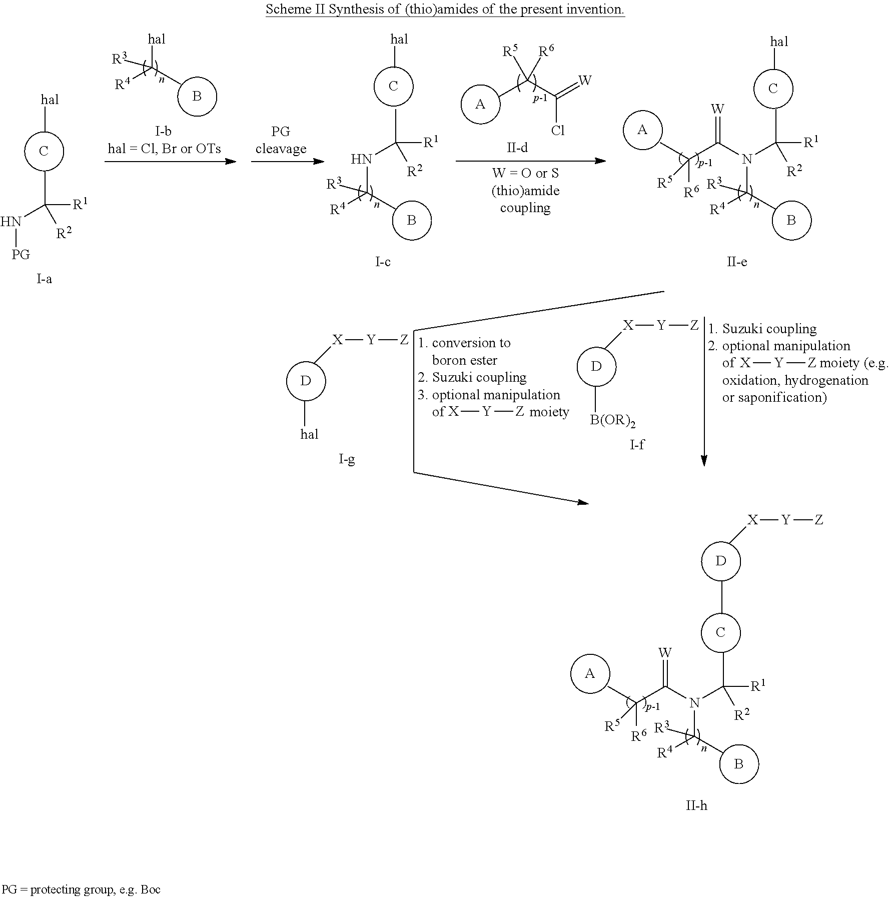

### Abbreviations

Ac acetyl
ACN acetonitrile
AIBN azobisisobutyronitrile
aq. aqueous
B₂Pin₂ 4,4,4′,4′,5,5,5′,5′-octamethyl-2,2′-bi-1,3,2-dioxaborolane
Boc tert-butyloxycarbonyl
BPO dibenzoyl peroxide
m-CPBA meta-chloroperbenzoic acid
Cy cyclohexyl
d day(s) or dublett (in the ¹H-NMR data)
DAST diethylaminosulfur trifluoride
dba dibenzylideneacetone
DCM dichloromethane
DIEA or DIPEA diisopropylethylamine

### DMAP 4-N,N-dimethylaminopyridine

### DMF N,N-dimethylformamide

dppf 1,1′-bis(diphenylphosphino)ferrocene
EA ethyl acetate
FCC flash column chromatography on silica gel
EDCI 1-ethyl-3-(3-dimethylaminopropyl)carbodiimide
h hour(s)
HATU O-(7-azabenzotriazole-1-yl)-N,N,N′,N′-tetramethyluronium
hexafluorophosphate
HOBt hydroxybenzotriazole
IBX 2-iodoxybenzoic acid
LiHMDS lithium bis(trimethylsilyl)amide
min minute(s)
MS mass spectrometry

### NBS N-bromosuccinimide

PCC pyridinium chlorochromate
Pin pinacolato (OCMe₂CMe₂O)
PE petroleum ether
prep preparative
sat. saturated (aqueous)
S-phos 2-dicyclohexylphosphino-2′,6′-dimethoxybiphenyl
TEA triethylamine
TFA trifluoroacetic acid
TFAA trifluoroacetic acid anhydride
THF tetrahydrofuran
TLC thin layer chromatography
XPhos 2-dicyclohexylphosphino-2′,4′,6′-triisopropylbiphenyl

### Preparative Example P1

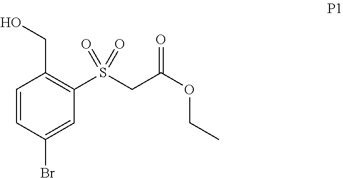 [SMILES: CCOC(=O)CS(=O)(=O)c1cc(Br)ccc1CO]

### Step 1: (4-Bromo-2-mercaptophenyl)methanol (P1a)

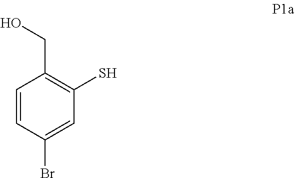

To a solution of 4-bromo-2-mercaptobenzoic acid (1.50 g, 6.50 mmol) in THF (30 mL) was added BH₃ (13 mL, 1M in THF). This mixture was stirred overnight and quenched with water (30 mL). EA (20 mL) was added and the organic layer was separated and the aq. layer was washed with EA (3×20 mL). The combined organic layer was washed with brine (30 mL), dried over Na₂SO₄ and concentrated to give compound P1a as a yellow solid.

### Step 2: Ethyl 2-((5-bromo-2-(hydroxymethyl)phenyl)thio)acetate (P1b)

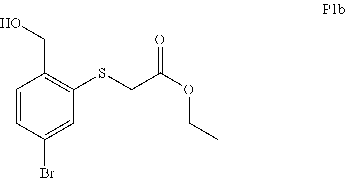 [SMILES: CCOC(=O)CSc1cc(Br)ccc1CO]

To a mixture of compound P1a (436 mg, 2.00 mmol) and ethyl 2-bromoacetate (306 mg, 2.00 mmol) in DMF (10 mL) was added Cs₂CO₃ (2.0 g, 6.0 mmol) and the mixture was stirred overnight, diluted with water (100 mL) and extracted with EA (3×30 mL). The combined organic layer was washed with brine (30 mL), dried over Na₂SO₄, concentrated and purified by FCC (PE:EA=5:1) to give compound P1b as a white solid.

### Step 3: Ethyl 2-((5-bromo-2-(hydroxymethyl)phenyl)sulfonyl)acetate (P1)

To a stirred solution of compound Pb (290 mg, 1.00 mmol) in DCM (5 mL) at 0° C. was added m-CPBA (610 mg, 3.00 mmol, 85%) and the mixture was stirred at rt for 16 h, diluted with aq. sat. NaHCO₃ solution and extracted with EA (3×20 mL). The combined organic layer was dried over Na₂SO₄, concentrated and purified by FCC (PE:EA=5:1) to give compound P1 as a white solid.

### Preparative Example P2

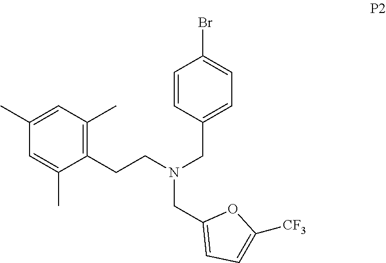 [SMILES: Cc1cc(C)c(CCN(Cc2ccc(Br)cc2)Cc2ccc(C(F)(F)F)o2)c(C)c1]

### Step 1: N-(4-Bromobenzyl)-2-mesitylethan-1-amine (P2a)

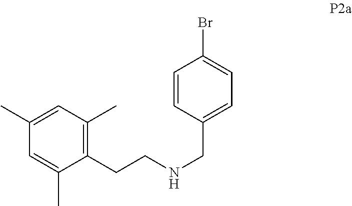 [SMILES: Cc1cc(C)c(CCNCc2ccc(Br)cc2)c(C)c1]

A solution of 2-mesitylethan-1-amine (300 mg, 1.84 mmol) and 4-bromobenzaldehyde (339 mg, 1.84 mmol) in MeOH (30 mL) was stirred at rt overnight. After adding NaBH₄ (105 mg, 2.76 mmol), the mixture was stirred at rt overnight, diluted with water, adjust to pH ˜11 by adding 1N NaOH, concentrated and extracted with EA (3×). The combined organic layer was washed with water and brine, dried over Na₂SO₄, filtered and concentrated to give compound P2a as a yellow oil.

### Step 2: N-(4-Bromobenzyl)-2-mesityl-N-((5-(trifluoromethyl)furan-2-yl)methyl)ethan-1-amine (P2)

To a solution of compound P2a (724 mg, 2.19 mmol), 2-(bromomethyl)-5-(trifluoromethyl)furan (499 mg, 2.19 mmol) and K₂CO₃ (604 mg, 4.37 mmol) in ACN (40 mL) was added KI (363 mg, 2.19 mmol) at rt. The mixture was stirred at 80° C. overnight, cooled, filtered, concentrated and purified by FCC (PE:EA=25:1) to give compound P2 as a yellow oil.

### Preparative Example P2/1 to P2/3

The following Preparative Examples were prepared similar as described for Preparative Example P2 using the appropriate building blocks.

| # | building blocks | structure |
|---|---|---|
| P2/1 | 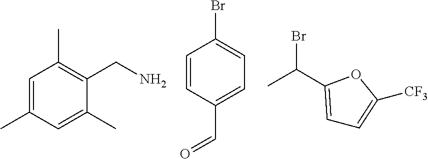 | 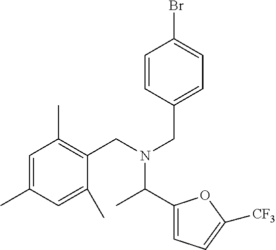 [SMILES: Cc1cc(C)c(CN(Cc2ccc(Br)cc2)C(C)c2ccc(C(F)(F)F)o2)c(C)c1] |
| P2/2 | 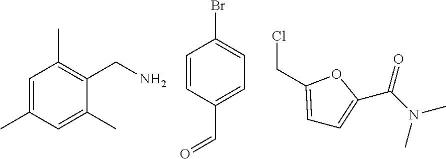 | 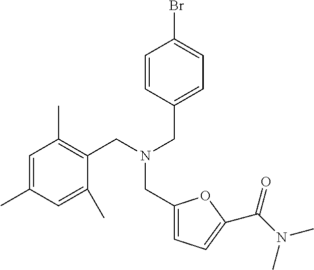 |
| P2/3 | 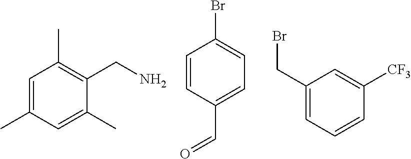 | 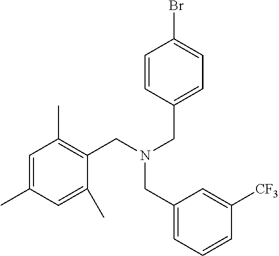 [SMILES: Cc1cc(C)c(CN(Cc2ccc(Br)cc2)Cc2cccc(C(F)(F)F)c2)c(C)c1] |

### Preparative Example P3

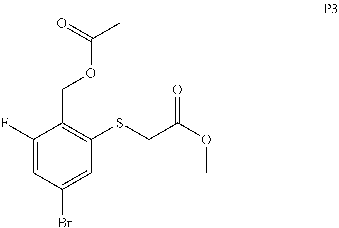 [SMILES: COC(=O)CSc1cc(Br)cc(F)c1COC(C)=O]

### Step 1: tert-Butyl 4-bromo-2,6-difluorobenzoate (P3a)

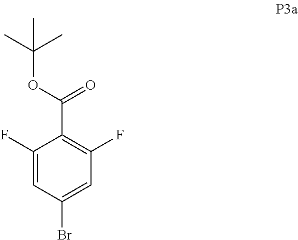 [SMILES: CC(C)(C)OC(=O)c1c(F)cc(Br)cc1F]

A mixture of 4-bromo-2,6-difluorobenzoic acid (25.0 g, 110 mmol), Boc₂O (50.0 g, 242 mmol) and DMAP (1.3 g, 11 mmol) in tert-BuOH (200 mL) was stirred at 40° C. overnight, concentrated and purified by FCC (PE:EA=50:1) to give compound P3a as a yellow oil. MS: 292 (M+1)⁺.

### Step 2: tert-Butyl 4-bromo-2-fluoro-6-((2-methoxy-2-oxoethyl)thio)benzoate (P3b)

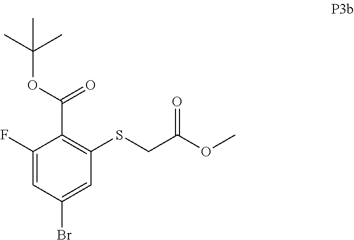 [SMILES: COC(=O)CSc1cc(Br)cc(F)c1C(=O)OC(C)(C)C]

To a solution of methyl 2-mercaptoacetate (11.2 g, 106 mmol) in dry DMF (50 mL) was added NaH (60%, 5.1 g, 130 mmol) at 0° C. The mixture was stirred 30 min. Then the mixture was added to a solution of compound P3a (31 g, 106 mmol) in dry DMF (100 mL). The mixture was stirred at rt for 2 h, diluted with H₂O (1000 mL) and extracted with EA (3×). The combined organic layer was washed with H₂O and brine, concentrated and purified by FCC (PE:EA=10:1) to give compound P3b as a yellow oil. MS: 378 (M+1)⁺.

### Step 3: 4-Bromo-2-fluoro-6-((2-methoxy-2-oxoethyl)thiobenzoic acid (P3c)

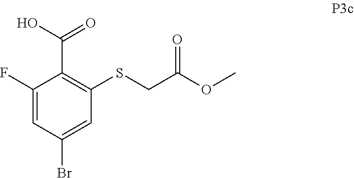 [SMILES: COC(=O)CSc1cc(Br)cc(F)c1C(=O)O]

A solution of compound P3b (18.0 g, 47.5 mmol) and TFA (30 mL) in DCM (60 mL) was stirred at rt overnight, concentrated, diluted with Et₂O and stirred for 30 min. The mixture was filtered to give compound P3c as a white solid.

### Step 4: Methyl 2-((5-bromo-3-fluoro-2-(hydroxymethyl)phenyl)thio)acetate (P3d)

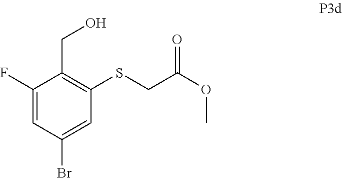 [SMILES: COC(=O)CSc1cc(Br)cc(F)c1CO]

To a solution of compound P3c (12.0 g, 37.3 mmol) in THF (100 mL) was added TEA (10 mL) at 0° C. Then isobutyl carbonochloridate (5.50 g, 41.0 mmol) was added slowly to the mixture at 0° C. The mixture was stirred at 0° C. for 30 min, filtered and washed with THF (100 mL).

The filtrate was cooled to 0° C. and NaBH₄ (2.80 g, 74.6 mmol) was added slowly. The mixture was allowed to warm to rt for 3 h. Sat. NH₄Cl (1000 mL) was added and the solution was extracted with EA (2×200 mL). The combined organic layer was successively washed with water (500 mL) and brine (200 mL), dried over Na₂SO₄, filtered, concentrated and purified by FCC (PE/EA=10:1) to give compound P3d as a white solid. ¹H-NMR (CDCl₃, 300 MHz) δ: 7.43 (t, J=1.6 Hz, 1H), 7.19 (dd, J=1.6, 8.4 Hz, 1H), 4.85 (d, J=2.0 Hz, 2H), 3.73 (s, 2H), 3.72 (s, 3H), 2.59 (br s, 1H); MS: 306.9/308.9 (M+1)⁺.

### Step 5: Methyl 2-((2-(acetoxymethyl)-5-bromo-3-fluorophenyl)thio)acetate (P3)

A solution of compound P3d (3.50 g, 11.4 mmol) in DCM (100 mL) was treated with catalytic amounts of DMAP (140 mg, 1.1 mmol) under N₂. To the mixture was added TEA (1.70 g, 17.1 mmol) and Ac₂O (1.40 g, 13.7 mmol) and the mixture was stirred at rt for 1 h, washed with 1N HCl (100 mL), water and brine, dried over Na₂SO₄, filtered and concentrated to give crude compound P3 as a white solid, which was used in the next step without further purification.

### Preparative Example P4

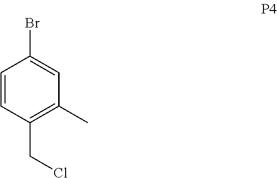

### 4-Bromo-1-(chloromethyl)-2-methylbenzene (P4)

To a solution of (4-bromo-2-methylphenyl)methanol (500 mg, 2.5 mmol) in DCM (20 mL) was added SOCl₂ (0.89 g, 7.5 mmol) at 0° C. under N₂. The mixture was stirred at rt for 1 h, then aq. Na₂CO₃ was added to adjust the pH to approx. 6. The organic layer was washed with brine, dried over Na₂SO₄, concentrated and purified by FCC (PE) to afford compound P4 as a colorless oil.

### Preparative Example P5

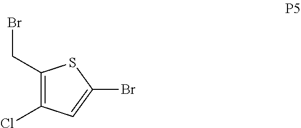

### 5-Bromo-2-(bromomethyl)-3-chlorothiophene (P5)

To a solution of (3-chlorothiophen-2-yl)methanol (1.0 g, 6.7 mmol) in AcOH (15 mL) was added Br₂ (1.2 g, 7.4 mmol) at 15° C. After warming up to rt, the mixture was stirred overnight, poured into water and extracted with EA (200 mL). The organic layer was washed with aq. Na₂SO₃ and brine, dried over Na₂SO₄, filtered and concentrated to give compound P5 as a yellow oil.

### Preparative Example P6

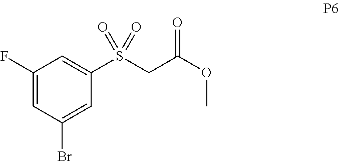 [SMILES: COC(=O)CS(=O)(=O)c1cc(F)cc(Br)c1]

### Step 1: Methyl 2-((3-bromo-5-fluorophenyl)thio)acetate (P6a)

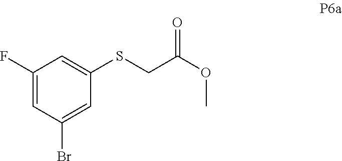

To a suspension of methyl 2-mercaptoacetate (2.8 g, 26 mmol) in dry DMF (30 mL) was added NaH (60% w/t in mineral oil, 2.0 g, 52 mmol) at 0° C. and the mixture was stirred at 0° C. for 10 min, then 1-bromo-3,5-difluorobenzene (5.0 g, 26 mmol) was added at 0° C. The solution was stirred at rt for 3 h, quenched with water (30 mL) and extracted with EA (3×50 mL). The combined organic layer was dried over Na₂SO₄, filtered, concentrated and purified by FCC (PE:EA=10:1) to give compound P6a as a yellow oil. ¹H-NMR (CDCl₃, 300 MHz) δ: 7.30 (s, 1H), 7.12-7.06 (m, 2H), 3.77 (s, 3H), 3.69 (s, 2H).

### Step 2: Methyl 2-((3-bromo-5-fluorophenyl)sulfonyl)acetate (P6)

To a solution of compound P6a (400 mg, 1.43 mmol) in DCM (300 mL) was added m-CPBA (616 mg, 3.6 mmol) under ice-bath cooling. The mixture was stirred at rt for 2 h, diluted with water (20 mL) and extracted with DCM (3×15 mL). The combined organic layer was washed with brine (20 mL), dried over Na₂SO₄, filtered and concentrated to afford crude compound P6 as a colorless oil. ¹H-NMR (CDCl₃, 300 MHz) δ: 7.92 (s, 1H), 7.65-7.58 (m, 2H), 4.17 (s, 2H), 3.77 (s, 3H).

### Preparative Example P7 and P7-1

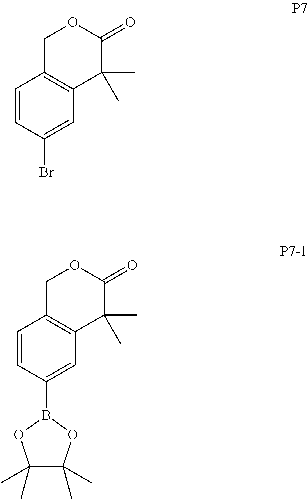

### Step 1: 4-Bromo-2-(bromomethyl)-1-methylbenzene (P7a)

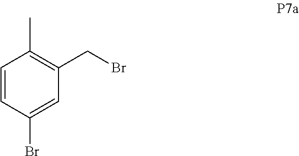

To a solution of (5-bromo-2-methylphenyl)methanol (2.7 g, 13 mmol) in THF (50 mL) was added PBr₃ (0.6 mL, 6.7 mmol) under ice-bath cooling. The mixture was stirred at 0° C. for 2 h, diluted with water (100 mL), basified to pH=7 with sat. NaHCO₃ and extracted with EA (3×50 mL). The combined organic layer was washed with brine (100 mL), dried over Na₂SO₄, filtered and concentrated to give compound P7a as a yellow oil.

### Step 2: 2-(5-Bromo-2-methylphenyl)acetonitrile (P7b)

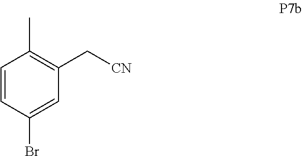

To a solution of compound P7a (3.5 g, 13 mmol) in DMF (50 mL) was added NaCN (715 mg, 14.6 mmol) at rt. The mixture was stirred at 60° C. for 5 h, diluted with water (100 mL) and extracted with EA (3×50 mL). The combined organic layer was washed with water (2×100 mL) and brine (100 mL), dried over Na₂SO₄, filtered and concentrated to give crude compound P7b as a white solid.

### Step 3: 2-(5-Bromo-2-methylphenyl)acetic acid P7c

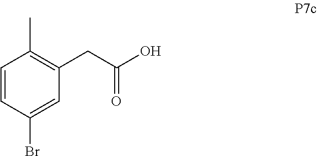

To a solution of compound P7b (1.6 g, 7.6 mmol) in water (50 mL) and EtOH (50 mL) was added KOH (4.3 g, 76 mmol) at rt. The mixture was stirred at reflux overnight, then the EtOH was evaporated. The solution was acidified to pH=3 with 1N HCl and extracted with EA (3×50 mL). The combined organic layer was washed with brine (100 mL), dried over Na₂SO₄, filtered and concentrated to give crude compound P7c as a white solid.

### Step 4: Methyl 2-(5-bromo-2-methylphenyl)acetate (P7d)

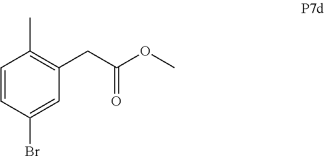

To a solution of compound P7c (1.5 g, 6.6 mmol) in MeOH (50 mL) was added conc. H₂SO₄ (0.3 mL) at rt. The mixture was stirred at reflux overnight, concentrated and dissolved in EA (50 mL) and water (20 mL). The mixture was basified to pH=7 with sat. NaHCO₃ and extracted with EA (2×50 mL). The combined organic layer was washed with brine (100 mL), dried over Na₂SO₄, filtered and concentrated to give crude compound P7d as a yellow oil.

### Step 5: Methyl 2-(5-bromo-2-methylphenyl)-2-methylpropanoate (P7e)

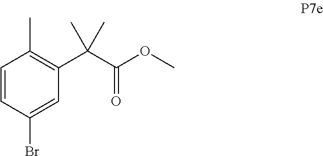

To a solution of compound P7d (9.5 g, 39 mmol) in dry DMF (100 mL) was added NaH (3.9 g, 60%, 98 mmol) under ice-bath cooling. The mixture was stirred for 10 min at 0° C., then 18-crown-6 (1.1 g, 7.8 mmol) and MeI (12.2 mL, 196 mmol) were added. The mixture was stirred at rt overnight, diluted with water (200 mL) and extracted with EA (3×100 mL). The combined organic layer was washed with water (2×200 mL) and brine (100 mL), dried over Na₂SO₄, filtered and concentrated. The procedure was repeated again and then the obtained residue was purified by FCC (PE:EA=20:1) to give crude compound P7e as a yellow oil.

### Step 6: Methyl 2-(5-bromo-2-(bromomethyl)phenyl)-2-methylpropanoate (P7f)

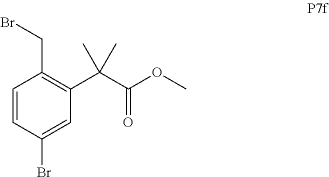 [SMILES: COC(=O)C(C)(C)c1cc(Br)ccc1CBr]

To a solution of compound P7e (9.0 g, 33 mmol) in CCl₄ (150 mL) was added NBS (6.5 g, 37 mmol) and BPO (0.80 g, 3.3 mmol) at rt under N₂. The mixture was stirred at reflux overnight and concentrated. The residue was dissolved in EA (200 mL), washed with water (100 mL) and brine (100 mL), dried over Na₂SO₄, filtered and concentrated to give crude compound P7f as a yellow oil.

### Step 7: Methyl 2-(2-(acetoxymethyl)-5-bromophenyl)-2-methylpropanoate (P7)

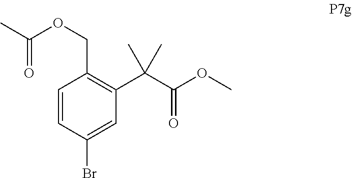 [SMILES: COC(=O)C(C)(C)c1cc(Br)ccc1COC(C)=O]

To a solution of compound P7f (11.0 g, 31.4 mmol) in DMF (100 mL) was added KOAc (6.2 g, 63 mmol) and KI (50 mg, 0.3 mmol) at rt. The mixture was stirred at rt for 2 h, diluted with water (200 mL) and extracted with EA (3×100 mL). The combined organic layer was washed with water (2×200 mL) and brine (100 mL), dried over Na₂SO₄, filtered, concentrated and purified by FCC (PE:EA=10:1) to give compound P7g as a yellow oil.

### Step 8: 6-Bromo-4,4-dimethylisochroman-3-one (P7)

To a solution of compound P7g (5.5 g, 17 mmol) in MeOH (50 mL) and water (50 mL) was added KOH (3.7 g, 63 mmol) at rt. The mixture was stirred at rt for 5 h and then concentrated.

The residue was acidified to pH=5 with 1N HCl, stirred at rt for 1 h and filtered. The filter cake was washed with PE/EA (20 mL, 10/1) to give compound P7 as a white solid. ¹H-NMR (CDCl₃, 400 MHz) δ: 7.50 (d, J=2.0 Hz, 1H), 7.42 (dd, J=8.0, 1.6 Hz, 1H), 7.05 (d, J=8.0 Hz, 1H), 5.36 (s, 2H), 1.58 (s, 6H); MS: 255 (M+1)⁺.

### Step 9: 4,4-Dimethyl-6-(4,4,5,5-tetramethyl-1,3,2-dioxaborolan-2-yl)isochroman-3-one (P7-1)

To a solution of compound P7 (900 mg, 3.53 mmol), B₂Pin₂ (986 mg, 3.88 mmol) and KOAc (1.04 g, 10.6 mmol) in 1,4-dioxane (20 mL) was added Pd(dppf)Cl₂ (284 mg, 0.35 mmol) at rt under N₂. The mixture was stirred at 100° C. overnight, cooled, filtered, concentrated and purified by FCC (PE:EA=20:1) to give compound P7-1 as a white solid.

### Preparative Example P8

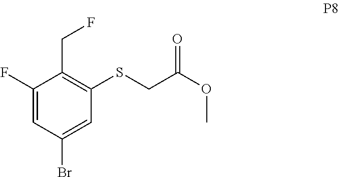 [SMILES: COC(=O)CSc1cc(Br)cc(F)c1CF]

### Methyl 2-((5-bromo-3-fluoro-2-(fluoromethyl)phenyl)thio)acetate (P8)

A mixture of compound P3d (500 mg, 1.62 mmol) in DCM (5 mL) under N₂ was cooled to −78° C., then bis(2-methoxyethyl)aminosulfur trifluoride (429 mg, 1.94 mmol) was added dropwise and the mixture was stirred at −78° C. for 3 h, quenched with water and extracted with EA (3×). The combined organic layer was washed with brine (10 mL), dried over Na₂SO₄, filtered, concentrated and purified by prep-TLC (PE:EA=10:1) to give compound P8 as a colorless oil.

### Preparative Example P9

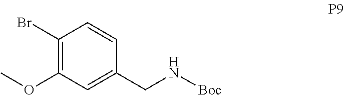 [SMILES: COc1cc(CNC(=O)OC(C)(C)C)ccc1Br]

### tert-Butyl (4-bromo-3-methoxybenzyl)carbamate P9

A solution of Boc₂O (1.70 g, 7.80 mmol) in CH₂Cl₂ (10 mL) was added to a suspension of (4-bromo-3-methoxyphenyl)methanamine (1.70 g, 7.80 mmol) and Et₃N (1.60 g, 15.6 mmol) in CH₂Cl₂ (20 mL) for 5 min at 0° C. under a CaCl₂ tube. The mixture was stirred overnight at rt, diluted with H₂O (500 mL) and the organic layer was separated. The aq. layer was extracted with CHCl₃ (3×50 mL). The combined organic layer was washed with H₂O (50 mL) and brine (50 mL), dried over Na₂SO₄, filtered, concentrated and purified by FCC (PE:EA=10:1) to give compound P9 as a white solid.

### Preparative Example P10

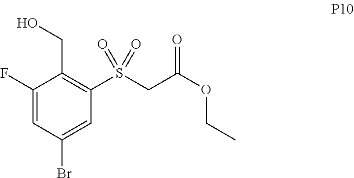 [SMILES: CCOC(=O)CS(=O)(=O)c1cc(Br)cc(F)c1CO]

### Step 1: 4-Bromo-2-((2-ethoxy-2-oxoethyl)thio)-6-fluorobenzoic acid (P10a)

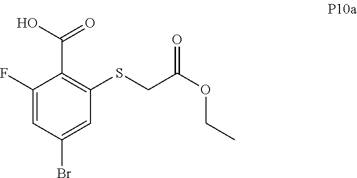 [SMILES: CCOC(=O)CSc1cc(Br)cc(F)c1C(=O)O]

To a mixture of 4-bromo-2,6-difluorobenzoic acid (10.0 g, 42.4 mmol) and ethyl 2-mercaptoacetate (5.10 g, 42.4 mmol) in DMF (100 mL) was added Cs₂CO₃ (41.5 g, 127 mmol) and the mixture was stirred at 80° C. overnight, diluted with water (1 L) and adjusted to pH=3 with 2M HCl and extracted with EA (3×300 mL). The combined organic layer was washed with brine (300 mL), dried over Na₂SO₄, filtered, concentrated and purified by FCC (PE:EA=1:1) to give compound P10a as a yellow oil.

### Step 2: Ethyl 2-((5-bromo-3-fluoro-2-(hydroxymethyl)phenyl)thio)acetate (P10b)

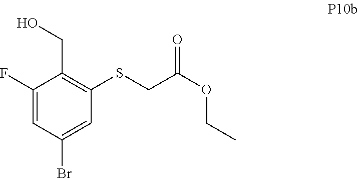 [SMILES: CCOC(=O)CSc1cc(Br)cc(F)c1CO]

To the solution of compound P10a (4.10 g, 12.2 mmol) in THF (40 mL) was added B₂H₆ (24.4 mL, 1M in THF). This mixture was stirred at 70° C. overnight, quenched with water (100 mL) and extracted with EA (4×40 mL). The combined organic layer was washed with brine (50 mL), dried over Na₂SO₄, filtered, concentrated and purified by FCC (PE:EA=5:1) to give compound P10b as a white solid.

### Step 3: Ethyl 2-((5-bromo-3-fluoro-2-(hydroxymethyl)phenylsulfonyl)acetate (P10)

To a stirred solution of compound P10b (1.00 g, 3.40 mmol) in DCM (30 mL) at 0° C. was added m-CPBA (1.80 g, 10.2 mmol, 85%) and the mixture was stirred at rt for 16 h, diluted with aq. sat. NaHCO₃ solution and extracted with EA (3×20 mL). The combined organic layer was dried over Na₂SO₄, concentrated and purified by FCC (PE:EA=5:1) to give compound P10 as a white solid.

### Preparative Example P11

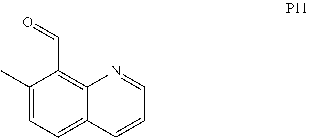

### 7-Methylquinoline-8-carbaldehyde (P11)

A solution of 8-bromo-7-methylquinoline (500 mg, 2.30 mmol) in THF (10 mL) was cooled to −78° C. n-BuLi (2.5M in hexane, 2.80 mmol) was added dropwise and the mixture was stirred at −78° C. for 1 h. Dry DMF (336 mg, 4.60 mmol) was added dropwise and the mixture was warmed to rt, quenched with sat. NH₄Cl (30 mL) and extracted with EA (3×20 mL). The combined organic layer was washed with brine (30 mL), dried over Na₂SO₄, filtered, concentrated and purified by FCC (PE:EA=2:1) to give compound P11 as a yellow solid. ¹H-NMR (500 MHz, DMSO-d₆) δ: 11.49 (s, 1H), 9.03 (dd, J=3.5 Hz, J=1.5 Hz, 1H), 8.47 (dd, J=8.5 Hz, J=2.0 Hz, 1H), 8.18 (d, J=8.0 Hz, 1H), 7.64-7.60 (m, 2H), 2.72 (s, 3H).

### Preparative Example P11/1 to P11/3

The following Preparative Examples were prepared similar as described for Preparative Example P11 using the appropriate building block.

| # | building block | structure | analytical data |
|---|---|---|---|
| P11/1 | 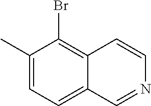 | 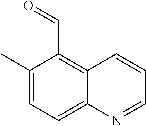 | |
| P11/2 | 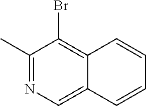 | 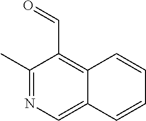 | |
| P11/3 | 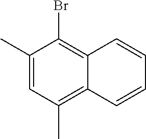 | 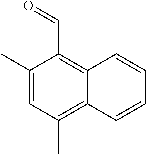 | ¹H-NMR (500 MHz, DMSO-d₆) δ: 10.83 (s, 1H), 9.02 (d, J = 8.5 Hz, 1H), 8.08 (d, J = 8.5 Hz, 1H), 7.67-7.64 (m, 1H), 7.60-7.57 (m, 1H), 7.36 (s, 1H), 2.75 (s, 3H), 2.69 (s, 3H). |

### Preparative Example P12

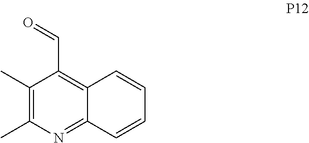

### Step 1: Methyl 2,3-dimethylquinoline-4-carboxylate (P12a)

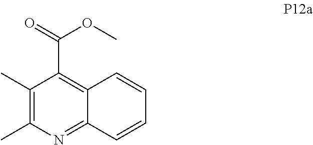 [SMILES: COC(=O)c1c(C)c(C)nc2ccccc12]

To a mixture of 2,3-dimethylquinoline-4-carboxylic acid (1.00 g, 5.00 mmol) in DMF (10 mL) was added Cs₂CO₃ (3.26 g, 10.0 mmol) and iodomethane (923 mg, 6.50 mmol). The mixture was stirred at rt overnight, diluted with water (50 mL) and extracted with EA (3×30 mL). The combined organic layer was washed with brine (30 mL), dried over Na₂SO₄, filtered, concentrated and purified by FCC (PE:EA=5:1) to give compound P12a as a white solid.

### Step 2: (2,3-Dimethylquinolin-4-yl)methanol (P12b)

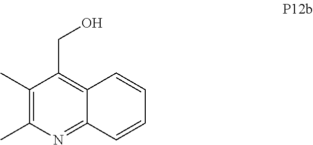

To a mixture of compound P12a (1.00 g, 4.65 mmol) in methanol (10 mL) was added NaBH₄ (532 mg, 14.0 mmol) at 0° C. and the mixture was stirred for 3 h, diluted with water (50 mL) and extracted with EA (3×30 mL). The combined organic layer was washed with brine (30 mL), dried over Na₂SO₄, filtered, concentrated and purified by FCC (PE:EA=2:1) to give compound P12b as a white solid.

### Step 3: 2,3-Dimethylquinoline-4-carbaldehyde (P12

To a mixture of compound P12b (400 mg, 2.10 mmol) in acetone (30 mL) was added IBX (2.4 g, 8.4 mmol) and the mixture was stirred at 50° C. for 12 h and filtered. The filtrate was concentrated and purified by FCC (PE:EA=4:1) to give compound P12 as a yellow solid.

### Preparative Example P12/1

The following Preparative Example was prepared similar as described for Preparative Example P12 using the appropriate building block.

| # | building block | structure |
|---|---|---|
| P12/1 | 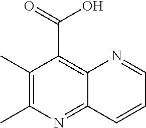 | 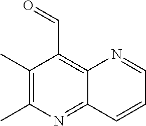 |

### Preparative Example P13

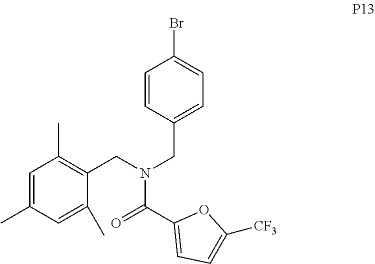 [SMILES: Cc1cc(C)c(CN(Cc2ccc(Br)cc2)C(=O)c2ccc(C(F)(F)F)o2)c(C)c1]

### N-(4-Bromobenzyl)-5-(trifluoromethyl)-N-(2,4,6-trimethylbenzl)furan-2-carboxamide (P13

To a solution of N-(4-bromobenzyl)-1-mesitylmethanamine (880 mg, 2.8 mmol), 5-(trifluoromethyl)furan-2-carboxylic acid (500 mg, 2.8 mmol) and DIEA (0.93 mL, 5.6 mmol) in DMF (20 mL) was added HATU (1.3 g, 3.4 mmol) at 0° C. The mixture was stirred at rt overnight, diluted with water and extracted with EA. The organic layer was washed with water and brine, dried over Na₂SO₄, filtered, concentrated and purified by FCC (PE:EA=30:1) to give compound P13 as a yellow solid.

### Preparative Example P14

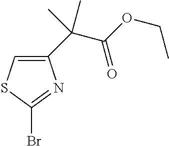

### Ethyl 2-(2-bromothiazol-4-yl)-2-methylpropanoate (P14)

To a solution of ethyl 2-(2-bromothiazol-4-yl)acetate (250 mg, 1.00 mmol) in dry DMF (20 mL) was added NaH (100 mg, 2.50 mmol) at 0° C. and the mixture was stirred for 15 min. To the mixture was added MeI (568 mg, 4.00 mmol) at 0° C. and then the mixture was stirred for further 4 h, poured into ice water and extracted with EA (3×). The combined organic layer washed with brine, dried over Na₂SO₄, filtered, concentrated and purified by FCC (PE:EA=20:1) to give compound P14 as a yellow oil.

### Preparative Example P14/1 to P14/2

The following Preparative Examples were prepared similar as described for Preparative Example P14 using the appropriate building block.

| # | building block | structure | analytical data |
|---|---|---|---|
| P14/1 | 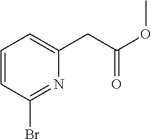 | 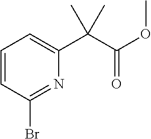 | MS: 258 (M + 1)⁺. |
| P14/2 | 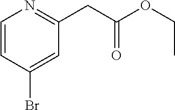 | 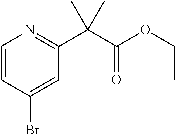 | MS: 272 (M + 1)⁺. |

### Preparative Example P15

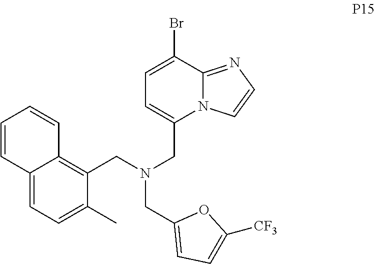 [SMILES: Cc1ccc2ccccc2c1CN(Cc1ccc(C(F)(F)F)o1)Cc1ccc(Br)c2nccn12]

### Step 1: (8-Bromoimidazo[1,2-a]pyridin-5-yl)methanol (P15a)

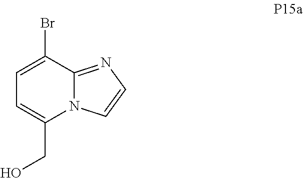 [SMILES: OCc1ccc(Br)c2nccn12]

To a solution of methyl 8-bromoimidazo[1,2-a]pyridine-5-carboxylate (3.0 g, 12 mmol; prepared as described in WO02011/075591) in EtOH (30 mL) was added NaBH₄ (1.3 g, 35 mmol) at rt. The mixture was stirred at rt for 12 h, quenched with 1N HCl (10 mL) and concentrated. The residue was neutralized with sat. K₂CO₃ to adjust the pH to approx. 8. The mixture was extracted with DCM/MeOH (3×50 mL, 10:1). The combined organic layer was concentrated and purified by FCC (PE:EA=2:1 to 0:1) to give compound P15a as a white solid.

### Step 2: Mixture of 8-bromo-5-(chloromethyl)imidazo[1,2-a]pyridine and (8-bromoimidazo[1,2-a]pyridin-5-yl)methyl methanesulfonate (P15b)

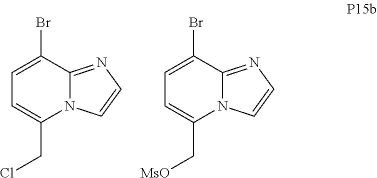 [SMILES: ClCc1ccc(Br)c2nccn12 ; CS(=O)(=O)OCc1ccc(Br)c2nccn12]

To a solution of compound P15a (1.3 g, 5.7 mmol) in DCM (30 mL) was added Et₃N (1.7 g, 17 mmol) and MsCl (786 mg, 6.9 mmol) at 0° C. The mixture was stirred for 3 h at rt and then diluted with water. The organic layer was dried over Na₂SO₄, filtered and concentrated to give mixture P15b as a white solid.

### Step 3: tert-Butyl ((2-methylnaphthalen-1-yl)methyl)carbamate (P15c)

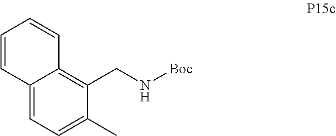 [SMILES: Cc1ccc2ccccc2c1CNC(=O)OC(C)(C)C]

A solution of (2-methylnaphthalen-1-yl)methanamine (2.4 g, 14 mmol), Boc₂O (3.0 g, 14 mmol) and TEA (2.8 g, 28 mmol) in DCM (50 mL) was stirred at rt for 2 h. The mixture was washed with water and brine. The organic layer was dried over Na₂SO₄, filtered, concentrated and purified by FCC (PE:EA=50:1 to 10:1) to give compound P15c as a yellow oil.

### Step 4: tert-Butyl ((2-methylnaphthalen-1-yl)methyl)((5-(trifluoromethyl)furan-2-yl)methyl)carbamate (P15d)

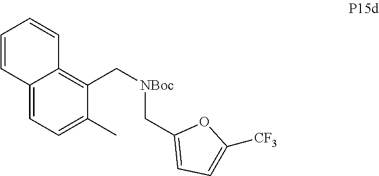 [SMILES: Cc1ccc2ccccc2c1CN(Cc1ccc(C(F)(F)F)o1)C(=O)OC(C)(C)C]

To a solution of compound P15c (2.2 g, 8.1 mmol) in dry DMF (25 mL) was added NaH (324 mg, 60%, 8.9 mmol) under ice-bath cooling. The mixture was stirred for 30 min at 0° C. To the solution was added 2-(bromomethyl)-5-(trifluoromethyl)furan (2.0 g, 8.9 mmol) and the mixture was stirred for 3 h at rt, poured into ice water and extracted with EA (3×50 mL). The combined organic layer was washed with water (3×100 mL) and brine (100 mL), dried over Na₂SO₄, filtered, concentrated and purified by FCC (PE:EA=20:1 to 5:1) to give compound P15d as a yellow oil.

### Step 5: 1-(2-Methylnaphthalen-1-yl)-N-((5-(trifluoromethyl)furan-2-yl)methyl)methanamine (P15e)

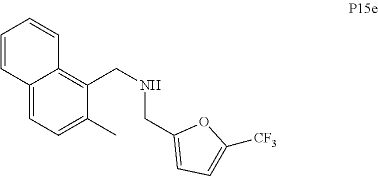 [SMILES: Cc1ccc2ccccc2c1CNCc1ccc(C(F)(F)F)o1]

To a solution of compound P15d (3.5 g, 8.3 mmol) in DCM (20 mL) was added TFA (4.7 g, 42 mmol) at rt. The mixture was stirred at rt for 4 h and adjusted to pH=11 with sat. Na₂CO₃. The organic layer was washed with brine, dried over Na₂SO₄, filtered and concentrated to give compound P15e as a yellow oil.

### Step 6: 1-(2-Methylnaphthalen-yl)-N-((5-trifluoromethyl)furan-2-yl)methyl)methanamine (P15)

The suspension of compound P15e (1.0 g, 3.1 mmol), mixture P15b (0.8 g), K₂CO₃ (0.9 g, 6.5 mmol) and KI (0.54 g, 3.2 mmol) in ACN (100 mL) was stirred at 80° C. overnight, cooled, filtered, concentrated and purified by FCC (PE:EA=3:1 to 1:1) to give compound P15 as a white solid.

### Preparative Example P16

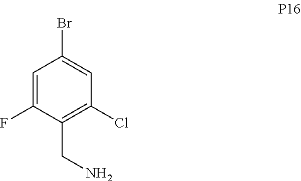 [SMILES: NCc1c(F)cc(Br)cc1Cl]

### Step 1: 2-(Azidomethyl)-5-bromo-1-chloro-3-fluorobenzene (P16a

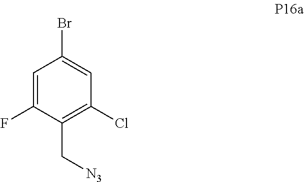 [SMILES: [N-]=[N+]=NCc1c(F)cc(Br)cc1Cl]

To a solution of 5-bromo-2-(bromomethyl)-1-chloro-3-fluorobenzene (1.0 g, 3.3 mmol) in DMF (30 mL) was added NaN₃ (0.26 g, 4.0 mmol) at 0° C. The mixture was stirred at rt overnight, diluted with water (100 mL) and extracted with EA (3×70 mL). The combined organic layer was washed with H₂O (2×70 mL) and brine (70 mL), dried over Na₂SO₄, filtered and concentrated to give compound P16a as a colorless oil.

### Step 2: (4-Bromo-2-chloro-6-fluorophenyl)methanamine (P16)

A suspension of compound P16a (800 mg, 2.6 mmol) and PPh₃ (1.4 g, 5.2 mmol) in H₂O/THF (15 mL/15 mL) was stirred overnight at rt, adjusted to pH=4 with aq. HCl, diluted with water (50 mL) and extracted with EA (3×70 mL). To the aq. layer was added Na₂CO₃ to adjust pH=10 and then extracted with EA (2×70 mL). The combined organic layer was dried over Na₂SO₄, filtered and concentrated to afford compound P16 as a yellow oil.

### Preparative Example P17

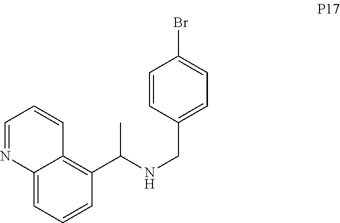 [SMILES: CC(NCc1ccc(Br)cc1)c1cccc2ncccc12]

### N-(4-Bromobenzyl)-1-(quinolin-5-yl)ethan-1-amine (P17)

To a solution of 1-(quinolin-5-yl)ethan-1-one (171 mg, 1.00 mmol) and 4-bromobenzylamine (0.28 g, 1.5 mmol) in THF (10 mL) was added Ti(i-PrO)₄ (852 mg, 3.00 mmol) at rt. The mixture was stirred at 100° C. for 3 h under microwave irradiation. To the mixture was added NaBH₄ (114 mg, 3.00 mmol) at rt and then the mixture was stirred 50° C. for 5 h, diluted with water (50 mL) and extracted with EA (3×50 mL). The combined organic layer was washed with water (2×100 mL) and brine (100 mL), dried over Na₂SO₄, filtered, concentrated and purified by FCC (PE:EA=4:1) to give compound P17 as a yellow oil.

### Preparative Example P18

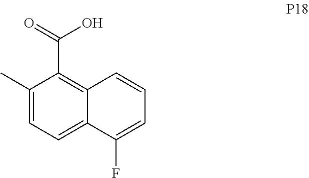

### 5-Fluoro-2-methyl-1-naphthoic acid P18)

To a stirred solution of 1-bromo-5-fluoro-2-methylnaphthalene (500 mg, 2.10 mmol) in THF (30 mL) was added n-butyl lithium (2.5M, 0.9 mL, 2.25 mmol) at −78° C. dropwise and the mixture was stirred for 2 h, then solid CO₂ (2.00 g) was added and stirred at −78° C. for 1 h and then at rt for 16 h. The mixture was quenched with water (2 mL) and the obtained solid was filtered. The solid was triturated with diethyl ether/n-pentane (10 mL/10 mL) and the solid was dried under vacuum to afford P18 as a white solid. ¹H-NMR (500 MHz, DMSO-d₆) δ: 13.67 (s, 1H), 8.05 (d, J=8.5 Hz, 1H), 7.65 (d, J=8.5 Hz, 1H), 7.59-7.53 (m, 2H), 7.35 (dd, J=10.5, 2.5 Hz, 1H), 2.50 (s, 3H).

### Preparative Example P18/1

The following Preparative Example was prepared similar as described for Preparative Example P18 using the appropriate building block.

| # | building block | structure |
|---|---|---|
| P18/1 | 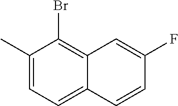 | 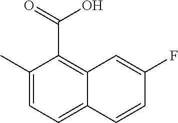 |

### Preparative Example P19

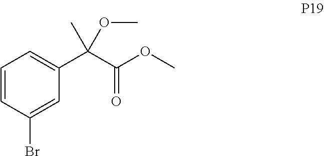

### Methyl 2-(3-bromophenyl)-2-methoxypropanoate (P19)

To a solution of methyl 2-(3-bromophenyl)-2-hydroxypropanoate (130 mg, 0.50 mmol) in THF (10 mL) and K₂CO₃ (276 mg, 2.00 mmol) was added MeI (284 mg, 2.00 mmol) and the mixture was stirred at rt for 4 h, diluted with water (20 mL) and extracted with EA (3×20 mL). The combined organic layer was washed with brine (30 mL), dried over Na₂SO₄, filtered and concentrated to give P19 as a colorless oil.

### Preparative Example P20

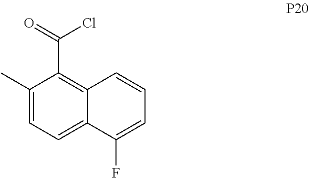

### 5-Fluoro-2-methyl-1-naphthoyl chloride (P20)

To a solution of compound P18 (204 mg, 1.00 mmol) in DCM (10 mL) was added SOCl₂ (1 mL) and the mixture was stirred at rt for 2 h and concentrated to give compound P20 as a yellow oil.

### Preparative Example P20/1

The following Preparative Example was prepared similar as described for Preparative Example P20 using the appropriate building block.

| # | building blocks | structure |
|---|---|---|
| P20/1 | 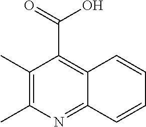 | 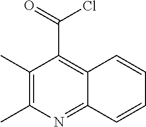 |

### Preparative Example P21

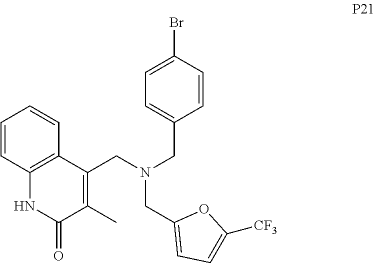 [SMILES: Cc1c(CN(Cc2ccc(Br)cc2)Cc2ccc(C(F)(F)F)o2)c2ccccc2[nH]c1=O]

### Step 1: Methyl 3-methyl-2-oxo-1,2-dihydroquinoline-4-carboxylate (P21a)

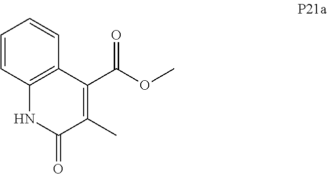 [SMILES: COC(=O)c1c(C)c(=O)[nH]c2ccccc12]

To a mixture of 3-methyl-2-oxo-1,2-dihydroquinoline-4-carboxylic acid (1.00 g, 5.00 mmol) in DMF (10 mL) was added Cs₂CO₃ (3.26 g, 10.0 mmol) and iodomethane (923 mg, 6.50 mmol).

The mixture was stirred at rt overnight, diluted with water (50 mL) and extracted with EA (3×30 mL). The combined organic layer was washed with brine (30 mL), dried over Na₂SO₄, filtered, concentrated and purified by FCC (PE:EA=5:1) to give compound P21a as a white solid.

### Step 2: 4-(Hydroxymethyl)-3-methylquinolin-2(1H)-one (P21b)

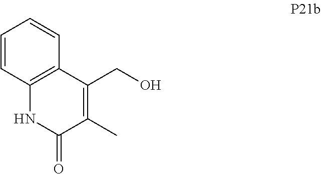

To a mixture of compound P21a (1.00 g, 4.65 mmol) in methanol (10 mL) was added NaBH₄ (532 mg, 14.0 mmol) at 0° C. and the mixture was stirred for 3 h, diluted with water (50 mL) and extracted with EA (3×30 mL). The combined organic layer was washed with brine (30 mL), dried over Na₂SO₄, filtered, concentrated and purified by FCC (PE:EA=2:1) to give compound P21b as a white solid.

### Step 3: 3-Methyl-2-oxo-1,2-dihydroquinoline-4-carbaldehyde (P21c)

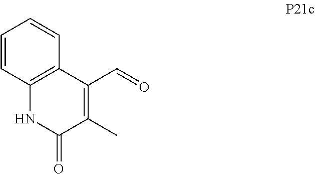

To a mixture of compound P21b (400 mg, 2.10 mmol) in acetone (30 mL) was added IBX (2.40 g, 8.40 mmol) and the mixture was stirred at 50° C. for 12 h and then filtered. The filtrate was concentrated and purified by FCC (PE:EA=4:1) to give compound P21c as a yellow solid.

### Step 4: 4-(((4-Bromobenzyl)((5-(trifluoromethyl)furan-2-yl)methyl)amino)methyl)-3-methyl-quinolin-2(1H-one (P21)

To a solution of compound P21c (300 mg, 1.60 mmol) in 1,2-dichloroethane (10 mL) was added N-(4-bromobenzyl)-1-(5-(trifluoromethyl)furan-2-yl)methanamine (534 mg, 1.60 mmol) and one drop AcOH. The mixture was stirred at rt for 0.5 h, then NaBH(OAc)₃ (1.78 g, 8.00 mmol) was added and the mixture was stirred at rt overnight, diluted with water (40 mL) and extracted with DCM (3×20 mL). The combined organic layer was washed with brine (30 mL), dried over Na₂SO₄, filtered, concentrated and purified by FCC (PE:EA=5:1) to give compound P21 as a colorless oil.

### Preparative Example P22

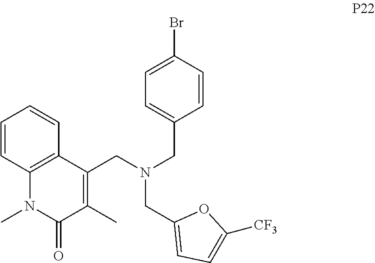 [SMILES: Cc1c(CN(Cc2ccc(Br)cc2)Cc2ccc(C(F)(F)F)o2)c2ccccc2n(C)c1=O]

### 4-(((4-Bromobenzyl)((5-(trifluoromethyl)furan-2-yl)methyl)amino)methyl)-1,3-dimethylquinolin-2(1H)-one (P22

To a mixture of compound P21 (200 mg, 0.40 mmol) in DMF (10 mL) was added Cs₂CO₃ (260 mg, 0.80 mmol) and iodomethane (86 mg, 0.60 mmol). The mixture was stirred at rt overnight, diluted with water (50 mL) and extracted with EA (3×30 mL). The combined organic layer was washed with brine (30 mL), dried over Na₂SO₄, filtered, concentrated and purified by FCC (PE:EA=5:1) to give compound P22 as a white solid.

### Preparative Example P23

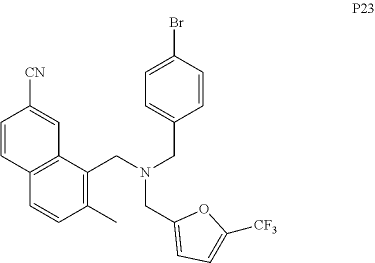 [SMILES: Cc1ccc2ccc(C#N)cc2c1CN(Cc1ccc(Br)cc1)Cc1ccc(C(F)(F)F)o1]

### 8-(((4-Bromobenzyl)((5-(trifluoromethyl)furan-2-yl)methyl)amino)methyl)-7-methyl-2-naphthonitrile (P23)

To a solution of 8-(((4-bromobenzyl)((5-(trifluoromethyl)furan-2-yl)methyl)amino)methyl)-7-methyl-2-naphthamide (intermediate from Example 27/25; 300 mg, 0.57 mmol) in DCM (10 mL) was added TFAA (359 mg, 1.71 mmol). The mixture was stirred at rt for 4 h, diluted with water (50 mL) and extracted with DCM (3×30 mL). The combined organic layer was washed with brine (30 mL), dried over Na₂SO₄, filtered, concentrated and purified by FCC (PE:EA=10:1) to give compound P23 as a colorless oil.

### Preparative Example P24

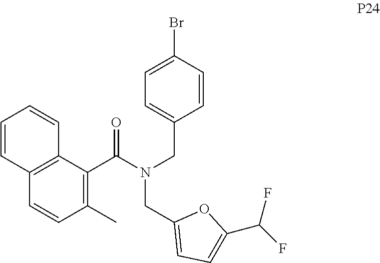 [SMILES: Cc1ccc2ccccc2c1C(=O)N(Cc1ccc(Br)cc1)Cc1ccc(C(F)F)o1]

### Step 1: (5-Formylfuran-2-yl)methyl methanesulfonate (P24a)

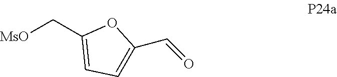 [SMILES: CS(=O)(=O)OCc1ccc(C=O)o1]

To a solution of 5-(hydroxymethyl)furan-2-carbaldehyde (10 g, 79 mmol) in DCM (150 mL) was added pyridine (12 g, 105 mmol) and a solution of MsCl (10 g, 88 mmol) in DCM (10 mL) at 0° C. The mixture was stirred at rt for 12 h, diluted with 1N HCl (200 mL) and extracted with DCM (200 mL). The organic layer was washed with brine, dried over Na₂SO₄, filtered, concentrated and purified by FCC (PE:EA=5:1) to give compound P24a as a yellow oil.

### Step 2: 5-(((4-Bromobenzyl)amino)methyl)furan-2-carbaldehyde (P24b

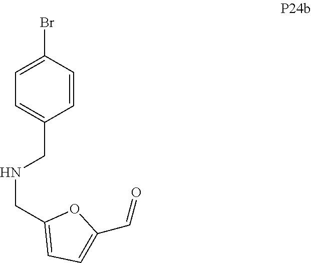 [SMILES: O=Cc1ccc(CNCc2ccc(Br)cc2)o1]

To a solution of (4-bromophenyl)methanamine (2.4 g, 13 mmol) in CH₃CN (125 mL) was added K₂CO₃ (1.8 g, 13 mmol) and compound P24a (1.0 g, 5.1 mmol) at rt. The mixture was stirred at 85° C. for 2 h and filtered. The filtrate was concentrated and purified by FCC (PE:EA=3:1) to give compound P24b as a yellow oil.

### Step 3: N-(4-Bromobenzyl)-N-((5-formylfuran-2-yl)methyl)-2-methyl-1-naphthamide (P24c)

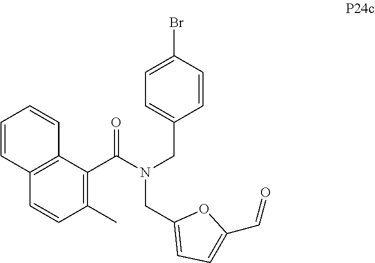 [SMILES: Cc1ccc2ccccc2c1C(=O)N(Cc1ccc(Br)cc1)Cc1ccc(C=O)o1]

To a solution of compound P24b (720 mg, 2.50 mmol) in CH₂Cl₂ (15 mL) was added Et₃N (757 mg, 7.50 mmol) and 2-methyl-1-naphthoyl chloride (523 mg, 2.57 mmol) under ice-bath cooling. The mixture was stirred at rt overnight, concentrated and purified by FCC (PE:EA=20:1 to 3:1) to give compound P24c as a white solid.

### Step 4: N-(4-Bromobenzyl)-N-((5-(difluoromethyl)furan-2-yl)methyl)-2-methyl-1-naphthamide (P24)

To a solution of compound P24c (500 mg, 1.08 mmol) in CH₂Cl₂ (20 mL) was added DAST (1 mL) at 0° C. The mixture was stirred at 0° C. for 30 min and then stirred at rt for 12 h, quenched with sat. NaHCO₃ (20 mL) and extracted with DCM. The organic layer was washed with brine, dried over Na₂SO₄, filtered, concentrated and purified by FCC (PE:EA=20:1 to 3:1) to give compound P24 as a white solid.

### Preparative Example P25

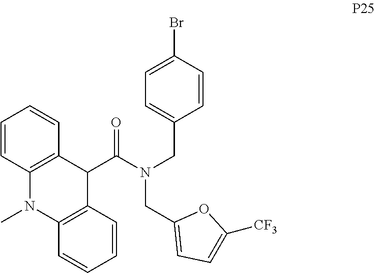 [SMILES: CN1c2ccccc2C(C(=O)N(Cc2ccc(Br)cc2)Cc2ccc(C(F)(F)F)o2)c2ccccc21]

### Step 1: Acridine-9-carbonyl chloride (P25a)

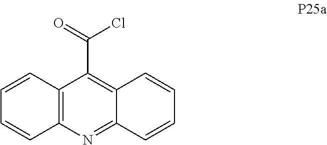 [SMILES: O=C(Cl)c1c2ccccc2nc2ccccc12]

To a solution of acridine-9-carboxylic acid (223 mg, 1.00 mmol) in DCM (10 mL) was added SOCl₂ (1 mL). The mixture was stirred at rt for 2 h and concentrated to give compound P25a as a yellow oil.

### Step 2: N-(4-Bromobenzyl)-N-((5-(trifluoromethyl)furan-2-yl)methyl)acridine-9-carboxamide (P25b

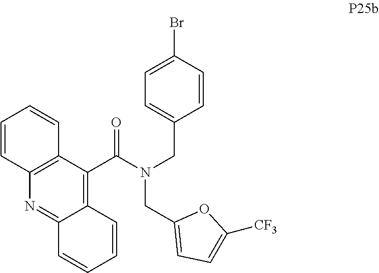 [SMILES: O=C(c1c2ccccc2nc2ccccc12)N(Cc1ccc(Br)cc1)Cc1ccc(C(F)(F)F)o1]

To a solution of the compound P25a (333 mg, 1.00 mmol) in DCM (5 mL) was added compound 3a (241 mg, 1.00 mmol) and Et₃N (113 mg, 1.10 mmol) and the mixture was stirred at rt for 12 h, diluted with water (50 mL) and extracted with DCM (3×20 mL). The combined organic layer was washed with brine (30 mL), dried over Na₂SO₄, filtered, concentrated and purified by FCC (PE:EA=3:1) to give compound P25b as a colorless oil

### Step 3: 9-((4-Bromobenzyl)((5-(trifluoromethyl)furan-2-yl)methyl)carbamol)-10-methylacridin-10-ium trifluoromethanesulfonate (P25c)

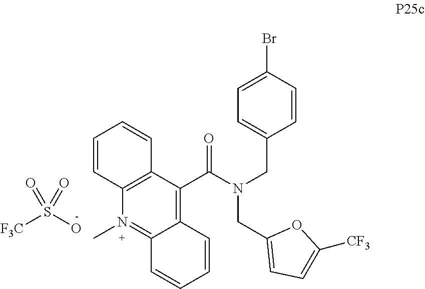

To a solution of the compound P25b (450 mg, 0.84 mmol) in DCM (10 mL) was added methyl trifluoromethanesulfonate (274 mg, 1.67 mmol). The mixture was stirred at rt for 24 h and concentrated to give compound P25c as a brown oil.

### Step 4: N-(4-Bromobenzyl)-10-methyl-N-((5-(trifluoromethyl)furan-2-yl)methyl)-9,10-dihydro-acridine-9-carboxamide (P25)

To a solution of the compound P25c (500 mg crude, 0.84 mmol) in EtOH (20 mL) was added NH₄Cl (180 mg, 3.36 mmol) and Zn (180 mg, 3.36 mmol) and the mixture was stirred at 80° C. for 30 min, filtered and the filtrate concentrated. The crude material was purified by FCC (PE:EA=3:1) to give compound P25 as a colorless oil.

### Preparative Example P26

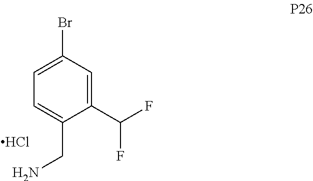 [SMILES: Cl ; NCc1ccc(Br)cc1C(F)F]

### Step 1: 4-Bromo-2-(difluoromethyl)benzonitrile (P26a)

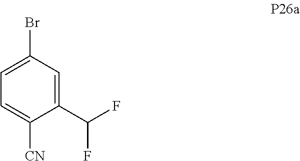 [SMILES: N#Cc1ccc(Br)cc1C(F)F]

To a solution of 4-bromo-2-formylbenzonitrile (3.5 g, 16 mmol) in DCM (35 mL) was added DAST (3.5 mL) at 0° C. The mixture was stirred at 0° C. for 30 min and then stirred at rt for 12 h, carefully quenched with aq. NaHCO₃ (50 mL) and extracted with DCM (3×50 mL). The combined organic layer was washed with brine (100 mL), dried over Na₂SO₄, concentrated and purified by FCC (PE:EA=5:1) to give compound P26a as a white solid.

### Step 2: tert-Butyl (4-bromo-2-(difluoromethyl)benzyl)carbamate (P26b)

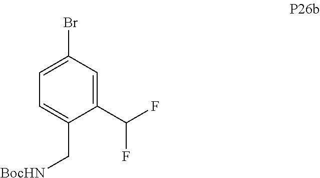 [SMILES: CC(C)(C)OC(=O)NCc1ccc(Br)cc1C(F)F]

To a solution of compound P26a (4.1 g, 17 mmol) in MeOH (100 mL) was added Boc₂O (7.8 g, 34 mmol) and NiCl₂.6H₂O (0.24 g, 1.0 mmol) at 0° C., followed by careful portionwise addition of NaBH₄ (3.8 g, 102 mmol). The resulting black mixture was stirred at 0° C. for 20 min. Then the ice bath was removed and the mixture was stirred at rt for 12 h, carefully quenched with H₂O (50 mL) and extracted with EA (3×50 mL). The combined organic layer was washed with brine (100 mL), dried over Na₂SO₄, concentrated and purified by FCC (PE:EA=5:1) to give compound P26b as a white solid.

### Step 3: (4-Bromo-2-(difluoromethyl)phenyl)methanamine hydrochloride (P26)

To a solution of compound P26b (4.8 g, 14 mmol) in EA (10 mL) was added HCl/EA (50 mL) at 0° C. The mixture was stirred at rt for 12 h and concentrated to give crude compound P26 as a white solid.

### Preparative Example P26/1 to P26/2

The following Preparative Examples were prepared similar as described for Preparative Example P26, Step 2 and 3, using the appropriate building block.

| # | building block | structure |
|---|---|---|
| P26/1 | 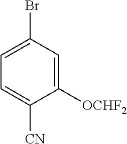 [SMILES: N#Cc1ccc(Br)cc1OC(F)F] | 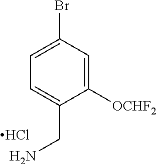 [SMILES: Cl ; NCc1ccc(Br)cc1OC(F)F] |
| P26/2 | 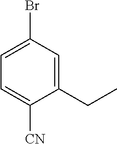 | 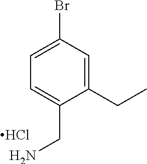 |

### Preparative Example P27

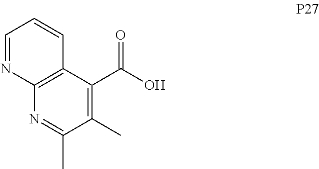

### Step 1: 1H-Pyrrolo[2,3-b]pyridine-2,3-dione (P27a)

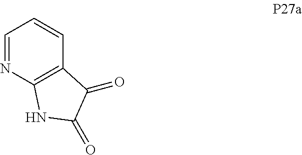 [SMILES: O=C1Nc2ncccc2C1=O]

PCC (45.7 g, 212 mmol) was compounded with silica gel (45.7 g, 100-200 mesh) and transferred to a 1-L round-bottom flask containing DCE (400 mL). To the resulting orange suspension was added a solution of 1H-pyrrolo[2,3-b]pyridine (10.0 g, 84.7 mmol) in DCE (50 mL) and AlCl₃ (1.5 g, 11 mmol). The mixture was stirred at 80° C. for 3 h, cooled to rt, filtered and the filter cake was washed with EA. The filtrate was concentrated and purified by FCC (PE:EA=5:1) to give compound P27a as a yellow solid.

### Step 2: 2,3-Dimethyl-1,8-naphthyridine-4-carboxylic acid (P27)

To a solution of compound P27a (700 mg, 4.7 mmol) in EtOH (10 mL) and H₂O (10 mL) was added KOH (795 mg, 14.2 mmol) and butan-2-one (680 mg, 9.5 mmol). The mixture was stirred at 80° C. overnight. The EtOH was removed in vacuo and the aq. layer was adjusted to pH=3-4 with 1N HCl. The resulting mixture was lyophilisized to give crude compound P27, which was used directly in the next step without further purification.

### Preparative Example P27/1 to P27/3

The following Preparative Examples were prepared similar as described for Preparative Example P27, Step 2, using the appropriate building block.

| # | building blocks | structure |
|---|---|---|
| P27/1 | 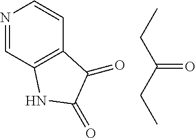 | 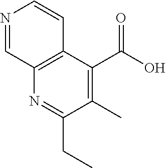 [SMILES: CCc1nc2cnccc2c(C(=O)O)c1C] |
| P27/2 | 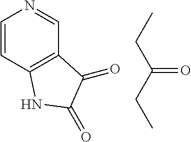 | 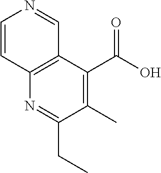 [SMILES: CCc1nc2ccncc2c(C(=O)O)c1C] |
| P27/3 | 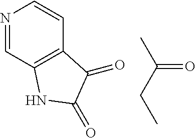 | 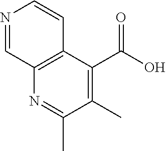 |

### Preparative Example P28

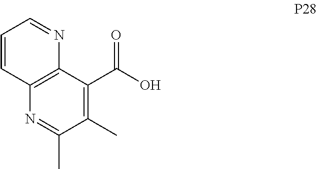

### Step 1: tert-Butyl (2-bromopyridin-3-yl)carbamate (P28a)

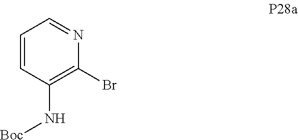 [SMILES: CC(C)(C)OC(=O)Nc1cccnc1Br]

A solution of 2-bromopyridin-3-amine (10 g, 58 mmol) in Boc₂O (100 mL) was stirred at 100° C. overnight, cooled to rt, diluted with water (20 mL) and extracted with EA (3×15 mL). The combined organic layer was dried over Na₂SO₄, concentrated and purified by FCC (PE:EA=20:1) to give compound P28a as a white solid.

### Step 2: Ethyl 2-(3-((tert-butoxycarbonyl)amino)pyridin-2-yl)-2-oxoacetate (P28b)

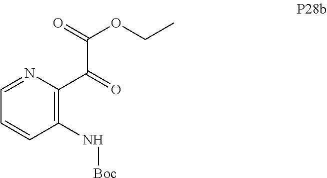 [SMILES: CCOC(=O)C(=O)c1ncccc1NC(=O)OC(C)(C)C]

To a solution of compound P28a (8.0 g, 29 mmol) in dry THF (60 mL) was added dropwise n-BuLi (29 mL of 2.5M solution in hexane) at −78° C. The mixture was allowed to warm to −20° C. for 2 h. After diethyl oxalate (8.5 mL, 62 mmol) was added dropwise to the mixture at −78° C., the mixture was stirred at rt for 2 h, quenched by NH₄Cl (50 mL) and extracted with EA (3×50 mL). The combined organic layer was washed with brine (2×20 mL), dried over Na₂SO₄, filtered, concentrated and purified by FCC (PE:EA=20:1) to give compound P28b as a white solid.

### Step 3: 2,3-Dimethyl-1,5-naphthyridine-4-carboxylic acid (P28)

To a solution of compound P28b (3.0 g, 10 mmol) in EtOH (50 mL) and H₂O (20 mL) was added KOH (1.7 g, 31 mmol) and butan-2-one (2.9 g, 41 mmol). The mixture was stirred at 80° C. overnight. Then the EtOH was removed in vacuo and the aq. layer was adjusted to pH=3-4 with 1N HCl. The resulting mixture was lyophilisized to give crude compound P28, which was used directly in the next step without further purification.

### Preparative Example P28/1

The following Preparative Example was prepared similar as described for Preparative Example P28, using the appropriate building blocks.

| # | building block(s) | structure |
|---|---|---|
| P28/1 | 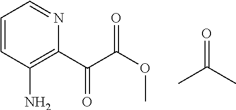 | 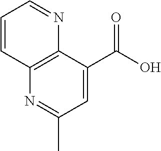 |
| P28/2 | 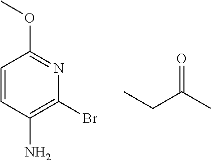 | 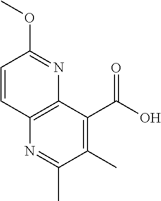 [SMILES: COc1ccc2nc(C)c(C)c(C(=O)O)c2n1] |

### Preparative Example P29

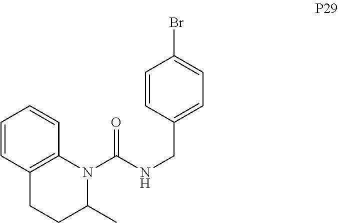 [SMILES: CC1CCc2ccccc2N1C(=O)NCc1ccc(Br)cc1]

### N-(4-Bromobenzyl)-2-methyl-3,4-dihydroquinoline-1(2H)-carboxamide (P29)

To a solution of 2-methyl-1,2,3,4-tetrahydroquinoline (147 mg, 1.00 mmol) in THF (10 mL) was added 1-bromo-4-(isocyanatomethyl)benzene (211 mg, 1.00 mmol). The mixture was stirred at rt for 2 h and concentrated to give compound P29 as a yellow oil.

### Preparative Example P30

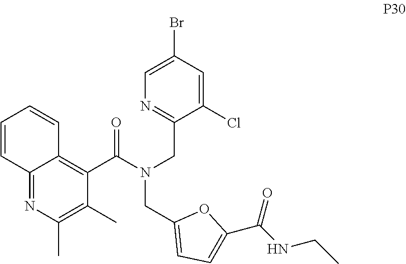 [SMILES: CCNC(=O)c1ccc(CN(Cc2ncc(Br)cc2Cl)C(=O)c2c(C)c(C)nc3ccccc23)o1]

### Step 1: Ethyl 5-((((5-bromo-3-chloropyridin-2-yl)methyl)amino)methyl)furan-2-carboxylate (P30a)

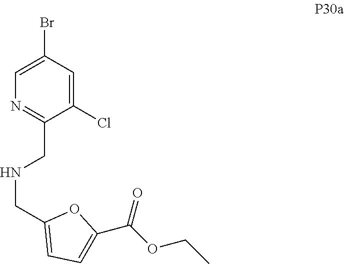 [SMILES: CCOC(=O)c1ccc(CNCc2ncc(Br)cc2Cl)o1]

To a solution of (5-bromo-3-chloropyridin-2-yl)methanamine hydrochloride (1.00 g, 3.90 mmol) in EtOH (50 mL) and DMF (10 mL) was added Et₃N (788 mg, 7.80 mmol) and ethyl 5-(chloromethyl)furan-2-carboxylate (733 mg, 3.90 mmol) at 0° C. and the mixture was stirred at 0° C. for 4 h, diluted with water (100 mL) and extracted with EA (3×30 mL). The combined organic layer was washed with brine (30 mL), dried over Na₂SO₄, filtered, concentrated and purified by FCC (PE:EA=2:1) to give compound P30a as a colorless oil.

### Step 2: Ethyl 5-((N-((5-bromo-3-chloropyridin-2-yl)methyl)-2,3-dimethylquinoline-4-carboxamido)methyl)furan-2-carboxylate (P30b)

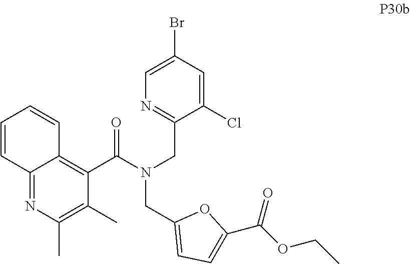 [SMILES: CCOC(=O)c1ccc(CN(Cc2ncc(Br)cc2Cl)C(=O)c2c(C)c(C)nc3ccccc23)o1]

To a solution of compound P30a (745 mg, 2.00 mmol) in DCM (10 mL) was added compound P20/1 (438 mg, 2.00 mmol) and Et₃N (226 mg, 2.20 mmol) and the mixture was stirred at rt for 12 h, diluted with water (50 mL) and extracted with DCM (3×20 mL). The combined organic layer was washed with brine (30 mL), dried over Na₂SO₄, filtered, concentrated and purified by FCC (PE:EA=3:1) to give compound P30b as a colorless oil.

### Step 3: 5-((N-((5-Bromo-3-chloropyridin-2-ylmethyl-2,3-dimethylquinoline-4-carboxamido)methyl)furan-2-carboxylic acid (P30c)

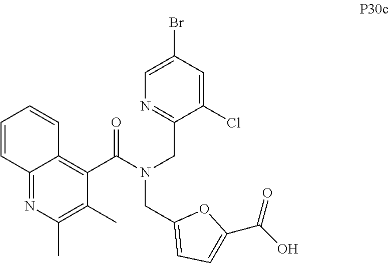 [SMILES: Cc1nc2ccccc2c(C(=O)N(Cc2ccc(C(=O)O)o2)Cc2ncc(Br)cc2Cl)c1C]

To a mixture of compound P30b (555 mg, 1.00 mmol) in MeOH (5 mL) and THF (5 mL) was added LiOH (2M, 2 mL) and the mixture was stirred at rt overnight, neutralized with 1N HCl and extracted with EA (3×). The combined organic layer was washed with brine, dried over Na₂SO₄, filtered and concentrated to give compound P30c as a colorless oil.

### Step 4: N-((5-Bromo-3-chloropyridin-2-yl)methyl)-N-((5-(ethylcarbamoyl)furan-2-yl)methyl)-2,3-dimethylquinoline-4-carboxamide (P30)

To a mixture of compound P30c (210 mg, 0.40 mmol) in DMF (5 mL) was added HOBt (58 mg, 0.40 mmol), EDCI*HCl (152 mg, 0.80 mmol), DIPEA (155 mg, 1.20 mmol) and ethanamine hydrochloride (49 mg, 0.60 mmol). The mixture was stirred at rt for 12 h, diluted with water (50 mL) and extracted with EA (3×30 mL). The combined organic layer was washed with brine (30 mL), dried over Na₂SO₄, filtered, concentrated and purified by FCC (PE:EA=1:1) to give compound P30 as a colorless oil.

### Preparative Example P30/1 to P30/3

The following Preparative Examples were prepared similar as described for Preparative Example P30, using the appropriate building block.

| # | building block(s) | structure |
|---|---|---|
| P30/1 | 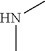 | 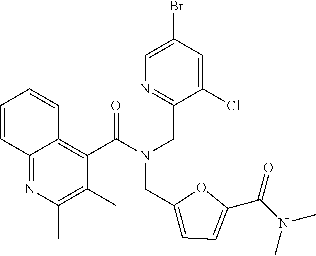 [SMILES: Cc1nc2ccccc2c(C(=O)N(Cc2ccc(C(=O)N(C)C)o2)Cc2ncc(Br)cc2Cl)c1C] |
| P30/2 | 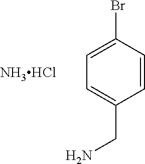 | 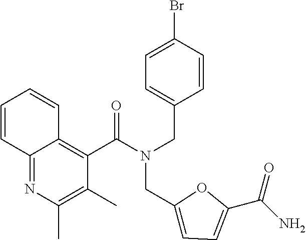 [SMILES: Cc1nc2ccccc2c(C(=O)N(Cc2ccc(Br)cc2)Cc2ccc(C(N)=O)o2)c1C] |
| P30/3 | 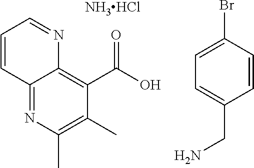 | 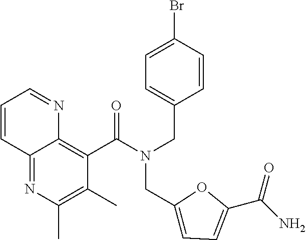 [SMILES: Cc1nc2cccnc2c(C(=O)N(Cc2ccc(Br)cc2)Cc2ccc(C(N)=O)o2)c1C] |

### Preparative Example P31

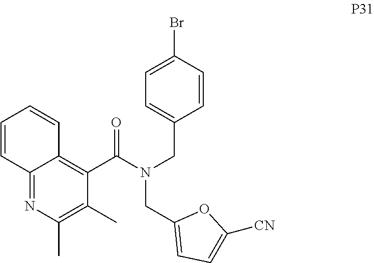 [SMILES: Cc1nc2ccccc2c(C(=O)N(Cc2ccc(Br)cc2)Cc2ccc(C#N)o2)c1C]

### N-(4-Bromobenzyl)-N-((5-cyanofuran-2-yl)methyl)-2,3-dimethylquinoline-4-carboxamide (P31)

To a solution of compound P30/2 (375 mg, 0.76 mmol) in CH₂Cl₂ (20 mL) and pyridine (2 mL) was added POCl₃ (1 mL) at 0° C. The mixture was stirred at 0° C. for 30 min and for 1 h at rt, quenched with aq. NaHCO₃ at 0° C., stirred for 15 min and extracted with EA (3×20 mL). The combined organic layer was dried over Na₂SO₄, filtered and concentrated to give compound P31 as a brown solid, which was directly used in the next step without further purification.

### Preparative Example P31/1

The following Preparative Example was prepared similar as described for Preparative Example P31, using the appropriate building block.

| # | building block | structure |
|---|---|---|
| P31/1 | 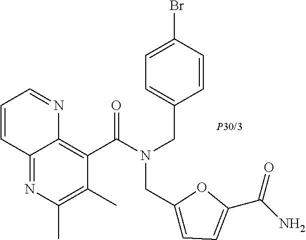 [SMILES: Cc1nc2cccnc2c(C(=O)N(Cc2ccc(Br)cc2)Cc2ccc(C(N)=O)o2)c1C] | 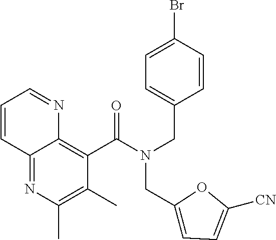 [SMILES: Cc1nc2cccnc2c(C(=O)N(Cc2ccc(Br)cc2)Cc2ccc(C#N)o2)c1C] |

### Preparative Example P32

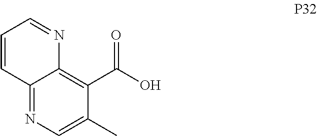

### 3-Methyl-1,5-naphthyridine-4-carboxylic acid (P32)

To a solution of compound ethyl 2-(3-aminopyridin-2-yl)-2-oxoacetate (2.00 g, 10.3 mmol) in sat. aq. KOH solution (30 mL) was added propionaldehyde oxime (3.80 g, 51.5 mmol) at rt and the mixture was stirred at 70° C. for 12 h, cooled to rt, adjusted to pH=5 with conc. HCl and extracted with EA (3×30 mL). The combined organic layer was dried over Na₂SO₄, filtered and concentrated to give compound P32 as a black solid, which was used in the next step without further purification.

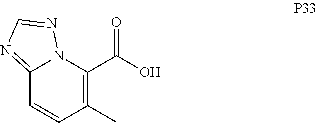

### Preparative Example P33

### Step 1: (E)-M-(6-Bromo-5-methylpyridin-2-yl)-N-dimethylformimidamide (P33a)

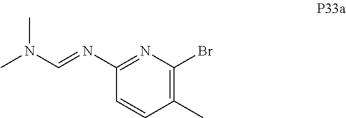

To a solution of 6-bromo-5-methylpyridin-2-amine (2.50 g, 13.4 mmol) in i-PrOH (25 mL) was added dimethylformamid-dimethylacetal (2.23 g, 18.7 mmol). The solution was stirred at 85° C. for 3 h under Ar, cooled to rt and used directly in the next step without further purification.

### Step 2: (E)-N-(6-Bromo-5-methylpyridin-2-yl)-N-hydoxyformimidamide hydrochloride (P33b)

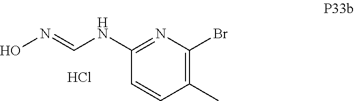

To a solution of compound P33a in i-PrOH (25 mL) was added NH₂OH.HCl (1.3 g, 19 mmol). The solution was stirred at 50° C. overnight and cooled to rt. The solid was collected by suction, washed with i-PrOH and dried to give compound P33b as a white solid.

### Step 3: 5-Bromo-6-methyl-[1,2,4]triazolo[1,5-a]pyridine (P33c)

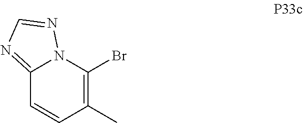

To a solution of compound P33b (2.46 g, 10.7 mmol) in THF (100 mL) was added TFAA (2.25 g, 10.7 mmol) dropwise at 0° C., then the mixture was allowed to warm to rt slowly and stirred overnight, quenched by aq. NaHCO₃ to adjust pH=8 and extracted with EA (2×100 mL). The combined organic layer was washed with brine, dried over Na₂SO₄, filtered, concentrated and purified by FCC (PE:EA=3:2 to 1:1) to give compound P33c as a white solid.

### Step 4: Methyl 6-methyl-[1,2,4]triazolo[1,5-a]pyridine-5-carboxylate (P33d)

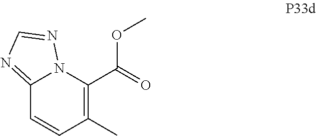

To a solution of compound P33c (790 mg, 3.72 mmol) in MeOH (60 mL) and DMF (30 mL) was added Pd(dppf)Cl₂ (1.09 g, 1.49 mmol) and Et₃N (1.60 mL, 11 mmol). The mixture was stirred at 55° C. under a CO atmosphere overnight, cooled, diluted with water (100 mL) and extracted with EA (2×50 mL). The combined organic layer was washed with brine (30 mL), dried over Na₂SO₄, filtered, concentrated and purified by FCC (PE:EA=1:1) to give compound P33d as a white solid.

### Step 5: 6-Methyl-[1,2,4]triazolo[1,5-a]pyridine-5-carboxylic acid (P33)

To a solution of compound P33d (240 mg, 1.25 mmol) in CH₃OH (10 mL), H₂O (5 mL) and THF (10 mL) was added LiOH.H₂O (260 mg, 6.28 mmol). The mixture was stirred at rt overnight, adjusted to pH=3-4 with 1N HCl and evaporated to give a solid, which was stirred in DCM and MeOH (55 mL, 10:1) for 15 min, filtered and concentrated to give crude compound P33 as a white solid, which was used in the next step without purification.

### Preparative Example P34

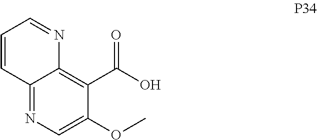

### 3-Methoxy-1,5-naphthyridine-4-carboxylic acid (P34)

To a solution of 3-methoxy-1,5-naphthyridine-4-carbaldehyde (376 mg, 2.0 mmol) in MeCN (10 mL) was added NaH₂PO₄ (94 mg, 0.60 mmol), NaClO₂ (252 mg, 2.80 mmol) and H₂O₂ (0.26 mL). The mixture was stirred at rt overnight and filtered. The filtrate was dried to afford compound P34 as a yellow solid.

### Example 1

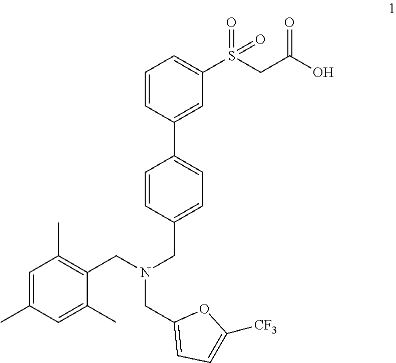 [SMILES: Cc1cc(C)c(CN(Cc2ccc(-c3cccc(S(=O)(=O)CC(=O)O)c3)cc2)Cc2ccc(C(F)(F)F)o2)c(C)c1]

### Step 1: tert-Butyl (4-bromobenzyl)((5-(trifluoromethyl)furan-2-yl)methyl)carbamate (1a)

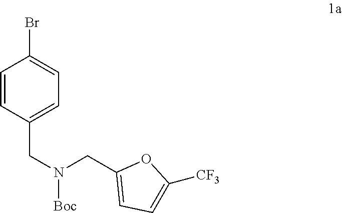 [SMILES: CC(C)(C)OC(=O)N(Cc1ccc(Br)cc1)Cc1ccc(C(F)(F)F)o1]

To a solution of tert-butyl (4-bromobenzyl)carbamate (8.6 g, 30 mmol) in dry DMF (120 mL) was added NaH (1.26 g, 31.6 mmol, 60% in mineral oil) at 0° C. under N₂. The mixture was stirred at 0° C. for 30 min, then a solution of 2-(bromomethyl)-5-(trifluoromethyl)furan (7.6 g, 33 mmol) in dry DMF (5 mL) was added to the mixture. The mixture was stirred at rt overnight, quenched with H₂O and extracted with EA (3×). The combined organic layer was washed with H₂O and brine, dried over Na₂SO₄, filtered, concentrated and purified by FCC (PE:EA=40:1) to obtain compound 1a as a pale yellow oil.

### Step 2: tert-Butyl (4-(4,4,5,5-tetramethyl-1,32-dioxaborolan-2-yl)benzyl)((5-(trifluoromethyl)furan-2-yl)methyl)carbamate (1b)

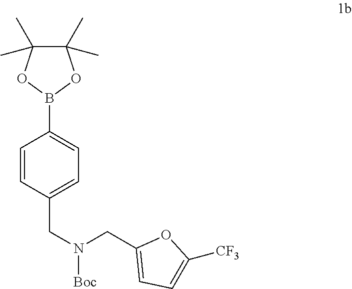 [SMILES: CC(C)(C)OC(=O)N(Cc1ccc(B2OC(C)(C)C(C)(C)O2)cc1)Cc1ccc(C(F)(F)F)o1]

A mixture of compound 1a (9.9 g, 23 mmol), Pd(dppf)Cl₂ (1.85 g, 2.28 mmol), B₂Pin₂ (7.53 g, 29.7 mmol) and KOAc (6.71 g, 68.4 mmol) in 1,4-dioxane (120 mL) was stirred at 105° C. under N₂ overnight, cooled and filtered. The filtrate was concentrated and purified by FCC (PE:EA=40:1 to 20:1) to obtain compound 1b as a yellow oil.

### Step 3: Methyl 2-((4′-(((tert-butoxycarbonyl)((5-(trifluoromethyl)furan-2-yl)methy)amino)methyl)-[1,1′-biphenyl]-3-yl)sulfonyl)acetate (1c)

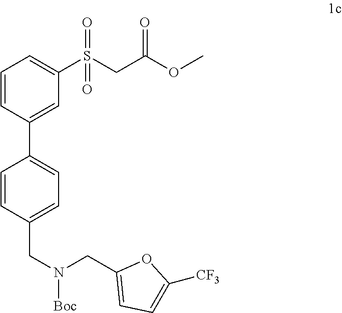 [SMILES: COC(=O)CS(=O)(=O)c1cccc(-c2ccc(CN(Cc3ccc(C(F)(F)F)o3)C(=O)OC(C)(C)C)cc2)c1]

A mixture of compound 1b (7.5 g, 16 mmol), methyl 2-((3-bromophenyl)sulfonyl)acetate (4.6 g, 16 mmol), Pd₂(dba)₃ (720 mg, 0.78 mmol), PPh₃ (613 mg, 2.34 mmol) and K₃PO₄ (10.1 g, 46.8 mmol) in 1,4-dioxane (100 mL) was stirred at 100° C. under N₂ overnight, cooled and filtered. The filtrate was concentrated and purified by FCC (PE:EA=10:1 to 5:1) to obtain compound 1c as a brown oil.

### Step 4: Methyl 2-((4′-((((5-(trifluoromethyl)furan-2-yl)methyl)amino)methyl)-[1,1′-biphenyl]-3-yl)sulfonyl)acetate (1d) and 1-(3′-(methylsulfonyl)-[1-biphenyl]-4-yl)-N-((5-(trifluoromethyl)furan-2-yl)methyl)methanamine (1d′)

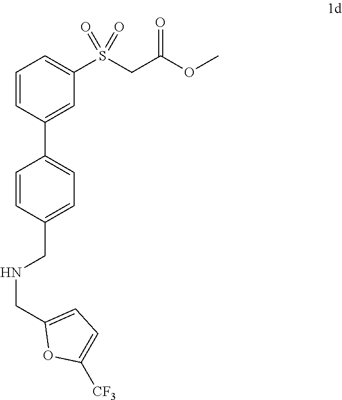 [SMILES: COC(=O)CS(=O)(=O)c1cccc(-c2ccc(CNCc3ccc(C(F)(F)F)o3)cc2)c1]

To a solution of compound 1c (8.6 g, 15 mmol) in DCM (120 mL) was added TFA (19.1 mL, 257 mmol) at 0° C. The solution was stirred at rt for 2 h, neutralized with sat. Na₂CO₃ and extracted with EA (3×). The combined organic layer was washed with brine, dried over Na₂SO₄ and concentrated to obtain a mixture of compound 1d and decarboxylated byproduct 1d′ as a brown oil.

### Step 5: Methyl 2-((4-((((5-(trifluoromethyl)furan-2-yl)methyl)(2,4,6-trimethyl-benzyl)amino)methyl)-[1,1-biphenyl]-3-yl)sulfonylacetate (1e)

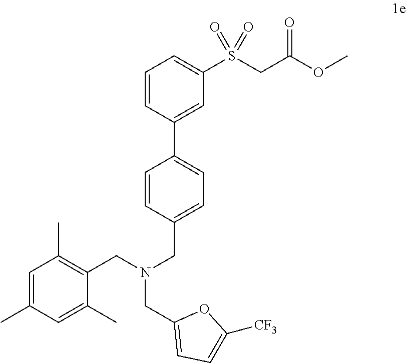

A mixture of compound 1d and decarboxylated byproduct (500 mg), 2-(bromomethyl)-1,3,5-trimethylbenzene (342 mg, 1.61 mmol) and K₂CO₃ (296 mg, 2.14 mmol) in ACN (20 mL) was stirred at 60° C. overnight, cooled and filtered. The filtrate was concentrated and purified by FCC (PE:EA=20:1 to 4:1) to obtain a mixture of compound 1e and decarboxylated byproduct 1-mesityl-N-((3′-(methylsulfonyl)-[1,1′-biphenyl]-4-yl)methyl)-N-((5-(trifluoromethyl)furan-2-yl)methyl)methanamine as a yellow oil.

### Step 6: 2-((4′-((((5-(Trifluoromethyl)furan-2-yl)methyl)(2,4,6-trimethylbenzyl)amino)methyl)-1,1′-biphenyl-3-yl)sulfonyl)acetic acid (1)

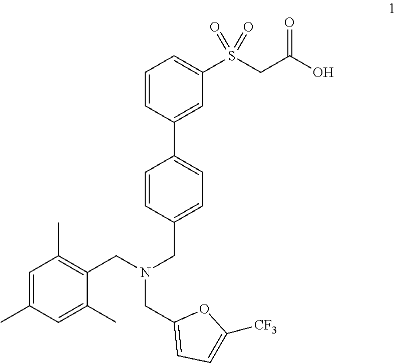 [SMILES: Cc1cc(C)c(CN(Cc2ccc(-c3cccc(S(=O)(=O)CC(=O)O)c3)cc2)Cc2ccc(C(F)(F)F)o2)c(C)c1]

A solution of a mixture of compound 1e and decarboxylated byproduct (450 mg), LiOH.H₂O (95 mg, 23 mmol) in THF (7 mL) and water (7 mL) was stirred at rt overnight, neutralized with 1N HCl to adjust the pH=5 to 6 and extracted with EA (3×). The combined organic layer was washed with brine, dried over Na₂SO₄, concentrated and purified by prep-HPLC to obtain compound 1 as a white solid. ¹H-NMR (CDCl₃, 300 MHz) δ: 8.02 (s, 1H), 7.78 (d, J=7.2 Hz, 1H), 7.55 (d, J=8.1 Hz, 1H), 7.36-7.28 (m, 3H), 7.19 (d, J=7.5 Hz, 2H), 6.79 (s, 2H), 6.65 (s, 1H), 6.15 (d, J=2.7 Hz, 1H), 4.14 (br s, 2H), 3.60 (s, 2H), 3.48 (s, 2H), 3.42 (s, 2H), 2.28 (s, 6H), 2.20 (s, 3H); MS: 586.2 (M+1)⁺.

### Example 2

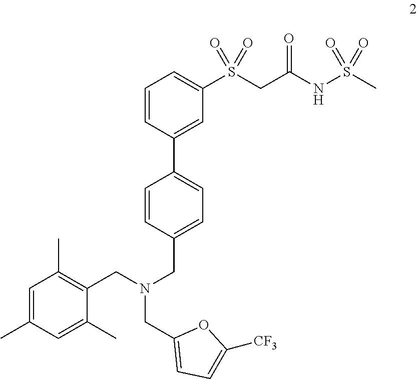

### N-(Methylsulfonyl)-2-((4′-((((5-(trifluoromethyl)furan-2-yl)methyl)(2,4,6-trimethyl-benzyl)amino)methyl-[1,1′-biphenyl]-3-yl)sulfonyl)acetamide (2)

To a solution of compound 1 (80 mg, 0.14 mmol), EDCI (36 mg, 0.19 mmol) and DMAP (17 mg, 0.14 mmol) in DMF (1.5 mL) was added methanesulfonamide (14 mg, 0.15 mmol) at rt. The mixture stirred at this temperature for 18 h, diluted with H₂O (20 mL) and extracted with EA (20 mL). The organic layer was washed with brine (10 mL), dried over Na₂SO₄, concentrated and purified by prep-HPLC to give compound 2 as a white solid. ¹H-NMR (500 MHz, DMSO-d₆) δ: 8.18 (t, J=1.8 Hz, 1H), 7.98-7.92 (m, 2H), 7.71-7.65 (m, 3H), 7.40 (d, J=8.0 Hz, 2H), 6.89-6.88 (m, 1H), 6.84 (s, 2H), 6.39 (d, J=3.5 Hz, 1H), 3.72 (s, 2H), 3.64 (s, 2H), 3.57 (s, 2H), 2.88 (s, 3H), 2.34 (s, 6H), 2.24 (s, 3H); MS: 663.2 (M+1)⁺.

### Example 2/1

The following Example was prepared similar as described for Example 2 using the appropriate building block.

| # | building block | structure | analytical data |
|---|---|---|---|
| 2/1 | 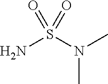 | 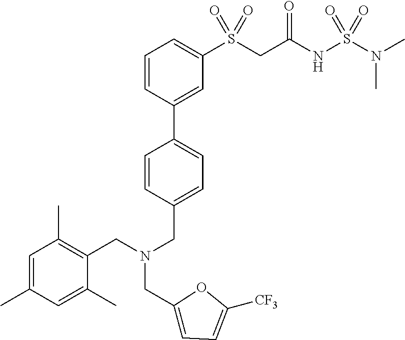 | ¹-NMR (500 MHz, CD₃OD) δ: 8.17 (t, J = 1.5 Hz, 1H), 8.01-7.92 (m, 2H), 7.72 (t, J = 2.8 Hz, 1H), 7.65 (d, J = 8.5 Hz, 2H), 7.41 (d, J = 8.0 Hz, 2H), 6.90-6.89 (m, 1H), 7.84 (s, 2H), 6.39 (d, J = 3.0 Hz, 1H), 3.72 (s, 2H), 3.64 (s, 2H), 3.57 (s, 2H), 2.78 (s, 6H), 2.34 (s, 6H), 2.24 (s, 3H); MS: 692.2 (M + 1)⁺. |

### Example 3

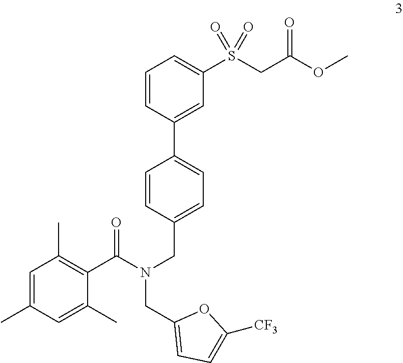

### Step 1: N-(4-Bromobenzyl)-1-(5-(trifluoromethyl)furan-2-yl)methanamine (3a)

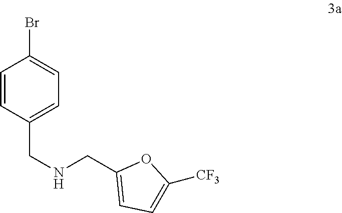 [SMILES: FC(F)(F)c1ccc(CNCc2ccc(Br)cc2)o1]

To a solution of compound 1a (13.6 g, 31.3 mmol) in DCM (150 mL) was added TFA (19.1 mL, 257 mmol) at 0° C. The solution was stirred at rt for 5 h, concentrated and neutralized with sat. Na₂CO₃ and extracted with EA (3×). The combined organic layer was washed with brine, dried over Na₂SO₄ and concentrated to obtain compound 3a as a brown oil.

### Step 2: N-(4-(4,4,5,5-Tetramethyl-1,3,2-dioxaborolan-2-yl)benzyl)-1-(5-(trifluoromethyl)furan-2-yl)methanamine (3b)

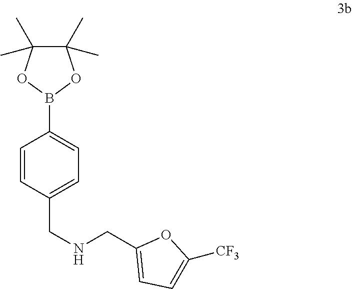 [SMILES: CC1(C)OB(c2ccc(CNCc3ccc(C(F)(F)F)o3)cc2)OC1(C)C]

A mixture of compound 3a (7.50 g, 22.5 mmol), Pd(dppf)Cl₂ (1.82 g, 2.25 mmol), B₂Pin₂ (7.42 g, 29.2 mmol) and KOAc (6.60 g, 67.3 mmol) in 1,4-dioxane (100 mL) was stirred at 105° C. under N₂ overnight, cooled and filtered. The filtrate was concentrated and purified by FCC (PE:EA=20:1 to 5:1) to obtain compound 3b as a brown oil.

### Step 3: 2,4,6-Trimethyl-N-(4-(4,4,5,5-tetramethyl-1,3,2-dioxaborolan-2-yl)benzyl)-N-((5-(tri-fluoromethyl)furan-2-yl)methyl)benzamide (3c)

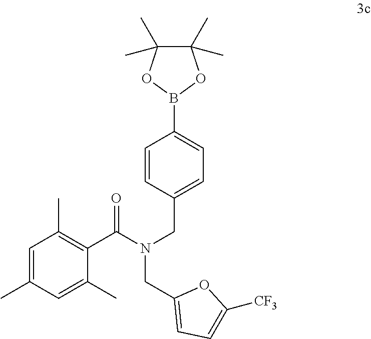 [SMILES: Cc1cc(C)c(C(=O)N(Cc2ccc(B3OC(C)(C)C(C)(C)O3)cc2)Cc2ccc(C(F)(F)F)o2)c(C)c1]

A solution of compound 3b (550 mg, 1.44 mmol), 2,4,6-trimethylbenzoyl chloride (289 mg, 1.58 mmol) and TEA (0.30 mL, 2.2 mmol) in THF (20 mL) was stirred at rt overnight, concentrated and purified by FCC (PE:EA=40:1 to 10:1) to obtain compound 3c as a colorless oil.

### Step 4: Methyl 2-((4′-((2,4,6-trimethyl-N-((5-(trifluoromethyl)furan-2-yl)methyl)benzamido)methyl)-[1,1′-biphenyl]-3-yl)sulfonylacetate (3)

A mixture of compound 3c (270 mg, 511 μmol), methyl 2-((3-bromophenyl)sulfonyl)acetate (165 mg, 562 μmol), Pd₂(dba)₃ (47 mg, 51 μmol), PPh₃ (40 mg, 153 μmol) and K₃PO₄ (330 mg, 1.53 mmol) in 1,4-dioxane (15 mL) was stirred at 90° C. under N₂ for 10 h, cooled and filtered. The filtrate was concentrated and purified by FCC (PE:EA=50:1 to 10:1) to obtain compound 3 as a yellow oil.

### Example 4

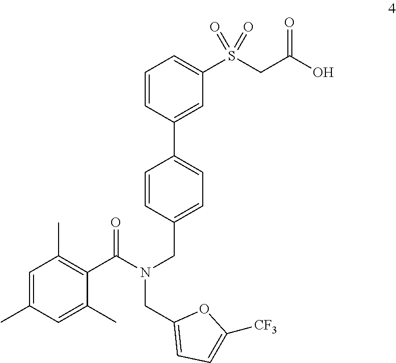 [SMILES: Cc1cc(C)c(C(=O)N(Cc2ccc(-c3cccc(S(=O)(=O)CC(=O)O)c3)cc2)Cc2ccc(C(F)(F)F)o2)c(C)c1]

### 2-((4′-((2,4,6-Trimethyl-N-((5-(trifluoromethyl)furan-2-yl)methyl)benzamido)methyl)-1,1′-biphenyl-3-yl)sulfonyl)acetic acid (4)

A solution of compound 3 (90 mg, 146 μmol) and LiOH.H₂O (18 mg, 439 μmol) in THF (5 mL) and water (5 mL) was stirred at rt overnight, neutralized with 1N HCl to pH=5-6 and extracted with EA (3×). The combined organic layer was washed with brine, dried over Na₂SO₄ and concentrated to obtain compound 4 as a yellow solid. ¹H-NMR (CDCl₃, 400 MHz, mixture of amide cis/trans isomers) δ: 8.16 (d, J=7.2 Hz, 1H), 7.92-7.85 (m, 2H), 7.64-7.56 (m, 3H), 7.43 (d, J=7.2 Hz, 1H), 7.18 (d, J=7.6 Hz, 1H), 6.85 (d, J=8.4 Hz, 2H), 6.75 (d, J=2.0 Hz, 0.5H), 6.67 (s, 0.5H), 6.40 (d, J=1.6 Hz, 0.5H), 6.10 (s, 0.5H), 4.80 (s, 1H), 4.71 (s, 1H), 4.35-4.15 (m, 4H), 2.74-2.17 (m, 9H); MS: 600.2 (M+1)⁺.

### Example 5

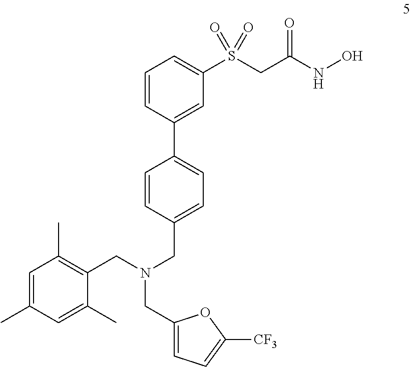 [SMILES: Cc1cc(C)c(CN(Cc2ccc(-c3cccc(S(=O)(=O)CC(=O)NO)c3)cc2)Cc2ccc(C(F)(F)F)o2)c(C)c1]

### N-Hydroxy-2-((4′-((((5-(trifluoromethyl)furan-2-yl)methyl)(2,46-trimethylbenzyl)amino)methyl)-[1,1′-biphenyl]-3-yl)sulfonyl)acetamide (5)

To a solution of compound 1 (80 mg, 0.14 mmol), EDCI (36 mg, 0.19 mmol), HOBt (26 mg, 0.19 mmol) and DIEA (36 mg, 0.28 mmol) in DMF (1.5 mL) was added NH₂OH*HCl (48 mg, 0.70 mmol) at rt. The mixture was stirred at this temperature for 18 h, diluted with H₂O (20 mL) and extracted with EA (20 mL). The organic layer was washed with brine (10 mL), dried over Na₂SO₄, concentrated and purified by prep-HPLC to give compound 5 as a white solid. ¹H-NMR (500 MHz, DMSO-d₆) δ: 10.42 (br s, 1H), 9.23 (br s, 1H), 8.09 (s, 1H), 8.02 (d, J=8.5 Hz, 1H), 7.83 (d, J=8.0 Hz, 1H), 7.73-7.68 (m, 3H), 7.36 (d, J=8.5 Hz, 2H), 7.14 (d, J=2.0 Hz, 1H), 6.82 (s, 2H), 6.54 (d, J=3.0 Hz, 1H), 4.22 (s, 2H), 3.63 (s, 2H), 3.60 (s, 2H), 3.51 (s, 2H), 2.28 (s, 6H), 2.18 (s, 3H); MS: 601.3 (M+1)⁺.

### Example 5/1 to 5/4

The following Examples were prepared similar as described for Example 5 using the appropriate building block(s).

| # | building block(s) | structure | analytical data |
|---|---|---|---|
| 5/1 | 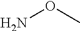 | 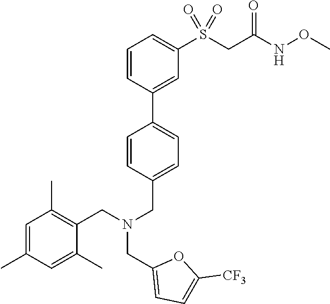 | ¹H-NMR (500 MHz, DMSO-d₆) δ: 11.34 (br s, 1H), 8.08-8.03 (m, 2H), 7.83 (d, J = 8.0 Hz, 1H), 7.75-7.62 (m, 3H), 7.37 (d, J = 7.0 Hz, 2H), 7.14-7.13 (m, 1H), 6.82 (s, 2H), 6.53 (d, J = 3.0 Hz, 1H), 4.23 (s, 2H), 3.63 (s, 2H), 3.60 (s, 2H), 3.51 (s, 2H), 3.48 (s, 3H), 2.28 (s, 6H), 2.18 (s, 3H); MS: 615.0 (M + 1)⁺. |
| 5/2 | 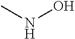 | 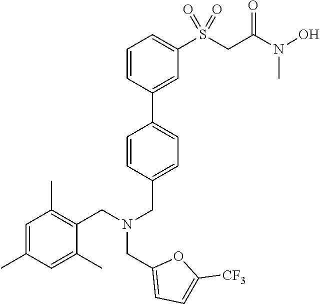 [SMILES: Cc1cc(C)c(CN(Cc2ccc(-c3cccc(S(=O)(=O)CC(=O)N(C)O)c3)cc2)Cc2ccc(C(F)(F)F)o2)c(C)c1] | ¹H-NMR (500 MHz, DMSO-d₆) δ: 10.27 (s, 1H), 8.12 (s, 1H), 8.01 (d, J = 8.0 Hz, 1H), 7.86 (d, J = 8.0 Hz, 1H), 7.72- 7.67 (m, 3H), 7.36 (d, J = 7.0 Hz, 2H), 7.13 (d, J = 2.0 Hz, 1H), 6.82 (s, 2H), 6.53 (d, J = 3.5 Hz, 1H), 4.66 (s, 2H), 3.63 (s, 2H), 3.60 (s, 2H), 3.51 (s, 2H), 3.05 (s, 3H), 2.28 (s, 6H), 2.18 (s, 3H); MS: 615.3 (M + 1)⁺. |
| 5/3 | 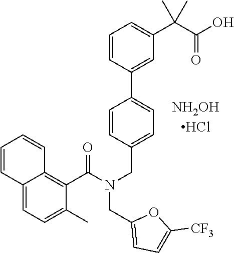 | 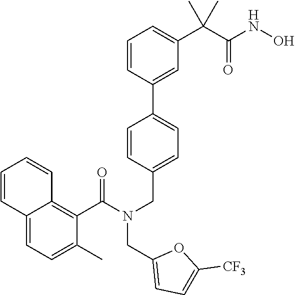 [SMILES: Cc1ccc2ccccc2c1C(=O)N(Cc1ccc(-c2cccc(C(C)(C)C(=O)NO)c2)cc1)Cc1ccc(C(F)(F)F)o1] | ¹H-NMR (500 MHz, CD₃OD) δ: 7.93- 7.90 (m, 2H), 7.78-7.64 (m, 2H), 7.59- 7.36 (m, 9H), 7.04 (d, J = 8.0 Hz, 1H), 7.00 (d, J = 2.0 Hz, 0.5H), 6.74 (d, J = 2.0 Hz, 0.5H), 6.55 (d, J = 3.5 Hz, 0.5H), 6.09 (d, J = 3.5 Hz, 0.5H), 5.04- 4.92 (m, 2H), 4.34-4.28 (m, 2H), 2.47, 2.44 (2 s, 3H), 1.67-1.59 (m, 6H); MS: 601.3 (M + 1)⁺. |
| 5/4 | 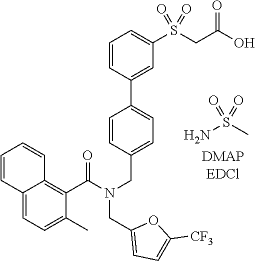 | 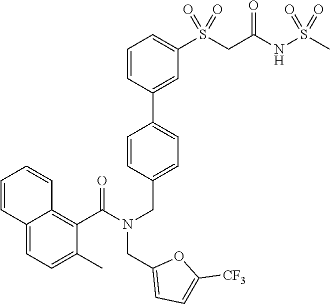 [SMILES: Cc1ccc2ccccc2c1C(=O)N(Cc1ccc(-c2cccc(S(=O)(=O)CC(=O)NS(C)(=O)=O)c2)cc1)Cc1ccc(C(F)(F)F)o1] | ¹H-NMR (500 MHz, CD₃OD) δ: 8.23 (t, J = 1.8 Hz, 0.5H), 8.12 (t, J = 1.5 Hz, 0.5H), 8.04-7.90 (m, 4H), 7.80-7.68 (m, 4H), 7.76-7.42 (m, 4H), 7.09 (d, J = 8.2 Hz, 1H), 7.01 (s, 0.5H), 6.76 (dd, J = 3.3, 1.3 Hz, 0.5H), 6.57 (d, J = 3.0 Hz, 0.5H), 6.12 (d, J = 3.0 Hz, 0.5H), 5.09- 4.94 (m, 2H), 4.41-4.28 (m, 2H), 2.94, 2.90 (2 s, 3H), 2.48, 2.44 (2 s, 3H); MS: 699.2 (M + 1)⁺. |

### Example 6

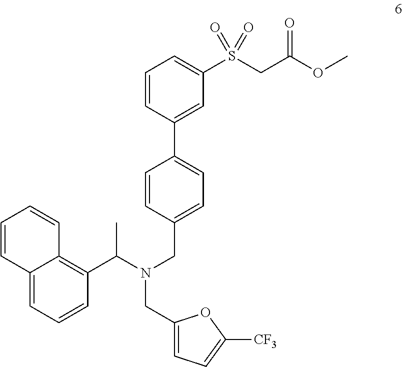 [SMILES: COC(=O)CS(=O)(=O)c1cccc(-c2ccc(CN(Cc3ccc(C(F)(F)F)o3)C(C)c3cccc4ccccc34)cc2)c1]

### Step 1: N-(4-Bromobenzyl)-1-(naphthalen-1-yl)-N-((5-(trifluoromethyl)furan-2-yl)ethan-1-amine (6a)

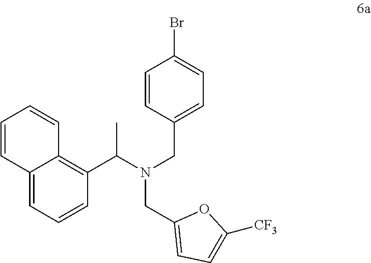 [SMILES: CC(c1cccc2ccccc12)N(Cc1ccc(Br)cc1)Cc1ccc(C(F)(F)F)o1]

To a solution of 1-(1-bromoethyl)naphthalene (700 mg, 2.98 mmol) and compound 3a (992 mg, 2.98 mmol) in ACN (40 mL) was added K₂CO₃ (822 mg, 5.96 mmol) and KI (495 mg, 2.98 mmol). Then the mixture stirred at 80° C. overnight, cooled and filtered. The filtrate was concentrated and purified by FCC (PE:EA=20:1) to give compound 6a as a yellow oil.

### Step 2: Methyl 2-((4′-(((1-(naphthalen-1-yl)ethyl)((5-(trifluoromethyl)furan-2-yl)methyl)amino)methyl)-[1,1′-biphenyl]-3-yl)sulfonyl)acetate (6)

A solution of compound 6a (561 mg, 1.15 mmol), methyl 2-((3-(4,4,5,5-tetramethyl-1,3,2-dioxaborolan-2-yl)phenyl)sulfonyl)acetate (392 mg, 1.15 mmol), Pd₂(dba)₃ (106 mg, 0.12 mmol), PPh₃ (91 mg, 0.35 mmol) and K₃PO₄ (743 mg, 3.46 mmol) in 1,4-dioxane (30 mL) was stirred at 85° C. under N₂ for 10 h, cooled, filtered, concentrated and purified by FCC (PE:EA=10:1 to 5:1) to afford compound 6 as a yellow oil.

### Example 7

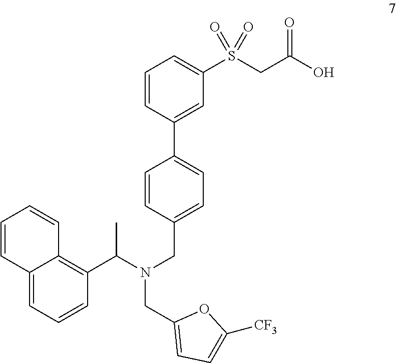 [SMILES: CC(c1cccc2ccccc12)N(Cc1ccc(-c2cccc(S(=O)(=O)CC(=O)O)c2)cc1)Cc1ccc(C(F)(F)F)o1]

### 2-((4′-(((1-(Naphthalen-1-yl)ethyl)((5-(trifluoromethyl)furan-2-yl)methyl)amino)methyl)-[1,1′-biphenyl]-3-yl)sulfonyl)acetic acid (7)

A solution of compound 6 (324 mg, 0.52 mmol) was saponified as described for Example 4 and purified by prep-HPLC to afford compound 7 as a white solid. ¹H-NMR (CDCl₃, 400 MHz) δ: 8.24 (d, J=8.4 Hz, 1H), 7.97 (s, 1H), 7.77-7.72 (m, 2H), 7.67 (d, J=8.4 Hz, 1H), 7.56 (d, J=7.2 Hz, 1H), 7.45-7.34 (m, 4H), 7.27-7.23 (m, 3H), 7.10 (d, J=8.0 Hz, 2H), 6.58 (d, J=2.0 Hz, 1H), 5.99 (d, J=3.2 Hz, 1H), 4.55 (q, J=6.8 Hz, 1H), 4.11 (br s, 2H), 3.66-3.47 (m, 4H), 1.49 (d, J=6.4 Hz, 3H); MS: 607.9 (M+1)⁺.

### Example 7/1 to 7/15

The following Examples were prepared similar as described for Example 6 using the appropriate building blocks and optionally saponified as described in Example 7.

| # | building blocks | structure | analytical data |
|---|---|---|---|
| 7/1 | 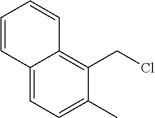 | 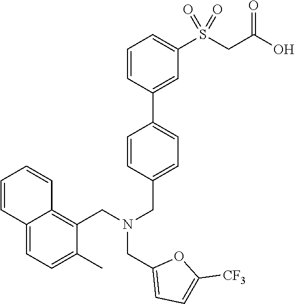 [SMILES: Cc1ccc2ccccc2c1CN(Cc1ccc(-c2cccc(S(=O)(=O)CC(=O)O)c2)cc1)Cc1ccc(C(F)(F)F)o1] | ¹H-NMR (CDCl₃, 400 MHz) δ: 8.16 (d, J = 8.0 Hz, 1H), 7.93 (s, 1H), 7.69 (d, J = 8.0 Hz, 2H), 7.59 (d, J = 8.8 Hz, 1H), 7.42-7.33 (m, 3H), 7.20-7.15 (m, 4H), 7.05 (d, J = 7.6 Hz, 2H), 6.63 (d, J = 1.2 Hz, 1H), 6.09 (d, J = 2.4 Hz, 1H), 4.08 (br s, 2H), 4.01 (s, 2H), 3.51 (s, 2H), 3.41 (s, 2H), 2.44 (s, 3H); MS: 607.9 (M + 1)⁺. |
| 7/2 | 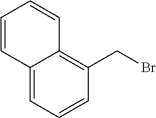 [SMILES: BrCc1cccc2ccccc12] | 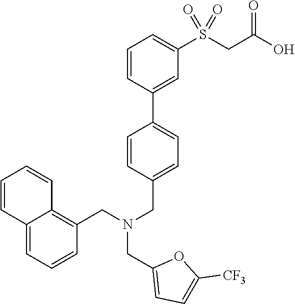 [SMILES: O=C(O)CS(=O)(=O)c1cccc(-c2ccc(CN(Cc3ccc(C(F)(F)F)o3)Cc3cccc4ccccc34)cc2)c1] | ¹H-NMR (CDCl₃, 400 MHz) δ: 8.10 (d, J = 8.4 Hz, 1H), 7.95 (s, 1H), 7.74-7.66 (m, 3H), 7.42-7.29 (m, 5H), 7.21 (d, J = 8.0 Hz, 2H), 7.14-7.10 (m, 3H), 6.61 (d, J = 2.0 Hz, 1H), 6.08 (d, J = 3.2 Hz, 1H), 4.13 (s, 2H), 3.90 (s, 2H), 3.46 (s, 2H), 3.43 (s, 2H); MS: 593.9 (M +1)⁺. |
| 7/3 | 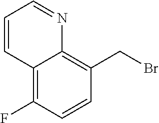 [SMILES: Fc1ccc(CBr)c2ncccc12] | 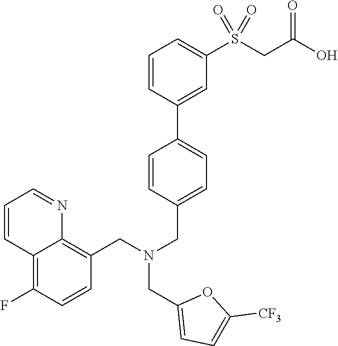 [SMILES: O=C(O)CS(=O)(=O)c1cccc(-c2ccc(CN(Cc3ccc(C(F)(F)F)o3)Cc3ccc(F)c4cccnc34)cc2)c1] | ¹H-NMR (CDCl₃, 400 MHz) δ: 8.85 (d, J = 4.0 Hz, 1H), 8.31 (d, J = 8.4 Hz, 1H), 7.99 (s, 1H), 7.86 (t, 1H), 7.74 (d, J = 7.2 Hz, 1H), 7.49 (d, J = 7.6 Hz, 1H), 7.37- 7.29 (m, 6H), 7.14 (d, J = 8.8 Hz, 1H), 6.61 (s, 1H), 6.24 (d, J = 2.4 Hz, 1H), 4.27 (s, 2H), 4.10 (s, 2H), 3.67 (s, 2H). 3.66 (s, 2H); MS: 612.9 (M +1)⁺. |
| 7/4 | 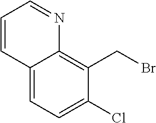 [SMILES: Clc1ccc2cccnc2c1CBr] | 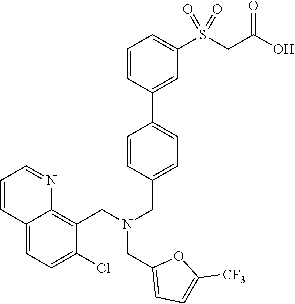 [SMILES: O=C(O)CS(=O)(=O)c1cccc(-c2ccc(CN(Cc3ccc(C(F)(F)F)o3)Cc3c(Cl)ccc4cccnc34)cc2)c1] | ¹H-NMR (CDCl₃, 400 MHz) δ: 8.83 (dd, J = 1.6, J = 4.0 Hz, 1H), 7.93-7.88 (m, 2H), 7.68 (d, J = 7.6 Hz, 1H), 7.48 (d, J = 8.8 Hz, 1H), 7.37 (d, J = 8.8 Hz, 2H), 7.27- 7.13 (m, 6H), 6.58 (d, J = 2.0 Hz, 1H), 6.26 (d, J = 3.2 Hz, 1H), 4.44 (s, 2H), 4.07 (s, 2H), 3.67 (s, 2H), 3.63 (s, 2H); MS: 628.9 (M +1)⁺. |
| 7/5 | 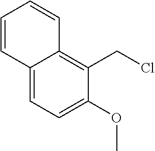 | 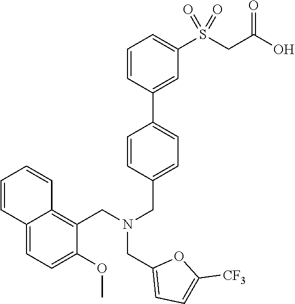 [SMILES: COc1ccc2ccccc2c1CN(Cc1ccc(-c2cccc(S(=O)(=O)CC(=O)O)c2)cc1)Cc1ccc(C(F)(F)F)o1] | ¹H-NMR (CDCl₃, 400 MHz) δ: 8.00 (d, J = 8.4 Hz, 2H), 7.74-7.67 (m, 3H), 7.51 (dd, J = 8.0, J = 0.4 Hz, 1H), 7.41 (t, J = 7.2 Hz, 1H), 7.29-7.25 (m, 4H), 7.21-7.14 (m, 3H), 6.65 (d, J = 2.0 Hz, 1H), 6.25 (d, J = 3.2 Hz, 1H), 4.14 (s, 2H), 4.07 (s, 2H), 3.85 (s, 3H), 3.67 (s, 2H), 3.60 (s, 2H); MS: 624.0 (M +1)⁺. |
| 7/6 | 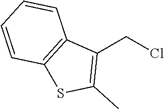 | 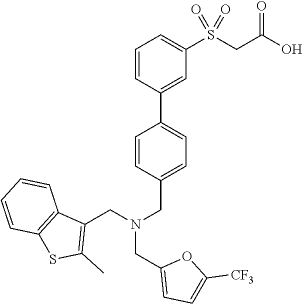 [SMILES: Cc1sc2ccccc2c1CN(Cc1ccc(-c2cccc(S(=O)(=O)CC(=O)O)c2)cc1)Cc1ccc(C(F)(F)F)o1] | ¹H-NMR (CDCl₃, 400 MHz) δ: 8.03 (s, 1H), 7.82-7.78 (m, 2H), 7.66 (d, J = 8.4 Hz, 1H), 7.59 (d, J = 6.8 Hz, 1H), 7.37- 7.21 (m, 7H), 6.66 (d, J = 2.0 Hz, 1H), 6.13 (d, J = 3.2 Hz, 1H), 4.12 (br s, 2H), 3.75 (s, 2H), 3.54 (s, 2H), 3.50 (s, 2H), 2.47 (s, 3H); MS: 613.9 (M +1)⁺. |
| 7/7 | 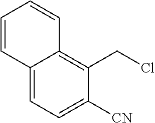 [SMILES: N#Cc1ccc2ccccc2c1CCl] | 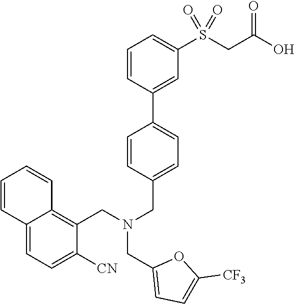 [SMILES: N#Cc1ccc2ccccc2c1CN(Cc1ccc(-c2cccc(S(=O)(=O)CC(=O)O)c2)cc1)Cc1ccc(C(F)(F)F)o1] | ¹H-NMR (CDCl₃, 300 MHz) δ: 8.14-8.11 (m, 2H), 7.98 (t, J = 1.4 Hz, 1H), 7.77- 7.73 (m, 2H), 7.57-7.49 (m, 4H), 7.33- 7.27 (m, 3H), 7.21-7.18 (m, 2H), 6.67 (d, J = 2.4 Hz, 1H), 6.18-6.16 (m, 1H), 4.12 (s, 2H), 3.96 (s, 2H), 3.54-3.51 (s, 4H); MS: 618.9 (M +1)⁺. |
| 7/8 | 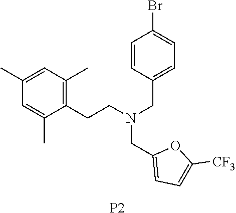 | 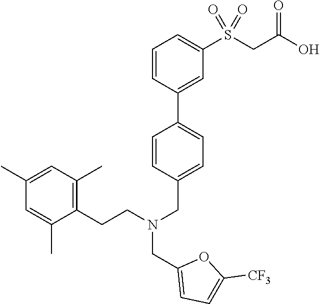 [SMILES: Cc1cc(C)c(CCN(Cc2ccc(-c3cccc(S(=O)(=O)CC(=O)O)c3)cc2)Cc2ccc(C(F)(F)F)o2)c(C)c1] | ¹H-NMR (CDCl₃, 400 MHz) δ: 8.14 (s, 1H), 7.89 (d, J = 8.0 Hz, 1H), 7.69 (d, J = 7.6 Hz, 1H), 7.49-7.43 (m, 3H), 7.35 (d, J = 8.0 Hz, 2H), 6.73-6.72 (m, 3H), 6.37 (d, J = 3.2 Hz, 1H), 4.19 (s, 2H), 3.90 (s, 2H), 3.80 (s, 2H), 2.85-2.81 (m, 2H), 2.61-2.57 (m, 2H), 2.17 (s, 3H), 2.10 (s, 6H); MS: 600.0 (M + 1). |
| 7/9 | 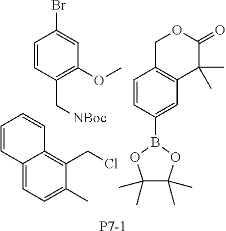 | 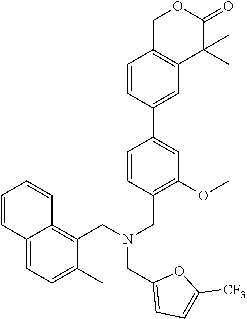 [SMILES: COc1cc(-c2ccc3c(c2)C(C)(C)C(=O)OC3)ccc1CN(Cc1ccc(C(F)(F)F)o1)Cc1c(C)ccc2ccccc12] | ¹H-NMR (CD₃OD, 400 MHz) δ: 8.21 (d, J = 8.4 Hz, 1H), 7.72 (dd, J = 1.6, 7.6 Hz, 1H), 7.64 (d, J = 8.4 Hz, 1H), 7.56 (d, J = 1.2 Hz, 1H), 7.49 (dd, J = 2.0, 8.0 Hz, 1H), 7.42-7.34 (m, 2H), 7.29-7.25 (m, 3H), 7.05-7.03 (m, 2H), 6.83-6.82 (m, 1H), 6.30 (d, J = 3.2 Hz, 1H), 5.48 (s, 2H), 4.13 (s, 2H), 3.73 (s, 3H), 3.67 (s, 2H), 3.65 (s, 2H), 2.51 (s, 3H), 1.59 (s, 6H); MS: 614.0 (M +1)⁺. |
| 7/10 | 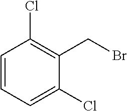 | 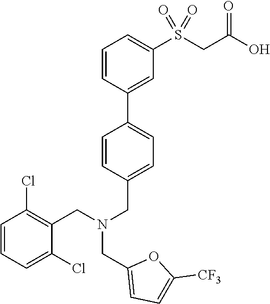 [SMILES: O=C(O)CS(=O)(=O)c1cccc(-c2ccc(CN(Cc3ccc(C(F)(F)F)o3)Cc3c(Cl)cccc3Cl)cc2)c1] | ¹H-NMR (CDCl₃, 400 MHz) δ: 8.08 (s, 1H), 7.87 (d, J = 7.6 Hz, 1H), 7.72 (d, J = 4.8 Hz, 1H), 7.51-4.47 (m, 1H), 7.42 (d, J = 7.6 Hz, 2H), 7.32 (d, J = 6.8 Hz, 2H), 7.27-7.24 (m, 2H), 7.08 (t, J = 8.2 Hz, 1H), 6.67 (s, 1H), 6.23 (d, J = 1.2 Hz, 1H), 4.19 (br s, 2H), 3.98 (s, 2H), 3.66 (s, 2H), 3.62 (s, 2H); MS: 612.0 (M +1)⁺. |
| 7/11 | 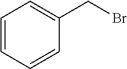 | 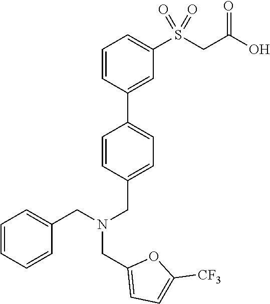 [SMILES: O=C(O)CS(=O)(=O)c1cccc(-c2ccc(CN(Cc3ccccc3)Cc3ccc(C(F)(F)F)o3)cc2)c1] | ¹H-NMR (CDCl₃, 400 MHz) δ: 7.98 (s, 1H), 7.74 (d, J = 8.0 Hz, 1H), 7.43 (d, J = 7.6 Hz, 1H), 7.31-7.16 (m, 10H), 6.63 (d, J = 2.0 Hz, 1H), 6.13 (d, J = 3.2 Hz, 1H), 4.12 (s, 2H), 4.48-4.42 (m, 6H); MS: 544.1 (M +1)⁺. |
| 7/12 | 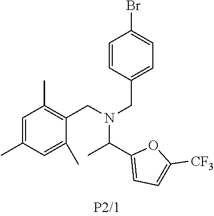 | 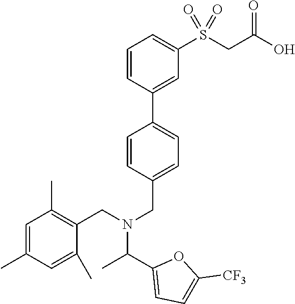 [SMILES: Cc1cc(C)c(CN(Cc2ccc(-c3cccc(S(=O)(=O)CC(=O)O)c3)cc2)C(C)c2ccc(C(F)(F)F)o2)c(C)c1] | ¹H-NMR (CDCl₃, 400 MHz) δ: 8.01 (s, 1H), 7.78 (d, J = 7.6 Hz, 1H), 7.57 (d, J = 8.0 Hz, 1H), 7.36-7.32 (m, 3H), 7.19 (d, J = 8.4 Hz, 2H), 6.76 (s, 2H), 6.68-6.67 (m, 1H), 6.15 (d, J = 3.2 Hz, 1H), 4.12 (s, 2H), 3.90-3.85 (m, 1H), 3.72 (d, J = 12.4 Hz, 1H), 3.48-3.37 (m, 3H), 2.26 (s, 6H), 2.18 (s, 3H), 1.38 (d, J = 6.8 Hz, 3H); MS: 600.0 (M +1)⁺. |
| 7/13 | 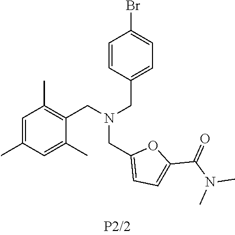 | 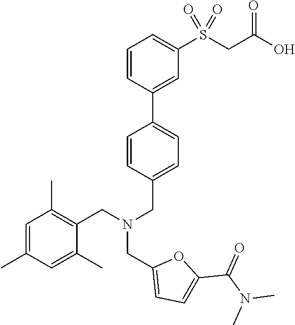 [SMILES: Cc1cc(C)c(CN(Cc2ccc(-c3cccc(S(=O)(=O)CC(=O)O)c3)cc2)Cc2ccc(C(=O)N(C)C)o2)c(C)c1] | ¹H-NMR (CDCl₃, 400 MHz) δ: 8.01 (s, 1H), 7.80 (d, J = 7.2 Hz, 1H), 7.52 (br s, 1H), 7.31-2.28 (m, 3H), 7.12 (d, J = 6.8 Hz, 2H), 6.88 (d, J = 3.6 Hz, 1H), 6.78 (s, 2H), 6.08 (d, J = 2.8 Hz, 1H), 4.17 (br s, 2H), 3.60 (s, 2H), 3.47 (s, 2H), 3.43 (br s, 2H), 3.20-3.13 (m, 3H), 3.06-2.99 (m, 3H), 2.28 (s, 6H), 2.19 (s, 3H); MS: 589.2 (M +1)⁺. |
| 7/14 | 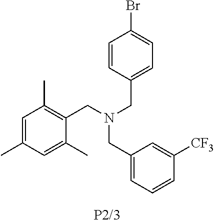 | 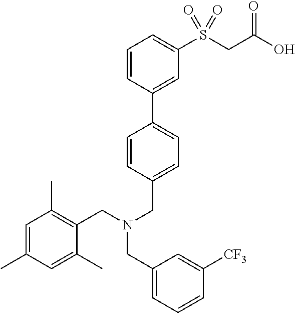 [SMILES: Cc1cc(C)c(CN(Cc2ccc(-c3cccc(S(=O)(=O)CC(=O)O)c3)cc2)Cc2cccc(C(F)(F)F)c2)c(C)c1] | MS: 596.0 (M +1)⁺. |
| 7/15 | 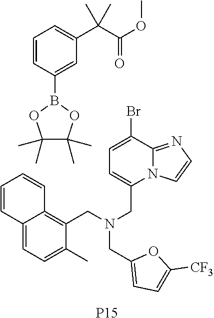 | 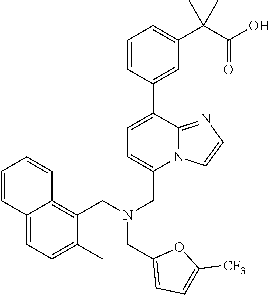 [SMILES: Cc1ccc2ccccc2c1CN(Cc1ccc(C(F)(F)F)o1)Cc1ccc(-c2cccc(C(C)(C)C(=O)O)c2)c2nccn12] | ¹H-NMR (CDCl₃, 400 MHz) δ: 8.05 (d, J = 10.0 Hz, 1H), 7.81-7.78 (m, 1H), 7.72 (d, J = 8.0 Hz, 1H), 7.66 (s, 1H), 7.54- 7.52 (m, 2H), 7.44 (dd, J = 3.2, 6.4 Hz, 2H), 7.38 (d, J = 5.2 Hz, 2H), 7.31 (d, J = 8.0 Hz, 1H), 7.19-7.17 (m, 2H), 6.86 (d, J = 6.8 Hz, 1H), 6.75 (d, J = 2.4 Hz, 1H), 6.28 (s, J = 3.2 Hz, 1H), 4.26 (s, 2H), 3.92 (s, 2H), 3.86 (s, 2H), 2.54 (s, 3H), 1.58 (s, 6H); MS: 612.0 (M +1)⁺. |

### Example 8

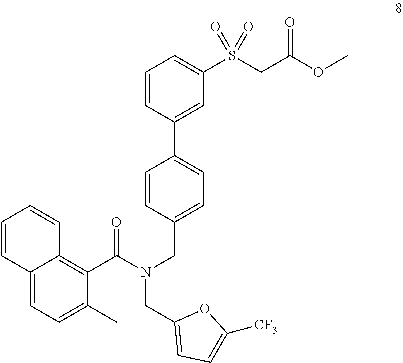 [SMILES: COC(=O)CS(=O)(=O)c1cccc(-c2ccc(CN(Cc3ccc(C(F)(F)F)o3)C(=O)c3c(C)ccc4ccccc34)cc2)c1]

### Step 1: N-(4-Bromobenzyl)-2-methyl-1-naphthamide (8a)

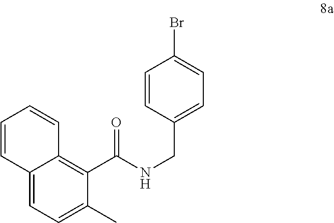 [SMILES: Cc1ccc2ccccc2c1C(=O)NCc1ccc(Br)cc1]

To a solution of 2-methyl-1-naphthoic acid (500 mg, 2.69 mmol) and (4-bromophenyl)methanamine (500 mg, 2.69 mmol) in DMF (20 mL) was added TEA (543 mg, 5.38 mmol) and HATU (1.23 g, 3.23 mmol) at 0° C. The mixture was stirred at rt overnight, diluted with H₂O and extracted with EA (3×). The combined organic layer was washed with brine, dried over Na₂SO₄, filtered and concentrated to give the crude compound 8a as a yellow solid.

### Step 2: N-(4-Bromobenzyl)-2-methyl-N-((5-(trifluoromethyl)furan-2-yl)methyl)-1-naphthamide (8b)

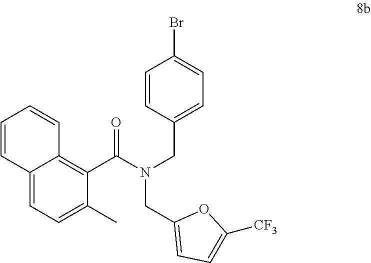 [SMILES: Cc1ccc2ccccc2c1C(=O)N(Cc1ccc(Br)cc1)Cc1ccc(C(F)(F)F)o1]

To a solution of compound 8a (706 mg, 2.00 mmol) in dry DMF (20 mL) was added NaH (96 mg, 60%, 4.0 mmol). The mixture was stirred at 0° C. for 15 min, then 2-(bromomethyl)-5-(trifluoromethyl)furan (912 mg, 4.00 mmol) was added and the mixture stirred at rt overnight, filtered, concentrated and purified by FCC (PE:EA=20:1 to 10:1) to give compound 8b as a yellow oil.

### Step 3: Methyl 2-((4′-((2-methyl-N-((5-(trifluoromethyl)furan-2-yl)methyl)-1-naphthamido methyl)-[1,1-biphenyl]-3-yl)sulfonyl)acetate (8)

To a solution of compound 8b (713 mg, 1.42 mmol), methyl 2-((3-(4,4,5,5-tetramethyl-1,3,2-dioxaborolan-2-yl)phenyl)sulfonyl)acetate (484 mg, 1.42 mmol), PPh₃ (112 mg, 0.43 mmol) and K₃PO₄ (918 mg, 4.27 mmol) in 1,4-dioxane (30 mL) was added Pd₂(dba)₃ (131 mg, 0.14 mmol). The mixture was stirred at 85° C. under N₂ for 10 h, cooled, filtered, concentrated and purified by FCC (PE:EA=10:1 to 5:1 to 3:1) to afford compound 8 as a yellow oil.

### Example 9

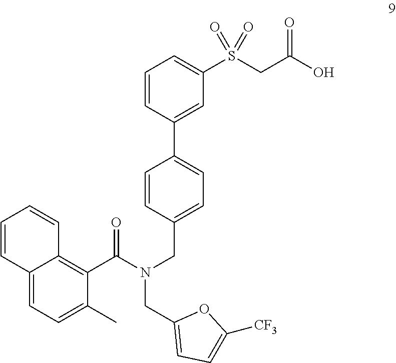 [SMILES: Cc1ccc2ccccc2c1C(=O)N(Cc1ccc(-c2cccc(S(=O)(=O)CC(=O)O)c2)cc1)Cc1ccc(C(F)(F)F)o1]

### 2-((4-((2-Methyl-N-((5-(trifluoromethyl)furan-2-yl)methyl)-1-naphthamido)methyl-[1,1′-biphenyl]-3-yl)sulfonyl)acetic acid (9)

To a solution of compound 8 (476 mg, 0.75 mmol) in THF (10 mL) and water (10 mL) was added LiOH.H₂O (63 mg, 1.50 mmol) at rt. The mixture was stirred at rt overnight and concentrated. The residue was acidified with 2N HCl to adjust to pH=6, filtered and then the solid was purified by prep-HPLC to obtain compound 9 as a white solid. ¹H-NMR (CDCl₃, 400 MHz, mixture of isomers) δ: 8.08 (s, 0.5H), 8.00 (s, 0.5H), 7.82-7.21 (m, 12H), 6.88-6.86 (m, 1H), 6.69 (s, 0.5H), 6.45 (s, 0.5H), 6.33 (s, 0.5H), 5.73 (s, 0.5H), 4.89-4.69 (m, 2H), 4.20-4.00 (m, 4H), 2.34 (s, 3H); MS: 621.9 (M+1)⁺.

### Example 9/1

The following Example was prepared similar as described for Example 8 using the appropriate building blocks and saponified as described in Example 9.

| # | building block | structure | analytical data |
|---|---|---|---|
| 9/1 | 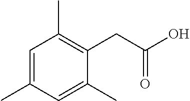 | 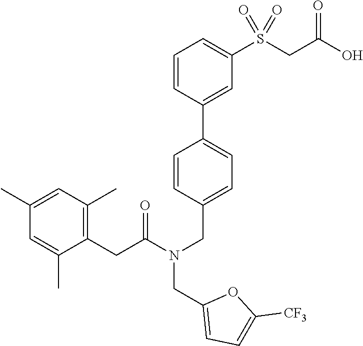 [SMILES: Cc1cc(C)c(CC(=O)N(Cc2ccc(-c3cccc(S(=O)(=O)CC(=O)O)c3)cc2)Cc2ccc(C(F)(F)F)o2)c(C)c1] | ¹H-NMR (CDCl₃, 400 MHz, mixture of isomers) δ: 8.08 (s, 0.5H), 8.01 (s, 0.5H), 7.82-7.34 (m, 5H), 7.17-7.14 (m, 2H), 6.77 (d, J = 9.2 Hz, 2H), 6.63 (s, 1H), 6.23 (s, 0.5H), 6.18 (s, 0.5H), 462 (s, 1H), 449 (s, 1H), 448 (s, 1H), 4.41 (s, 1H), 4.13 (br s, 2H), 3.77 (s, 1H), 3.56 (s, 1H), 2.18 (s, 3H), 2.14 (s, 3H), 2.06-2.00 (m, 3H); MS: 614.2 (M + 1)⁺. |

### Example 10

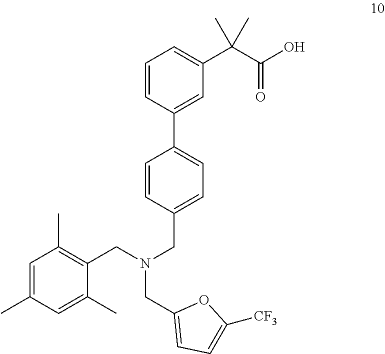 [SMILES: Cc1cc(C)c(CN(Cc2ccc(-c3cccc(C(C)(C)C(=O)O)c3)cc2)Cc2ccc(C(F)(F)F)o2)c(C)c1]

### Step 1: N-(4-Bromobenzyl)-1-(5-(trifluoromethyl)furan-2-yl)methanamine hydrogenchloride (10a)

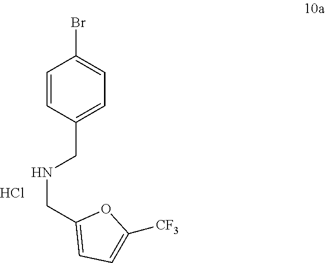 [SMILES: Cl ; FC(F)(F)c1ccc(CNCc2ccc(Br)cc2)o1]

To a solution of compound 1a (2.00 g, 4.60 mmol) in 1,4-dioxane (10 mL) was added HCl (5 mL, 6M in 1,4-dioxane) and the mixture was stirred at rt for 2 h. The solvent was evaporated to give compound 10a as a white solid.

### Step 2: N-(4-Bromobenzyl)-1-mesityl-N-((5-(trifluoromethyl)furan-2-yl)methyl)methanamine (10b)

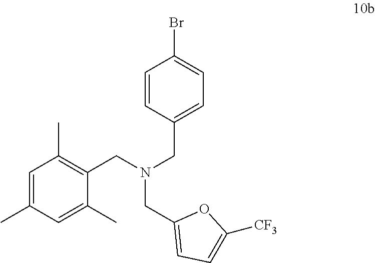 [SMILES: Cc1cc(C)c(CN(Cc2ccc(Br)cc2)Cc2ccc(C(F)(F)F)o2)c(C)c1]

To a solution of compound 10a (740 mg, 2.00 mmol) in 1,2-dichloroethane (20 mL) was added 2,4,6-trimethylbenzaldehyde (326 mg, 2.20 mmol) and one drop AcOH. The mixture was stirred at rt for 0.5 h. Then NaBH(OAc)₃ (848 mg, 4.00 mmol) was added and the mixture was stirred at rt overnight, diluted with water (40 mL) and extracted with DCM (3×20 mL). The combined organic layer was washed with brine (30 mL), dried over Na₂SO₄, filtered, concentrated and purified by FCC (PE:EA=50:1) to give compound 10b as a colorless oil.

### Step 3: 1-Mesityl-N-(4-(4,4,5,5-tetramethyl-1,3,2-dioxaborolan-2-yl)benzyl)-N-((5-(trifluoromethyl)furan-2-yl)methyl)methanamine (10c)

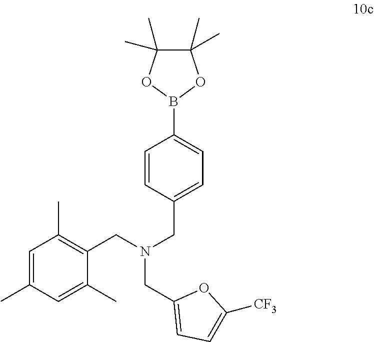 [SMILES: Cc1cc(C)c(CN(Cc2ccc(B3OC(C)(C)C(C)(C)O3)cc2)Cc2ccc(C(F)(F)F)o2)c(C)c1]

To a solution of compound 10b (400 mg, 0.86 mmol) in 1,4-dioxane (10 mL) was added B₂Pin₂ (220 mg, 0.86 mmol), KOAc (170 mg, 1.72 mmol) and Pd(dppf)Cl₂ (40 mg). The mixture was stirred at 90° C. for 3 h, diluted with water (40 mL) and extracted with EA (3×20 mL). The combined organic layer was washed with brine (30 mL), dried over Na₂SO₄, filtered, concentrated and purified by FCC (PE:EA=50:1) to give compound 10c as a white solid.

### Step 4: 2-ethyl-2-(4′-((((5-(trifluoromethyl)furan-2-yl)methyl)(2,4,6-trimethyl-benzyl)amino)methyl)-[1,1′-biphenyl]-3-yl)propanoic acid (10)

A mixture of compound 10c (300 mg, 585 μmol), 2-(3-bromophenyl)-2-methylpropanoic acid (142 mg, 585 μmol), S-phos (24 mg, 59 μmol), Pd(OAc)₂ (7.0 mg, 29 μmol) and K₃PO₄ (310 mg, 1.46 mmol) in ACN/H₂O (15 mL/5 mL) was heated to 90° C. under N₂ for 10 h, cooled, filtered, concentrated and purified by prep-HPLC to afford compound 10 as a white solid. ¹H-NMR (CDCl₃, 400 MHz) δ: 7.55 (s, 1H), 7.47 (d, J=8.0 Hz, 2H), 7.41 (br s, 1H), 7.33-7.29 (m, 4H), 6.81 (s, 2H), 6.69 (d, J=2.0 Hz, 1H), 6.20 (d, J=2.8 Hz, 1H), 3.67 (s, 2H), 3.59 (s, 2H), 3.53 (s, 2H), 2.33 (s, 6H), 2.23 (s, 3H), 1.59 (s, 6H); MS: 550.2 (M+1)⁺.

### Example 10/1 to 10/6

The following Examples were prepared similar as described for Example 10 using the appropriate building blocks.

| # | building blocks | structure | analytical data |
|---|---|---|---|
| 10/1 | 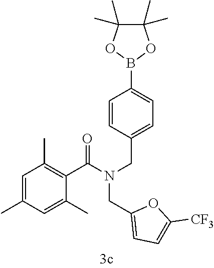 | 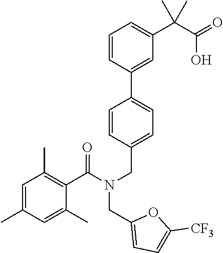 [SMILES: Cc1cc(C)c(C(=O)N(Cc2ccc(-c3cccc(C(C)(C)C(=O)O)c3)cc2)Cc2ccc(C(F)(F)F)o2)c(C)c1] | ¹H-NMR (CDCl₃, 400 MHz, mixture of isomers) δ: 7.60-7.47 (m, 3H), 7.44-7.40 (m, 4H), 7.16 (d, J = 8.0 Hz, 1H), 7.86 (d, J = 6.8 Hz, 2H), 6.74 (d, J = 2.0 Hz, 0.5H), 6.66 (d, J = 1.6 Hz, 0.5H), 6.39 (d, J = 3.2 Hz, 0.5H), 6.07 (d, J = 2.8 Hz, 0.5H), 4.83 (s, 1H), 4.75 (s, 1H), 4.34 (s, 1H), 4.20 (s, 1H), 2.28, 2.27 (2 s, 3H), 2.24, 2.22 (2 s, 6H), 1.66, 1.65 (2 s, 6H); MS: 564.2 (M + 1)⁺. |
| 10/2 | 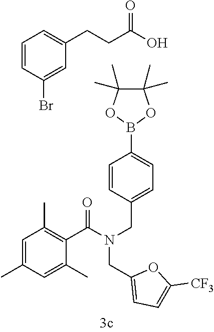 | 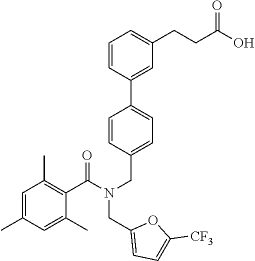 [SMILES: Cc1cc(C)c(C(=O)N(Cc2ccc(-c3cccc(CCC(=O)O)c3)cc2)Cc2ccc(C(F)(F)F)o2)c(C)c1] | ¹H-NMR (CDCl₃, 400 MHz, mixture of isomers) δ: 7.58-7.52 (m, 2H), 7.44-7.36 (m, 4H), 7.21 (d, J = 6.8 Hz, 1H), 7.16 (d, J = 8.0 Hz, 1H), 7.86 (d, J = 6.4 Hz, 2H), 6.75 (d, J = 2.0 Hz, 0.5H), 6.67 (d, J = 2.4 Hz, 0.5H), 6.39 (d, J = 3.2 Hz, 0.5H), 6.07 (d, J = 2.8 Hz, 0.5H), 4.82 (s, 1H), 4.75 (s, 1H), 4.34 (s, 1H), 4.20 (s, 1H), 3.05-3.00 (m, 2H), 2.75-2.70 (m, 2H), 2.28, 2.27 (2 s, 3H), 2.23, 2.22 (2 s, 6H); MS: 550.2 (M + 1)⁺. |
| 10/3 | 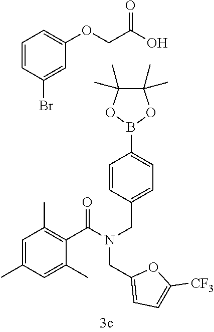 | 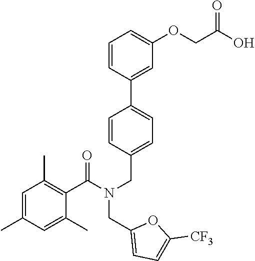 [SMILES: Cc1cc(C)c(C(=O)N(Cc2ccc(-c3cccc(OCC(=O)O)c3)cc2)Cc2ccc(C(F)(F)F)o2)c(C)c1] | ¹H-NMR (CDCl₃, 400 MHz, mixture of isomers) δ: 7.42-7.39 (m, 4H), 733 (d, J = 8.4 Hz, 1H), 7.07 (d, J = 8.0 Hz, 1H), 6.93- 6.90 (m, 2H), 6.82, 6.81 (2 s, 2H), 6.71 (d, J = 2.0 Hz, 0.5H), 6.61 (d, J = 1.2 Hz, 0.5H), 6.35 (d, J = 3.2 Hz, 0.5H), 6.02 (d, J = 3.2, 0.5H), 4.73 (s, 1H), 4.68 (s, 1H), 4.51-4.49 (m, 2H), 4.28 (s, 1H), 4.13 (s, 1H), 2.24, 2.23 (2 s, 3H), 2.17 (s, 6H); MS: 552.2 (M + 1)⁺. |
| 10/4 | 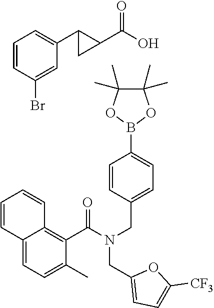 | 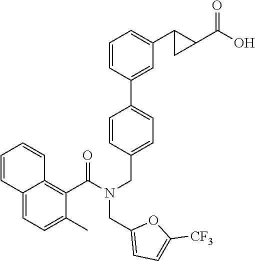 | ¹H-NMR (CDCl₃, 400 MHz) δ: 8.26 (d, J = 8.4 Hz, 1H), 7.77 (d, J = 8.0 Hz, 1H), 7.67 (d, J = 8.8 Hz, 1H), 7.50-7.38 (m, 5H), 7.35-7.27 (m, 5H), 7.06 (d, J = 7.6 Hz, 1H), 6.72 (s, 1H), 6.22 (d, J = 2.0 Hz, 1H), 4.17 (s, 2H), 3.71 (s, 2H), 3.63 (s, 2H), 2.67- 2.62 (m, 1H), 2.56 (s, 3H), 1.97-1.93 (m, 1H), 1.70-1.65 (m, 1H), 1.47-1.43 (m, 1H); MS: 570.0 (M + 1)⁺. |
| 10/5 | 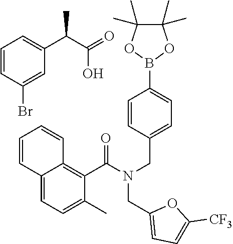 | 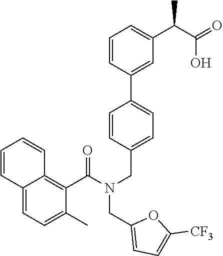 [SMILES: Cc1ccc2ccccc2c1C(=O)N(Cc1ccc(-c2cccc([C@@H](C)C(=O)O)c2)cc1)Cc1ccc(C(F)(F)F)o1] | ¹H-NMR (CDCl₃, 400 MHz, mixture of isomers) δ: 7.83-7.69 (m, 3H), 7.63-7.27 (m, 10H), 7.07 (d, J = 8.0 Hz, 1H), 6.81- 6.80 (m, 0.5H), 6.57-6.56 (m, 0.5H), 6.44 (d, J = 2.8 Hz, 0.5H), 5.85 (d, J = 3.2 Hz, 0.5H), 5.05-4.82 (m, 2H), 4.26, 4.15 (2 s, 2H), 3.84-3.77 (m, 1H), 2.46 (s, 3H), 1.60- 1.55 (m, 3H); MS: 572.0 (M + 1)⁺. |
| 10/6 | 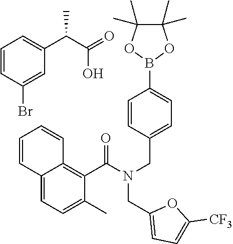 | 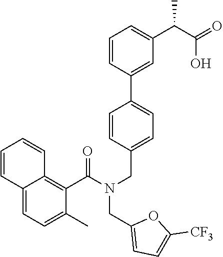 [SMILES: Cc1ccc2ccccc2c1C(=O)N(Cc1ccc(-c2cccc([C@H](C)C(=O)O)c2)cc1)Cc1ccc(C(F)(F)F)o1] | ¹H-NMR (CDCl₃, 400 MHz, mixture of isomers) δ: 7.83-7.69 (m, 3H), 7.63-7.27 (m, 10H), 7.07 (d, J = 8.0 Hz, 1H), 6.81- 6.80 (m, 0.5H), 6.57-6.56 (m, 0.5H), 6.44 (d, J = 2.8 Hz, 0.5H), 5.85 (d, J = 3.2 Hz, 0.5H), 5.05-4.82 (m, 2H), 4.26, 4.15 (2 s, 2H), 3.84-3.77 (m, 1H), 2.46 (s, 3H), 1.60- 1.55 (m, 3H); MS: 572.0 (M + 1)⁺. |

### Example 11

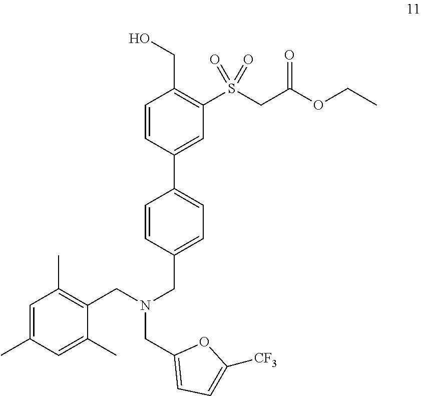 [SMILES: CCOC(=O)CS(=O)(=O)c1cc(-c2ccc(CN(Cc3ccc(C(F)(F)F)o3)Cc3c(C)cc(C)cc3C)cc2)ccc1CO]

### Ethyl 2-((4-(hydroxymethyl)-4′-((((5-(trifluoromethyl)furan-2-yl)methyl(2,4,6-trimethyl-benzyl)amino)methyl)-[1,1′-biphenyl]-3-yl)sulfonyl)acetate (11)

To a solution of compound 10c (200 mg, 0.39 mmol) in 1,4-dioxane (10 mL) and water (1 mL) was added compound P1 (130 mg, 0.39 mmol), Na₂CO₃ (83 mg, 0.78 mmol) and Pd(dppf)Cl₂ (20 mg). The mixture was stirred at 90° C. for 3 h, cooled, diluted with water (40 mL) and extracted with EA (3×20 mL). The combined organic layer was washed with brine (30 mL), dried over Na₂SO₄, filtered, concentrated and purified by FCC (PE:EA=10:1) to give compound 11 as a white solid.

### Example 12

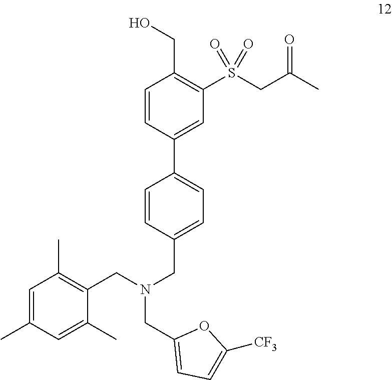 [SMILES: CC(=O)CS(=O)(=O)c1cc(-c2ccc(CN(Cc3ccc(C(F)(F)F)o3)Cc3c(C)cc(C)cc3C)cc2)ccc1CO]

### 2-((4-(Hydroxvmethyl)-4′-((((5-(trifluoromethyl)furan-2-yl)methyl)(2,4,6-trimethyl-benzyl)amino)methyl)-[1,1′-biphenyl]-3-yl)sulfonyl)acetic acid (12)

Compound 11 (120 mg, 0.19 mmol) was saponified as described in Example 7 to obtain compound 12 as a white solid. ¹H-NMR (500 MHz, CD₃OD) δ: 8.25 (d, J=2.0 Hz, 1H), 7.97 (dd, J=8.0, 1.5 Hz, 1H), 7.82 (d, J=7.5 Hz, 1H), 7.62 (d, J=8.0 Hz, 2H), 7.39 (d, J=8.0 Hz, 2H), 6.88 (d, J=2.0 Hz, 1H), 6.84 (s, 2H), 6.38 (d, J=3.5 Hz, 1H), 5.08 (s, 2H), 4.43 (s, 2H), 3.73 (s, 2H), 3.64 (s, 2H), 3.58 (s, 2H), 2.34 (s, 6H), 2.24 (s, 3H); MS: 616.2 (M+H)⁺.

### Example 12/1 to 12/4

The following Examples were prepared similar as described for Example 11 using the appropriate building blocks and optionally saponified as described in Example 12.

| # | building blocks | structure | analytical data |
|---|---|---|---|
| 12/1 | 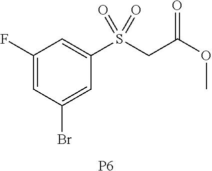 | 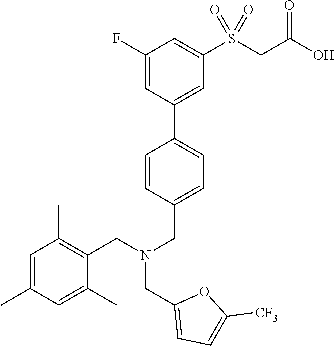 [SMILES: Cc1cc(C)c(CN(Cc2ccc(-c3cc(F)cc(S(=O)(=O)CC(=O)O)c3)cc2)Cc2ccc(C(F)(F)F)o2)c(C)c1] | ¹H-NMR (CD₃OD, 400 MHz) δ: 8.02 (s, 1H), 8.75 (d, J = 10.4 Hz, 1H), 7.68-7.62 (m, 3H), 7.40 (d, J = 8.4 Hz, 2H), 6.87 (dd, 1.2, 3.2 Hz, 1H), 6.82 (s, 2H), 6.38 (d, J = 2.8 Hz, 1H), 4.38 (br s, 2H), 3.71 (s, 2H), 3.63 (s, 2H), 3.57 (s, 2H), 2.31 (s, 6H), 2.21 (s, 3H); MS: 604.1 (M + H)⁺. |
| 12/2 | 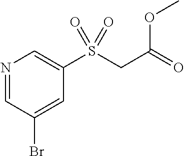 | 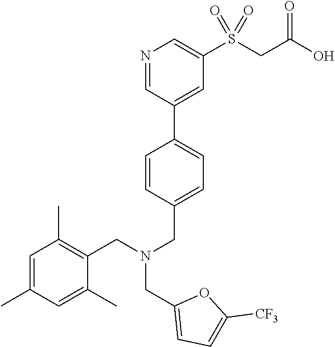 [SMILES: Cc1cc(C)c(CN(Cc2ccc(-c3cncc(S(=O)(=O)CC(=O)O)c3)cc2)Cc2ccc(C(F)(F)F)o2)c(C)c1] | ¹H-NMR (CDCl₃, 400 MHz) δ: 9.01 (s, 1H), 8.82 (s, 1H), 8.29 (s, 1H), 7.37 (d, J = 7.6 Hz, 2H), 7.26-7.23 (m, 2H), 6.78 (s, 2H), 6.65 (d, J = 2.0 Hz, 1H), 6.14 (d, J = 2.8 Hz, 1H), 4.22 (s, 2H), 3.60 (s, 2H), 3.49 (s, 2H), 3.43 (s, 2H), 2.27 (s, 6H), 2.19 (s, 3H); MS: 587.1 (M + H)⁺. |
| 12/3 | 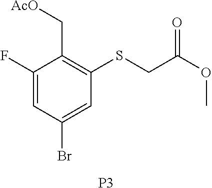 | 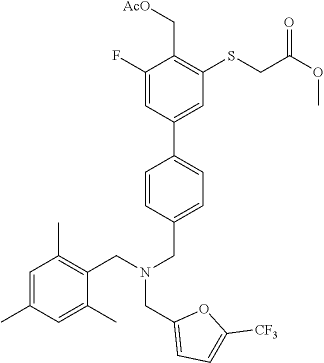 | |
| 12/4 | 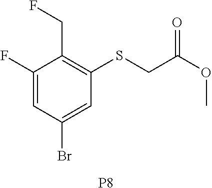 | 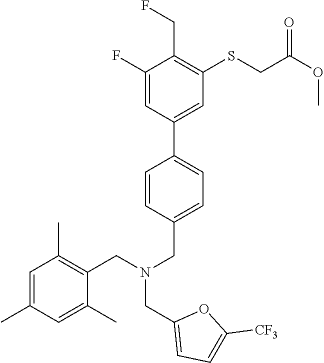 | |

### Example 13

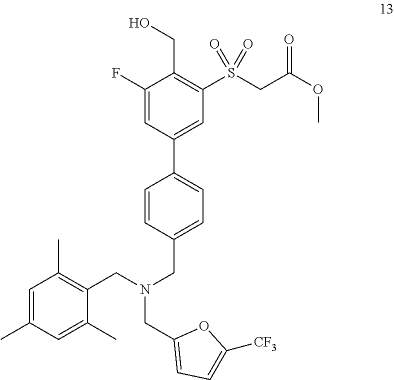

### Methyl 2-((5-fluoro-4-(hydroxymethyl)-4′-((((5-(trifluoromethyl)furan-2-yl)methyl)(2,4,6-tri-methylbenzyl)amino)methyl-[1,1′-biphenyl]-3-yl)sulfonyl)acetate (13)

To a solution of compound 20/1 (240 mg, 0.38 mmol) in THF (20 mL) was added K₂CO₃ (52 mg, 0.38 mmol) and MeI (110 mg, 0.76 mmol) at rt. The mixture was stirred at 60° C. overnight, cooled, filtered and concentrated. The residue was purified by prep-HPLC to give compound 13 as a white solid. ¹H-NMR (CDCl₃, 400 MHz) δ: 8.09 (s, 1H), 7.61 (dd, J=1.6, 10.4 Hz, 1H), 7.52 (d, J=8.4 Hz, 2H), 7.38 (d, J=8.0 Hz, 2H), 6.83 (s, 2H), 6.71 (d, J=2.0 Hz, 1H), 6.22 (d, J=2.8 Hz, 1H), 5.09-5.08 (m, 2H), 4.44 (s, 2H), 3.71 (s, 3H), 3.68 (s, 2H), 3.60 (s, 2H), 3.56 (s, 2H), 2.74-2.72 (m, 1H), 2.34 (s, 6H), 2.24 (s, 3H); MS: 648.0 (M+1)⁺.

### Example 14

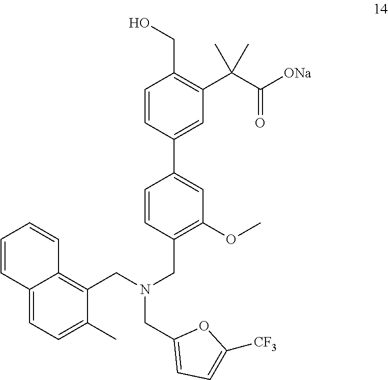

### Sodium 2-(4-(hydroxymethyl)-3′-methoxy-4′-((((2-meth(naphthalen-1-yl)methyl)((5-(trifluoromethyl)furan-2-yl)methyl)amino)methyl)-[1,1′-biphenyl]-3-yl)-2-methylpropanoate (14)

To a solution of compound 7/9 (150 mg, 0.24 mmol) in MeOH (10 mL) and water (10 mL) was added NaOH (10 mg, 0.48 mmol) at rt. The mixture was stirred at rt overnight and concentrated. The residue was washed with H₂O to give compound 14 as a white solid. The compound tends to cyclisize back to lacton 7/9 upon standing. ¹H-NMR (CD₃OD, 400 MHz) δ: 8.22 (d, J=8.0 Hz, 1H), 7.74 (dd, J=2.0, 7.6 Hz, 1H), 7.65 (d, J=8.0 Hz, 1H), 7.57 (d, J=1.6 Hz, 1H), 7.52-7.50 (m, 1H), 7.42-7.35 (m, 3H), 7.31-7.26 (m, 2H), 7.07-7.05 (m, 2H), 6.83-6.82 (m, 1H), 6.32-6.31 (m, 1H), 4.67 (s, 2H), 4.15 (s, 2H), 3.75 (s, 3H), 3.69 (s, 2H), 3.67 (s, 2H), 2.53 (s, 3H), 1.61 (s, 3H), 1.55 (s, 3H); MS: 632.0 (M+1)⁺.

### Example 14/1 to 14/3

The following Examples were saponified similar as described for Example 14 using the appropriate building block.

| # | building block | structure | analytical data |
|---|---|---|---|
| 14/1 | 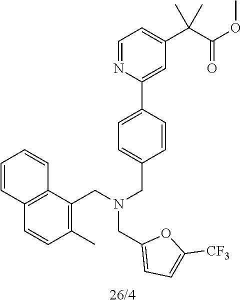 | 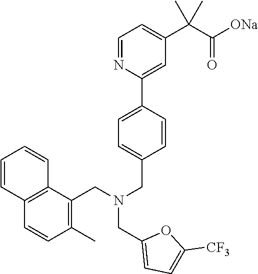 | ¹H-NMR (CD₃OD, 400 MHz) δ: 8.43 (d, J = 5.2 Hz, 1H), 8.24 (d, J = 8.4 Hz, 1H), 7.79- 7.75 (m, 4H), 7.67 (d, J = 8.4 Hz, 1H), 7.46- 7.37 (m, 3H), 7.32-7.28 (m, 3H), 6.88 (dd, J = 3.2 Hz, J = 1.2 Hz, 1H), 6.36 (d, J = 3.2 Hz, 1H), 4.17 (s, 2H), 3.70 (s, 2H), 3.61 (s, 2H), 2.54 (s, 3H), 1.54 (s, 6H); MS: 573.0 (M − Na + 2)⁺. |
| 14/2 | 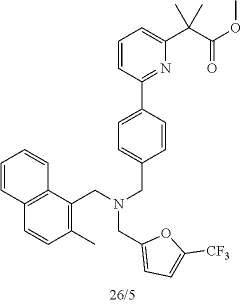 | 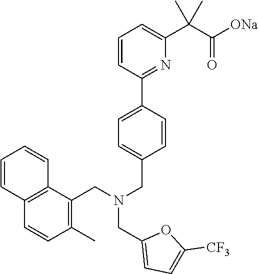 | ¹H-NMR (CD₃OD, 400 MHz) δ: 8.26 (d, J = 8.0 Hz, 1H), 7.98 (d, J = 8.4 Hz, 2H), 7.77 (d, J = 7.6 Hz, 1H), 7.69-7.64 (m, 2H), 7.56 (d, J = 7.6 Hz, 1H), 7.46-7.40 (m, 2H), 7.31- 7.27 (m, 4H), 6.88 (d, J = 2.4 Hz, 1H), 6.36 (d, J = 3.2 Hz, 1H), 4.18 (s, 2H), 3.71 (s, 2H), 3.60 (s, 2H), 2.55 (s, 3H), 1.58 (s, 6H); MS: 573.0 (M − Na + 2)⁺. |
| 14/3 | 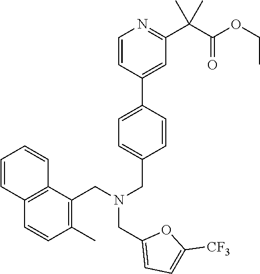 | 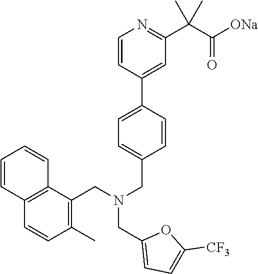 | ¹H-NMR (CD₃OD, 400 MHz) δ: 8.41 (d, J = 4.8 Hz, 1H), 8.24 (d, J = 8.4 Hz, 1H), 7.76 (dd, J = 8.0, 0.8 Hz, 1H), 7.66 (dd, J = 8.4, 1.2 Hz, 2H), 7.58 (d, J = 8.4 Hz, 2H), 7.47- 7.38 (m, 3H), 7.31-7.28 (m, 3H), 6.87 (dd, J = 3.6, 1.2 Hz, 1H), 6.36 (d, J = 3.6 Hz, 1H), 4.17 (s, 2H), 3.71 (s, 2H), 3.60 (s, 2H), 2.54 (s, 3H), 1.57 (s, 6H); MS: 573.0 (M − Na + 2)⁺. |

### Example 15

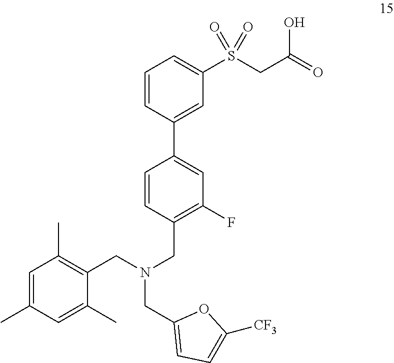 [SMILES: Cc1cc(C)c(CN(Cc2ccc(C(F)(F)F)o2)Cc2ccc(-c3cccc(S(=O)(=O)CC(=O)O)c3)cc2F)c(C)c1]

### Step 1: 1-Mesityl-N-((5-(trifluoromethyl)furan-2-yl)methyl)methanamine (15a)

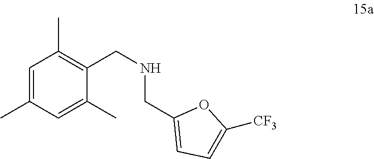 [SMILES: Cc1cc(C)c(CNCc2ccc(C(F)(F)F)o2)c(C)c1]

To a solution of mesitylmethanamine (5.13 g, 34.4 mmol) and TEA (19.2 mL, 138 mmol) in THF (150 mL) was added 2-(bromomethyl)-5-(trifluoromethyl)furan (7.88 g, 34.4 mmol) at rt. The mixture was stirred under N₂ at 85° C. overnight, concentrated and purified by FCC (PE:EA=10:1 with 1% TEA) to obtain compound 15a as a yellow oil.

### Step 2: N-(4-Bromo-2-fluorobenzyl)-1-mesityl-N-((5-(trifluoromethyl)furan-2-yl)methyl)methanamine (15)

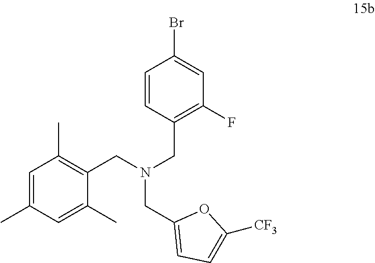 [SMILES: Cc1cc(C)c(CN(Cc2ccc(C(F)(F)F)o2)Cc2ccc(Br)cc2F)c(C)c1]

To a solution of compound 15a (500 mg, 1.68 mmol) in ACN (20 mL) was added 4-bromo-1-(bromomethyl)-2-fluorobenzene (541 mg, 2.02 mmol) and K₂CO₃ (464 mg, 3.36 mmol). The mixture was stirred at 70° C. overnight, cooled, filtered, concentrated and purified by FCC (PE:EA=10:1) to give compound 15b as a colorless oil.

### Step 3: 2-((3′-Fluoro-4′-((((5-(trifluoromethyl)furan-2-yl)methyl)(2,4,6-trimethylbenzyl)amino)methyl)-[1,1′-biphenyl]-3-yl)sulfonyl)acetic acid (15)

Compound 15a was coupled and saponified as described in Example 6, Step 2 and Example 7 to afford compound 15. ¹H-NMR (CDCl₃, 400 MHz) δ: 8.11 (s, 1H), 7.92 (d, J=6.4 Hz, 1H), 7.80-7.78 (m, 1H), 7.60 (br s, 2H), 7.41-7.39 (m, 1H), 7.31-7.26 (m, 1H), 6.89-6.80 (m, 4H), 4.39 (s, 2H), 4.34 (s, 2H), 4.16 (s, 2H), 4.12 (s, 2H), 2.26 (s, 9H); MS: 604.2 (M+H)⁺.

### Example 15/1 to 15/4

The following Examples were prepared similar as described for Example 15 using the appropriate building blocks.

| # | building block | structure | analytical data |
|---|---|---|---|
| 15/1 | 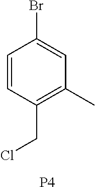 | 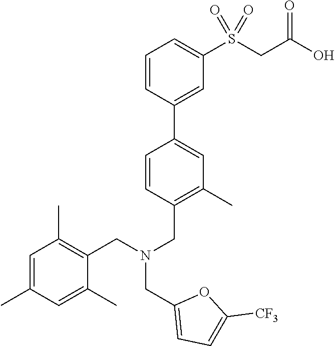 [SMILES: Cc1cc(C)c(CN(Cc2ccc(C(F)(F)F)o2)Cc2ccc(-c3cccc(S(=O)(=O)CC(=O)O)c3)cc2C)c(C)c1] | ¹H-NMR (DMSO-d₆, 400 MHz) δ: 8.13 (s, 1H), 7.92 (d, J = 8.0 Hz, 1H), 7.83 (d, J = 8.0 Hz, 1H), 7.63 (t, J = 7.6 Hz, 1H), 7.52-7.49 (m, 2H), 7.40 (d, J = 8.0 Hz, 1H), 7.13 (d, J = 2.0 Hz, 1H), 6.81 (s, 2H), 6.55 (d, J = 3.2 Hz, 1H), 4.05 (s, 2H), 3.58 (s, 2H), 3.56 (s, 2H), 3.51 (s, 2H), 2.22 (s, 6H), 2.18 (s, 3H), 2.11 (s, 3H); MS: 600.2 (M + H)⁺. |
| 15/2 | 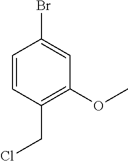 | 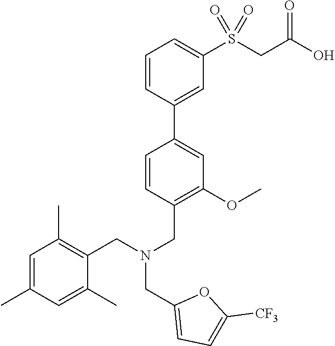 [SMILES: COc1cc(-c2cccc(S(=O)(=O)CC(=O)O)c2)ccc1CN(Cc1ccc(C(F)(F)F)o1)Cc1c(C)cc(C)cc1C] | ¹H-NMR (CDCl₃, 400 MHz) δ: 8.00 (s, 1H), 7.75 (d, J = 6.4 Hz, 1H), 7.51 (dd, J = 1.2, 8.0 Hz, 1H), 7.26-7.24 (m, 2H), 6.92 (d, J = 8.0 Hz, 1H), 6.84 (s, 1H), 6.74 (s, 2H), 6.62 (d, J = 2.0 Hz, 1H), 6.16 (d, J = 2.8 Hz, 1H), 4.15 (br s, 2H), 3.63 (s, 2H), 3.61 (s, 2H), 3.58 (s, 2H), 3.48 (s, 3H), 2.24 (s, 6H), 2.15 (s, 3H); MS: 616.2 (M + 1)⁺. |
| 15/3 | 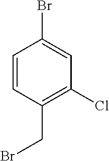 | 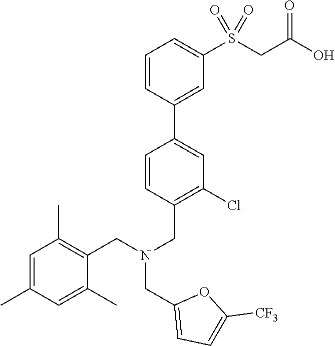 [SMILES: Cc1cc(C)c(CN(Cc2ccc(C(F)(F)F)o2)Cc2ccc(-c3cccc(S(=O)(=O)CC(=O)O)c3)cc2Cl)c(C)c1] | ¹H-NMR (CDCl₃, 300 MHz) δ: 8.00 (s, 1H), 7.83 (d, J = 9.0 Hz, 1H), 7.54 (d, J = 9.0 Hz, 1H), 7.42-7.36 (m, 3H), 7.28-7.25 (m, 1H), 6.79 (s, 2H), 6.65 (d, J = 1.8 Hz, 1H), 6.20 (d, J = 3.0 Hz, 1H), 4.17 (s, 2H), 3.63 (s, 2H), 3.58 (s, 2H), 3.53 (s, 2H), 2.27 (s, 6H), 2.20 (s, 3H); MS: 620.1 (M + 1)⁺. |
| 15/4 | 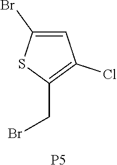 | 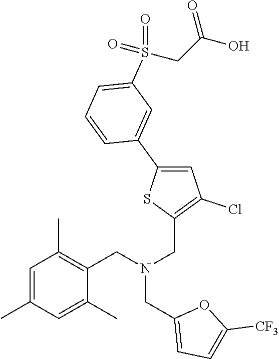 [SMILES: Cc1cc(C)c(CN(Cc2ccc(C(F)(F)F)o2)Cc2sc(-c3cccc(S(=O)(=O)CC(=O)O)c3)cc2Cl)c(C)c1] | ¹H-NMR (CDCl₃, 400 MHz) δ: 7.96 (s, 1H), 7.74 (d, J = 7.6 Hz, 1H), 7.47 (d, J = 8.0 Hz, 1H), 7.31-7.27 (m, 1H), 6.97 (s, 1H), 6.79 (s, 2H), 6.67 (d, J = 2.0 Hz, 1H), 6.23 (d, J = 3.2 Hz, 1H), 4.18 (s, 2H), 3.64 (s, 2H), 3.61 (s, 2H), 3.57 (s, 2H), 2.28 (s, 6H), 2.19 (s, 3H); MS: 626.1 (M + H)⁺. |

### Example 16

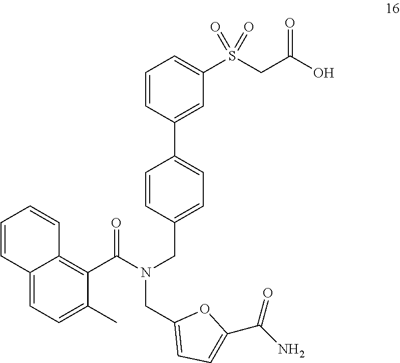 [SMILES: Cc1ccc2ccccc2c1C(=O)N(Cc1ccc(-c2cccc(S(=O)(=O)CC(=O)O)c2)cc1)Cc1ccc(C(N)=O)o1]

### 2-((4′-((N-((5-Carbamoylfuran-2-yl)methyl)-2-methyl-1-naphthamido)methyl)-[1,1′-biphenyl]-3-yl)sulfonyl)acetic acid 16

To a solution of compound 2712 (180 mg, 0.30 mmol) in THF (5 mL) and water (5 mL) was added LiOH.H₂O (26 mg, 0.60 mmol) at rt. The mixture was stirred at rt overnight, concentrated and purified by prep-HPLC to afford compound 16 as a white solid. ¹H-NMR (CD₃OD, 400 MHz, mixture of isomers) δ: 8.22, 8.10 (2 s, 1H), 8.01-7.86 (m, 4H), 7.74-7.63 (m, 4H), 7.51-7.47 (m, 3H), 7.41 (t, J=8.0 Hz, 1H), 7.14-6.83 (m, 2H), 6.56 (d, J=3.6 Hz, 0.5H), 5.92 (d, J=3.2 Hz, 0.5H), 5.19-4.96 (m, 2H), 4.39-4.29 (m, 4H), 2.42, 2.39 (2 s, 3H); MS: 597.0 (M+H)⁺.

### Example 17

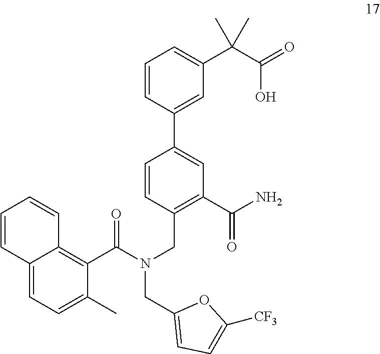 [SMILES: Cc1ccc2ccccc2c1C(=O)N(Cc1ccc(C(F)(F)F)o1)Cc1ccc(-c2cccc(C(C)(C)C(=O)O)c2)cc1C(N)=O]

### Step 1: N-(4-Bromo-2-carbamoylbenzyl)-2-methyl-N-((5-(trifluoromethyl)furan-2-yl)methyl)-1-naphthamide (17a)

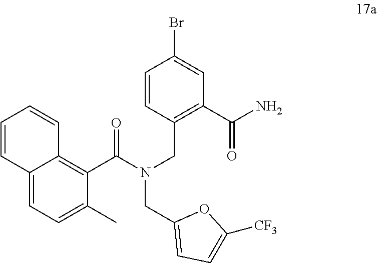 [SMILES: Cc1ccc2ccccc2c1C(=O)N(Cc1ccc(C(F)(F)F)o1)Cc1ccc(Br)cc1C(N)=O]

To a solution of N-(4-bromo-2-cyanobenzyl)-2-methyl-N-((5-(trifluoromethyl)furan-2-yl)methyl)-1-naphthamide (intermediate from Example 27/7, 238 mg, 0.44 mmol) in EtOH/H₂O (15 mL/3 mL) was added KOH (323 mg, 0.44 mmol) at rt. The mixture was stirred at 60° C. overnight, diluted with water (100 mL) and extracted with EA (3×70 mL). The combined organic layer was washed with brine (70 mL), dried over Na₂SO₄ and concentrated to give compound 17a as a yellow solid.

### Step 2. 2-((4′-((N-((5-Carbamoylfuran-2-yl)methyl)-2-methyl-1-naphthamido)methyl)-[1,1′-biphenyl]-3-yl)sulfonyl)acetic acid (17)

To a solution of compound 17a (227 mg, 0.42 mmol) and 2-methyl-2-(3-(4,4,5,5-tetramethyl-1,3,2-dioxaborolan-2-yl)phenyl)propanoic acid (122 mg, 0.42 mmol) in ACN/H₂O (9 mL/3 mL) was added S-phos (17 mg, 40 μmol), Pd(OAc)₂ (5 mg, 20 μmol) and K₃PO₄ (233 mg, 1.1 mmol) at rt under N₂. The mixture was stirred at 90° C. under N₂ overnight, adjusted to pH=4 with aq. HCl, filtered and purified by prep-HPLC to give compound 17 as a white solid. ¹H-NMR (CDCl₃, 400 MHz) δ: 7.82-7.59 (m, 5H), 7.48-7.32 (m, 7H), 7.16-7.05 (m, 2H), 6.85-6.68 (m, 1H), 6.48 (br s, 0.5H), 5.37 (d, J=2.8 Hz, 0.5H), 5.93-5.79 (m, 1H), 5.20-4.90 (m, 2H), 4.64-4.49 (m, 1H), 4.37 (s, 1H), 2.42, 2.39 (2 s, 3H), 1.67, 1.64 (2 s, 6H); MS: 629.3 (M+H)⁺.

### Example 18

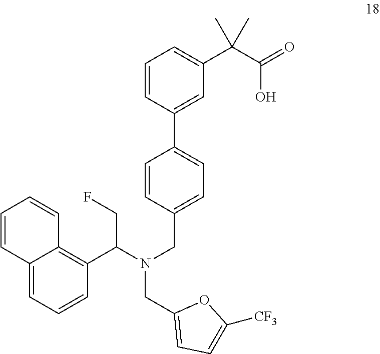 [SMILES: CC(C)(C(=O)O)c1cccc(-c2ccc(CN(Cc3ccc(C(F)(F)F)o3)C(CF)c3cccc4ccccc34)cc2)c1]

### Step 1: Ethyl 2-bromo-2-(naphthalen-1-yl)acetate (18a)

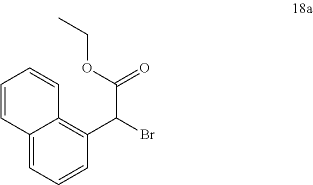 [SMILES: CCOC(=O)C(Br)c1cccc2ccccc12]

To a solution of ethyl 2-(naphthalen-1-yl)acetate (2.1 g, 9.8 mmol) in CCl₄ (20 mL) was added NBS (2.0 g, 11 mmol) and AIBN (82 mg). The mixture was stirred at 80° C. for 5 h, cooled to rt, diluted with water (50 mL) and extracted with DCM (2×). The combined organic layer was washed with brine, dried over Na₂SO₄, filtered and concentrated to give compound 18a as a yellow oil.

### Step 2: Ethyl 2-((4-bromobenzyl)((5-(trifluoromethyl)furan-2-yl)methyl)amino)-2-(naphthalen-1-ylacetate (18b)

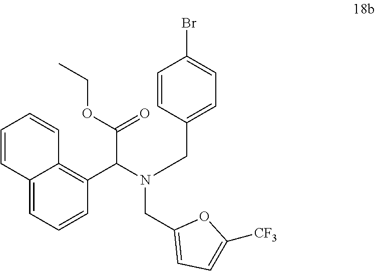 [SMILES: CCOC(=O)C(c1cccc2ccccc12)N(Cc1ccc(Br)cc1)Cc1ccc(C(F)(F)F)o1]

The solution of compound 18a (600 mg, 2.0 mmol) and N-(4-bromobenzyl)-1-(5-(trifluoromethyl)furan-2-yl)methanamine (753 mg, 2.2 mmol) in EtOH (10 mL) was refluxed overnight under N₂, cooled, concentrated, diluted with water (5 mL) and extracted with EA (2×25 mL). The combined organic layer was washed with brine, dried over Na₂SO₄, filtered, concentrated and purified by prep-TLC (PE:EA=20:1) to give compound 18b as a yellow oil. ¹H-NMR (CDCl₃, 400 MHz) δ: 8.10 (d, J=9.2 Hz, 1H), 7.84-7.79 (m, 2H), 7.53-7.50 (m, 2H), 7.41-7.39 (m, 2H), 7.33-7.31 (m, 2H), 7.02 (d, J=8.4 Hz, 2H), 6.66 (d, J=2.0 Hz, 1H), 6.07 (d, J=2.4 Hz, 1H), 5.28 (s, 1H), 4.31-4.24 (m, 2H), 3.87 (s, 2H), 3.84 (s, 2H), 1.27 (t, J=7.2 Hz, 3H).

### Step 3: 2-((4-Bromobenzyl)((5-(trifluoromethyl)furan-2-yl)methyl)amino)-2-(naphthalen-1-yl)ethan-1-ol (18c)

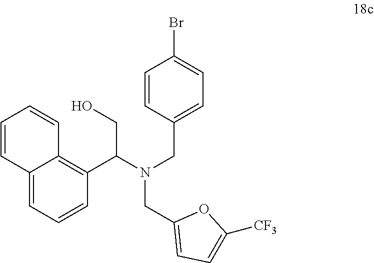 [SMILES: OCC(c1cccc2ccccc12)N(Cc1ccc(Br)cc1)Cc1ccc(C(F)(F)F)o1]

A solution of LiAlH₄ in dry THF (0.7 mL, 1M, 0.7 mmol) was added dropwise to a solution of compound 18b (310 mg, 0.55 mmol) in dry THF (8 mL) under N₂ at rt. The mixture was stirred overnight, diluted with a sat. aq. solution of NH₄Cl (10 mL) and extracted with EA (2×10 mL). The combined organic layer was washed with brine, dried over Na₂SO₄, filtered, concentrated and purified by prep-TLC (PE:EA=10:1) to give compound 18c as a yellow oil.

### Step 4: N-(4-Bromobenzyl)-2-fluoro-1-(naphthalen-1-yl)-N-((5-(trifluoromethyl)furan-2-yl)methyl)ethan-1-amine (18d)

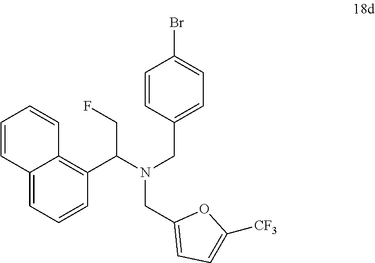 [SMILES: FCC(c1cccc2ccccc12)N(Cc1ccc(Br)cc1)Cc1ccc(C(F)(F)F)o1]

To a solution of compound 18c (300 mg, 0.60 mol) in DCM (3 mL) was added DAST (0.6 mL). The mixture was stirred at rt overnight, quenched with ice and extracted with EA (2×10 mL). The combined organic layer was washed with brine, dried over Na₂SO₄, filtered, concentrated and purified by prep-TLC (PE:EA=10:1) to give compound 18d as a yellow oil.

### Step 5: 2-(4′-(((2-Fluoro-1-(naphthalen-1-yl)ethyl)((5-(trifluoromethyl)furan-2-yl)methyl)amino)methyl)-[1,1′-biphenyl]-3-yl)-2-methylpropanoic acid (18)

A solution of compound 18d (160 mg, 0.17 mmol), 2-(3-boronophenyl)-2-methylpropanoic acid (79 mg, 0.38 mmol), K₂CO₃ (131 mg, 0.95 mmol) and Pd(dppf)Cl₂ (20 mg) in 1,4-dioxane/H₂O (2/1; 3 mL) under N₂ was stirred for 50 min at 110° C., cooled to rt, adjusted to pH=1 using 1N HCl and extracted with EA (2×10 mL). The combined organic layer was washed with brine, dried over Na₂SO₄, filtered, concentrated and purified by prep-HPLC to give compound 18 as a white solid. ¹H-NMR (CDCl₃, 400 MHz) δ: 7.83-7.78 (m, 2H), 7.60-7.57 (m, 2H), 7.53-7.38 (m, 10H), 7.31-7.25 (m, 1H), 6.73 (d, J=1.6 Hz, 1H), 6.75-6.30 (m, 2H), 4.00-3.94 (m, 3H), 3.75 (d, J=13.2 Hz, 1H), 3.15-3.10 (m, 2H), 1.67 (s, 6H); MS: 590.2 (M+H)⁺.

### Example 19

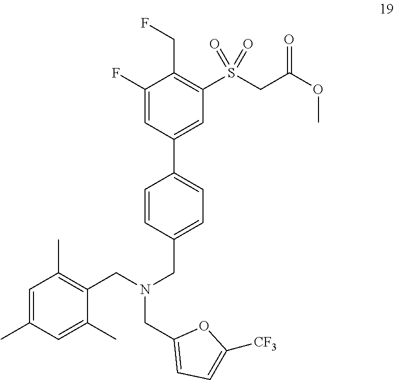

### Methyl 2-((5-fluoro-4-(fluoromethyl)-4′-((((5-(trifluoromethyl)furan-2-yl)methyl)(2,4,6-trimethyl-benzyl)amino)methyl-[1,1′-biphenyl]-3-yl)sulfonyl)acetate (19)

To a mixture of compound 12/4 (120 mg, 194 μmol) in DCM (5 mL) was added m-CPBA (118 mg, 583 μmol) and the mixture was stirred at rt overnight, quenched with aq. NaHSO₃ and extracted with EA (3×). The combined organic layer washed with brine (10 mL), dried over Na₂SO₄, filtered, concentrated and purified by prep-TLC (PE:EA=5:1) to give compound 19 as a white solid.

### Example 19-1

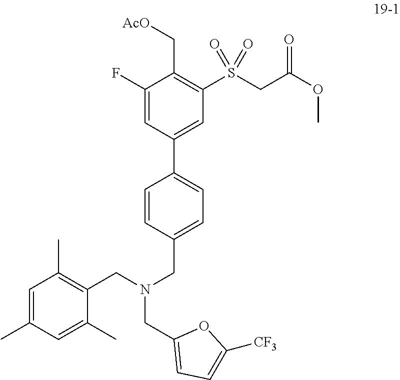

### Methyl 2-((4-(acetoxymethyl)-5-fluoro-4′-((((5-trifluoromethyl)furan-2-yl)methyl)(2,4,6-tri-methylbenzyl)amino)methyl)-[1,1′-biphenyl]-3-yl)sulfonyl)acetate (19-1)

Similar as described for Example 19, compound 12/3 (180 mg, 274 μmol) was oxidized to afford compound 19-1 as a white solid.

### Example 20

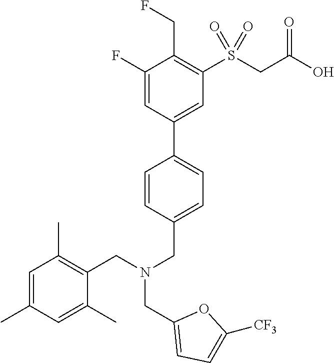 [SMILES: Cc1cc(C)c(CN(Cc2ccc(-c3cc(F)c(CF)c(S(=O)(=O)CC(=O)O)c3)cc2)Cc2ccc(C(F)(F)F)o2)c(C)c1]

### 2-((5-Fluoro-4-(fluoromethyl)-4′-((((5-(trifluoromethyl)furan-2-yl)methyl)(2,4,6-trimethyl-benzyl)amino)methyl)-[1,1′-biphenyl]-3-yl)sulfonyl)acetic acid (20)

Compound 19 (60 mg, 92 μmol) was saponified as described in Example 9 to give compound 20 as a white solid. ¹H-NMR (CDCl₃, 400 MHz) δ: 8.04 (s, 1H), 7.38-7.34 (m, 3H), 7.26-7.23 (m, 2H), 6.80 (s, 2H), 6.67 (d, J=2.4 Hz, 1H), 6.17 (d, J=2.8 Hz, 1H), 5.86 (br s, 1H), 5.74 (br s, 1H), 4.28 (br s, 2H), 3.62 (s, 2H), 3.52 (s, 2H), 3.45 (s, 2H), 2.28 (s, 6H), 2.20 (s, 3H); MS: 636.2 (M+H)⁺.

### Example 20/1

The following Example was saponified similar as described for Example 20.

| # | building block | structure | analytical data |
|---|---|---|---|
| 20/1 | 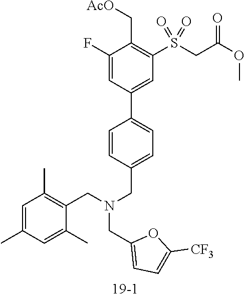 | 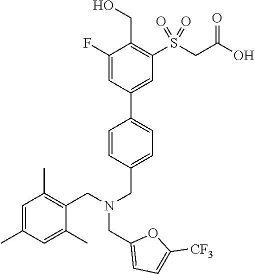 [SMILES: Cc1cc(C)c(CN(Cc2ccc(-c3cc(F)c(CO)c(S(=O)(=O)CC(=O)O)c3)cc2)Cc2ccc(C(F)(F)F)o2)c(C)c1] | ¹H-NMR (CDCl₃, 400 MHz) δ: 7.88 (s, 1H), 7.26-7.23 (m, 2H), 7.16-7.12 (m, 3H), 6.75 (s, 2H), 6.61 (d, J = 1.6 Hz, 1H), 6.10 (d, J = 3.2 Hz, 1H), 4.88 (br s, 2H), 4.33 (br s, 2H), 3.55 (s, 2H), 3.43 (s, 2H), 3.36 (s, 2H), 2.24 (s, 6H), 2.16 (s, 3H); MS: 634.2 (M + H)⁺. |

### Example 21

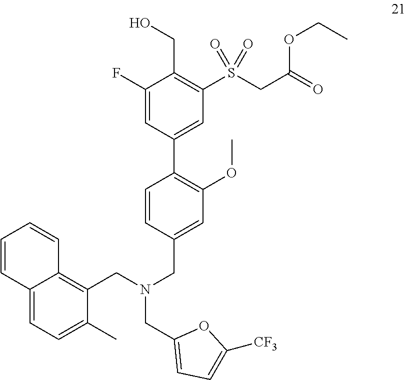 [SMILES: CCOC(=O)CS(=O)(=O)c1cc(-c2ccc(CN(Cc3ccc(C(F)(F)F)o3)Cc3c(C)ccc4ccccc34)cc2OC)cc(F)c1CO]

### Step 1: N-(4-Bromo-3-methoxybenz)-1-(2-methylnaphthalen-1-yl)-N-((5-(trifluoromethyl)furan-2-yl)methyl)methanamine (21a)

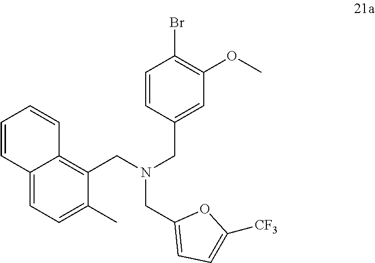 [SMILES: COc1cc(CN(Cc2ccc(C(F)(F)F)o2)Cc2c(C)ccc3ccccc23)ccc1Br]

Compound 21a was prepared from tert-butyl (4-bromo-3-methoxybenzyl)carbamate P9, 2-(bromomethyl)-5-(trifluoromethyl)furan and 2-methyl-1-naphthaldehyde similar as described in Example 1, Step 1 and Example 10, Step 1 and Step 2 to afford compound 21a as a colorless oil.

### Step 2: Ethyl 2-((5-fluoro-4-(hydroxymethyl)-2′-methoxy-4′-((((2-methylnaphthalen-1-yl)methyl)((5-(trifluoromethyl)furan-2-yl)methyl)amino)methyl)-[1,1′-biphenyl]-3-yl)sulfonyl)acetate (21)

To a solution of compound 21a (200 mg, 0.39 mmol) in 1,4-dioxane (10 mL) and water (1 mL) was added compound P10 (137 mg, 0.39 mmol), B₂Pin₂ (99 mg, 0.39 mmol), KOAc (77 mg, 0.78 mmol) and Pd(dppf)Cl₂ (20 mg). The mixture was stirred at 90° C. for 3 h under N₂, cooled, diluted with water (40 mL) and extracted with EA (3×20 mL). The combined organic layer was washed with brine (30 mL), dried over Na₂SO₄, filtered, concentrated and purified by FCC (PE:EA=5:1) to give compound 21 as a white solid.

### Example 21/1 to 21/8

The following Examples were synthesized similar as described for Example 21 or Example 6 using the appropriate building blocks.

| # | building blocks | structure |
|---|---|---|
| 21/1 | 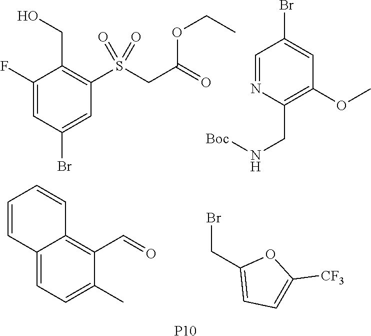 | 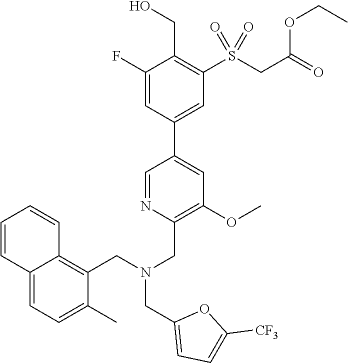 [SMILES: CCOC(=O)CS(=O)(=O)c1cc(-c2cnc(CN(Cc3ccc(C(F)(F)F)o3)Cc3c(C)ccc4ccccc34)c(OC)c2)cc(F)c1CO] |
| 21/2 | 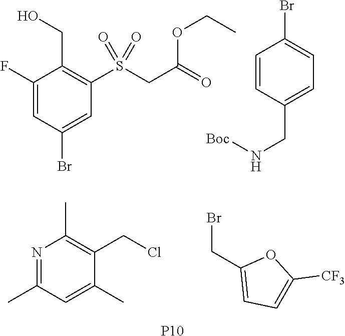 | 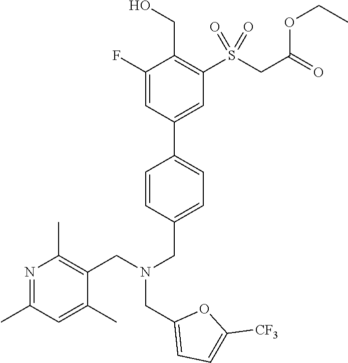 [SMILES: CCOC(=O)CS(=O)(=O)c1cc(-c2ccc(CN(Cc3ccc(C(F)(F)F)o3)Cc3c(C)cc(C)nc3C)cc2)cc(F)c1CO] |
| 21/3 | 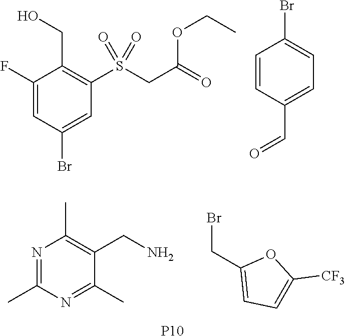 | 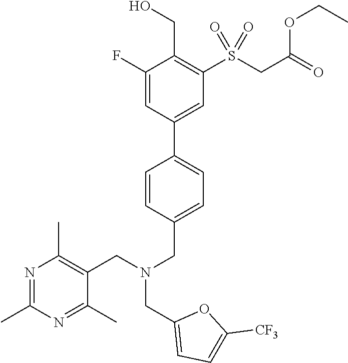 [SMILES: CCOC(=O)CS(=O)(=O)c1cc(-c2ccc(CN(Cc3ccc(C(F)(F)F)o3)Cc3c(C)nc(C)nc3C)cc2)cc(F)c1CO] |
| 21/4 | 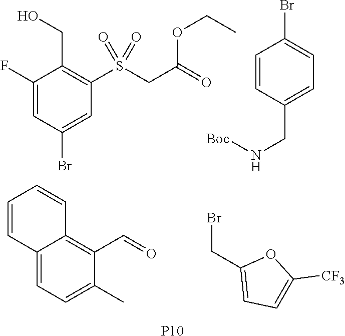 | 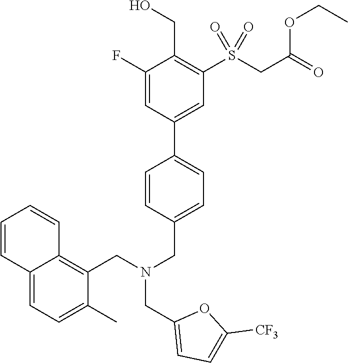 [SMILES: CCOC(=O)CS(=O)(=O)c1cc(-c2ccc(CN(Cc3ccc(C(F)(F)F)o3)Cc3c(C)ccc4ccccc34)cc2)cc(F)c1CO] |
| 21/5 | 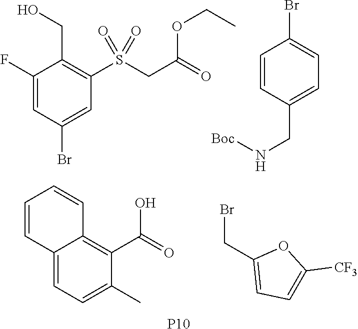 | 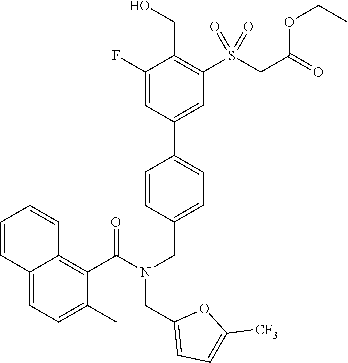 [SMILES: CCOC(=O)CS(=O)(=O)c1cc(-c2ccc(CN(Cc3ccc(C(F)(F)F)o3)C(=O)c3c(C)ccc4ccccc34)cc2)cc(F)c1CO] |
| 21/6 | 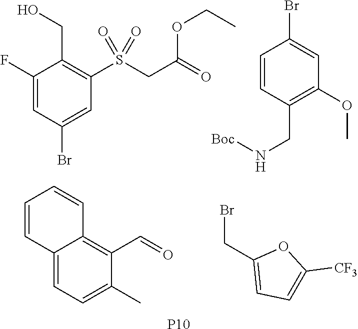 | 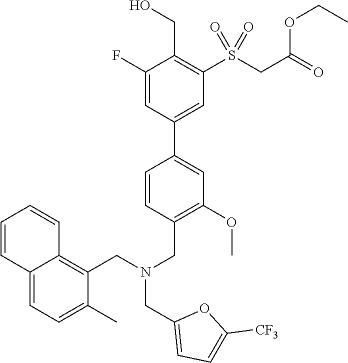 [SMILES: CCOC(=O)CS(=O)(=O)c1cc(-c2ccc(CN(Cc3ccc(C(F)(F)F)o3)Cc3c(C)ccc4ccccc34)c(OC)c2)cc(F)c1CO] |
| 21/7 | 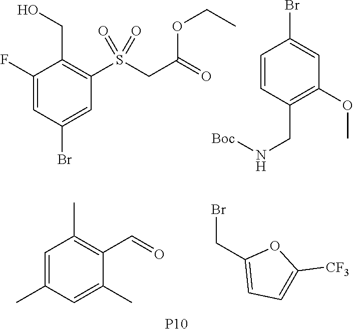 | 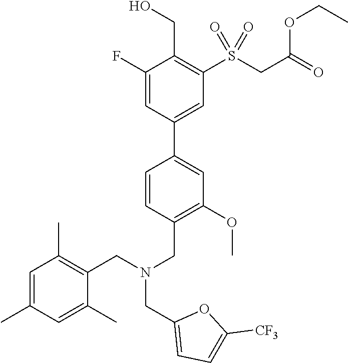 [SMILES: CCOC(=O)CS(=O)(=O)c1cc(-c2ccc(CN(Cc3ccc(C(F)(F)F)o3)Cc3c(C)cc(C)cc3C)c(OC)c2)cc(F)c1CO] |
| 21/8 | 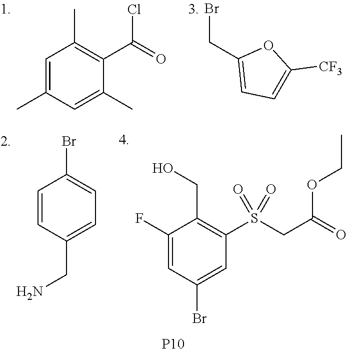 | 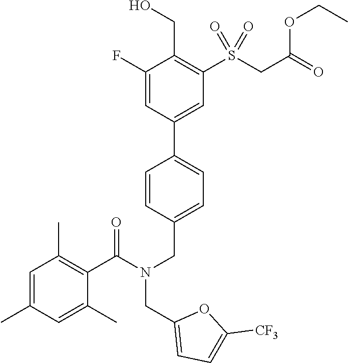 [SMILES: CCOC(=O)CS(=O)(=O)c1cc(-c2ccc(CN(Cc3ccc(C(F)(F)F)o3)C(=O)c3c(C)cc(C)cc3C)cc2)cc(F)c1CO] |

### Example 21-1

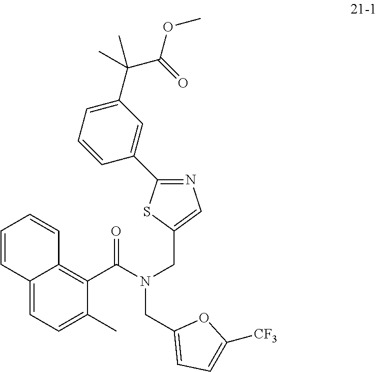 [SMILES: COC(=O)C(C)(C)c1cccc(-c2ncc(CN(Cc3ccc(C(F)(F)F)o3)C(=O)c3c(C)ccc4ccccc34)s2)c1]

### Step 1: 1-(2-Chlorothiazol-5-yl)-N-((2-methylnaphthalen-1-yl)methyl)-N-((5-(trifluoromethyl)furan-2-yl)methyl)methanamine (21-1a)

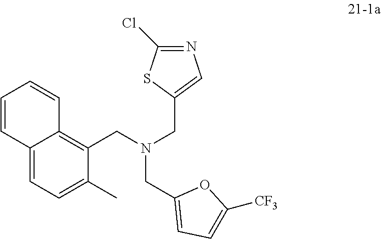 [SMILES: Cc1ccc2ccccc2c1CN(Cc1ccc(C(F)(F)F)o1)Cc1cnc(Cl)s1]

Using tert-butyl ((2-chlorothiazol-5-yl)methyl)carbamate, 2-(bromomethyl)-5-(trifluoromethyl)furan and 2-methyl-1-naphthaldehyde similar as described in Example 21, compound 21-1a was prepared as a colorless oil.

### Step 2: Methyl 2-methyl-2-(3-(5-((((2-methylnaphthalen-1-yl)methyl)((5-(trifluoromethyl)furan-2-)methyl)amino)methyl)thiazol-2-yl)phenyl)propanoate (21-1)

Compound 21-1a (200 mg, 0.44 mmol) was coupled similar as described in Example 23 to afford compound 21-1 as a white solid.

### Example 21-1/1 to 21-1/3

The following Examples were synthesized similar as described for Example 21 using the appropriate building blocks.

| # | building blocks | structure |
|---|---|---|
| 21-1/1 | 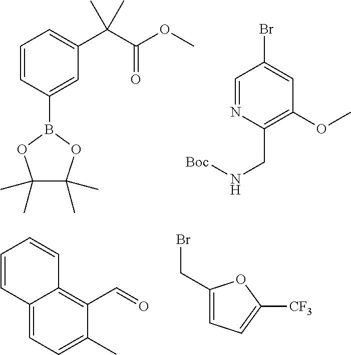 | 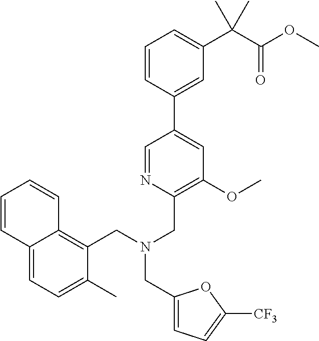 [SMILES: COC(=O)C(C)(C)c1cccc(-c2cnc(CN(Cc3ccc(C(F)(F)F)o3)Cc3c(C)ccc4ccccc34)c(OC)c2)c1] |
| 21-1/2 | 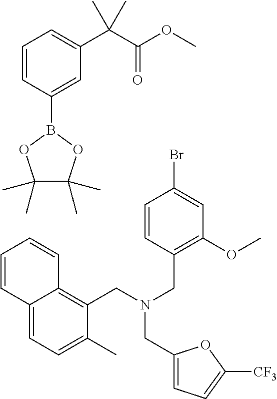 | 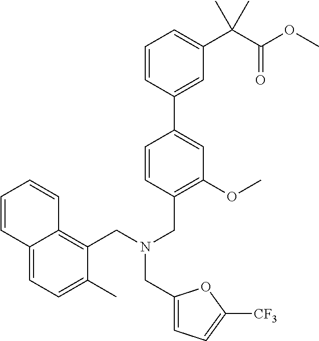 [SMILES: COC(=O)C(C)(C)c1cccc(-c2ccc(CN(Cc3ccc(C(F)(F)F)o3)Cc3c(C)ccc4ccccc34)c(OC)c2)c1] |
| 21-1/3 | 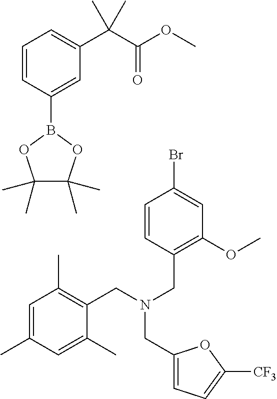 | 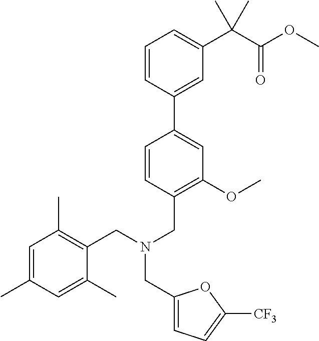 |

### Example 22

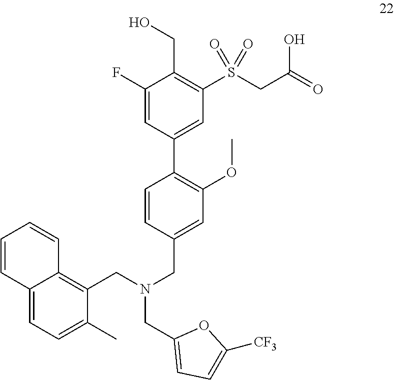 [SMILES: COc1cc(CN(Cc2ccc(C(F)(F)F)o2)Cc2c(C)ccc3ccccc23)ccc1-c1cc(F)c(CO)c(S(=O)(=O)CC(=O)O)c1]

### 2-((5-Fluoro-4-(hydroxymethyl)-2′-methoxy-4′-((((2-methylnaphthalen-1-yl)methyl)((5-(trifluoromethyl)furan-2-yl)methyl)amino)methyl)-[1,1′-biphenyl]-3-yl)sulfonyl)acetic acid (22)

Compound 21 (120 mg, 0.17 mmol) was saponified as described in Example 7 to give compound 22 as a white solid. ¹H-NMR (500 MHz, CD₃OD) δ: 8.02 (s, 2H), 7.86 (d, J=8.0 Hz, 1H), 7.81 (d, J=8.5 Hz, 1H), 7.66 (dd, J=8.5, 1.0 Hz, 1H), 7.53-7.46 (m, 2H), 7.37 (d, J=9.0 Hz, 1H), 7.30 (d, J=8.0 Hz, 1H), 7.05 (br s, 2H), 6.99 (d, J=8.0 Hz, 1H), 6.71 (br s, 1H), 5.09 (d, J=1.0 Hz, 2H), 4.66 (s, 2H), 4.62 (br s, 2H), 4.24 (br s, 2H), 4.06 (br s, 2H), 3.74 (s, 3H), 2.57 (s, 3H); MS: 686.2 (M+H)⁺.

### Example 22/1 to 22/13

The following Examples were saponified similar as described for Example 22.

| # | building block(s) | structure | analytical data |
|---|---|---|---|
| 22/1 | 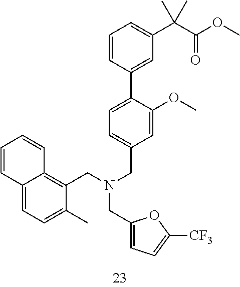 | 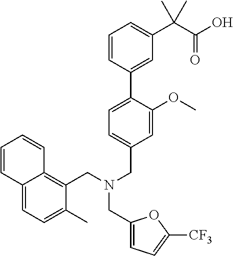 [SMILES: COc1cc(CN(Cc2ccc(C(F)(F)F)o2)Cc2c(C)ccc3ccccc23)ccc1-c1cccc(C(C)(C)C(=O)O)c1] | ¹H-NMR (500 MHz, CD₃OD) δ: 8.28 (d, J = 8.5 Hz, 1H), 7.79 (d, J = 8.0 Hz, 1H), 7.70 (d, J = 8.5 Hz, 1H), 7.47-7.39 (m, 3H), 7.32-7.28 (m, 4H), 7.11 (d, J = 7.5 Hz, 1H), 6.93 (d, J = 2.5 Hz, 1H), 6.90 (s, 1H), 6.83 (d, J = 7.5 Hz, 1H), 6.44 (d, J = 3.0 Hz, 1H), 4.20 (s, 2H), 3.77 (s, 2H), 3.62 (s, 3H), 3.58 (s, 2H), 2.58 (s, 3H), 1.57 (s, 6H); MS: 601.9 (M + H)⁺. |
| 22/2 | 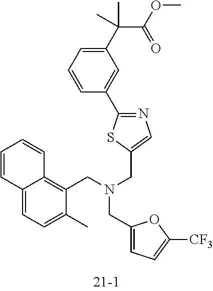 | 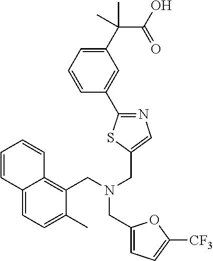 [SMILES: Cc1ccc2ccccc2c1CN(Cc1ccc(C(F)(F)F)o1)Cc1cnc(-c2cccc(C(C)(C)C(=O)O)c2)s1] | ¹H-NMR (500 MHz, CD₃OD) δ: 8.25 (d, J = 8.5 Hz, 1H), 7.87 (s, 1H), 7.79 (d, J = 8.0 Hz, 1H), 7.71 (d, J = 8.0 Hz, 1H), 7.65 (d, J = 7.5 Hz, 1H), 7.55 (s, 1H), 7.51-7.40 (m, 4H), 7.32 (d, J = 8.0 Hz, 1H), 6.91 (s, 1H), 6.43 (d, J = 2.5 Hz, 1H), 4.25 (s, 2H), 3.86 (s, 4H), 2.56 (s, 3H), 1.61 (s, 6H); MS: 578.8 (M + H)⁺. |
| 22/3 | 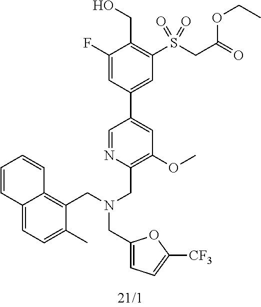 | 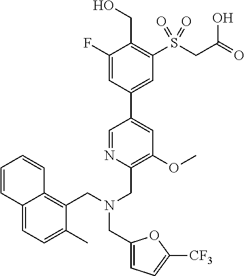 [SMILES: COc1cc(-c2cc(F)c(CO)c(S(=O)(=O)CC(=O)O)c2)cnc1CN(Cc1ccc(C(F)(F)F)o1)Cc1c(C)ccc2ccccc12] | ¹H-NMR (500 MHz, CD₃OD) δ: 8.13 (d, J = 8.5 Hz, 1H), 7.95 (s, 1H), 7.72 (dd, J = 1.8, 10.0 Hz, 1H), 7.65-7.59 (m, 4H), 7.47 (t, J = 7.3 Hz, 1H), 7.39 (d, J = 1.0 Hz, 1H), 7.25 (d, J = 8.0 Hz, 1H), 7.15 (d, J = 2.5 Hz, 1H), 6.94 (d, J = 3.0 Hz, 1H), 5.13 (d, J = 1.5 Hz, 2H), 4.79-4.76 (m, 6H), 4.33 (s, 2H), 3.77 (s, 3H), 2.59 (s, 3H); MS: 687.2 (M + H)⁺. |
| 22/4 | 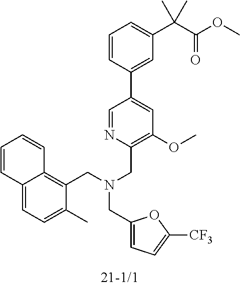 | 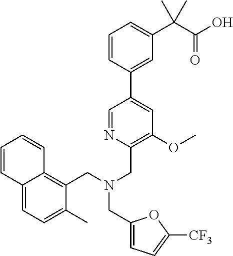 [SMILES: COc1cc(-c2cccc(C(C)(C)C(=O)O)c2)cnc1CN(Cc1ccc(C(F)(F)F)o1)Cc1c(C)ccc2ccccc12] | ¹H-NMR (500 MHz, CD₃OD) δ: 8.19 (d, J = 8.0 Hz, 1H), 8.08 (d, J = 1.5 Hz, 1H), 7.73-7.71 (m, 1H), 7.65 (d, J = 8.5 Hz, 1H), 7.56 (s, 1H), 7.46 (s, 3H), 7.39-7.26 (m, 4H), 6.81 (d, J = 2.5 Hz, 1H), 6.31 (d, J = 3.5 Hz, 1H), 4.23 (s, 2H), 3.90 (s, 2H), 3.87 (s, 2H), 3.58 (s, 3H), 2.52 (s, 3H), 1.63 (s, 6H); MS: 602.9 (M + H)⁺. |
| 22/5 | 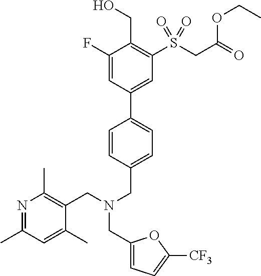 | 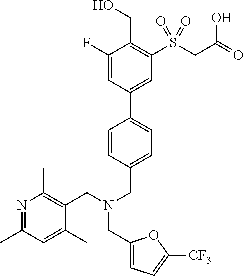 | ¹H-NMR (500 MHz, CD₃OD) δ: 8.11 (d, J = 1.5 Hz, 1H), 7.71 (dd, J = 1.3, 10.7 Hz, 1H), 7.58 (d, J = 8.0 Hz, 2H), 7.36 (d, J = 8.0 Hz, 2H), 7.30 (s, 1H), 6.93 (dd, J = 1.3, 3.3 Hz, 1H), 6.48 (d, J = 3.0 Hz, 1H), 5.08 (d, J = 1.5 Hz, 2H), CH₂ signal at 4.6 ppm not resolved, 3.87 (s, 2H), 3.79 (s, 2H), 3.70 (s, 2H), 2.67 (s, 3H), 2.55 (s, 3H), 2.52 (s, 3H); MS: 602.9 (M + H)⁺. |
| 22/6 | 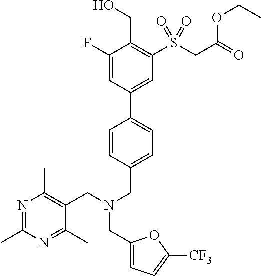 | 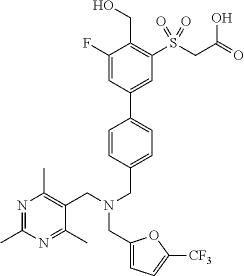 [SMILES: Cc1nc(C)c(CN(Cc2ccc(-c3cc(F)c(CO)c(S(=O)(=O)CC(=O)O)c3)cc2)Cc2ccc(C(F)(F)F)o2)c(C)n1] | ¹H-NMR (500 MHz, CD₃OD) δ: 8.15 (s, 1H), 7.79 (dd, J = 2.0, 10.5 Hz, 1H), 7.63 (d, J = 8.5 Hz, 2H), 7.42 (d, J = 8.0 Hz, 2H), 6.90 (d, J = 1.5 Hz, 1H), 6.43 (d, J = 3.0 Hz, 1H), 5.11 (d, J = 1.0 Hz, 2H), 4.60 (s, 2H), 3.84 (s, 2H), 3.74 (s, 2H), 3.69 (s, 2H), 2.55 (s, 6H), 2.52 (s, 3H); MS: 636.2 (M + H)⁺. |
| 22/7 | 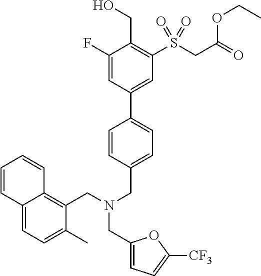 | 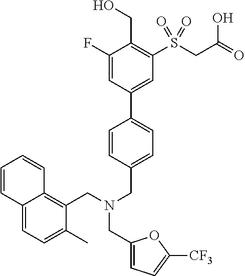 [SMILES: Cc1ccc2ccccc2c1CN(Cc1ccc(-c2cc(F)c(CO)c(S(=O)(=O)CC(=O)O)c2)cc1)Cc1ccc(C(F)(F)F)o1] | ¹H-NMR (500 MHz, CD₃OD) δ: 8.14 (s, 1H), 8.06 (br s, 1H), 7.78-7.84 (m, 3H), 7.64 (d, J = 7.5 Hz, 2H), 7.43-7.51 (m, 4H), 7.37 (d, J = 8.5 Hz, 1H), 7.00 (s, 1H), 6.60 (s, 1H), 5.11 (d, J = 1.5 Hz, 2H), 4.69 (s, 2H), 4.51 (br s, 2H), 4.09 (br s, 2H), 3.97 (br s, 2H), 2.55 (s, 3H); MS: 655.8 (M + H)⁺. |
| 22/8 | 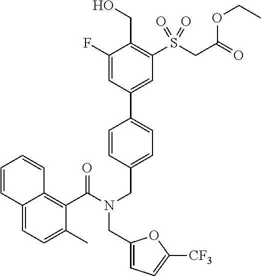 | 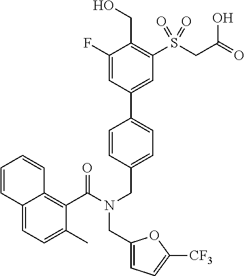 [SMILES: Cc1ccc2ccccc2c1C(=O)N(Cc1ccc(-c2cc(F)c(CO)c(S(=O)(=O)CC(=O)O)c2)cc1)Cc1ccc(C(F)(F)F)o1] | ¹H-NMR (500 MHz, CD₃OD, mixture of isomers) δ: 8.21, 8.09 (2 s, 1H), 7.42-7.92 (m, 10H), 7.01-7.10 (m, 1H), 7.01 (d, J = 2.0 Hz, 0.5H), 6.74 (d, J = 2.5 Hz, 0.5H), 6.57 (d, J = 3.5 Hz, 0.5H), 6.10 (d, J = 3.5 Hz, 0.5H), 4.89-5.13 (m, 4H), 4.31-4.43 (m, 4H), 2.47, 2.44 (2 s, 3H); MS: 670.2 (M + H)⁺. |
| 22/9 | 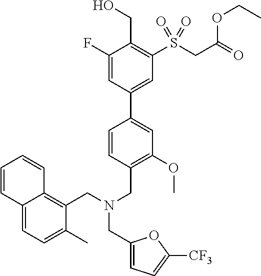 | 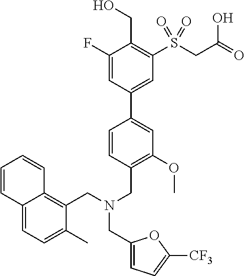 [SMILES: COc1cc(-c2cc(F)c(CO)c(S(=O)(=O)CC(=O)O)c2)ccc1CN(Cc1ccc(C(F)(F)F)o1)Cc1c(C)ccc2ccccc12] | ¹H-NMR (500 MHz, CD₃OD) δ: 8.24 (d, J = 8.5 Hz, 1H), 8.12 (s, 1H), 7.67-7.77 (m, 3H), 7.34-7.44 (m, 3H), 7.30 (d, J = 8.5 Hz, 1H), 7.14 (d, J = 6.5 Hz, 2H), 6.88 (d, J = 2.5 Hz, 1H), 6.37 (d, J = 3.5 Hz, 1H), 5.09 (s, 2H), 4.39 (s, 2H), 4.20 (s, 2H), 3.79 (s, 3H), 3.75 (s, 2H), 3.71 (s, 2H), 2.55 (s, 3H); MS: 686.2 (M + H)⁺. |
| 22/10 | 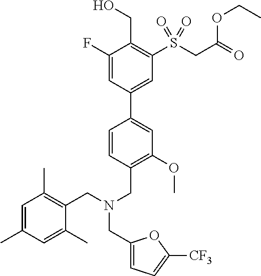 | 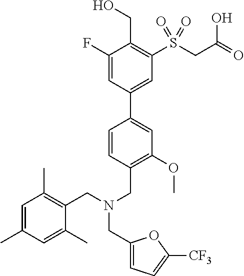 [SMILES: COc1cc(-c2cc(F)c(CO)c(S(=O)(=O)CC(=O)O)c2)ccc1CN(Cc1ccc(C(F)(F)F)o1)Cc1c(C)cc(C)cc1C] | ¹H-NMR (500 MHz, CD₃OD) δ: 8.03 (s, 1H), 7.65-7.67 (m, 1H), 7.31 (d, J = 8.0 Hz, 1H), 7.12 (d, J = 8.0 Hz, 1H), 7.09 (s, 1H), 6.75 (d, J = 2.5 Hz, 1H), 6.70 (s, 2H), 6.29 (d, J = 3.5 Hz, 1H), 4.98 (s, 2H), 4.35-4.37 (m, 2H), 3.76 (s, 3H), 3.62 (s, 2H), 3.56 (s, 2H), 3.53 (s, 2H), 2.19 (s, 6H), 2.11 (s, 3H); MS: 664.2 (M + H)⁺. |
| 22/11 | 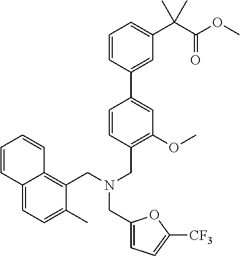 | 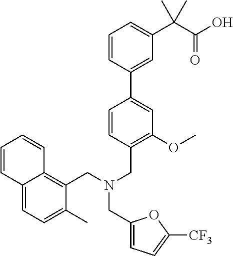 [SMILES: COc1cc(-c2cccc(C(C)(C)C(=O)O)c2)ccc1CN(Cc1ccc(C(F)(F)F)o1)Cc1c(C)ccc2ccccc12] | ¹H-NMR (500 MHz, CD₃OD) δ: 8.24 (d, J = 8.5 Hz, 1H), 7.77 (d, J = 7.5 Hz, 1H), 7.68 (d, J = 8.5 Hz, 1H), 7.57 (s, 1H), 7.38-7.46 (m, 5H), 7.30 (d, J = 8.5 Hz, 2H), 7.04-7.06 (m, 2H), 6.87-6.86 (m, 1H), 6.36 (d, J = 3.0 Hz, 1H), 4.18 (s, 2H), 3.76 (s, 3H), 3.73 (s, 2H), 3.70 (s, 2H), 2.55 (s, 3H), 1.61 (s, 6H); MS: 601.9 (M + H)⁺. |
| 22/12 | 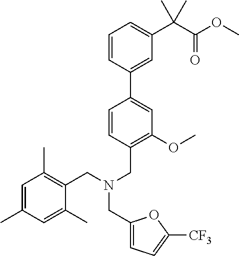 | 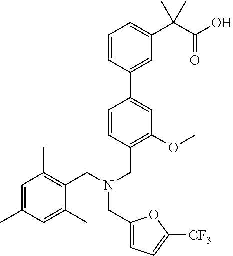 [SMILES: COc1cc(-c2cccc(C(C)(C)C(=O)O)c2)ccc1CN(Cc1ccc(C(F)(F)F)o1)Cc1c(C)cc(C)cc1C] | ¹H-NMR (500 MHz, CD₃OD) δ: 7.60 (s, 1H), 7.34-7.49 (m, 4H), 7.12 (dd, J = 1.5, 7.5 Hz, 1H), 7.08 (d, J = 1.5 Hz, 1H), 6.85 (d, J = 2.0 Hz, 1H), 6.81 (s, 2H), 6.36 (d, J = 3.0 Hz, 1H), 3.84 (s, 3H), 3.70 (s, 2H), 3.62 (s, 2H), 3.61 (s, 2H), 2.31 (s, 6H), 2.23 (s, 3H), 1.62 (s, 6H); MS: 580.3 (M + H)⁺. |
| 22/13 | 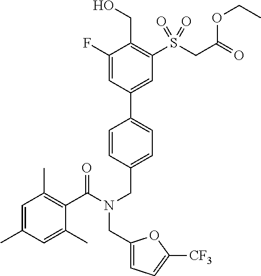 | 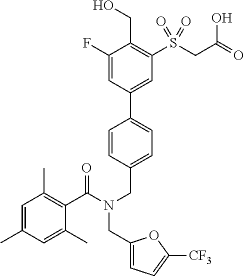 [SMILES: Cc1cc(C)c(C(=O)N(Cc2ccc(-c3cc(F)c(CO)c(S(=O)(=O)CC(=O)O)c3)cc2)Cc2ccc(C(F)(F)F)o2)c(C)c1] | ¹H-NMR (500 MHz, CD₃OD, mixture of isomers) δ: 8.15 (dd, J = 9.8, 1.3 Hz, 1H), 7.81 (ddd, J = 10.6, 4.5, 1.8 Hz, 1H), 7.70-7.66 (m, 2H), 7.54 (d, J = 8.5 Hz, 1H), 7.24 (d, J = 8.0 Hz, 1H), 6.97-6.96 (m, 2.5H), 6.85 (dd, J = 3.5, 1.0 Hz, 0.5H), 6.51 (d, J = 3.0 Hz, 0.5H), 6.32 (d, J = 3.5 Hz, 0.5H), 5.12 (dd, J = 4.0, 1.7 Hz, 2H), 4.87 (d, J = 3.0 Hz, 2H), 4.70 (d, J = 3.0 Hz, 2H), 4.43, 4.38 (2 s, 2H), 2.32, 2.31 (2 s, 3H), 2.25, 2,20 (2 s, 6H); MS: 648.2 (M + H)⁺. |

### Example 23

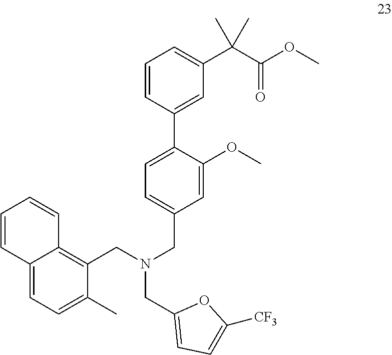 [SMILES: COC(=O)C(C)(C)c1cccc(-c2ccc(CN(Cc3ccc(C(F)(F)F)o3)Cc3c(C)ccc4ccccc34)cc2OC)c1]

### Methyl 2-(2′-methoxy-4′-((((2-methylnaphthalen-1-yl)methyl)((5-(trifluoromethyl)furan-2-yl)methyl)amino)methyl)-[1,1′-biphenyl]-3-yl)-2-methylpropanoate (23)

To a solution of compound 21a (200 mg, 0.39 mmol) in 1,4-dioxane (10 mL) and water (1 mL) was added methyl 2-methyl-2-(3-(4,4,5,5-tetramethyl-1,3,2-dioxaborolan-2-yl)phenyl)propanoate (142 mg, 0.47 mmol), Na₂CO₃ (83 mg, 0.78 mmol) and Pd(dppf)Cl₂ (20 mg) and the mixture was stirred at 90° C. for 3 h under N₂, cooled, diluted with water (40 mL) and extracted with EA (3×20 mL). The combined organic layer was washed with brine (30 mL), dried over Na₂SO₄, filtered, concentrated and purified by FCC (PE:EA=10:1) to give compound 23 as a white solid.

### Example 24

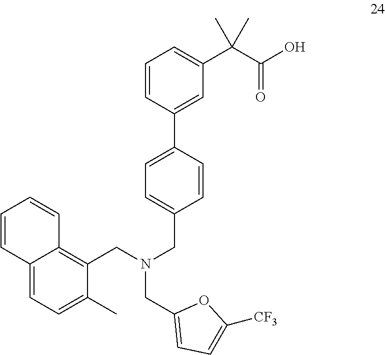 [SMILES: Cc1ccc2ccccc2c1CN(Cc1ccc(-c2cccc(C(C)(C)C(=O)O)c2)cc1)Cc1ccc(C(F)(F)F)o1]

### Step 1: Methyl 2-(4′-(((tert-butoxycarbonyl)amino)methyl)-[1,1′-biphenyl]-3-yl)-2-methylpropanoate (24a)

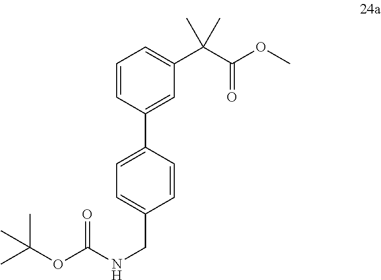 [SMILES: COC(=O)C(C)(C)c1cccc(-c2ccc(CNC(=O)OC(C)(C)C)cc2)c1]

To a solution of tert-butyl (4-(4,4,5,5-tetramethyl-1,3,2-dioxaborolan-2-yl)benzyl)carbamate (1.46 g, 4.40 mmol) in 1,4-dioxane (20 mL) and water (2 mL) was added methyl 2-(3-bromo-phenyl)-2-methylpropanoate (1.13 g, 4.40 mmol), Na₂CO₃ (1.20 g, 8.80 mmol) and Pd(dppf)Cl₂ (150 mg) and the mixture was stirred at 90° C. for 3 h under N₂, cooled, diluted with water (40 mL) and extracted with EA (3×20 mL). The combined organic layer was washed with brine (30 mL), dried over Na₂SO₄, filtered, concentrated and purified by FCC (PE:EA=10:1) to give compound 24a as a white solid.

### Step 2: Methyl 2-(4′-(((tert-butoxycarbonyl)((5-(trifluoromethyl)furan-2-yl)methyl)amino)methyl)-[1,1′-biphenyl]-3-yl)-2-methylpropanoate (24b)

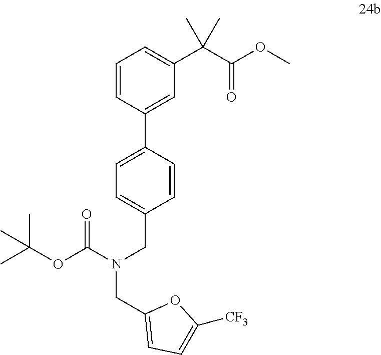 [SMILES: COC(=O)C(C)(C)c1cccc(-c2ccc(CN(Cc3ccc(C(F)(F)F)o3)C(=O)OC(C)(C)C)cc2)c1]

To a solution of compound 24a (957 mg, 2.50 mmol) in dry DMF (20 mL) was added NaH (200 mg, 5.00 mmol, 60% in oil) and 2-(bromomethyl)-5-(trifluoromethyl)furan (570 mg, 2.50 mmol) at 0° C. The mixture was stirred at rt overnight, diluted with water (200 mL) and extracted with EA (3×20 mL). The combined organic layer was washed with brine (30 mL), dried over Na₂SO₄, filtered, concentrated and purified by FCC (PE:EA=50:1) to give compound 24b as a colorless oil.

### Step 3: Methyl 2-methyl-2-(4′-((((5-(trifluoromethyl)furan-2-ylmethyl)amino)methyl)-[1,1′-biphenyl]-3-yl)propanoate (24c)

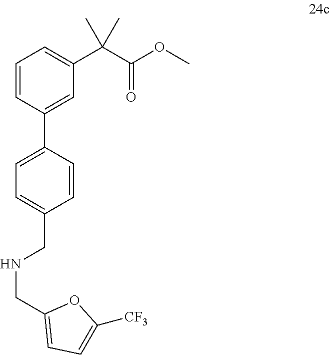 [SMILES: COC(=O)C(C)(C)c1cccc(-c2ccc(CNCc3ccc(C(F)(F)F)o3)cc2)c1]

To a solution of compound 24b (1.20 g, 2.30 mmol) in 1,4-dioxane (10 mL) was added HCl (5 mL, 6M in 1,4-dioxane) and the mixture was stirred at rt for 2 h, diluted with water (50 mL), adjusted to pH=8 with NaHCO₃ and extracted with EA (3×30 mL). The combined organic layer was washed with brine (40 mL), dried over Na₂SO₄, filtered and concentrated to give compound 24c as a yellow oil.

### Step 4: Methyl 2-methyl-2-(4-((((2-methylnaphthalen-1-yl)methyl)((5-(trifluoromethyl)furan-2-yl)methyl)amino)methyl)-[1,1′-biphenyl]-3-yl)propanoate (24d)

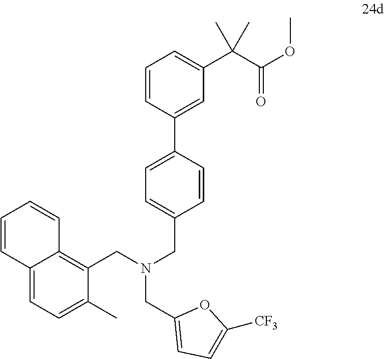 [SMILES: COC(=O)C(C)(C)c1cccc(-c2ccc(CN(Cc3ccc(C(F)(F)F)o3)Cc3c(C)ccc4ccccc34)cc2)c1]

To a solution of compound 24c (100 mg, 0.23 mmol) in 1,2-dichloroethane (5 mL) was added 2-methyl-1-naphthaldehyde (40 mg, 0.23 mmol) and one drop AcOH. The mixture was stirred at rt for 0.5 h. Then NaBH(OAc)₃ (195 mg, 0.92 mmol) was added and the mixture was stirred at rt overnight, diluted with water (40 mL) and extracted with DCM (3×20 mL). The combined organic layer was washed with brine (30 mL), dried over Na₂SO₄, filtered, concentrated and purified by FCC (PE:EA=50:1) to give compound 24d as a colorless oil.

### Step 5: 2-Methyl-2-(4′-((((2-methylnaphthalen-1-yl)methyl)((5-(trifluoromethyl)furan-2-yl)methyl)amino)methyl)-[1,1′-biphenyl]-3-yl)propanoic acid (24)

To a mixture of compound 24d (100 mg, 0.17 mmol) in MeOH (2 mL) and THF (1 mL) was added aq. LiOH (2M, 0.3 mL) and the mixture was stirred at rt overnight, neutralized with 1N HCl and extracted with EA (3×). The combined organic layer was washed with brine, dried over Na₂SO₄, filtered, concentrated and purified by prep-HPLC to give compound 24 as a white solid. ¹H-NMR (500 MHz, CD₃OD) δ: 7.91-7.83 (m, 3H), 7.64-7.62 (m, 3H), 7.51-7.39 (m, 8H), 7.04 (s, 1H), 6.70 (s, 1H), 4.68 (br s, 2H), 4.27 (br s, 2H), 4.16 (s, 2H), 2.54 (s, 3H), 1.63 (s, 6H); MS: 571.9 (M+H)⁺.

### Example 24/1 to 24/6

The following Examples were prepared and saponified similar as described for Example 24.

| # | building block | structure | analytical data |
|---|---|---|---|
| 24/1 | 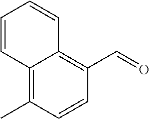 | 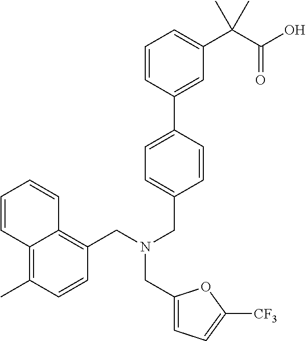 [SMILES: Cc1ccc(CN(Cc2ccc(-c3cccc(C(C)(C)C(=O)O)c3)cc2)Cc2ccc(C(F)(F)F)o2)c2ccccc12] | ¹H-NMR (500 MHz, CD₃OD) δ: 8.14 (d, J = 8.0 Hz, 1H), 7.74 (d, J = 8.0 Hz, 2H), 7.67-7.55 (m, 7H), 7.48-7.43 (m, 4H), 7.12 (d, J = 2.5 Hz, 1H), 6.89 (s, 1H), 4.71 (s, 2H), 4.47 (s, 2H), 4.40 (s, 2H), 2.74 (s, 3H), 1.64 (s, 6H); MS: 571.9 (M + H)⁺. |
| 24/2 | 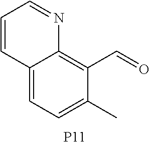 | 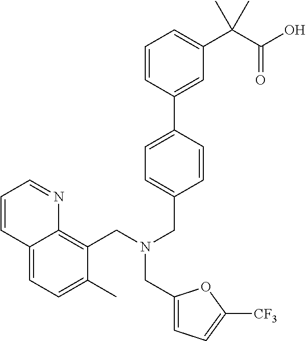 [SMILES: Cc1ccc2cccnc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 8.95 (dd, J = 9.5, 1.5 Hz, 1H), 8.43 (d, J = 8.0 Hz, 1H), 7.88 (d, J = 8.5 Hz, 1H), 7.66 (dd, J = 8.0, 4.3 Hz, 1H), 7.57-7.50 (m, 6H), 7.44 (s, 3H), 6.92 (d, J = 2.5 Hz, 1H), 6.77 (d, J = 3.5 Hz, 1H), 4.98 (s, 2H), 4.70 (s, 2H), 4.64 (s, 2H), 2.64 (s, 3H), 1.63 (s, 6H); MS: 573.3 (M + H)⁺. |
| 24/3 | 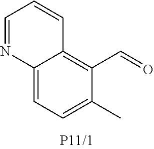 | 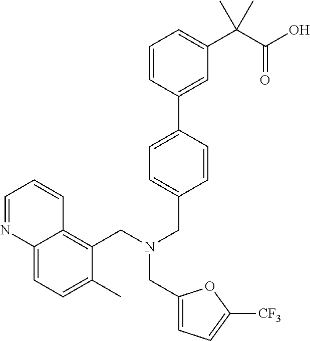 [SMILES: Cc1ccc2ncccc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 9.32 (d, J = 8.5 Hz, 1H), 9.01 (d, J = 5.0 Hz, 1H), 7.95 (d, J = 9.0 Hz, 1H), 7.89-7.86 (m, 2H), 7.54 (s, 1H), 7.45-7.37 (m, 5H), 7.25 (d, J = 8.5 Hz, 2H), 6.89 (d, J = 2.5 Hz, 1H), 6.42 (d, J = 3.0 Hz, 1H), 4.31 (s, 2H), 3.83 (s, 2H), 3.73 (s, 2H), 2.70 (s, 3H), 1.61 (s, 6H); MS: 573.2 (M + H)⁺. |
| 24/4 | 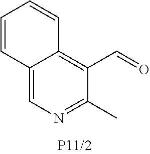 | 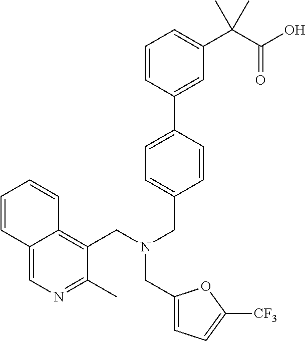 [SMILES: Cc1ncc2ccccc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 9.42 (s, 1H), 8.59 (d, J = 9.0 Hz, 1H), 8.32 (d, J = 8.5 Hz, 1H), 8.18-8.15 (m, 1H), 7.95 (t, J = 7.5 Hz, 1H), 7.48 (s, 1H), 7.39 (s, 3H), 7.32 (d, J = 8.5 Hz, 2H), 7.21 (d, J = 8.0 Hz, 2H), 6.92 (d, J = 2.0 Hz, 1H), 6.48 (d, J = 3.0 Hz, 1H), 4.36 (s, 2H), 3.94 (s, 2H), 3.80 (s, 2H), 2.91 (s, 3H), 1.62 (s, 6H); MS: 573.3 (M + H)⁺. |
| 24/5 | 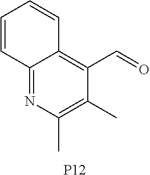 | 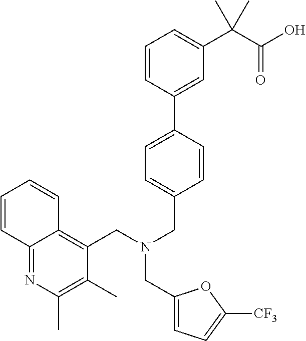 [SMILES: Cc1nc2ccccc2c(CN(Cc2ccc(-c3cccc(C(C)(C)C(=O)O)c3)cc2)Cc2ccc(C(F)(F)F)o2)c1C] | ¹H-NMR (500 MHz, CD₃OD) δ: 8.63 (d, J = 8.5 Hz, 1H), 8.04-7.98 (m, 2H), 7.88 (t, J = 7.3 Hz, 1H), 7.51 (s, 1H), 7.41-7.39 (m, 3H), 7.31 (d, J = 8.0 Hz, 2H), 7.16 (d, J = 7.5 Hz, 2H), 6.93 (d, J = 2.5 Hz, 1H), 6.50 (d, J = 3.5 Hz, 1H), 4.45 (s, 2H), 3.97 (s, 2H), 3.80 (s, 2H), 2.86 (s, 3H), 2.65 (s, 3H), 1.62 (s, 6H); MS: 587.3 (M + H)⁺. |
| 24/6 | 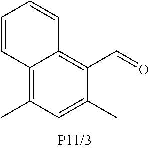 | 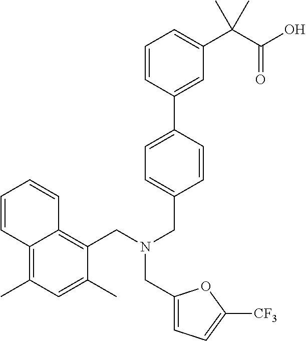 [SMILES: Cc1cc(C)c2ccccc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 8.29-8.27 (m, 1H), 7.98-7.96 (m, 1H), 7.57 (s, 1H), 7.48-7.36 (m, 7H), 7.27 (d, J = 7.5 Hz, 2H), 7.17 (s, 1H), 6.89 (d, J = 2.5 Hz, 1H), 6.37 (d, J = 2.5 Hz, 1H), 4.16 (s, 2H), 3.72 (s, 2H), 3.61 (s, 2H), 2.63 (s, 3H), 2.53 (s, 3H), 1.60 (s, 6H); MS: 586.2 (M + H)⁺. |

### Example 25

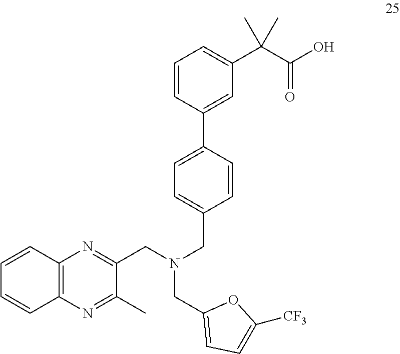 [SMILES: Cc1nc2ccccc2nc1CN(Cc1ccc(-c2cccc(C(C)(C)C(=O)O)c2)cc1)Cc1ccc(C(F)(F)F)o1]

### Step 1: Methyl 2-methyl-2-(4′-((((3-methylquinoxalin-2-yl)methyl)(5-(trifluoromethyl)furan-2-yl)methyl)amino)methyl)-[1,1′-biphenyl]-3-yl)propanoate (25a)

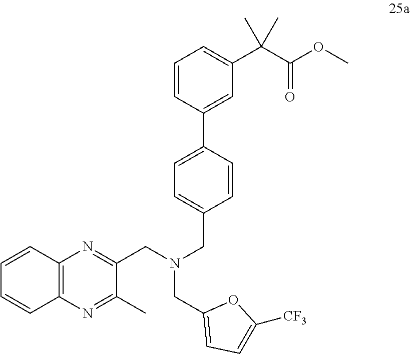 [SMILES: COC(=O)C(C)(C)c1cccc(-c2ccc(CN(Cc3ccc(C(F)(F)F)o3)Cc3nc4ccccc4nc3C)cc2)c1]

To a solution of compound 24c (100 mg, 0.23 mmol) in DMF (5 mL) was added 2-(chloro-methyl)-3-methylquinoxaline (90 mg, 0.46 mmol) and Cs₂CO₃ (225 mg, 0.69 mmol) and the mixture was stirred at rt for 2 d, diluted with water (50 mL) and extracted with EA (3×20 mL).

The combined organic layer was washed with brine (30 mL), dried over Na₂SO₄, filtered, concentrated and purified by FCC (PE:EA=10:1) to give compound 25a as a colorless oil.

### Step 2: 2-Methyl-2-(4-((((3-methylquinoxalin-2-yl)methyl)((5-(trifluoromethyl)furan-2-yl)methyl)amino)methyl)-[1,1′-biphenyl]-3-yl)propanoic acid (25)

Compound 25a (85 mg, 0.23 mmol) was saponified and purified as described in Example 24, Step 5 to afford compound 25 as a white solid. ¹H-NMR (500 MHz, CD₃OD) δ: 8.07-8.05 (m, 1H), 7.92-7.90 (m, 1H), 7.77-7.75 (m, 2H), 7.47-7.36 (m, 8H), 6.90 (d, J=2.0 Hz, 1H), 6.62 (s, 1H), 4.37 (br s, 2H), 4.19 (br s, 2H), 4.08 (br s, 2H), 2.71 (s, 3H), 1.59 (s, 6H); MS: 573.9 (M+H)⁺.

### Example 25/1 to 25/2

The following Examples were prepared and saponified similar as described for Example 25.

| # | building block | structure | analytical data |
|---|---|---|---|
| 25/1 | 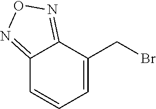 [SMILES: BrCc1cccc2nonc12] | 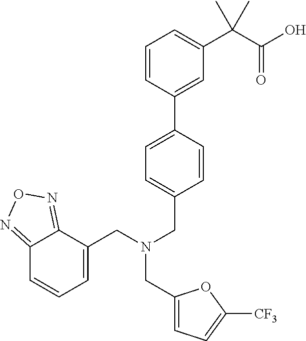 [SMILES: CC(C)(C(=O)O)c1cccc(-c2ccc(CN(Cc3ccc(C(F)(F)F)o3)Cc3cccc4nonc34)cc2)c1] | ¹H-NMR (500 MHz, CD₃OD) δ: 7.79 (d, J = 9.0 Hz, 1H), 7.59-7.39 (m, 10H), 6.90 (d, J = 2.0 Hz, 1H), 6.53 (d, J = 3.0 Hz, 1H), 4.21 (s, 2H), 3.96 (s, 2H), 3.94 (s, 2H), 1.62 (s, 6H); MS: 549.8 (M + H)⁺. |
| 25/2 | 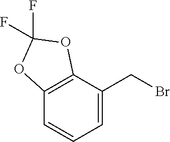 [SMILES: FC1(F)Oc2cccc(CBr)c2O1] | 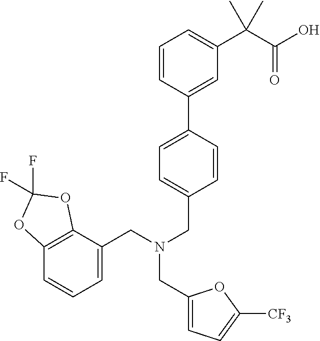 [SMILES: CC(C)(C(=O)O)c1cccc(-c2ccc(CN(Cc3ccc(C(F)(F)F)o3)Cc3cccc4c3OC(F)(F)O4)cc2)c1] | ¹H-NMR (500 MHz, CD₃OD) δ: 7.61 (s, 1H), 7.56 (d, J = 8.0 Hz, 2H), 7.50-7.48 (m, 3H), 7.41-7.39 (m, 2H), 7.27 (d, J = 8.0 Hz, 1H), 7.15 (t, J = 8.0 Hz, 1H), 7.09 (d, J = 7.5 Hz, 1H), 6.91 (d, J = 2.5 Hz, 1H), 6.47 (d, J = 3.0 Hz, 1H), 3.81 (s, 2H), 3.80 (s, 2H), 3.77 (s, 2H), 1.62 (s, 6H); MS: 587.8 (M + H)⁺. |

### Example 26/1 to 26/8

The following Examples were coupled similar as described in Example 3, Step 4 and then optionally saponified similar as described for Example 9.

| # | building blocks | structure | analytical data |
|---|---|---|---|
| 26/1 | 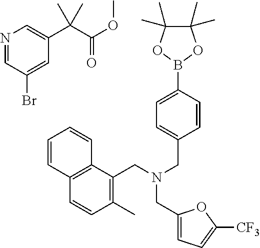 | 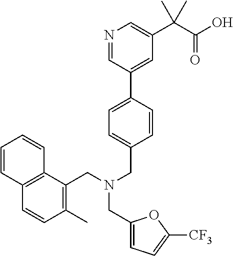 [SMILES: Cc1ccc2ccccc2c1CN(Cc1ccc(-c2cncc(C(C)(C)C(=O)O)c2)cc1)Cc1ccc(C(F)(F)F)o1] | ¹H-NMR (CDCl₃, 400 MHz) δ: 8.68 (s, 1H), 8.63 (d, J = 1.6 Hz, 1H), 8.26 (d, J = 8.8 Hz, 1H), 7.91 (s, 1H), 7.76 (d, J = 8.0 Hz, 1H), 7.66 (d, J = 8.4 Hz, 1H), 7.49- 7.41 (m, 4H), 7.30 (d, J = 8.0 Hz, 3H), 6.72 (d, J = 2.0 Hz, 1H), 6.22 (d, J = 2.8 Hz, 1H), 4.16 (s, 2H), 3.69 (s, 2H), 3.61 (s, 2H), 2.55 (s, 3H), 1.67 (s, 6H); MS: 573.0 (M + H)⁺. |
| 26/2 | 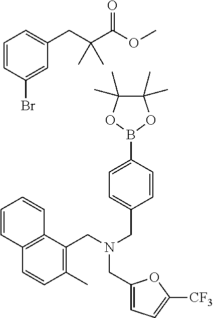 | 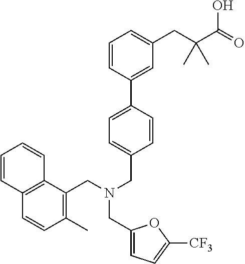 [SMILES: Cc1ccc2ccccc2c1CN(Cc1ccc(-c2cccc(CC(C)(C)C(=O)O)c2)cc1)Cc1ccc(C(F)(F)F)o1] | ¹H-NMR (CDCl₃, 400 MHz) δ: 8.25 (d, J = 8.8 Hz, 1H), 7.76 (d, J = 8.0 Hz, 1H), 7.66 (d, J = 8.4 Hz, 1H), 7.51-7.38 (m, 6H), 7.33-7.26 (m, 4H), 7.11 (d, J = 7.6 Hz 1H), 6.72 (s, 1H), 6.22 (s, 1H), 4.16 (br s, 2H), 3.70 (br s, 2H), 3.61 (br s, 2H), 2.92 (s, 2H), 2.54 (s, 3H), 1.21 (s, 6H); MS: 586.0 (M + H)⁺. |
| 26/3 | 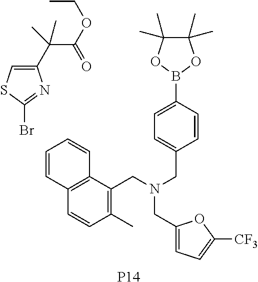 | 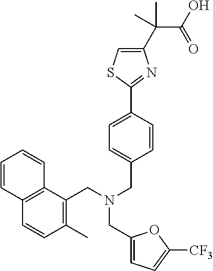 [SMILES: Cc1ccc2ccccc2c1CN(Cc1ccc(-c2nc(C(C)(C)C(=O)O)cs2)cc1)Cc1ccc(C(F)(F)F)o1] | ¹H-NMR (CDCl₃, 400 MHz) δ: 8.25 (d, J = 8.4 Hz, 1H), 7.77 (d, J = 7.6 Hz, 3H), 7.68 (d, J = 8.4 Hz, 1H), 7.53-7.41 (m, 2H), 7.31-7.28 (m, 3H), 7.09 (s, 1H), 6.73 (d, J = 3.2 Hz, 1H), 6.23 (d, J = 2.8 Hz, 1H), 4.17 (s, 2H), 3.70 (s, 2H), 3.61 (s, 2H), 2.55 (s, 3H), 1.67 (s, 6H); MS: 579.0 (M + H)⁺. |
| 26/4 | 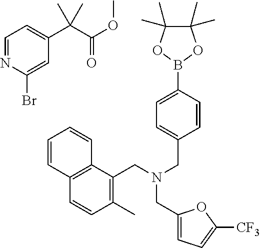 | 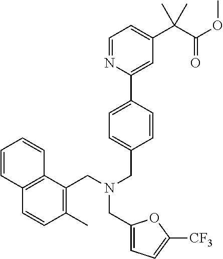 [SMILES: COC(=O)C(C)(C)c1ccnc(-c2ccc(CN(Cc3ccc(C(F)(F)F)o3)Cc3c(C)ccc4ccccc34)cc2)c1] | MS: 587 (M + 1)⁺. |
| 26/5 | 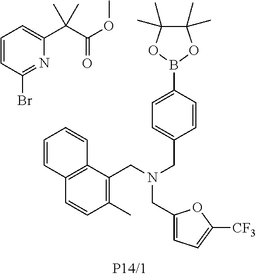 | 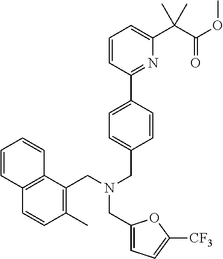 [SMILES: COC(=O)C(C)(C)c1cccc(-c2ccc(CN(Cc3ccc(C(F)(F)F)o3)Cc3c(C)ccc4ccccc34)cc2)n1] | MS: 587 (M + 1)⁺. |
| 26/6 | 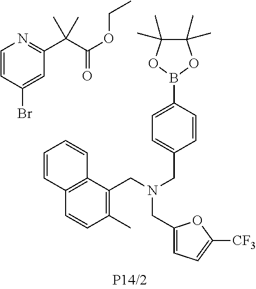 | 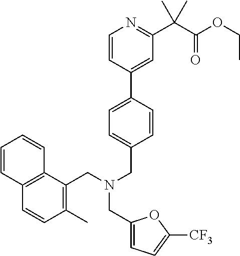 [SMILES: CCOC(=O)C(C)(C)c1cc(-c2ccc(CN(Cc3ccc(C(F)(F)F)o3)Cc3c(C)ccc4ccccc34)cc2)ccn1] | MS: 601 (M + 1)⁺. |
| 26/7 | 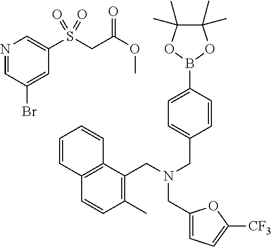 | 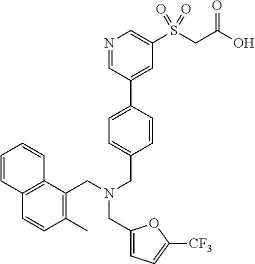 [SMILES: Cc1ccc2ccccc2c1CN(Cc1ccc(-c2cncc(S(=O)(=O)CC(=O)O)c2)cc1)Cc1ccc(C(F)(F)F)o1] | ¹H-NMR (CD₃OD, 400 MHz) δ: 9.01 (dd, J = 2.0, 9.4 Hz, 2H), 8.51 (t, J = 2.0 Hz, 1H), 8.25 (d, J = 8.8 Hz, 1H), 7.77 (d, J = 8.0 Hz, 1H), 7.68 (d, J = 8.4 Hz, 1H), 7.59 (d, J = 8.0 Hz, 2H), 7.34-7.40 (m, 4H), 7.30 (d, J = 8.4 Hz, 1H), 6.90 (d, J = 2.0 Hz, 1H), 6.40 (d, J = 3.6 Hz, 1H), 4.19 (s, 2H), 3.74 (s, 2H), 3.63 (s, 2H), 2.56 (s, 3H); MS: 609.0 (M + 1)⁺. |
| 26/8 | 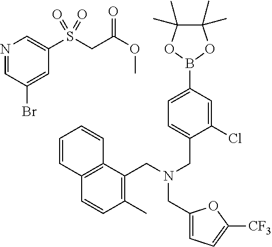 | 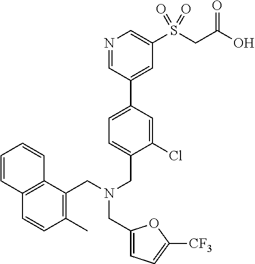 [SMILES: Cc1ccc2ccccc2c1CN(Cc1ccc(C(F)(F)F)o1)Cc1ccc(-c2cncc(S(=O)(=O)CC(=O)O)c2)cc1Cl] | ¹H-NMR (CD₃OD, 400 MHz) δ: 9.01 (d, J = 13.2 Hz, 2H), 8.49 (s, 1H), 8.21 (d, J = 8.4 Hz, 1H), 7.73 (d, J = 7.6 Hz, 1H), 7.67-7.64 (m, 2H), 7.49-7.37 (m, 4H), 7.28 (d, J = 8.4 Hz , 1H), 6.88 (d, J = 2.4 Hz, 1H), 6.42 (d, J = 3.2 Hz. 1H), 4.22 (s, 2H), 3.79 (s, 4H), 2.55 (s, 3H); MS: 642.9 (M + 1)⁺. |

### Example 27

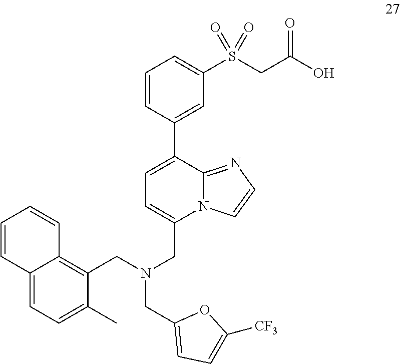 [SMILES: Cc1ccc2ccccc2c1CN(Cc1ccc(C(F)(F)F)o1)Cc1ccc(-c2cccc(S(=O)(=O)CC(=O)O)c2)c2nccn12]

### Step 1: Methyl 2-((3-(5-((((2-methylnaphthalen-1-yl)methyl)((5-(trifluoromethyl)furan-2-yl)methyl)amino)methyl)imidazol[1,2-a]pyridin-8-yl)phenyl)sulfonyl)acetate (27a)

To a solution of compound P15 (250 mg, 0.47 mmol), methyl 2-((3-(4,4,5,5-tetramethyl-1,3,2-dioxaborolan-2-yl)phenyl)sulfonyl)acetate (210 mg, 0.62 mmol), K₃PO₄ (303 mg, 1.41 mmol) and XPhos (114 mg, 0.24 mmol) in 1,4-dioxane (20 mL) was added Pd/XPhos (170 mg, 0.24 mmol) at rt under N₂. The mixture was stirred at 90° C. for 8 h, cooled, filtered, concentrated and purified by FCC (PE:EA=1:1) to give compound 27a as a yellow oil.

### Step 2: 2-((3-(5-((((2-Methylnaphthalen-1-yl)methyl)((5-(trifluoromethyl)furan-2-yl)methyl)amino)methyl)imidazo[1,2-a]pyridin-8-yl)phenyl)sulfonyl)acetic acid (27)

Compound 27a (50 mg, 80 μmol) was treated as described in Example 7 to give compound 27 as a white solid. ¹H-NMR (CDCl₃, 400 MHz) δ: 8.25 (s, 1H), 8.03-7.97 (m, 2H), 7.79-7.70 (m, 3H), 7.60-7.44 (m, 4H), 7.30-7.28 (m, 1H), 7.18-7.15 (m, 2H), 6.84-6.83 (m, 1H), 6.76 (s, 1H), 6.30 (s, 1H), 4.24 (s, 2H), 4.11 (s, 2H), 3.89 (s, 2H), 3.85 (s, 2H), 2.53 (s, 3H); MS: 648.0 (M+1)⁺.

### Example 27/1 to 27/137

The following Examples were synthesized similar as described above using the shown building blocks and sequence. The acid chlorides depicted were prepared similar as described in Preparative Example P20. If necessary, the esters were saponified as described above. The tertiary carboxamide containing examples occur as mixture of cis/trans-isomers.

| # | building blocks | structure | analytical data |
|---|---|---|---|
| 27/1 | 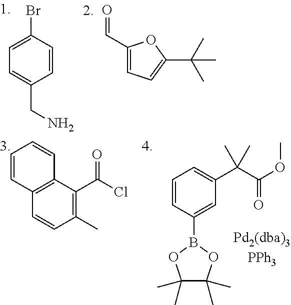 | 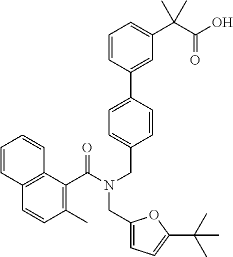 [SMILES: Cc1ccc2ccccc2c1C(=O)N(Cc1ccc(-c2cccc(C(C)(C)C(=O)O)c2)cc1)Cc1ccc(C(C)(C)C)o1] | ¹H-NMR (CDCl₃, 400 MHz) δ: 7.82-7.75 (m, 3H), 7.64- 7.30 (m, 10H), 7.07 (d, n = 8.0 Hz, 1H), 6.22 (d, J = 3.2 Hz, 0.5H), 5.96 (d, J = 3.2 Hz, 0.5H), 5.79-5.76 (m, 1H), 4.98-4.77 (m, 2H), 4.23 (s, 1H), 4.09-4.08 (d, J = 3.2 Hz, 1H), 2.52, 2.50 (2 s, 3H), 1.68, 1.65 (2 s, 6H), 1.36, 1.22 (2 s, 9H); MS: 574.1 (M + H)⁺. |
| 27/2 | 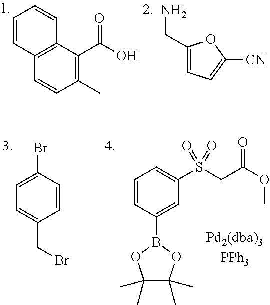 | 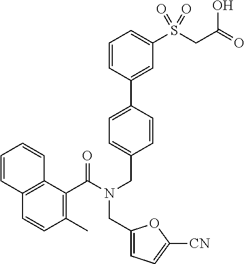 [SMILES: Cc1ccc2ccccc2c1C(=O)N(Cc1ccc(-c2cccc(S(=O)(=O)CC(=O)O)c2)cc1)Cc1ccc(C#N)o1] | ¹H-NMR (CD₃OD, 400 MHz) δ: 8.24, 8.12 (2 s, 1H), 7.99-7.86 (m, 4H), 7.76-7.61 (m, 4H), 7.55-7.48 (m, 3H), 7.42 (d, J = 7.6 Hz, 1H), 7.31 (d, J = 3.2 Hz, 0.5H), 7.08- 7.05 (m, 1H), 7.01 (d, J = 3.6 Hz, 0.5H), 6.59 (d, J = 3.6 Hz, 0.5H), 6.07 (d, J = 3.6 Hz, 0.5H), 5.09-4.89 (m, 2H), 4.34, 4.30 (2 s, 2H), 4.19, 4.16 (2 s, 2H), 2.45, 2.43 (2 s, 3H); MS: 579.0 (M + H)⁺. |
| 27/3 | 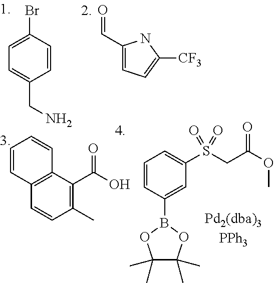 | 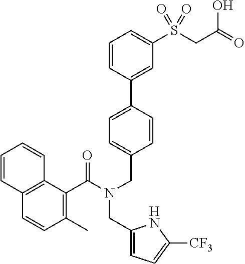 [SMILES: Cc1ccc2ccccc2c1C(=O)N(Cc1ccc(-c2cccc(S(=O)(=O)CC(=O)O)c2)cc1)Cc1ccc(C(F)(F)F)[nH]1] | ¹H-NMR (CDCl₃, 400 MHz) δ: 9.80 (s, 1H), 8.06-8.00 (m, 1H), 7.77- 7.53 (m, 5H), 7.42-7.18 (m, 8H), 6.89 (d, J = 7.6 Hz, 1H), 6.41, 6.23 (2 s, 1H), 5.94, 5.66 (2 s, 1H), 4.61-3.83 (m, 6H), 2.23, 2.20 (2 s, 3H); MS: 621.0 (M + H)⁺. |
| 27/4 | 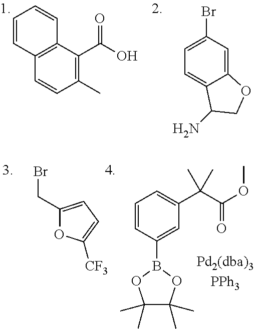 | 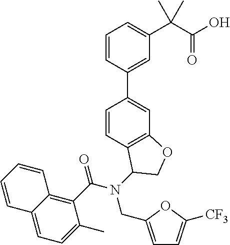 [SMILES: Cc1ccc2ccccc2c1C(=O)N(Cc1ccc(C(F)(F)F)o1)C1COc2cc(-c3cccc(C(C)(C)C(=O)O)c3)ccc21] | ¹H-NMR (CDCl₃, 400 MHz) δ: 7.80-7.75 (m, 2H), 7.71 (d, J = 8.4 Hz, 1H), 7.50-7.39 (m, 4H), 7.33 (s, 3H), 7.22 (d, J = 8.4 Hz, 1H), 7.09 (d, J = 7.2 Hz, 1H), 7.00 (s, 1H), 6.39 (d, J = 6.8 Hz, 1H), 5.92 (d, J = 2.8 Hz, 1H), 4.84 (t, J = 8.8 Hz, 1H), 4.55-4.30 (m, 4H), 2.45 (s, 3H), 1.57 (s, 6H). |
| 27/5 | 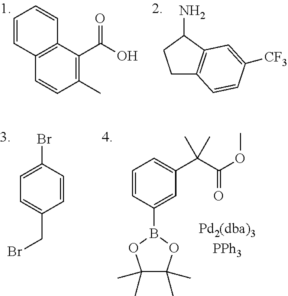 | 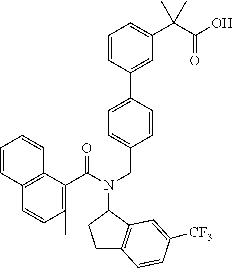 [SMILES: Cc1ccc2ccccc2c1C(=O)N(Cc1ccc(-c2cccc(C(C)(C)C(=O)O)c2)cc1)C1CCc2ccc(C(F)(F)F)cc21] | ¹H-NMR (CDCl₃, 400 MHz) δ: 8.02 (d, J = 8.4 Hz, 0.5H), 7.89- 7.70 (m, 2.5H), 7.59-7.28 (m, 11H), 7.25-7.17 (m, 2.5H), 6.78-6.71 (m, 0.5H), 5.19- 4.20 (m, 2.5H), 3.11-2.44 (m, 5.5H), 2.26-1.94 (m, 2H), 1.67, 1.63 (2 s, 6H); MS: 622.4 (M + H)⁺. |
| 27/6 | 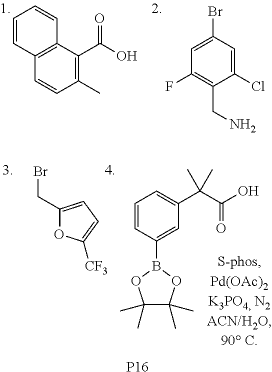 [SMILES: Cc1ccc2ccccc2c1C(=O)O ; NCc1c(F)cc(Br)cc1Cl ; FC(F)(F)c1ccc(CBr)o1 ; "P16" ; CC(C)(C(=O)O)c1cccc(B2OC(C)(C)C(C)(C)O2)c1] | 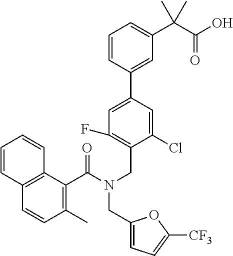 [SMILES: Cc1ccc2ccccc2c1C(=O)N(Cc1ccc(C(F)(F)F)o1)Cc1c(F)cc(-c2cccc(C(C)(C)C(=O)O)c2)cc1Cl] | ¹H-NMR (CDCl₃, 400 MHz) δ: 7.80-7.28 (m, 10H), 7.24-6.24 (m, 4H), 5.66-3.48(m, 4H), 2.43- 2.17 (m, 3H), 1.66-1.52 (m, 6H); MS: 635.9 (M − H)⁻. |
| 27/7 | 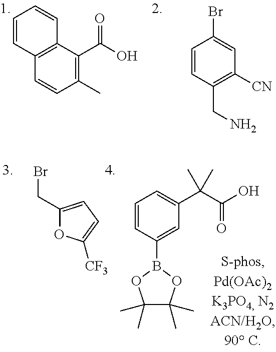 [SMILES: Cc1ccc2ccccc2c1C(=O)O ; N#Cc1cc(Br)ccc1CN ; FC(F)(F)c1ccc(CBr)o1 ; CC(C)(C(=O)O)c1cccc(B2OC(C)(C)C(C)(C)O2)c1] | 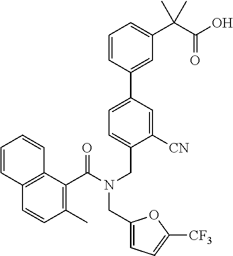 [SMILES: Cc1ccc2ccccc2c1C(=O)N(Cc1ccc(C(F)(F)F)o1)Cc1ccc(-c2cccc(C(C)(C)C(=O)O)c2)cc1C#N] | ¹H-NMR (CDCl₃, 400 MHz) δ: 7.96 (d, J = 8.0 Hz, 0.5H), 7.84- 7.68 (m, 4H),7.64-7.30 (m, 8H), 7.04 (d, J = 8.0 Hz, 0.5H), 6.76 (d, J = 2.0 Hz, 0.5H), 6.47 (d, J = 2.8 Hz, 1H), 6.08 (d, J = 3.2 Hz, 0.5H), 5.32-5.02 (m, 2H), 4.59-4.30 (m, 2H), 2.50, 2.45 (2 s, 3H), 1.69. 1.66 (2 s, 6H); MS: 608.9 (M − H)⁻. |
| 27/8 | 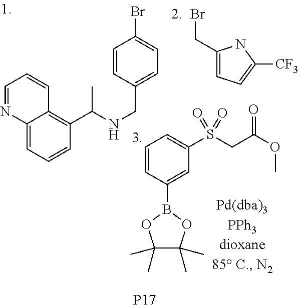 | 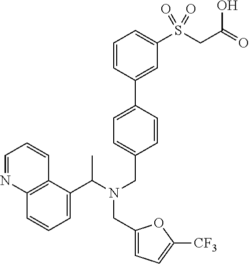 [SMILES: CC(c1cccc2ncccc12)N(Cc1ccc(-c2cccc(S(=O)(=O)CC(=O)O)c2)cc1)Cc1ccc(C(F)(F)F)o1] | ¹H-NMR (CDCl₃, 400 MHz) δ: 8.79- 8.74 (m, 2H), 7.96 (d, J = 8.4 Hz, 1H), 7.87(s, 1H), 7.77 (d, J = 8.0 Hz, 1H), 7.58 (t,J = 7.8 Hz, 1H), 7.52 (d, J = 6.8 Hz, 1H), 7.43 (d, J = 7.6 Hz, 1H), 7.36 (dd, J = 7.4, 4.2 Hz, 1H), 7.30 (t, J = 7.8 Hz, 1H), 6.95 (d, J = 7.2 Hz, 2H), 6.69-6.67 (m, 3H), 6.18 (s, 1H), 4.57-4.54 (m, 1H), 4.15-4.05 (m, 2H), 3.85-3.73 (m, 2H), 3.55 (d, J = 14.4 Hz, 1H), 3.39 (d, J = 14.4 Hz, 1H), 1.56 (d, J = 6.4 Hz, 3H); |
| | | | MS: 609.0 (M + H)⁺. |
| 27/9 | 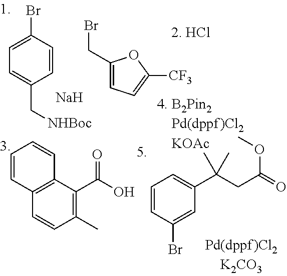 | 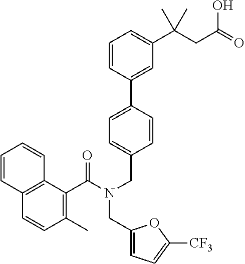 [SMILES: Cc1ccc2ccccc2c1C(=O)N(Cc1ccc(-c2cccc(C(C)(C)CC(=O)O)c2)cc1)Cc1ccc(C(F)(F)F)o1] | ¹H-NMR (500 MHz, CD₃OD) δ: 7.81-7.78 (m, 2H), 7.65-7.24 (m, 11H), 6.93(d, J = 8.5 Hz, 1H), 6.88 (d, J = 2.5 Hz, 0.5H), 6.62 (d, J = 1.5 Hz, 0.5H), 6.42 (d, J = 3.5 Hz, 0.5H), 5.98 (d, J = 3.0 Hz, 0.5H), 4.96-4.82 (m, 2H), 4.23-4.17 (m, 2H), 2.61, 2.58 (2 s, 2H), 2.36, 2.33 (2 s, 3H), 1.43, 1.39 (2 s, 6H): MS: 600.1 (M + H)⁺. |
| 27/10 | 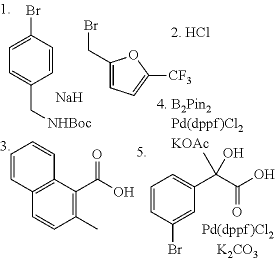 | 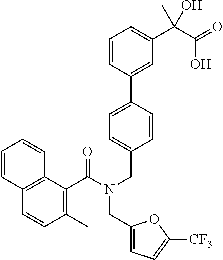 [SMILES: Cc1ccc2ccccc2c1C(=O)N(Cc1ccc(-c2cccc(C(C)(O)C(=O)O)c2)cc1)Cc1ccc(C(F)(F)F)o1] | ¹H-NMR (500 MHz, CD₃OD) δ: 7.81-7.55 (m, 5H), 7.50-7.28 (m, 8H), 6.92 (d, J = 7.5 Hz, 1H), 6.88 (d, J = 2.0 Hz, 0.5H), 6.62 (d, J = 2.0 Hz, 0.5H), 6.42 (d, J = 3.0 Hz, 0.5H), 5.98 (d, J = 3.5 Hz, 0.5H), 4.95-4.80 (m, 2H), 4.19 (s, 2H), 2.35, 2.32 (2 s, 3H), 1.72, 1.68 (2 s, 3H); MS: 588.2 (M + H)⁺. |
| 27/11 | 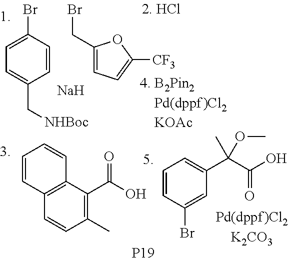 | 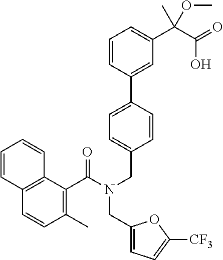 [SMILES: COC(C)(C(=O)O)c1cccc(-c2ccc(CN(Cc3ccc(C(F)(F)F)o3)C(=O)c3c(C)ccc4ccccc34)cc2)c1] | ¹H-NMR (500 MHz, CD₃OD) δ: 7.78-7.77 (m, 2H), 7.67-7.30 (m, 11H), 6.91-6.87 (m, 1.5H), 6.61 (s, 0.5H), 6.41 (s, 0.5H), 5.96 (s, 0.5H), 4.94-4.78 (m, 2H), 4.17 (s, 2H), 3.21, 3.18 (2 s, 3H), 2.35, 2.31 (2 s, 3H), 1.74, 1.70 (2 s, 3H); MS: 602.2 (M + H)⁺. |
| 27/12 | 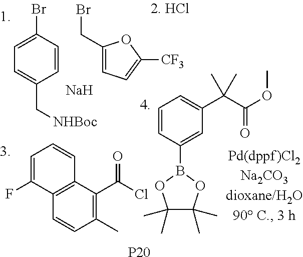 | 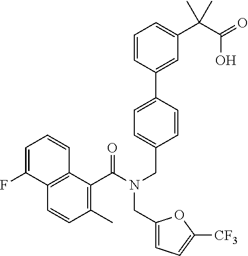 [SMILES: Cc1ccc2c(F)cccc2c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 8.12 (d, J = 8.6 Hz, 1H), 7.65-7.39 (m, 10H), 7.24-7.21 (m, 1H), 7.03-6.99 (m, 1.5H), 6.74 (dd, J = 3.3, 1.3 Hz, 0.5H), 6.55 (d,J = 3.0 Hz, 0.5H), 6.12 (d, J = 3.0 Hz, 0.5H). 5.02-4.90 (m, 2H), 4.35-4.28 (m, 2H), 2.49, 2.46 (2 s, 3H), 1.64, 1.61 (2 s, 6H); MS: 604.0 (M + H)⁺. |
| 27/13 | 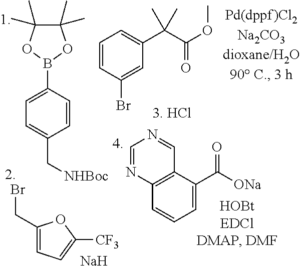 | 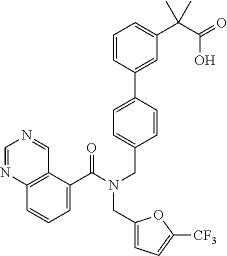 [SMILES: CC(C)(C(=O)O)c1cccc(-c2ccc(CN(Cc3ccc(C(F)(F)F)o3)C(=O)c3cccc4ncncc34)cc2)c1] | ¹H-NMR (500 MHz, DMSO-d₆) δ: 9.38 (d, J = 5.0 Hz, 1H), 9.30 (s, 1H), 8.13-8.03 (m, 2H), 7.87-7.81 (m, 1H), 7.64- 7.32 (m, 7H), 7.23 (d, J = 2.0 Hz, 0.5H), 7.05 (d, J = 8.0 Hz, 1H), 6.95 (d, J = 2.0 Hz, 0.5H), 6.73 (d, J = 3.0 Hz, 0.5H), 6.38 (d, J = 3.5 Hz, 0.5H), 4.97-4.89 (m, 2H), 4.53-4.46 (m, 2H), 1.53, 1.51 (2 s, 6H); MS: 574.0 (M + H)⁺. |
| 27/14 | 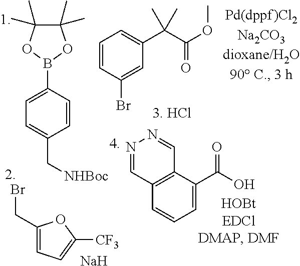 | 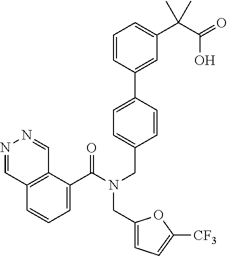 [SMILES: CC(C)(C(=O)O)c1cccc(-c2ccc(CN(Cc3ccc(C(F)(F)F)o3)C(=O)c3cccc4cnncc34)cc2)c1] | ¹H-NMR (500 MHz, CD₃CD) δ: 9.92, 9.82 (2 s, 1H), 9.72, 9.55 (2 s, 1H), 8.47-8.23 (m, 3H), 7.65- 7.35 (m, 7H), 7.04 (d, J = 2.0 Hz, 0.5H), 6.99 (d, J = 8.5 Hz, 1H), 6.76 (d, J = 2.5 Hz, 0.5H), 6.69 (d, J = 3.0 Hz, 0.5H), 6.28 (d, J = 3.0 Hz, 0.5H), 5.10, 5.01 (2 s, 2H), 4.58,4.55 (2 s, 2H), 1.64, 1.62 (2 s, 6H); MS: 574.2 (M + H)⁺. |
| 27/15 | 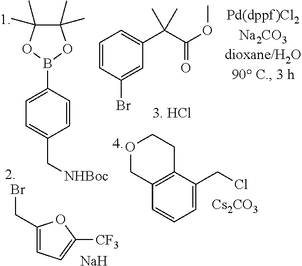 | 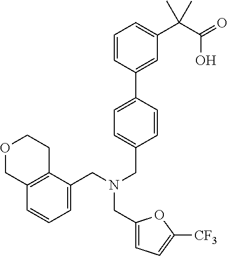 [SMILES: CC(C)(C(=O)O)c1cccc(-c2ccc(CN(Cc3ccc(C(F)(F)F)o3)Cc3cccc4c3CCOC4)cc2)c1] | ¹H-NMR (500 MHz, CD₃OD) δ: 7.71 (d, J = 8.0 Hz, 2H), 7.64 (s, 1H), 7.56-7.53(m, 3H), 7.46- 7.42 (m, 3H), 7.30 (t, J = 7.5 Hz, 1H), 7.14-7.12 (m, 2H), 6.86 (s, 1H), 4.75 (s, 2H), 4.39 (br s, 2H), 4.29 (br s, 2H), 4.21 (br s, 2H), 3.87 (t, J = 5.5 Hz, 2H), 2.53 (br s, 2H), 1.63 (s, 6H): MS: 564.3 (M + H)⁺. |
| 27/16 | 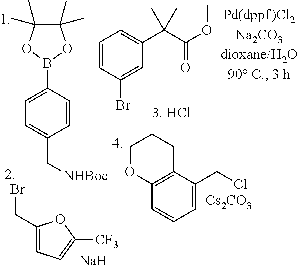 | 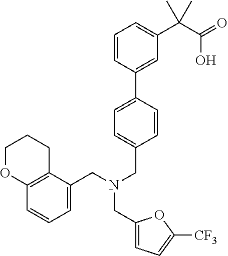 [SMILES: CC(C)(C(=O)O)c1cccc(-c2ccc(CN(Cc3ccc(C(F)(F)F)o3)Cc3cccc4c3CCCO4)cc2)c1] | ¹H-NMR (500 MHz, CD₃OD) δ: 7.73 (d, J = 8.0 Hz, 2H), 7.64 (s, 1H), 7.58-7.53 (m, 3H), 7.46- 7.45 (m, 2H), 7.20 (t, J = 7.8 Hz, 1H), 7.13 (d, J = 2.0 Hz, 1H), 7.08 (d. J = 7.0 Hz, 1H), 6.90 (s, 1H), 6.86 (d, J = 8.0 Hz, 1H), 4.45 (br s, 2H), 4.34 (br s, 2H), 4.22 (br s, 2H), 4.11 (t, J = 5.3 Hz, 2H), 2.43 (t, J = 5.5 Hz, 2H), 1.90 (t, J = 5.5 Hz, 2H), 1.63 (s, 6H); MS: 564.3 (M + H)⁺. |
| 27/17 | 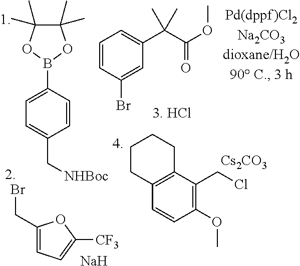 | 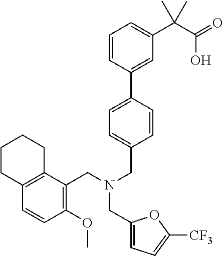 [SMILES: COc1ccc2c(c1CN(Cc1ccc(-c3cccc(C(C)(C)C(=O)O)c3)cc1)Cc1ccc(C(F)(F)F)o1)CCCC2] | ¹H-NMR (500 MHz, CD₃OD) δ: 7.72 (d, J = 8.5 Hz, 2H), 7.63 (s, 1H), 7.59 (d, J = 8.0 Hz, 2H), 7.55-7.53 (m, 1H), 7.47 (d, J = 4.0 Hz, 2H), 7.15-7.13 (m, 2H), 6.98 (d, J = 3.0 Hz, 1H), 6.86 (d, J = 8.5 Hz, 1H), 4.64-4.42 (m, 6H), 3.85 (s, 3H), 2.70-2.68 (m, 2H), 2.48 (br s, 2H), 1.73- 1.70 (m, 4H), 1.64 (s, 6H); MS: 592.3 (M + H)⁺. |
| 27/18 | 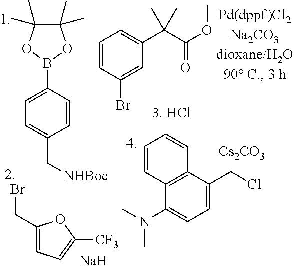 | 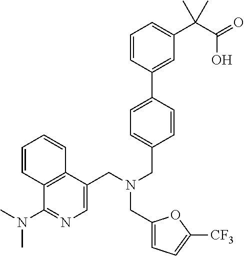 [SMILES: CN(C)c1ncc(CN(Cc2ccc(-c3cccc(C(C)(C)C(=O)O)c3)cc2)Cc2ccc(C(F)(F)F)o2)c2ccccc12] | ¹H-NMR (500 MHz, CD₃OD) δ: 8.37 (d, J = 8.0 Hz, 1H), 8.23 (d, J = 7.5 Hz, 1H), 7.97-7.94 (m, 1H), 7.80-7.77 (m, 1H), 7.59 (s, 1H), 7.53 (s, 1H), 7.44-7.38 (m, 5H), 7.29 (d, J = 8.0 Hz, 2H), 6.96 (dd, J = 3.0, 1.0 Hz, 1H), 6.53 (d, J = 3.5 Hz, 1H), 4.03 (s, 2H), 3.96 (s, 2H), 3.84 (s, 2H), 3.41 (s, 6H), 1.62 (s, 6H); MS: 602.3 (M + H)⁺. |
| 27/19 | 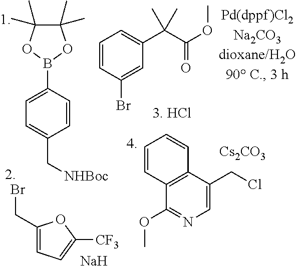 | 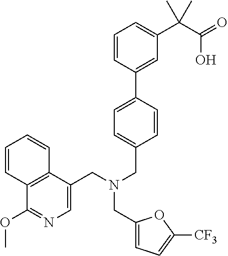 [SMILES: COc1ncc(CN(Cc2ccc(-c3cccc(C(C)(C)C(=O)O)c3)cc2)Cc2ccc(C(F)(F)F)o2)c2ccccc12] | ¹H-NMR (500 MHz, CD₃OD) δ: 8.30 (d, J = 8.5 Hz, 1H), 8.13 (s, 1H), 7.70-7.64 (m, 6H), 7.53-7.51 (m, 3H), 7.45- 7.44 (m, 2H), 7.08 (d, J = 2.5 Hz, 1H), 6.79 (s, 1H), 4.48 (br s, 2H), 4.35 (br s, 2H), 4.27 (br s, 2H), 4.16 (s, 3H), 1.64 (s, 6H); MS: 589.3 (M + H)⁺. |
| 27/20 | 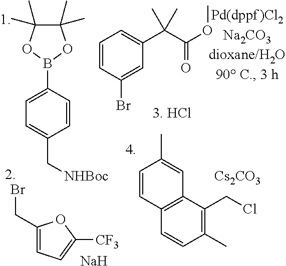 | 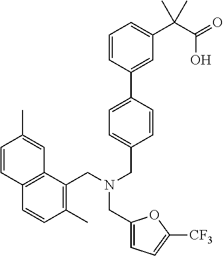 [SMILES: Cc1ccc2ccc(C)c(CN(Cc3ccc(-c4cccc(C(C)(C)C(=O)O)c4)cc3)Cc3ccc(C(F)(F)F)o3)c2c1] | ¹H-NMR (500 MHz, CD₃OD) δ: 7.83 (d, J = 9.0 Hz, 1H), 7.78 (d, J = 8.0 Hz, 1H), 7.74 (d, J = 8.5 Hz, 2H), 7.65 (s, 1H), 7.59-7.54 (m, 3H), 7.46-7.45 (m, 2H), 7.37- 7.32 (m. 3H), 7.09 (s, 1H), 6.80 (s, 1H), 4.85 (br s, 2H), 4.45 (br s, 2H), 4.36 (br s, 2H), 2.57 (s, 3H), 2.40 (s, 3H), 1.64 (s, 6H); MS: 586.2 (M + H)⁺. |
| 27/21 | 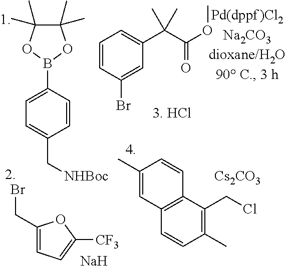 | 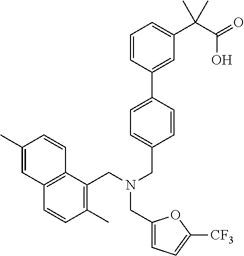 [SMILES: Cc1ccc2c(CN(Cc3ccc(-c4cccc(C(C)(C)C(=O)O)c4)cc3)Cc3ccc(C(F)(F)F)o3)c(C)ccc2c1] | ¹H-NMR (500 MHz, CD₃OD) δ: 7.80 (d, J = 8.5 Hz, 1H), 7.70-7.64 (m, 5H), 7.53-7.50 (m, 3H), 7.47-7.45 (m, 2H), 7.39- 7.34 (m, 2H), 7.08 (s, 1H), 6.79 (s, 1H), 4.79 (br s, 2H), 4.41 (br s, 2H), 4.32 (br s, 2H), 2.50 (s, 6H), 1.63 (s, 6H); MS: 586.3 (M + H)⁺. |
| 27/22 | 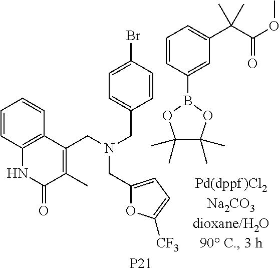 | 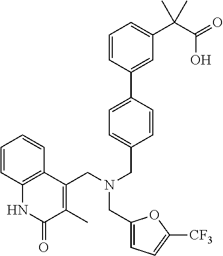 [SMILES: Cc1c(CN(Cc2ccc(-c3cccc(C(C)(C)C(=O)O)c3)cc2)Cc2ccc(C(F)(F)F)o2)c2ccccc2[nH]c1=O] | ¹H-NMR (500 MHz, DMSO-d₆) δ: 12.33 (br s, 1H), 11.72 (s, 1H), 7.88 (d, J = 8.0 Hz, 1H), 7.53-7.38 (m, 6H), 7.32-7.25 (m, 4H), 7.16-7.09 (m, 2H), 6.55 (d, J = 2.0 Hz, 1H), 3.97 (s, 2H), 3.75 (s, 2H), 3.64 (s, 2H), 2.18 (s, 3H), 1.52 (s, 6H); MS: 589.3 (M + H)⁺. |
| 27/23 | 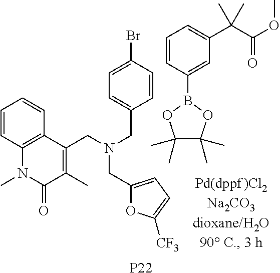 | 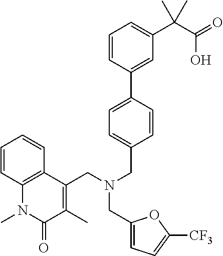 [SMILES: Cc1c(CN(Cc2ccc(-c3cccc(C(C)(C)C(=O)O)c3)cc2)Cc2ccc(C(F)(F)F)o2)c2ccccc2n(C)c1=O] | ¹H-NMR (500 MHz, CD₃OD) δ: 7.97 (d, J = 8.0 Hz, 1H), 7.61-7.58 (m, 1H), 7.55 (s, 1H), 7.49 (d, J = 9.0 Hz, 1H), 7.45-7.37 (m, 5H), 7.29-7.26 (m, 3H), 6.95 (d, J = 2.0 Hz, 1H), 6.51 (d, J = 3.0 Hz, 1H), 4.17 (s, 2H), 3.93 (s, 2H), 3.81 (s, 2H), 3.67 (s, 3H), 2.32 (s, 3H), 1.62 (s, 6H); MS: 603.3 (M + H)⁺. |
| 27/24 | 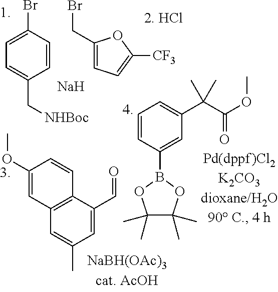 | 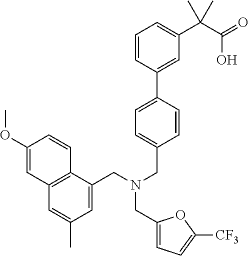 [SMILES: COc1ccc2c(CN(Cc3ccc(-c4cccc(C(C)(C)C(=O)O)c4)cc3)Cc3ccc(C(F)(F)F)o3)cc(C)cc2c1] | ¹H-NMR (400 MHz, CD₃OD) δ: 7.73-7.66 (m, 4H), 7.57-7.44 (m, 6H), 7.39 (s, 1H), 7.24 (d, J = 2.4 Hz, 1H), 7.12 (d, J = 2.4 Hz, 1H), 7.03 (dd, J = 9.2, 2.4 Hz, 1H), 6.85 (d, J = 2.4 Hz, 1H), 4.64 (s, 2H), 4.45 (br s, 2H), 4.38 (br s, 2H), 3.90 (br s, 3H), 2.52 (s, 3H), 1.64 (s, 6H); MS: 602.2 (M + H)⁺. |
| 27/25 | 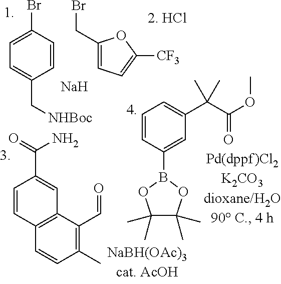 | 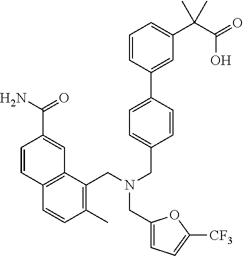 [SMILES: Cc1ccc2ccc(C(N)=O)cc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 8.92 (br s, 1H), 7.92-7.87 (m, 2H), 7.82 (d. J = 9.0 Hz, 1H), 7.57 (s, 1H), 7.51-7.35 (m, 8H), 6.90 (s, 1H), 6.48 (d, J = 1.6 Hz, 1H), 4.47 (br s, 2H), 3.90 (br s, 4H), 2.57 (s, 3H), 1.62 (s, 6H); MS: 615.2 (M + H)⁺. |
| 27/26 | 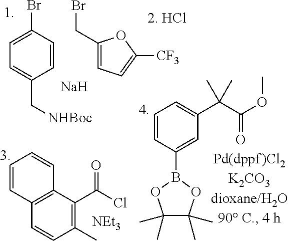 | 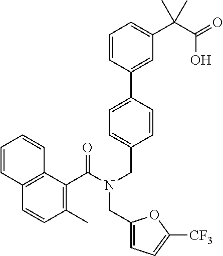 [SMILES: Cc1ccc2ccccc2c1C(=O)N(Cc1ccc(-c2cccc(C(C)(C)C(=O)O)c2)cc1)Cc1ccc(C(F)(F)F)o1] | ¹H-NMR (500 MHz, C₃OD) δ: 7.93-7.90 (m, 2H), 7.77-7.39 (m, 11H), 7.04 (d, J = 8.0 Hz, 1H), 6.99 (d, J = 2.5 Hz, 0.5H), 7.34 (d, J = 2.0 Hz, 0.5H), 6.54 (d, J = 3.5 Hz, 0.5H), 6.09 (d, J = 3.5 Hz, 0.5H), 5.08- 4.91 (m, 2H), 4.35-4.26 (m, 2H), 2.48, 2.45 (2 s, 3H), 1.64, 1.61 (2 s, 6H); MS: 585.8 (M + H)⁺. |
| 27/27 | 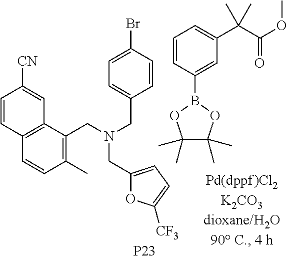 | 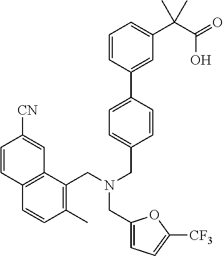 [SMILES: Cc1ccc2ccc(C#N)cc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 8.53 (br s, 1H), 7.95 (d, J = 8.0 Hz, 1H), 7.84 (d, J = 8.5 Hz, 1H), 7.64-7.61 (m, 4H), 7.54-7.50 (m, 2H), 7.44-7.38 (m, 4H), 6.92 (s, 1H), 6.49 (s, 1H), 4.41 (brs, 2H), 3.99 (br s, 2H), 3.95 (br s, 2H), 2.60 (s, 3H), 1.62 (s, 6H); MS: 597.3 (M + H)⁺. |
| 27/28 | 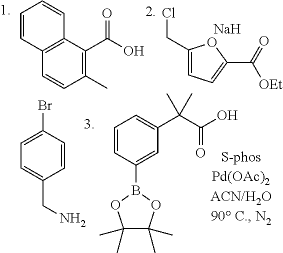 | 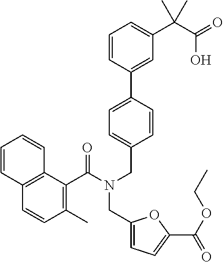 [SMILES: CCOC(=O)c1ccc(CN(Cc2ccc(-c3cccc(C(C)(C)C(=O)O)c3)cc2)C(=O)c2c(C)ccc3ccccc23)o1] | ¹H-NMR (CDCl₃, 400 MHz) δ: 7.83-7.31 (m, 13H), 7.19 (d, J = 3.6 Hz, 0.5H), 7.08 (d, J = 7.6 Hz, 1H), 6.97 (d, J = 3.6 Hz, 0.5H), 6.51 (d, J = 3.2 Hz, 0.5H); 5.90 (d, J = 3.2 Hz, 0.5H), 5.06-4.85 (m, 2H), 4.41-4.13 (m, 4H), 2.49 (d, J = 6.8 Hz, 3H), 1.67, 1.64 (2 s, 6H), 1.41-1.36 (m, 3H); MS: 590.0 (M + H)⁺. |
| 27/29 | 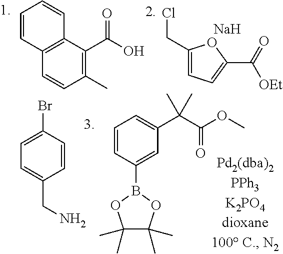 | 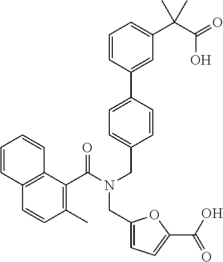 [SMILES: Cc1ccc2ccccc2c1C(=O)N(Cc1ccc(-c2cccc(C(C)(C)C(=O)O)c2)cc1)Cc1ccc(C(=O)O)o1] | MS: 562.3 (M + H)⁺. |
| 27/30 | 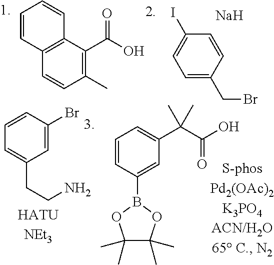 | 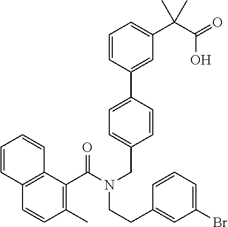 [SMILES: Cc1ccc2ccccc2c1C(=O)N(CCc1cccc(Br)c1)Cc1ccc(-c2cccc(C(C)(C)C(=O)O)c2)cc1] | MS: 619.9 (M + H)⁺. |
| 27/31 | 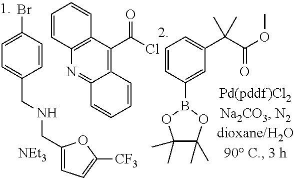 | 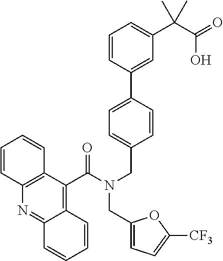 [SMILES: CC(C)(C(=O)O)c1cccc(-c2ccc(CN(Cc3ccc(C(F)(F)F)o3)C(=O)c3c4ccccc4nc4ccccc34)cc2)c1] | ¹H-NMR (500 MHz, CD₃OD) δ: 8.33 (d, J = 8.5 Hz, 1H), 8.30 (d, J = 8.5 Hz, 1H), 8.20-8.13 (m, 4H), 7.86-7.81 (m, 2H), 7.70-7.38 (m, 6H), 7.23 (d, J = 8.5 Hz, 1H), 7.08 (s, 0.6H), 6.79 (d, J = 8.0 Hz, 1H), 6.70 (s, 0.4H), 6.65 (d, J = 3.5 Hz, 0.4H), 6.15 (s, 0.4H), 5.21, 5.12 (2 s, 2H), 4.36, 4.30 (2 s, 2H), 1.66, 1.61 (2 s, 6H); MS: 620.9 (M − H)⁻. |
| 27/32 | 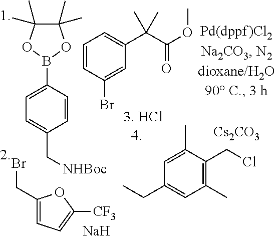 | 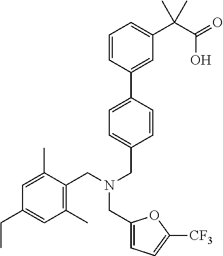 [SMILES: CCc1cc(C)c(CN(Cc2ccc(-c3cccc(C(C)(C)C(=O)O)c3)cc2)Cc2ccc(C(F)(F)F)o2)c(C)c1] | ¹H-NMR (400 MHz, CD₃OD) δ: 7.69 (d, J = 7.6 Hz, 2H), 7.64 (s, 1H), 7.52-7.43 (m, 5H), 7.06 (br s, 1H), 6.97 (s, 2H), 6.77 (br s, 1H), 4.35-4.11 (m, 6H), 2.58 (q, J = 7.6 Hz, 2H), 2.25 (s, 6H), 1.63 (s, 6H), 1.21 (t, J = 7.6 Hz, 3H); MS: 564.3 (M + H)⁺. |
| 27/33 | 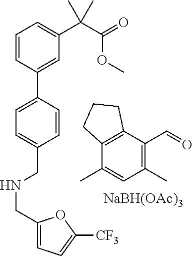 | 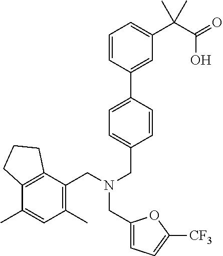 [SMILES: Cc1cc(C)c(CN(Cc2ccc(-c3cccc(C(C)(C)C(=O)O)c3)cc2)Cc2ccc(C(F)(F)F)o2)c2c1CCC2] | ¹H-NMR (500 MHz, CD₃OD) δ: 7.72 (d, J = 8.0 Hz, 2H), 7.64 (s, 1H), 7.55- 7.54 (m, 3H), 7.47-7.45 (m, 2H), 7.11 (s, 1H), 6.92 (s, 1H), 6.87 (br s, 1H), 4.42-4.32 (m, 6H), 2.84 (dd, J = 16.8, 7.8 Hz, 4H), 2.23 (s, 3H), 2.22 (s, 3H), 2.07 (p, J = 7.5 Hz, 2H), 1.63 (s, 6H); MS: 576.3 (M + H)⁺. |
| 27/34 | 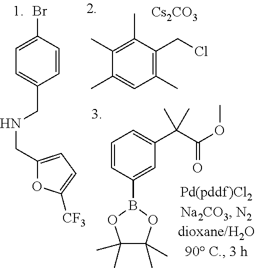 | 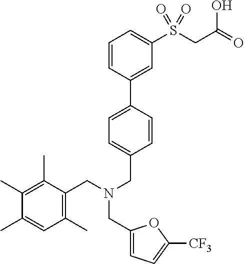 [SMILES: Cc1cc(C)c(CN(Cc2ccc(-c3cccc(S(=O)(=O)CC(=O)O)c3)cc2)Cc2ccc(C(F)(F)F)o2)c(C)c1C] | ¹H-NMR (500 MHz, CD₃OD) δ: 8.23 (t, J = 1.5 Hz, 1H), 8.04 (d, J = 8.0 Hz, 1H), 8.00 (d, J = 8.0 Hz, 1H), 7.89-7.74 (m, 3H), 7.56 (d, J = 8.0 Hz, 2H), 7.07 (s, 1H), 6.95 (s, 1H), 6.79 (s, 1H), 4.40-4.23 (m, 8H), 2.27 (s, 3H), 2.24 (s, 3H), 2.22 (s, 3H), 2.19 (s, 3H); MS: 600.3 (M + H)⁺. |
| 27/35 | 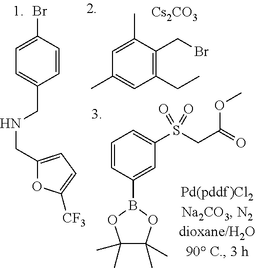 | 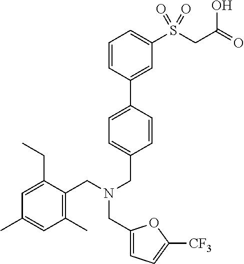 [SMILES: CCc1cc(C)cc(C)c1CN(Cc1ccc(-c2cccc(S(=O)(=O)CC(=O)O)c2)cc1)Cc1ccc(C(F)(F)F)o1] | ¹H-NMR (500 MHz, CD₃OD) δ: 8.23 (t, J = 1.8 Hz, 1H), 8.04 (d, J = 7.5 Hz, 1H), 8.00 (d, J = 8.0 Hz, 1H), 7.78-7.74 (m, 3H), 7.55 (d, J = 8.0 Hz, 2H), 7.06 (s, 1H), 6.96 (d, J = 1.5 Hz, 2H), 6.75 (br s, 1H), 4.40 (s, 2H), 4.22-4.12 (m, 6H), 2.61-2.55 (m, 2H), 2.32 (s, 3H), 2.29 (s, 3H), 1.07 (t, J = 7.5 Hz, 3H); MS: 600.2 (M + H)⁺. |
| 27/36 | 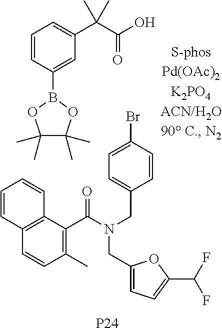 | 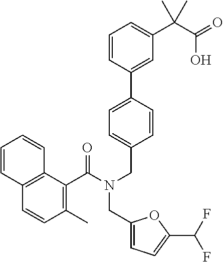 [SMILES: Cc1ccc2ccccc2c1C(=O)N(Cc1ccc(-c2cccc(C(C)(C)C(=O)O)c2)cc1)Cc1ccc(C(F)F)o1] | ¹H-NMR (CDCl₃, 400 MHz) δ: 7.82-7.38 (m, 12H), 7.31 (t, J = 8.6 Hz, 1H), 7.07 (d, J = 8.0 Hz, 1H), 6.79-6.29 (m, 2.5H), 5.85 (d, J = 3.2 Hz, 0.5H), 5.05-4.81 (m, 2H), 4.25 (s, 1H), 4.14 (s, 1H) 2.47, 2.46 (2 s, 3H), 1.68, 1.65 (2 s, 6H); MS: 568.3 (M + H)⁺. |
| 27/37 | 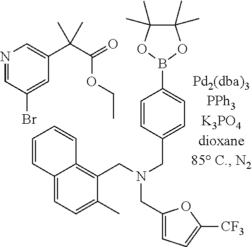 | 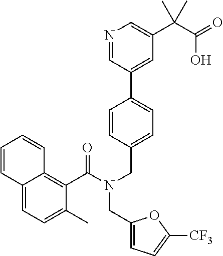 [SMILES: Cc1ccc2ccccc2c1C(=O)N(Cc1ccc(-c2cncc(C(C)(C)C(=O)O)c2)cc1)Cc1ccc(C(F)(F)F)o1] | ¹H-NMR (CDCl₃, 400 MHz) δ: 8.78-8.67 (m, 2H), 8.00-7.31 (m, 11H), 7.10 (d, J = 8.0 Hz, 1H), 6.80 (s, 0.5H), 6.56 (s, 0.5H), 6.45 (d. J = 2.8 Hz, 0.5H), 5.84 (d, J = 2.4 Hz, 0.5H), 5.08-4.86 (m, 2H), 4.27, 4.15 (2 s, 2H), 2.45 (s, 3H), 1.72, 1.69 (2 s, 6H); MS: 587.0 (M + H)⁺. |
| 27/38 | 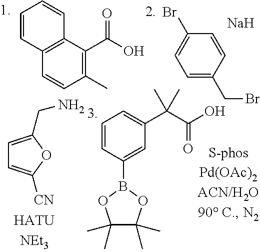 | 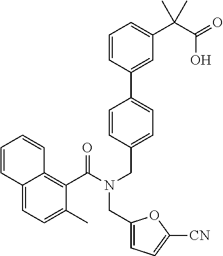 [SMILES: Cc1ccc2ccccc2c1C(=O)N(Cc1ccc(-c2cccc(C(C)(C)C(=O)O)c2)cc1)Cc1ccc(C#N)o1] | ¹H-NMR (CDCl₃, 400 MHz) δ: 7.84-7.29 (m, 13H), 7.12 (d, J = 3.6 Hz, 0.5H), 7.07 (d, J = 8.0 Hz, 1H), 6.84 (d, J = 3.6 Hz, 0.5H), 6.54 (d, J = 3.2 Hz, 0.5H), 5.82 (d, J = 3.6 Hz, 0.5H), 5.11-4.84 (m, 2H), 4.29-4.15 (m, 2H), 2.46, 2.45 (2 s, 3H), 1.68, 1.65 (2 s, 6H); MS: 543.0 (M + H)⁺. |
| 27/39 | 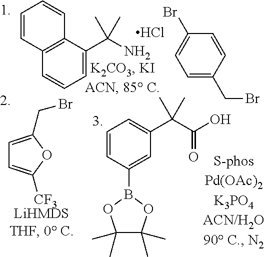 | 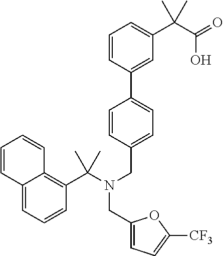 [SMILES: CC(C)(C(=O)O)c1cccc(-c2ccc(CN(Cc3ccc(C(F)(F)F)o3)C(C)(C)c3cccc4ccccc34)cc2)c1] | ¹H-NMR (CDCl₃, 400 MHz) δ: 9.60 (d, J = 8.8 Hz, 1H), 7.84 (d, J = 8.0 Hz, 1H), 7.76 (d, J = 8.0 Hz, 1H), 7.56-7.50 (m, 4H), 7.41-7.25 (m, 6H), 7.17 (d, J = 8.0 Hz, 2H), 6.44 (d, J = 1.6 Hz, 1H), 5.68 (d, J = 2.8 Hz, 1H), 3.81 (s, 4H), 1.73 (s, 6H), 1.63 (s, 6H); MS: 584.0 (M + H)⁺. |
| 27/40 | 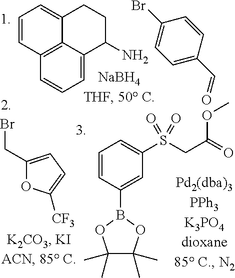 | 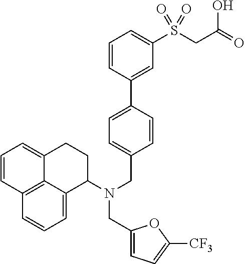 [SMILES: O=C(O)CS(=O)(=O)c1cccc(-c2ccc(CN(Cc3ccc(C(F)(F)F)o3)C3CCc4cccc5cccc3c45)cc2)c1] | ¹H-NMR (CDCl₃, 400 MHz) δ: 8.01 (d, J = 7.2 Hz, 1H), 7.97 (s, 1H), 7.72 (d, J = 8.8 Hz, 1H), 7.63 (d, J = 8.0 Hz, 1H), 7.59 (d, J = 8.4 Hz, 1H), 7.48- 7.41 (m, 2H), 7.35-7.22 (m, 6H), 7.12 (d, J = 6.8 Hz, 1H), 6.63 (d, J = 1.6 Hz, 1H), 6.31 (s, 1H), 4.13-4.06 (m, 3H), 3.78-3.69 (m, 2H), 3.60-3.53 (m, 2H), 3.13-3.09 (m, 1H), 2.94-2.84 (m, 1H), 2.42- 2.22 (m, 1H), 1.78-1.74 (m, 1H); |
| | | | MS: 620.2 (M + H)⁺. |
| 27/41 | 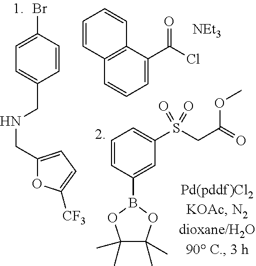 | 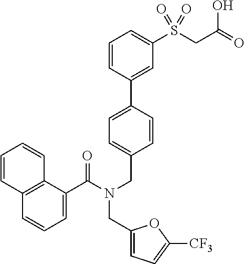 [SMILES: O=C(O)CS(=O)(=O)c1cccc(-c2ccc(CN(Cc3ccc(C(F)(F)F)o3)C(=O)c3cccc4ccccc34)cc2)c1] | ¹H-NMR (500 MHz, CD₃OD) δ: 8.24, 8.15 (2 s, 1H), 8.04-7.94 (m, 4H), 7.86 (d, J = 9.0 Hz, 1H), 7.77-7.70 (m, 2H), 7.61-7.54 (m, 6H), 7.20 (d, J = 8.5 Hz, 1H), 7.02 (d, J = 2.0 Hz, 0.5H), 6.75 (d, J = 2.0 Hz, 0.5H), 6.59 (d, J = 3.0 Hz, 0.5H), 6.18 (d, J = 3.0 Hz, 0.5H), 5.35-4.97 (m, 2H), 4.60-4.34 (m, 4H); MS: 608.2 (M + H)⁺. |
| 27/42 | 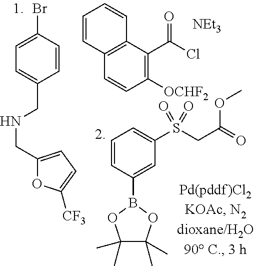 | 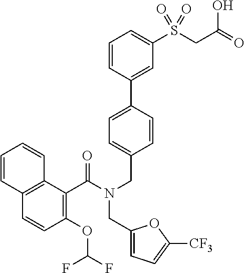 [SMILES: O=C(O)CS(=O)(=O)c1cccc(-c2ccc(CN(Cc3ccc(C(F)(F)F)o3)C(=O)c3c(OC(F)F)ccc4ccccc34)cc2)c1] | ¹H-NMR (500 MHz, CD₃OD) δ: 8.24 (t, J = 1.5 Hz, 0.5H), 8.12-7.91 (m, 4.5H), 7.78-7.51 (m, 8H), 7.29-6.69 (m, 2.5H), 6.68 (d, J = 1.0 Hz, 0.5H), 6.57 (d, J = 3.0 Hz, 0.5H), 6.08 (d, J = 3.5 Hz, 0.5H), 5.41-4.66 (m, 2H), 4.44-4.32 (m, 4H); MS: 674.2 (M + H)⁺. |
| 27/43 | 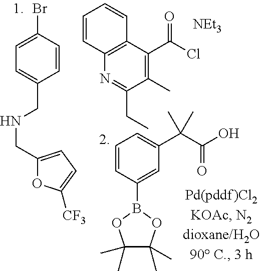 | 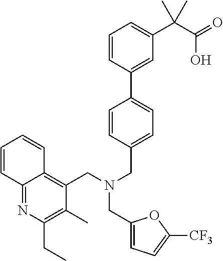 | ¹H-NMR (400 MHz, CD₃OD) δ: 8.22-8.17 (m, 1H), 8.05-7.81 (m, 3H), 7.66-7.39 (m, 7H), 7.05-7.04 (m, 0.5H), 6.99 (d, J = 8.4 Hz, 1H), 6.81- 6.79 (m, 0.5H), 6.61 (d, J = 2.8 Hz, 0.5H), 6.34 (d, J = 3.2 Hz, 0.5H), 5.13- 5.10 (m, 1.5H), 4.91-4.87 (m, 0.5H), 4.41 (d. J = 6.4 Hz, 2H), 3.30-3.24 (m, 2H), 2.60, 2.53 (2 s, 3H), 1.65, 1.62 (2 s, 6H), 1.49-1.43 (m, 3H); MS: 615.2 (M + H)⁺. |
| 27/44 | 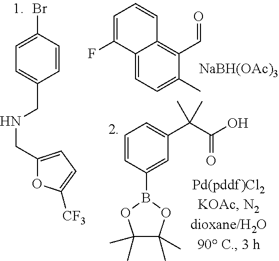 | 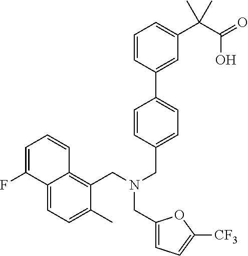 [SMILES: Cc1ccc2c(F)cccc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 8.07 (d, J = 8.5 Hz, 1H), 7.74 (br s, 1H), 7.64-7.62 (m, 3H), 7.52-7.41 (m, 7H), 7.19 (dd, J = 10.3, 7.8 Hz, 1H), 7.03 (s, 1H), 6.69 (s, 1H), 4.66 (br s, 2H), 4.25 (br s, 2H), 4.15 (br s, 2H), 2.56 (s, 3H), 1.63 (s, 6H); MS: 590.2 (M + H)⁺. |
| 27/45 | 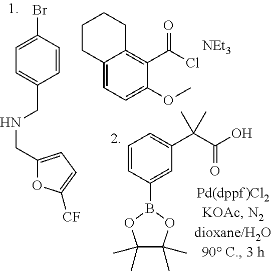 | 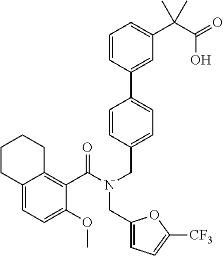 [SMILES: COc1ccc2c(c1C(=O)N(Cc1ccc(-c3cccc(C(C)(C)C(=O)O)c3)cc1)Cc1ccc(C(F)(F)F)o1)CCCC2] | ¹H-NMR (500 MHz, CD₃OD) δ: 7.62-7.58 (m, 2H), 7.53-7.40 (m, 5H), 7.17-7.13 (m, 2H), 7.96-7.95 (m, 0.5H), 6.89 (t, J = 8.5 Hz, 1H), 6.84-6.83 (m, 0.5H), 6.51 (d, J = 3.0 Hz, 0.5H), 6.18 (d, J = 3.0 Hz, 0.5H), 5.17 (d, J = 15.5 Hz, 0.5H), 5.04 (d, J = 15.5 Hz, 0.5H), 4.63-4.26 (m, 3H), 3.87, 3.84 (2 s, 3H), 2.81-2.24 (m, 4H), 1.87-1.73 (m, 4H), 1.64, 1.62 (2 s; 6H); MS: 606.3 (M + H)⁺. |
| 27/46 | 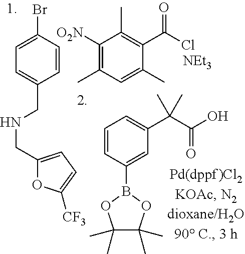 | 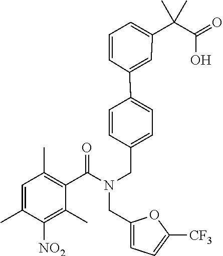 [SMILES: Cc1cc(C)c([N+](=O)[O-])c(C)c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 7.61-7.40 (m, 7H), 7.22 (s, 1H), 7.09 (d, J = 7.5 Hz, 1H), 6.97 (s, 0.5H), 6.87 (d, J = 1.5 Hz, 0.5H), 6.55 (s, 0.5H), 6.34 (s, 0.5H), 4.99-4.78 (m, 2H), 4.45-4.36 (m, 2H), 2.31-2.04 (m, 9H), 1.63 (s, 6H); MS: 606.9 (M − H)⁻. |
| 27/47 | 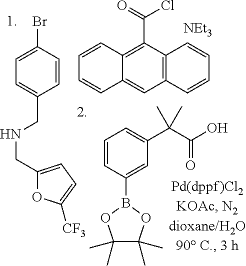 | 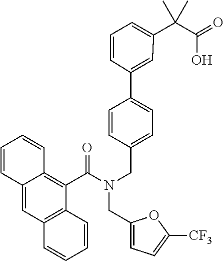 [SMILES: CC(C)(C(=O)O)c1cccc(-c2ccc(CN(Cc3ccc(C(F)(F)F)o3)C(=O)c3c4ccccc4cc4ccccc34)cc2)c1] | ¹H-NMR (500 MHz, CD₃OD) δ: 8.63 (s, 1H), 8.12-8.09 (m, 2H), 7.98- 7.89 (m, 2H), 7.69-7.23 (m, 11H), 7.02 (d, J = 2.5 Hz, 0.5H), 6.82 (d, J = 8.0 Hz, 1H), 6.59-6.58 (m, 0.5H), 6.56 (d, J = 3.0 Hz, 0.5H), 5.82 (d, J = 3.0 Hz, 0.5H), 5.10, 5.08 (2 s, 2H), 4.21, 4.15 (2 s, 2H), 1.66, 1.60 (2 s, 6H); MS: 622.0 (M + H)⁺. |
| 27/48 | 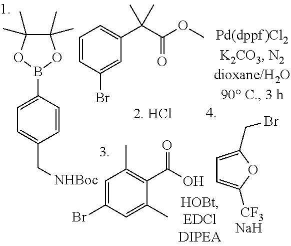 | 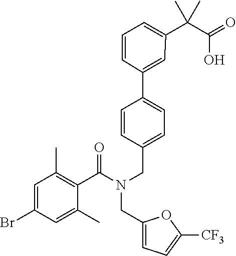 [SMILES: Cc1cc(Br)cc(C)c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 7.61-7.56 (m, 3H), 7.51-7.40 (m, 4H), 7.34 (d, J = 4.0 Hz, 2H), 7.17 (d, J = 7.5 Hz, 1H), 6.96-6.95 (m, 0.5H), 6.87-6.86 (m, 0.5H), 6.51 (d, J = 3.0 Hz, 0.5H), 6.34 (d, J = 3.0 Hz, 0.5H), 4.99-4.86 (m, 2H), 4.41, 4.37 (2 s, 2H), 2.28, 2.23 (2 s, 6H), 1.63, 1.62 (2 s, 6H); MS: 625.8 (M − H)⁻. |
| 27/49 | 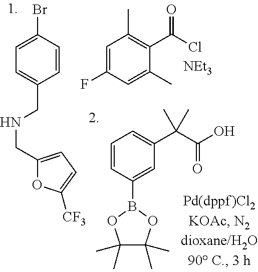 | 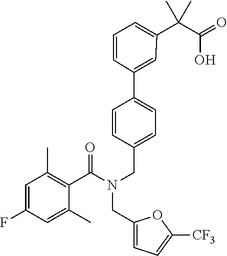 [SMILES: Cc1cc(F)cc(C)c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 7.6-7.58 (m, 3H), 7.56-7.40 (m, 4H), 7.17 (d, J = 8.0 Hz, 1H), 6.96-6.86 (m, 3H), 6.51 (d, J = 3.5 Hz, 0.5H), 6.33 (d, J = 3.5 Hz, 0.5H), 4.90-4.86 (m, 2H), 4.41, 4.37 (2 s, 2H), 2.29, 2.24 (2 s, 6H), 1.63, 1.62 (2 s, 6H); MS: 565.9 (M − H)⁻. |
| 27/50 | 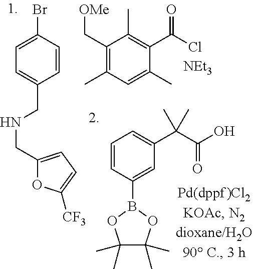 | 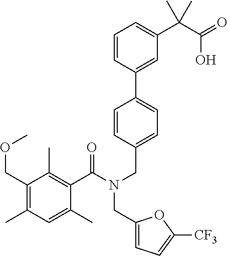 [SMILES: COCc1c(C)cc(C)c(C(=O)N(Cc2ccc(-c3cccc(C(C)(C)C(=O)O)c3)cc2)Cc2ccc(C(F)(F)F)o2)c1C] | ¹H-NMR (500 MHz, CD₃OD) δ: 7.61-7.40 (m, 7H), 7.15 (d, J = 8.0 Hz, 1H), 7.02 (s, 1H), 6.95-6.94 (m, 0.5H), 6.85 (d, J = 2.0 Hz, 0.5H), 6.50 (d, J = 3.0 Hz, 0.5H), 6.29 (d, J = 3.5 Hz, 0.5H), 4.90-4.81 (m, 2H), 4.53, 4.52 (2 s, 2H), 4.39-4.32 (m, 2H), 3.42, 3.41 (2 s, 3H), 2.40 (s, 3H), 2.30, 2.26 (2 s, 3H), 2.23, 2.20 (2 s, 3H), 1.63, 1.62 (2 s, 6H); MS: 608.3 (M + H)⁺. |
| 27/51 | 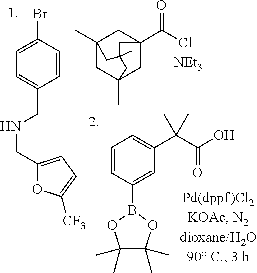 | 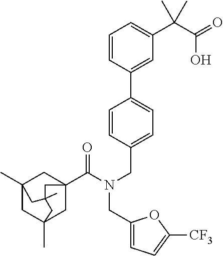 [SMILES: CC12CC3(C)CC(C)(C1)CC(C(=O)N(Cc1ccc(-c4cccc(C(C)(C)C(=O)O)c4)cc1)Cc1ccc(C(F)(F)F)o1)(C2)C3] | ¹H-NMR (500 MHz, CD₃OD) δ: 7.61-7.59 (m, 3H), 7.50-7.49 (m, 1H), 7.44-7.38 (m, 2H), 7.28 (d, J = 8.0 Hz, 2H), 6.90-6.89 (m, 1H), 6.40 (d, J = 3.0 Hz, 1H), 4.84 (br s, 2H), 4.66 (br s, 2H), 1.68 (s, 6H), 1.63 (s, 6H), 1.20-1.11 (m, 6H), 0.89 (s, 9H); MS: 620.0 (M − H)⁻. |
| 27/52 | 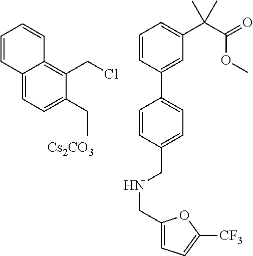 | 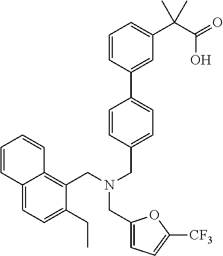 [SMILES: CCc1ccc2ccccc2c1CN(Cc1ccc(-c2cccc(C(C)(C)C(=O)O)c2)cc1)Cc1ccc(C(F)(F)F)o1] | ¹H-NMR (500 MHz, CD₃OD) δ: 7.92-7.88 (m, 3H), 7.67-7.63 (m, 3H), 7.53-7.44 (m, 8H), 7.07 (d, J = 2.0 Hz, 1H), 6.77 (s, 1H), 4.77 (br s, 2H), 4.37 (br s, 2H), 4.25 (br s, 2H), 2.81 (br s, 2H), 1.63 (s, 6H), 1.18 (t, J = 7.5 Hz, 3H); MS: 586.3 (M + H)⁺. |
| 27/53 | 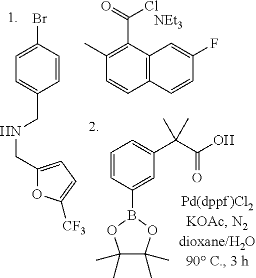 | 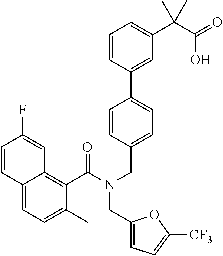 [SMILES: Cc1ccc2ccc(F)cc2c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 7.98-7.91 (m, 2H), 7.64-7.25 (m, 10H), 6.99-6.97 (m, 1.5H), 6.74 (s, 0.5H), 6.57 (s, 0.5H), 6.14 (s, 0.5H), 5.12- 4.85 (m, 2H), 4.34-4.29 (m, 2H), 2.48, 2.44 (2 s, 3H), 1.64, 1.61 (2 s, 6H); MS: 601.9 (M − H)⁻. |
| 27/54 | 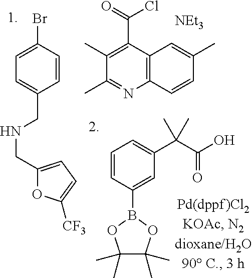 | 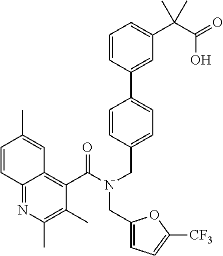 [SMILES: Cc1ccc2nc(C)c(C)c(C(=O)N(Cc3ccc(-c4cccc(C(C)(C)C(=O)O)c4)cc3)Cc3ccc(C(F)(F)F)o3)c2c1] | ¹H-NMR (500 MHz, CD₃OD) δ: 8.06-7.82 (m, 2H), 7.69-7.35 (m, 8H), 7.07- 7.06 (m, 0.5H), 6.95 (d, J = 8.5 Hz, 1H),6.85 (d, J = 2.0 Hz, 0.5H), 6.66 (d, J = 3.0 Hz, 0.5H), 6.40 (d, J = 3.5 Hz, 0.5H), 5.28- 4.99 (m, 2H), 4.48-4.36 (m, 2H), 2.93, 2.92 (2 s, 3H), 2.54-2.49 (m, 6H), 1.65- 1.82 (m, 6H); MS: 612.9 (M − H)⁻. |
| 27/55 | 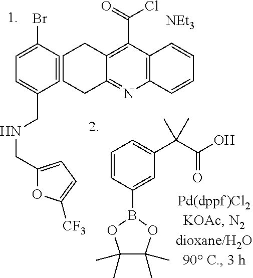 | 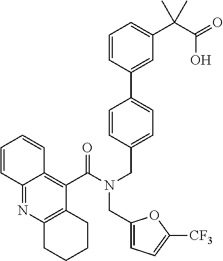 [SMILES: CC(C)(C(=O)O)c1cccc(-c2ccc(CN(Cc3ccc(C(F)(F)F)o3)C(=O)c3c4c(nc5ccccc35)CCCC4)cc2)c1] | ¹H-NMR (500 MHz, CD₃OD) δ: 8.16 (t, J = 8.3 Hz, 1H), 8.09-7.97 (m, 2H), 7.87-7.84 (m, 1H), 7.64 (d, J = 7.5 Hz, 2H), 7.54 (d, J = 7.5 Hz, 2H), 7.51-7.42 (m, 3H), 7.05 (d, J = 2.0 Hz, 0.5H), 6.98 (d, J = 7.5 Hz, 1H), 6.81 (d, J = 2.5 Hz, 0.5H), 6.61 (d, J = 3.5 Hz, 0.5H), 6.37 (d, J = 3.5 Hz, 0.5H), 5.21-4.82 (m, 2H), 4.45-4.36 (m, 2H), 3.39- 3.33 (m, 2H), 3.08-2.78 (m, 2H), 2.09-1.91 (m, 4H), 1.65, 1.62 (2 s, 6H); |
| | | | MS: 624.9 (M − H)⁻. |
| 27/56 | 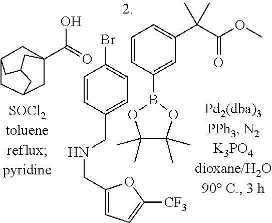 | 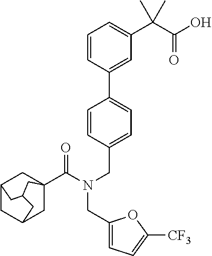 [SMILES: CC(C)(C(=O)O)c1cccc(-c2ccc(CN(Cc3ccc(C(F)(F)F)o3)C(=O)C34CC5CC(CC(C5)C3)C4)cc2)c1] | ¹H-NMR (CDCl₃, 400 MHz) δ: 7.59-7.55 (m, 3H), 7.47-7.41 (m, 3H), 7.26-7.24 (m, 2H), 6.71 (d, J = 2.0 Hz, 1H), 6.26 (d, J = 3.6 Hz, 1H), 4.85 (s, 2H), 4.53 (s, 2H), 2.09- 2.05 (m, 9H), 1.73 (br s, 6H), 1.67 (s, 6H); MS: 580.0 (M + 1)⁺. |
| 27/57 | 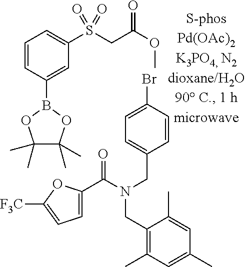 | 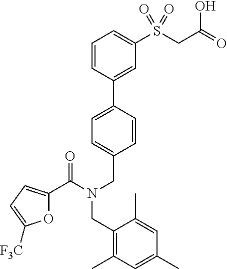 [SMILES: Cc1cc(C)c(CN(Cc2ccc(-c3cccc(S(=O)(=O)CC(=O)O)c3)cc2)C(=O)c2ccc(C(F)(F)F)o2)c(C)c1] | ¹H-NMR (CDCl₃, 400 MHz) δ: 8.05 (s, 1H), 7.84 (d, J = 7.6 Hz, 1H), 7.65 (d, J = 8.0 Hz, 1H), 7.43-7.39 (m, 3H), 7.26 (s, 1H), 7.04-6.94 (m, 2H), 6.78-6.71 (m, 3H), 4.86 (s, 2H), 4.46 (br s, 2H), 4.14 (s, 2H), 2.22 (s, 3H), 1.99 (s, 6H); MS: 600.1 (M + 1)⁺. |
| 27/58 | 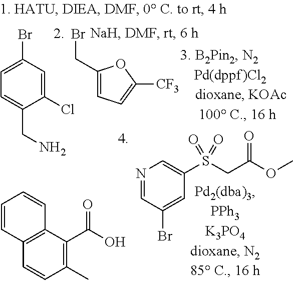 | 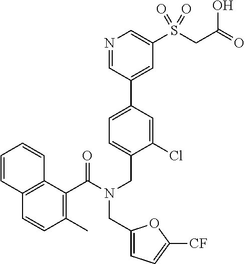 | MS: 656.9 (M + 1)⁺. |
| 27/59 | 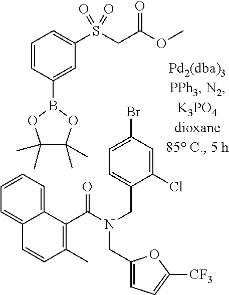 | 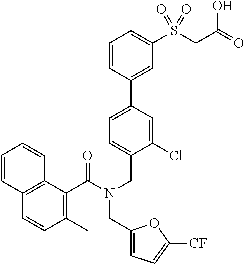 | ¹H-NMR (CDCl₃, 400 MHz) δ: 8.04, 7.95 (2 s, 1H), 7.85-7.81 (m, 1H), 7.75-7.56 (m, 4H), 7.49- 7.18 (m, 6.5H), 6.93 (d, J = 8.0 Hz, 0.5H), 6.69 (d, J = 2.0 Hz, 0.5H), 6.42-6.41 (m, 0.5H), 6.36 (d, J = 3.2 Hz, 0.5H), 5.76 (d, J = 2.8 Hz, 0.5H), 5.06-4.91 (m, 1H), 4.82-4.73 (m, 1H), 4.35-4.06 (m, 4H), 2.38, 2.31 (2 s, 3H); MS: 655.9 (M + 1)⁺. |
| 27/60 | 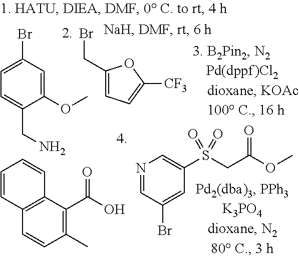 | 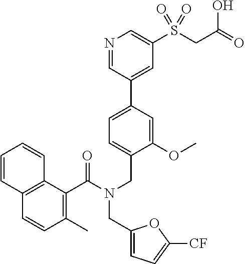 | ¹H-NMR (CDCl₃, 400 MHz) δ: 9.00 (d, J = 9.2 Hz, 1H), 8.85, 8.73 (2 s, 1H), 8.37, 8.22 (2 s, 1H), 7.69-7.44 (m, 5H), 7.34-6.62 (m, 4.5H), 6.44 (s, 0.5H), 6.34 (d, J = 2.0 Hz, 0.5H), 5.73 (s, 0.5H), 4.84-4.73 (m, 2H), 4.28-4.05 (m, 4H), 3.72- 3.42 (m, 3H), 2.31-2.18 (m, 3H); MS: 653.2 (M + 1)⁺. |
| 27/61 | 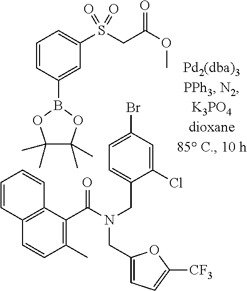 | 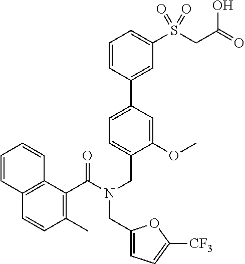 [SMILES: COc1cc(-c2cccc(S(=O)(=O)CC(=O)O)c2)ccc1CN(Cc1ccc(C(F)(F)F)o1)C(=O)c1c(C)ccc2ccccc12] | ¹H-NMR (CDCl₃, 400 MHz) δ: 8.10, 7.99 (2 s, 1H), 7.84-7.33 (m, 8.5H), 7.24-7.18 (m, 1H), 7.06-7.00 (m, 1H), 6.82-6.79 (m, 1H), 6.71 (d, J = 2.8 Hz, 0.5H), 6.62 (d, J = 3.6 Hz, 0.5H), 6.47 (m, 0.5H), 6.35 (d, J = 3.2 Hz, 0.5H), 5.75 (d, J = 2.8 Hz, 0.5H), 4.91-4.76 (m, 2H), 4.19-4.08 (m, 4H), 3.76, 3.51 (2 s, 3H), 2.32, 2.27 (2 s, 3H); MS: 651.9 (M + 1)⁺. |
| 27/62 | 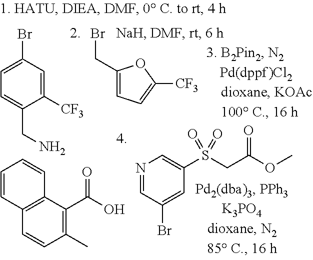 | 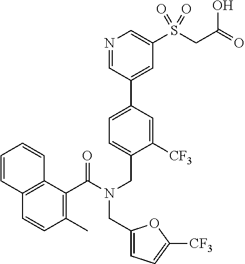 [SMILES: Cc1ccc2ccccc2c1C(=O)N(Cc1ccc(C(F)(F)F)o1)Cc1ccc(-c2cncc(S(=O)(=O)CC(=O)O)c2)cc1C(F)(F)F] | MS: 690.9 (M + 1)⁺. |
| 27/63 | 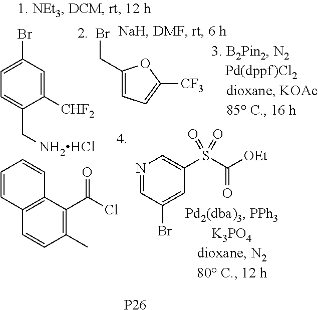 | 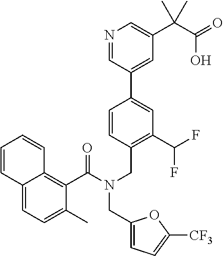 [SMILES: Cc1ccc2ccccc2c1C(=O)N(Cc1ccc(C(F)(F)F)o1)Cc1ccc(-c2cncc(C(C)(C)C(=O)O)c2)cc1C(F)F] | ¹H-NMR (CD₃OD, 400 MHz) δ: 8.71 (d, J = 2.4 Hz, 0.5H), 8.62 (t, J = 2.2 Hz, 1H), 8.59 (d, J = 1.6 Hz, 0.5H), 8.09-7.43 (m, 10H), 7.39-5.93 (m, 3H), 5.35-5.04 (m, 2H), 4.66-4.37 (m, 2H), 2.50, 2.41 (2 s, 3H), 1.69, 1.66 (2 s, 6H); MS: 637.3 (M + 1)⁺. |
| 27/64 | 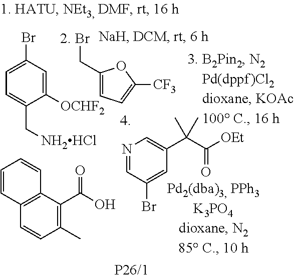 | 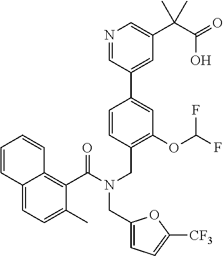 [SMILES: Cc1ccc2ccccc2c1C(=O)N(Cc1ccc(C(F)(F)F)o1)Cc1ccc(-c2cncc(C(C)(C)C(=O)O)c2)cc1OC(F)F] | ¹H-NMR (DMSO-d₆, 400 MHz) δ: 8.80-8.58 (m, 2H), 7.99-7.85 (m, 3H), 7.69-6.92 (m, 9H), 6.64 (d, J = 3.2 Hz, 0.5H), 6.17 (d, J = 3.2 Hz, 0.5H), 5.06-4.86 (m, 2H), 4.35-4.27 (m, 2H), 2.40, 2.31 (2 s, 3H), 1.60, 1.57 (2 s, 6H); MS: 653.0 (M + 1)⁺. |
| 27/65 | 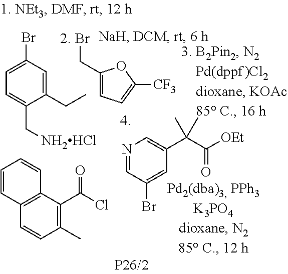 | 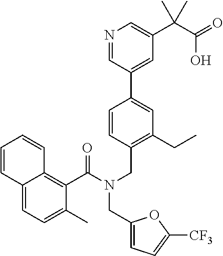 [SMILES: CCc1cc(-c2cncc(C(C)(C)C(=O)O)c2)ccc1CN(Cc1ccc(C(F)(F)F)o1)C(=O)c1c(C)ccc2ccccc12] | ¹H-NMR (CDCl₃, 400 MHz) δ: 8.74, 8.66 (2 s, 1H), 8.55 (d, J = 10.8 Hz, 1H), 7.97-7.84 (m, 3H), 7.71 (d, J = 8.8 Hz, 1H), 7.56-7.25 (m, 6H), 7.22 (d, J = 2.4 Hz, 0.5H), 6.68 (d, J = 2.0 Hz, 0.5H), 6.63 (d, J = 3.6 Hz, 0.5H), 6.08 (d, J = 3.2 Hz, 0.5H), 5.15- 4.83 (m, 2H), 4.37-4.24 (m, 2H), 2.84-2.76 (m, 1H), 2.46, 2.33 (2 s, 3H), 2.26-2.19 (m, 1H), 1.59, 1.56 (2 s, 6H), 1.27-1.24 (m, |
| | | | 1.5H), 1.07-1.03 (m, |
| | | | 0.5H), 0.78-0.74 |
| | | | (m, 1H); |
| | | | MS: 615.0 (M + 1)⁺. |
| 27/66 | 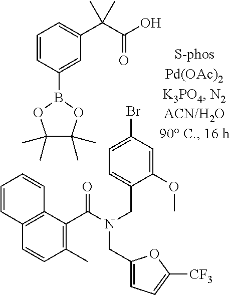 | 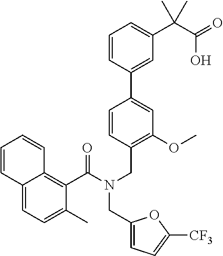 [SMILES: COc1cc(-c2cccc(C(C)(C)C(=O)O)c2)ccc1CN(Cc1ccc(C(F)(F)F)o1)C(=O)c1c(C)ccc2ccccc12] | ¹H-NMR (CDCl₃, 400 MHz) δ: 7.80-7.69 (m, 3H), 7.62-7.58 (m, 1H), 7.50-7.38 (m, 6H), 7.33-7.28 (m, 1H), 7.21-6.90 (m, 2H), 6.79-5.85 (m, 2H), 5.11-4.91 (m, 2H), 4.32, 4.18 (2 s, 2H), 3.94, 3.69 (2 s, 3H), 2.43, 2.38 (2 s, 3H), 1.67, 1.64 (2 s, 6H); MS: 616.2 (M + 1)⁺. |
| 27/67 | 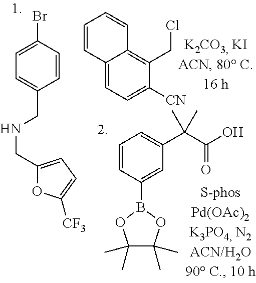 | 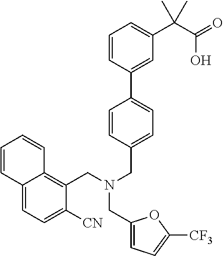 [SMILES: CC(C)(C(=O)O)c1cccc(-c2ccc(CN(Cc3ccc(C(F)(F)F)o3)Cc3c(C#N)ccc4ccccc34)cc2)c1] | ¹H-NMR (CDCl₃, 400 MHz) δ: 8.31 (d, J = 8.4 Hz, 1H), 8.25 (d, J = 7.6 Hz, 1H), 7.87 (d, J = 7.2 Hz , 1H), 7.70-7.37 (m, 11H), 6.74 (dd, J = 3.4, 1.0 Hz, 1H), 6.25 (d, J = 3.2 Hz, 1H), 4.14 (s, 2H), 3.72 (s, 4H), 1.64 (s, 6H); MS: 583.0 (M + 1)⁺. |
| 27/68 | 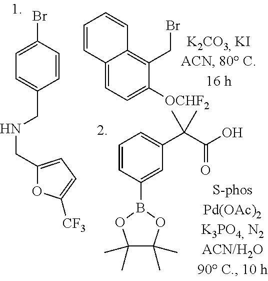 | 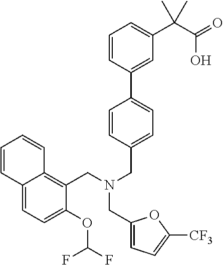 [SMILES: CC(C)(C(=O)O)c1cccc(-c2ccc(CN(Cc3ccc(C(F)(F)F)o3)Cc3c(OC(F)F)ccc4ccccc34)cc2)c1] | ¹H-NMR (CDCl₃, 400 MHz) δ: 8.24 (d, J = 8.4 Hz, 1H), 7.79 (t, J = 9.0 Hz, 2H), 7.55- 7.26 (m, 11H), 6.71 (d, J = 2.0 Hz, 1H), 6.61 (t, J = 74.2 Hz, 1H), 6.27 (d, J = 2.8 Hz, 1H), 4.19 (s, 2H), 3.70 (s, 2H), 3.65 (s, 2H), 1.64 (s, 6H); MS: 624.0 (M + 1)⁺. |
| 27/69 | 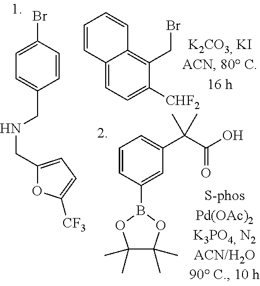 | 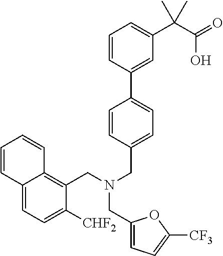 [SMILES: CC(C)(C(=O)O)c1cccc(-c2ccc(CN(Cc3ccc(C(F)(F)F)o3)Cc3c(C(F)F)ccc4ccccc34)cc2)c1] | ¹H-NMR (CDCl₃, 400 MHz) δ: 8.39 (d, J = 7.6 Hz, 1H), 7.89-7.85 (m, 2H), 7.72 (d, J = 8.8 Hz, 1H), 7.60-7.20 (m, 11H), 6.73 (d, J = 2.0 Hz, 1H), 6.24 (br s, 1H), 4.29 (s, 2H), 3.70 (s, 2H), 3.62 (s, 2H), 1.64 (s, 6H); MS: 608.0 (M + 1)⁺. |
| 27/70 | 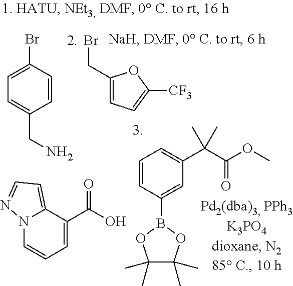 | 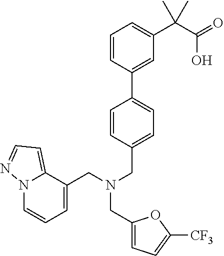 | ¹H-NMR (CDCl₃, 400 MHz) δ: 8.56 (d, J = 6.8 Hz, 1H), 8.02 (d, J = 2.4 Hz, 1H), 7.59- 7.17 (m, 9H), 6.80-6.41 (m, 4H), 4.77 (br s, 2H), 4.49 (br s, 2H), 1.66 (s, 6H); MS: 562.0 (M + 1)⁺. |
| 27/71 | 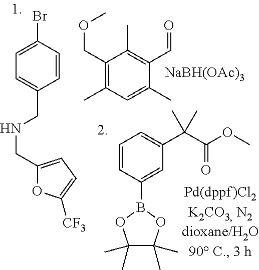 | 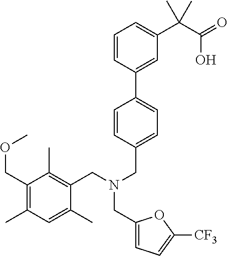 [SMILES: COCc1c(C)cc(C)c(CN(Cc2ccc(-c3cccc(C(C)(C)C(=O)O)c3)cc2)Cc2ccc(C(F)(F)F)o2)c1C] | ¹H-NMR (500 MHz, CD₃OD) δ: 7.69 (d, J = 8.0 Hz, 2H), 7.64 (s, 1H), 7.54- 7.42 (m, 5H), 7.08 (s, 1H), 7.01 (s, 1H), 6.79 (br s, 1H), 4.52 (s, 2H), 4.37-4.21 (m, 6H), 3.44 (s, 3H), 2.38 (s, 3H), 2.33 (s, 3H), 2.26 (s, 3H), 1.63 (s, 6H); MS: 594.3 (M + 1)⁺. |
| 27/72 | 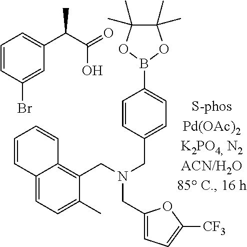 | 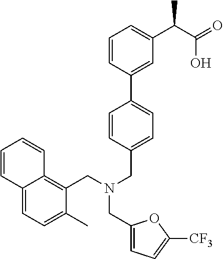 [SMILES: Cc1ccc2ccccc2c1CN(Cc1ccc(-c2cccc([C@@H](C)C(=O)O)c2)cc1)Cc1ccc(C(F)(F)F)o1] | ¹H-NMR (400 MHz, CDCl₃) δ: 8.26 (d, J = 8.8 Hz, 1H), 7.77 (d, J = 8.0 Hz, 1H), 7.67 (d, J = 8.4 Hz, 1H), 7.51-7.27 (m, 11H), 6.72 (d, J = 2.0 Hz, 1H), 6.22 (d, J = 2.8 Hz, 1H), 4.16 (s, 2H), 3.79 (q, J = 7.2 Hz, 1H), 3.70 (s, 2H), 3.62 (s, 2H), 2.55 (s, 3H), 1.54 (d, J = 7.2 Hz, 3H); MS: 558.0 (M + 1)⁺. |
| 27/73 | 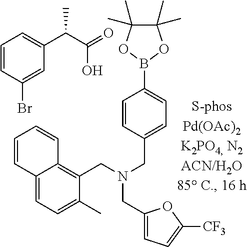 | 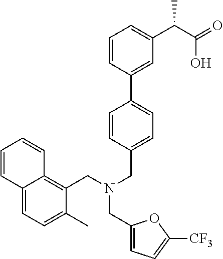 [SMILES: Cc1ccc2ccccc2c1CN(Cc1ccc(-c2cccc([C@H](C)C(=O)O)c2)cc1)Cc1ccc(C(F)(F)F)o1] | ¹H-NMR (400 MHz, CDCl₃) δ: 8.26 (d, J = 8.4 Hz, 1H), 7.77 (d, J = 7.6 Hz, 1H), 7.67 (d, J = 8.0 Hz, 1H), 7.51-7.26 (m, 11H), 6.72 (dd, J = 1.2, 3.2 Hz, 1H), 6.22 (d, J = 3.2 Hz, 1H), 4.16 (s, 2H), 3.79 (q, J = 7.2 Hz, 1H), 3.70 (s, 2H), 3.62 (s, 2H), 2.55 (s, 3H), 1.54 (d, J = 7.6 Hz, 3H); MS: 558.0 (M + 1)⁺. |
| 27/74 | 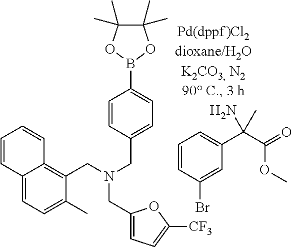 | 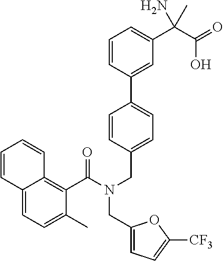 [SMILES: Cc1ccc2ccccc2c1C(=O)N(Cc1ccc(-c2cccc(C(C)(N)C(=O)O)c2)cc1)Cc1ccc(C(F)(F)F)o1] | ¹H-NMR (500 MHz, CD₃OD) δ: 7.92 (d, J = 6.5 Hz, 2H), 7.85-7.43 (m, 11H), 7.08 (d, J = 7.5 Hz, 1H), 7.01 (d, J = 2.0 Hz, 0.5H), 6.74 (s, 0.5H), 6.56 (d, J = 3.0 Hz, 0.5H), 6.10 (d, J = 3.0 Hz, 0.5H), 5.10-4.95 (m, 2H), 4.39- 4.30 (m, 2H), 2.47, 2.44 (2 s, 3H), 2.07, 2.04 (2s, 3H); MS: 587.3 (M + 1)⁺. |
| 27/75 | 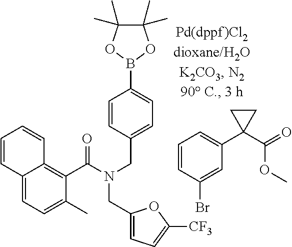 | 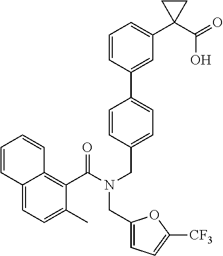 [SMILES: Cc1ccc2ccccc2c1C(=O)N(Cc1ccc(-c2cccc(C3(C(=O)O)CC3)c2)cc1)Cc1ccc(C(F)(F)F)o1] | ¹H-NMR (500 MHz, CD₃OD) δ: 7.93-7.90 (m, 2H), 7.79-7.34 (m, 11H), 7.04 (d, J = 8.5 Hz, 1H), 7.00 (dd, J = 2.0 Hz, 0.5H), 6.74 (s, 0.5H), 6.55 (d, J = 2.5 Hz, 0.5H), 6.09 (s, 0 .5H), 5.07-4.92 (m, 2H), 4.42-4.22 (m, 2H), 2.48, 2.45 (2 s, 3H), 1.67-1.62 (m, 2H), 1.31-1.25 (m, 2H); MS: 584.0 (M + 1)⁺. |
| 27/76 | 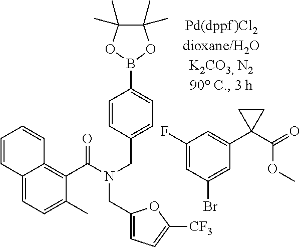 | 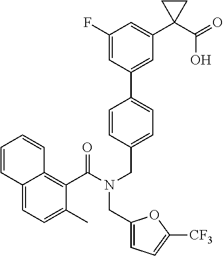 [SMILES: Cc1ccc2ccccc2c1C(=O)N(Cc1ccc(-c2cc(F)cc(C3(C(=O)O)CC3)c2)cc1)Cc1ccc(C(F)(F)F)o1] | ¹H-NMR (500 MHz, CD₃OD) δ: 7.91-7.88 (m, 2H), 7.77-7.03 (m, 10H), 7.01-6.98 (m, 1.5H), 6.72 (d, J = 1.0 Hz, 0.5H), 6.53 (d, J = 3.5 Hz, 0.5H), 6.07 (d, J = 3.0 Hz, 0.5H), 5.05-4.89 (m, 2H), 4.33-4.23 (m, 2H), 2.46, 2.43 (2 s, 3H), 1.67-1.62 (m, 2H), 1.32-1.24 (m, 2H); MS: 602.0 (M + 1)⁺. |
| 27/77 | 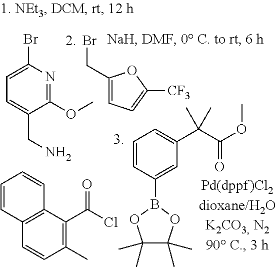 | 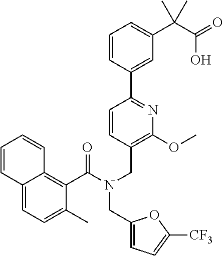 [SMILES: COc1nc(-c2cccc(C(C)(C)C(=O)O)c2)ccc1CN(Cc1ccc(C(F)(F)F)o1)C(=O)c1c(C)ccc2ccccc12] | ¹H-NMR (500 MHz, CD₃OD) δ: 8.21, 8.03 (2 s, 1H), 7.92-7.38 (m, 10H), 7.10 (d, J = 7.5 Hz, 0.5H), 7.00 (s, 0.5H), 6.87 (d, J = 7.5 Hz, 0.5H), 6.72 (s, 0.5H), 6.56 (s, 0.5H), 6.05 (s, 0.5H), 5.19-4.92 (m, 2H), 4.46-4.24 (m, 2H), 4.16, 3.89 (2 s, 3H), 2.44, 2.35 (2 s, 3H), 1.66, 1.62 (2 s, 6H); MS: 617.0 (M + 1)⁺. |
| 27/78 | 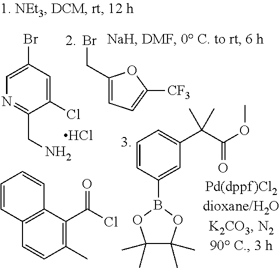 | 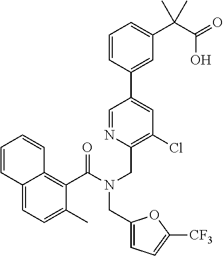 [SMILES: Cc1ccc2ccccc2c1C(=O)N(Cc1ccc(C(F)(F)F)o1)Cc1ncc(-c2cccc(C(C)(C)C(=O)O)c2)cc1Cl] | ¹H-NMR (500 MHz, CD₃OD) δ: 8.81-7.31 (m, 12H), 7.02 (d, J = 3.0 Hz, 0.5H), 6.73 (d, J = 2.5 Hz, 0.5H), 6.65 (d, J = 3.0 Hz, 0.5H), 6.07 (d, J = 3.5 Hz, 0.5H), 5.34-5.10 (m, 2H), 4.60-4.52 (m, 2H), 2.59, 2.39 (2 s, 3H), 1.66, 1.64 (2 s, 6H); MS: 621.2 (M + 1)⁺. |
| 27/79 | 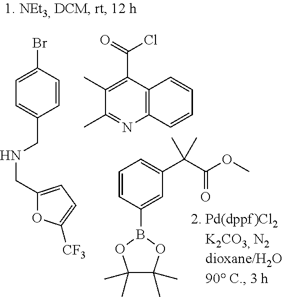 | 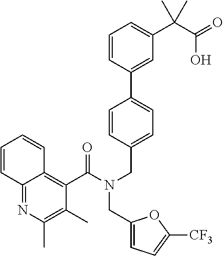 [SMILES: Cc1nc2ccccc2c(C(=O)N(Cc2ccc(-c3cccc(C(C)(C)C(=O)O)c3)cc2)Cc2ccc(C(F)(F)F)o2)c1C] | ¹H-NMR (500 MHz, CD₃OD) δ: 8.01 (d, J = 8.5 Hz, 1H), 7.77-7.39 (m, 10H), 7.03-7.02 (m, 0.5H), 6.97 (d, J = 8.0 Hz, 1H), 6.76- 6.75 (m, 0.5H), 6.58 (d, J = 3.0 Hz, 0.5H), 6.17 (d, J = 4.0 Hz, 0.5H), 5.09-4.92 (m, 2H), 4.38-4.28 (m, 2H), 2.75, 2.71 (2 s, 3H), 2.44, 2.37 (2 s, 3H), 1.65, 1.61 (2 s, 6H); MS: 601.3 (M + 1)⁺. |
| 27/80 | 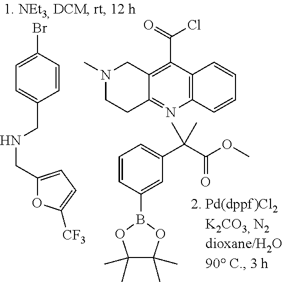 | 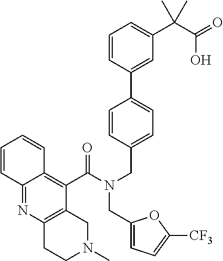 [SMILES: CN1CCc2nc3ccccc3c(C(=O)N(Cc3ccc(-c4cccc(C(C)(C)C(=O)O)c4)cc3)Cc3ccc(C(F)(F)F)o3)c2C1] | ¹H-NMR (500 MHz, CD₃OD) δ: 8.09 (dd, J = 6.5, 7.5 Hz, 1H), 7.90-7.81 (m, 2H), 7.68- 7.41 (m, 8H), 7.04 (d, J = 2.0 Hz, 0.5H), 6.99 (d, J = 8.0 Hz, 1H), 6.82 (d, J = 2.0 Hz, 0.5H), 6.58 (d, J = 2.5 Hz, 0.5H), 6.35 (d, J = 3.5 Hz, 0.5H), 5.31- 4.36 (m, 6H), 3.99-3.52 (m, 4H), 3.15, 3.12 (2 s, 3H), 1.65, 1.62 (2 s, 6H); MS: 642.0 (M + 1)⁺. |
| 27/81 | 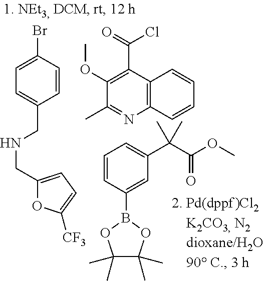 | 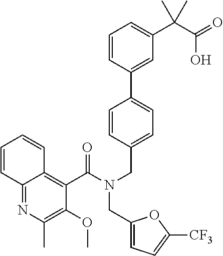 [SMILES: COc1c(C)nc2ccccc2c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 8.07-7.37 (m, 11H), 7.09 (d, J = 8.5 Hz, 1H), 7.02 (d, J = 2.0 Hz, 0.5H), 6.72 (d, J = 2.0 Hz, 0.5H), 6.58 (d, J = 3.5 Hz, 0.5H), 6.20 (d, J = 3.0 Hz, 0.5H), 5.27 (d, J = 14.5 Hz, 0.5H), 5.01 (s, 1H), 4.75 (d, J = 14.5 Hz, 0.5H), 4.49-4.37 (m, 2H), 4.04, 4.03 (2 s, 3H), 2.86-2.85 (m, 3H), 1.65, 1.62 (2 s, 6H); MS: 617.0 (M + 1)⁺. |
| 27/82 | 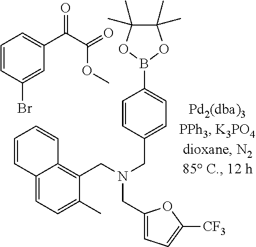 | 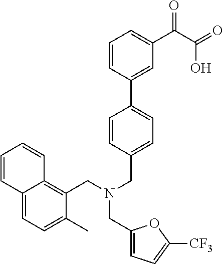 [SMILES: Cc1ccc2ccccc2c1CN(Cc1ccc(-c2cccc(C(=O)C(=O)O)c2)cc1)Cc1ccc(C(F)(F)F)o1] | ¹H-NMR (400 MHz, CD₃Cl) δ: 8.16-7.07 (m, 14H), 6.64 (s, 1H), 6.13 (s, 1H), 4.07 (s, 2H), 3.58 (s, 2H), 3.47 (s, 2H), 2.45 (s, 3H); MS: 558.2 (M + 1)⁺. |
| 27/83 | 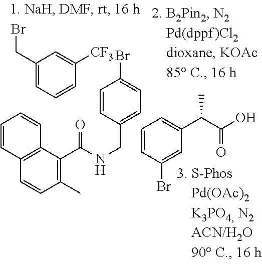 | 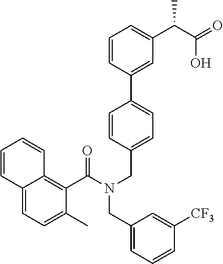 [SMILES: Cc1ccc2ccccc2c1C(=O)N(Cc1ccc(-c2cccc([C@H](C)C(=O)O)c2)cc1)Cc1cccc(C(F)(F)F)c1] | ¹H-NMR (CDCl₃, 400 MHz) δ: 7.82- 6.99 (m, 18H), 5.14- 5.04 (m, 1H), 4.81-4.66 (m, 1H), 4.29-4.12 (m, 2H), 3.87-3.76 (m, 1H), 2.47, 2.44 (2 s, 3H), 1.60-1.54 (m, 3H); MS: 582.0 (M + 1)⁺. |
| 27/84 | 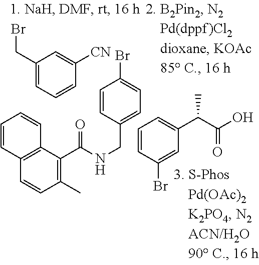 | 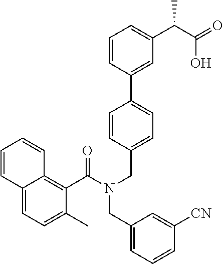 [SMILES: Cc1ccc2ccccc2c1C(=O)N(Cc1ccc(-c2cccc([C@H](C)C(=O)O)c2)cc1)Cc1cccc(C#N)c1] | ¹H-NMR (CDCl₃, 400 MHz) δ: 7.83-7.00 (m, 18H), 5.17-5.03 (m, 1H), 4.72-4.65 (m, 1H), 4.29-4.13 (m, 2H), 3.87-3.79 (m, 1H), 2.46, 2.43(2 s, 3H), 1.61-1.55 (m, 3H); MS: 539.0 (M + 1)⁺. |
| 27/85 | 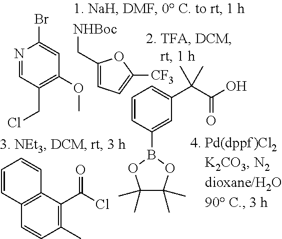 | 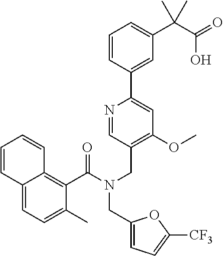 [SMILES: COc1cc(-c2cccc(C(C)(C)C(=O)O)c2)ncc1CN(Cc1ccc(C(F)(F)F)o1)C(=O)c1c(C)ccc2ccccc12] | ¹H-NMR (500 MHz, CD₃OD) δ: 8.76 (s, 0.5H), 7.96-7.31 (m, 11.5H), 7.07 (dd, J = 3.5, 1.0 Hz, 0.5H), 6.75-6.71 (m, 1H), 6.05 (d, J = 3.5 Hz, 0.5H), 5.44-4.98 (m, 2H), 4.58-4.44 (m, 2H), 4.34, 4.06 (2 s, 3H), 2.43 (s, 3H), 1.70, 1.69 (2 s, 6H); MS: 617.0 (M + 1)⁺. |
| 27/86 | 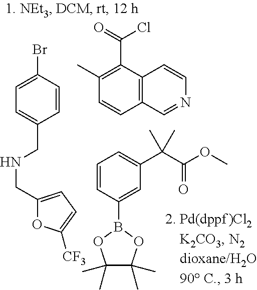 | 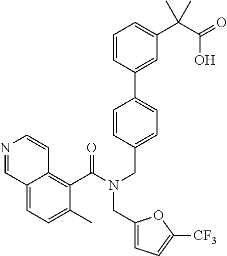 [SMILES: Cc1ccc2cnccc2c1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 9.65, 9.57 (2 s, 1H), 8.56 (d, J = 6.5 Hz, 0.5H), 8.44-8.38 (m, 1.5H), 8.01-7.90 (m, 2H), 7.68-7.34 (m, 7H), 7.04 (d, J = 2.0 Hz, 0.5H), 6.92 (d, J = 8.5 Hz, 1H), 6.76 (d, J = 2.5 Hz, 0.5H), 6.64 (d, J = 3.0 Hz, 0.5H), 6.24 (d, J = 3.0 Hz, 0.5H), 5.26 (d, J = 15.5 Hz, 0.5H), 5.20 (d, J = 14.5 Hz, 0.5H), 5.01 (d, J = 15.5 Hz, 0.5H), 4.84 (d, J = 14.5 Hz, 0.5H), 4.46-4.33 (m, 2H), 2.65, 2.61 (2 s, 3H), 1.65, 1.61 (2 s, 6H); |
| | | | MS: 587.0 (M + 1)⁺. |
| 27/87 | 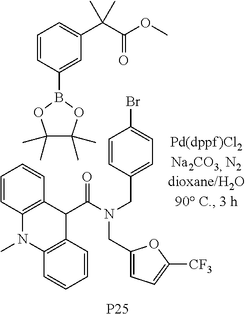 | 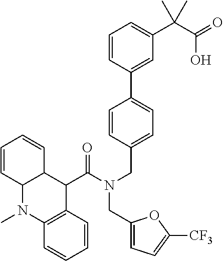 [SMILES: CN1c2ccccc2C(C(=O)N(Cc2ccc(-c3cccc(C(C)(C)C(=O)O)c3)cc2)Cc2ccc(C(F)(F)F)o2)C2C=CC=CC21] | ¹H-NMR (500 MHz, CD₃OD) δ: 7.58 (s, 1H), 7.54-7.40 (m, 5H), 7.28- 6.85 (m, 11H), 6.29 (d, J = 3.0 Hz, 1H), 5.77, 5.56 (2 s, 1H), 4.93, 4.85 (2 s, 2H), 4.66, 4.65 (2 s, 2H), 3.42, 3.37 (2 s, 3H), 1.62 (s, 6H). MS: 622.8 (M − CH₄ + 1)⁺. |
| 27/88 | 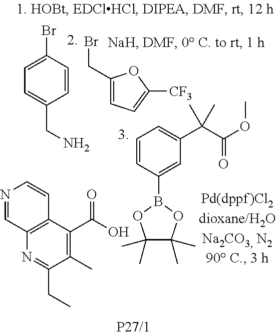 | 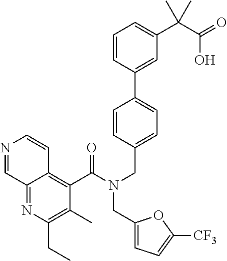 [SMILES: CCc1nc2cnccc2c(C(=O)N(Cc2ccc(-c3cccc(C(C)(C)C(=O)O)c3)cc2)Cc2ccc(C(F)(F)F)o2)c1C] | ¹H-NMR (500 MHz, CD₃OD) δ: 9.54, 9.48 (2 s, 1H), 8.59 (d, J = 5.5 Hz, 0.5H), 8.50 (d, J = 5.5 Hz, 0.5H), 7.85 (d, J = 6.0 Hz, 0.5H), 7.82 (d, J = 6.0 Hz, 0.5H), 7.66- 7.35 (m, 7H), 7.05 (d, J = 2.0 Hz, 0.5H), 6.91 (d, J = 8.0 Hz, 1H), 6.77 (d, J = 2.0 Hz, 0.5H), 6.64 (d, J = 3.0 Hz, 0.5H), 6.26 (d, J = 3.5 Hz, 0.5H), 5.21 (d, J = 15.0 Hz, 0.5H), 5.15 (d, J = 14.5 Hz, 0.5H), 5.06-4.84 (m, 1H), 4.43-4.34 (m, 2H), 3.19- 3.11 (m, 2H), 2.57, 2.49 (2 s, 3H), 1.65, 1.62 (2 s, 6H), 1.49-1.44 |
| | | | (m, 3H); |
| | | | MS: 616.0 (M + 1)⁺. |
| 27/89 | 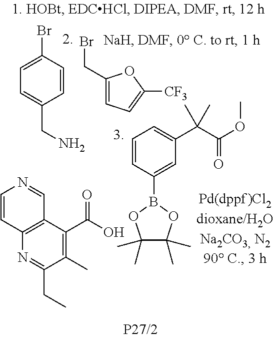 | 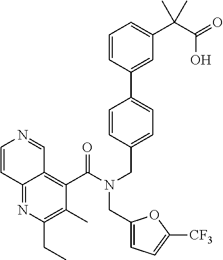 [SMILES: CCc1nc2ccncc2c(C(=O)N(Cc2ccc(-c3cccc(C(C)(C)C(=O)O)c3)cc2)Cc2ccc(C(F)(F)F)o2)c1C] | ¹H-NMR (500 MHz, CD₃OD) δ: 9.01, 8.92 (2 s, 1H), 8.68 (d, J = 6.5 Hz, 0.5H), 8.59 (d, J = 6.0 Hz, 0.5H), 7.96 (d, J = 6.0 Hz, 0.5H), 7.88 (d, J = 6.0 Hz, 0.5H), 7.68-7.35 (m, 6H), 7.31 (d, J = 8.0 Hz, 1H), 7.04 (d, J = 2.5 Hz, 0.5H), 6.89 (d, J = 8.5 Hz, 1H), 6.77 (d, J = 2.5 Hz, 0.5H), 6.66 (d, J = 3.5 Hz, 0.5H), 6.24 (d, J = 3.0 Hz, 0.5H), 5.33 (d, J = 15.5 Hz, 0.5H), 5.06 (d, J = 14.0 Hz, 0.5H), 5.00-4.92 (m, 1H), 4.48-4.37 (m, 2H), 3.14-3.09 (m, 2H), 2.52, 2.46 (2 s, 3H), 1.64, |
| | | | 1.62 (2 s, 6H), 1.45- |
| | | | 1.41 (m, 3H); |
| | | | MS: 616.0 (M + 1)⁺. |
| 27/90 | 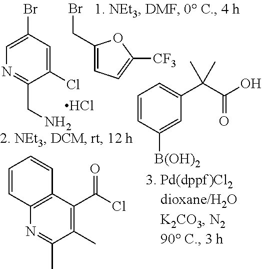 | 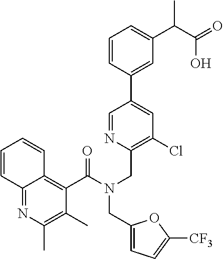 [SMILES: Cc1nc2ccccc2c(C(=O)N(Cc2ccc(C(F)(F)F)o2)Cc2ncc(-c3cccc(C(C)C(=O)O)c3)cc2Cl)c1C] | ¹H-NMR (500 MHz, CD₃OD) δ: 8.83 (d, J = 1.5 Hz, 0.5H), 8.64 (d, J = 1.5 Hz, 0.5H), 8.31 (d, J = 8.5 Hz, 0.5H), 8.13 (d, J = 1.5 Hz, 0.5H), 8.04 (d, J = 8.5 Hz, 0.5H), 7.91 (d, J = 8.0 Hz, 0.5H), 7.80- 7.37 (m, 7H), 7.03 (d, J = 2.0 Hz, 0.5H), 6.75 (d, J = 2.5 Hz, 0.5H), 6.68 (d, J = 3.5 Hz, 0.5H), 6.17 (d, J = 3.0 Hz, 0.5H), |
| | | | 5.36-5.13 (m, 2H), |
| | | | 4.63-4.51 (m, 2H), |
| | | | 3.89-3.83 (m, 1H), |
| | | | 2.79, 2.69 (2 s, 3H), |
| | | | 2.60, 3.35 (2 s, 3H), |
| | | | 1.55 (t, J = 7.8 Hz, 3H); |
| | | | MS: 621.9 (M + 1)⁺. |
| 27/91 | 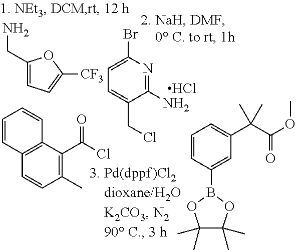 | 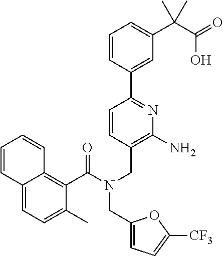 [SMILES: Cc1ccc2ccccc2c1C(=O)N(Cc1ccc(C(F)(F)F)o1)Cc1ccc(-c2cccc(C(C)(C)C(=O)O)c2)nc1N] | ¹H-NMR (500 MHz, CD₃OD) δ: 7.99-7.26 (m, 11H), 7.08-6.05 (m, 3H), 5.12-4.88 (m, 2H), 4.35-4.26 (m, 2H), 2.46 (s, 3H), 1.65, 1.61 (2 s, 6H); MS: 602.0 (M + 1)⁺. |
| 27/92 | 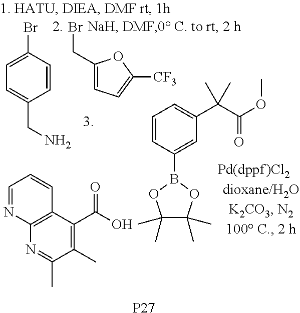 | 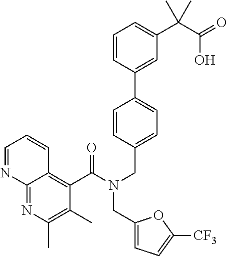 [SMILES: Cc1nc2ncccc2c(C(=O)N(Cc2ccc(-c3cccc(C(C)(C)C(=O)O)c3)cc2)Cc2ccc(C(F)(F)F)o2)c1C] | ¹H-NMR (400 MHz, CD₃OD) δ: 9.01 (dd, J = 1.6, 3.6 Hz, 0.5H), 8.96 (dd, J = 1.4, 3.3 Hz, 0.5H), 8.17-8.12 (m, 1H), 7.66 (d, J = 6.4 Hz, 1H), 7.60-7.34 (m, 7H), 7.04 (dd, J = 1.2, 2.8 Hz, 0.5H), 6.90 (d, J = 6.4 Hz, 1H), 6.76 (dd, J = 0.8, 1.2 Hz, 0.5H), 6.62 (d, J = 2.4 Hz, 0.5H), 6.23 (d, J = 2.4 Hz, 0.5H), 5.17-4.83 (m, 2H), 4.39-4.35 (m, 2H), 2.81, 2.79 (2 s, 3H), 2.48, 2.43 (2s, 3H), 1.64, 1.62 (2 s, 6H); |
| | | | MS: 602.2 (M + 1)⁺. |
| 27/93 | 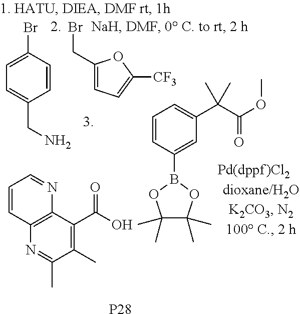 | 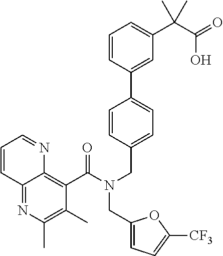 [SMILES: Cc1nc2cccnc2c(C(=O)N(Cc2ccc(-c3cccc(C(C)(C)C(=O)O)c3)cc2)Cc2ccc(C(F)(F)F)o2)c1C] | ¹H-NMR(400 MHz, CD₃OD) δ: 8.99 (d, J = 4.8 Hz, 0.5H), 8.96 (d, J = 3.6 Hz, 0.5H), 8.39 (dd, J = 1.2, 6.8 Hz, 1H), 8.37-7.39 (m, 8H), 7.06 (d, J = 6.4 Hz, 1H), 7.02 (d, J = 3.6 Hz, 0.5H), 6.78 (dd, J = 0.8, 1.2 Hz, 0.5H), 6.72 (d, J = 2.4 Hz, 0.5H), 6.13 (d, J = 2.4 Hz, 0.5H), 5.34 (d, J = 12.4 Hz, 0.5H), 5.14 (d, J = 12.0 Hz, 0.5H), 4.92 (d, J = 13.6 Hz, 0.5H), 4.66 (d, J = 12.8 Hz, 0.5H), 4.43-4.28 (m, 2H), 2.78, 2.72 (2 s, 3H), 2.49, |
| | | | 2.38 (2s, 3H), 1.64, 1.61 |
| | | | (2 s, 6H); |
| | | | MS: 602.2 (M + 1)⁺. |
| 27/94 | 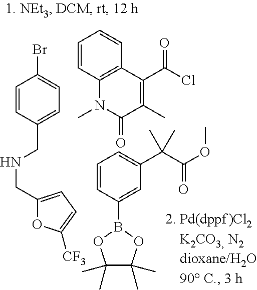 | 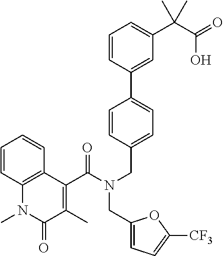 [SMILES: Cc1c(C(=O)N(Cc2ccc(-c3cccc(C(C)(C)C(=O)O)c3)cc2)Cc2ccc(C(F)(F)F)o2)c2ccccc2n(C)c1=O] | ¹H-NMR(500 MHz, CD₃OD) δ: 7.67- 7.40 (m, 10H), 7.31 (dd, J = 6.5, 7.5 Hz, 1H), 7.10 (d, J = 8.5 Hz, 1H), 7.00 (d, J = 2.0 Hz, 0.5H), 6.78 (d, J = 2.5 Hz, 0.5H), 6.54 (d, J = 3.5 Hz, 0.5H), 6.29 (d, J = 3.0 Hz, 0.5H), 5.04-4.84 (m, 2H), 4.50- 4.39 (m, 2H), 3.82 (2 s, 3H), 2.21, 2.18 (2 s, 3H), 1.64, 1.61 (2 s, 6H); MS: 617.3 (M + 1)⁺. |
| 27/95 | 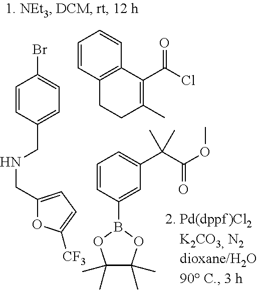 | 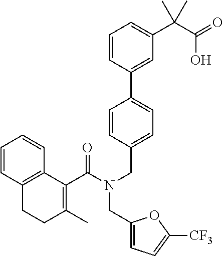 [SMILES: CC1=C(C(=O)N(Cc2ccc(-c3cccc(C(C)(C)C(=O)O)c3)cc2)Cc2ccc(C(F)(F)F)o2)c2ccccc2CC1] | ¹H-NMR (500 MHz, CD₃OD) δ: 7.62-7.40 (m, 7H), 7.20-7.13 (m, 4H), 7.03-6.94 (m, 1.5H), 6.80 (d, J = 2.5 Hz, 0.5H), 6.47 (d, J = 3.5 Hz, 0.5H), 6.26 (d, J = 3.5 Hz, 0.5H), 4.95-4.71 (m, 2H), 4.51- 4.50 (m, 2H), 2.89-2.83 (m, 2H), 2.42-2.29 (m, 2H), 1.94, 1.93 (2 s, 3H), 1.63, 1.62 (2 s, 6H); MS: 588.3 (M + 1)⁺. |
| 27/96 | 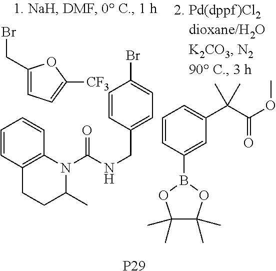 | 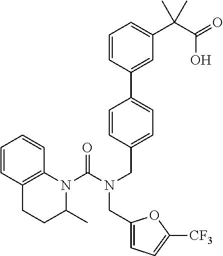 [SMILES: CC1CCc2ccccc2N1C(=O)N(Cc1ccc(-c2cccc(C(C)(C)C(=O)O)c2)cc1)Cc1ccc(C(F)(F)F)o1] | ¹H-NMR (500 MHz, CD₃OD) δ: 7.60-7.54 (m, 3H), 7.49-6.93 (m, 10H), 6.40 (d, J = 3.0 Hz, 1H), 4.70 (d, J = 16.5 Hz, 1H), 4.39 (d, J = 15.5 Hz, 1H), 4.28 (d, J = 16.5 Hz, 1H), 4.25-4.20 (m, 1H), 4.13 (d, J = 15.0 Hz, 1H), 2.73-2.68 (m, 1H), 2.60- 2.55 (m, 1H), 1.81-1.72 (m, 1H), 1.69-1.61 (m, 7H), 1.20, 1.18 (2 s, 3H); MS: 591.3 (M + 1)⁺. |
| 27/97 | 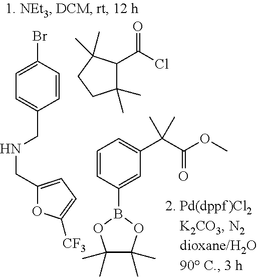 | 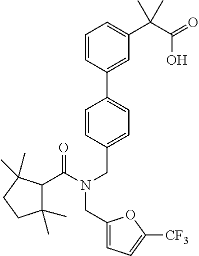 [SMILES: CC(C)(C(=O)O)c1cccc(-c2ccc(CN(Cc3ccc(C(F)(F)F)o3)C(=O)C3C(C)(C)CCC3(C)C)cc2)c1] | ¹H-NMR (500 MHz, CD₃OD) δ: 7.63-7.31 (m, 8H), 6.91 (s, 1H), 6.46 (d, J = 3.0 Hz, 0 0.5H), 6.43 (d, J = 3.5 Hz, 0.5H), 4.80-4.70 (m, 4H), 2.97, 2.77 (2 8, 1H), 1.81-1.51 (m, 10H), 1.19, 1.15 (2 s, 6H), 1.09, 1.03 (2 s, 6H); MS: 570.2 (M + 1)⁺. |
| 27/98 | 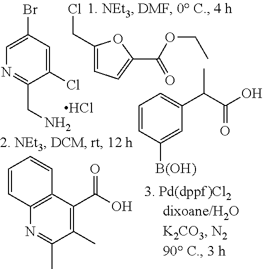 | 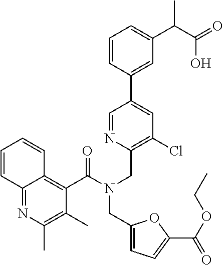 [SMILES: CCOC(=O)c1ccc(CN(Cc2ncc(-c3cccc(C(C)C(=O)O)c3)cc2Cl)C(=O)c2c(C)c(C)nc3ccccc23)o1] | ¹H-NMR (500 MHz, CD₃OD) δ: 9.08-6.17 (m, 12H), 5.47-5.05 (m, 2H), 4.71-4.51 (m, 2H), 4.43-4.22 (m, 2H), 3.92-3.77 (m, 1H), 3.11-2.50 (m, 6H), 1.59-1.48 (m, 3H), 1.40-1.29 (m, 3H); MS: 626.2 (M + 1)⁺. |
| 27/99 | 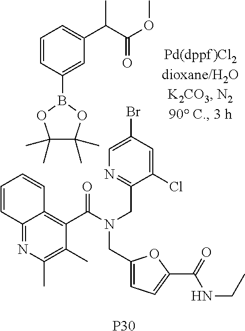 | 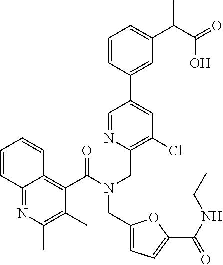 [SMILES: CCNC(=O)c1ccc(CN(Cc2ncc(-c3cccc(C(C)C(=O)O)c3)cc2Cl)C(=O)c2c(C)c(C)nc3ccccc23)o1] | ¹H-NMR (500 MHz, CD₃OD) δ: 8.85 (d, J = 2.0 Hz, 0.5H), 8.66 (d, J = 2.0 Hz, 0.5H), 8.31 (d, J = 8.0 Hz, 0.5H), 8.14 (d, J = 2.0 Hz, 0.5H), 8.04 (d, J = 8.5 Hz, 0.5H), 7.90 (d, J = 8.5 Hz, 0.5H), 7.78- 7.34 (m, 7H), 7.130 (d, J = 3.5 Hz, 0.5H), 6.84 (d, J = 3.0 Hz, 0.5H), 6.67 (d, J = 3.5 Hz, 0.5H), 6.04 (d, J = 3.5 Hz, 0.5H), 5.38-5.21 (m, 2H), 4.69-4.52 (m, 2H), 3.86-3.79 (m, 1H), 3.47-3.34 (m, 2H), 2.78, 2.68 (2 s, 3H), 2.58, 2.32 (2 s, 3H), 1.56- |
| | | | 1.52 (m, 3H), 1.25-1.17 |
| | | | (m, 3H); |
| | | | MS: 625.3 (M + 1)⁺. |
| 27/ 100 | 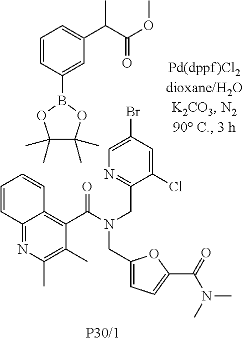 | 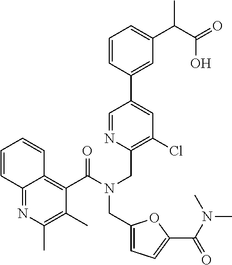 [SMILES: Cc1nc2ccccc2c(C(=O)N(Cc2ccc(C(=O)N(C)C)o2)Cc2ncc(-c3cccc(C(C)C(=O)O)c3)cc2Cl)c1C] | ¹H-NMR (500 MHz, DMSO-d₆) δ: 8.93 (d, J = 2.0 Hz, 0.5H), 8.78 (d, J = 2.0 Hz, 0.5H), 8.29 (d, J = 1.5 Hz, 0.5H), 8.22 (d, J = 8.0 Hz, 0.5H), 7.96 (d, J = 8.0 Hz, 0.5H), 7.93 (d, J = 2.0 Hz, 0.5H), 7.86 (d, J = 8.0 Hz, 0.5H), 7.74- 7.35 (m, 6.5H), 7.00 (d, J = 3.5 Hz, 0.5H), 6.79 (d, J = 3.5 Hz, 0.5H), 6.63 (d, J = 3.0 Hz, 0.5H), 6.24 (d, J = 3.0 Hz, 0.5H), 5.19-4.96 (m, 2H), 4.52-4.37 (m, 2H), 3.81-3.76 (m, 1H), 3.23-2.95 (m, 6H), |
| | | | 2.68, 2.57 (2 s, 3H), |
| | | | 2.43, 2.20 (2 s, 3H), |
| | | | 1.46-1.42 (m, 3H); |
| | | | MS: 625.3 (M + 1)⁺. |
| 27/ 101 | 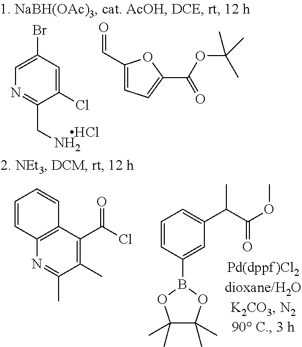 | 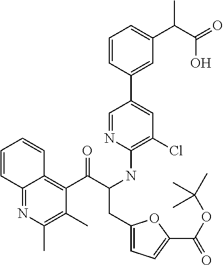 [SMILES: Cc1nc2ccccc2c(C(=O)C(Cc2ccc(C(=O)OC(C)(C)C)o2)Nc2ncc(-c3cccc(C(C)C(=O)O)c3)cc2Cl)c1C] | ¹H-NMR (500 MHz, CD₃OD) δ: 8.96-7.42 (m, 10H), 7.12-6.27 (m, 2H), 5.38-5.10 (m, 2H), 4.64-4.55 (m, 2H), 3.90-3.84 (m, 1H), 3.03-2.57 (m, 6H), 1.61-1.50 (m, 12H); MS: 654.1 (M + 1)⁺. |
| 27/ 102 | 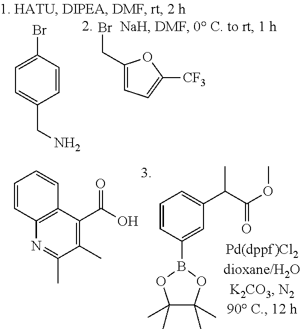 | 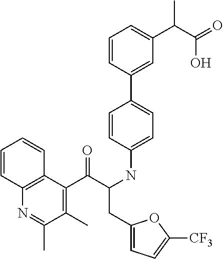 [SMILES: Cc1nc2ccccc2c(C(=O)C(Cc2ccc(C(F)(F)F)o2)Nc2ccc(-c3cccc(C(C)C(=O)O)c3)cc2)c1C] | ¹H-NMR (400 MHz, CD₃OD) δ: 8.01 (d, J = 8.4 Hz, 1H), 7.76-7.30 (m, 10H), 7.02-7.01 (m, 0.5H), 6.96 (d, J = 8.0 Hz, 1H), 6.76 (d, J = 3.2 Hz, 0.5H), 6.57 (d, J = 3.2 Hz, 0.5H), 6.16 (d, J = 3.6 Hz, 0.5H), 5.08-4.93 (m, 2H), 4.37-4.27 (m, 2H), 3.83-3.74 (m, 1H), 2.74, 2.70 (2 s, 3H), 2.43, 2.36 (2 s, 3H), 1.55-1.50 (m, 3H); MS: 587.2 (M + 1)⁺. |
| 27/ 103 | 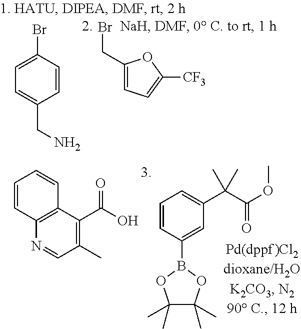 | 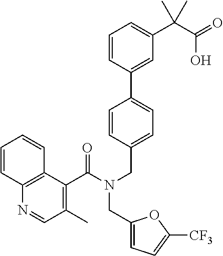 [SMILES: Cc1cnc2ccccc2c1C(=O)N(Cc1ccc(-c2cccc(C(C)(C)C(=O)O)c2)cc1)Cc1ccc(C(F)(F)F)o1] | ¹H-NMR (400 MHz, CD₃OD) δ: 8.83 (d, J = 8.8 Hz, 1H), 8.08 (d, J = 8.4 Hz, 1H), 7.80-7.76 (m, 2H), 7.65-7.39 (m, 8H), 7.01-6.99 (m, 1.5H), 6.75 (s, 0.5H), 6.56 (s, 0.5H), 6.17 (s, 0.5H), 5.11-4.89 (m, 2H), 4.37-4.30 (m, 2H), 2.51, 2.46 (2 s, 3H), 1.64, 1.61 (2 s, 6H); MS: 587.3 (M + 1)⁺. |
| 27/ 104 | 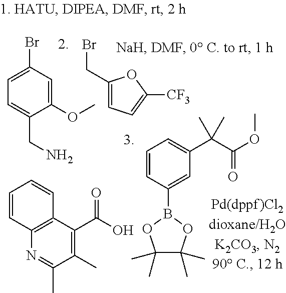 | 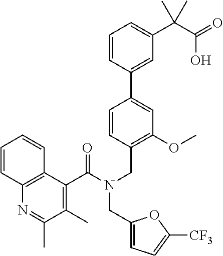 [SMILES: COc1cc(-c2cccc(C(C)(C)C(=O)O)c2)ccc1CN(Cc1ccc(C(F)(F)F)o1)C(=O)c1c(C)c(C)nc2ccccc12] | ¹H-NMR (400 MHz, DMSO-d₆) δ: 7.94 (d, J = 8.0 Hz, 1H), 7.70- 7.22 (m, 9H), 6.97-6.81 (m, 2H), 6.61-6.22 (m, 1H), 5.01-4.83 (m, 2H), 4.33-4.20 (m, 2H), 3.96, 3.58 (2 s, 3H), 2.64, 2.61 (2 s, 3H), 2.30, 2.19 (2 s, 3H), 1.54, 1.51 (2 s, 6H); MS: 631.3 (M + 1)⁺. |
| 27/ 105 | 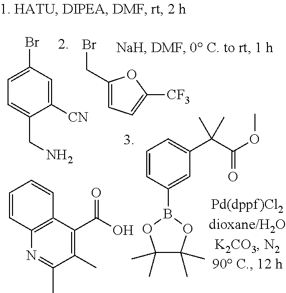 | 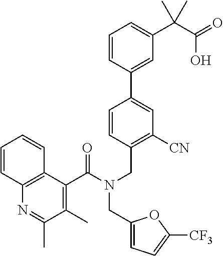 [SMILES: Cc1nc2ccccc2c(C(=O)N(Cc2ccc(C(F)(F)F)o2)Cc2ccc(-c3cccc(C(C)(C)C(=O)O)c3)cc2C#N)c1C] | ¹H-NMR (400 MHz, DMSO-d₆) δ: 12.39 (br s, 1H), 8.12-7.38 (m, 11H), 7.26-6.91 (m, 2H), 6.74 (d, J = 2.8 Hz, 0.5H), 6.27 (d, J = 3.2 Hz, 0.5H), 5.22-5.03 (m, 2H), 4.58-4.39 (m, 2H), 2.67, 2.59 (2 s, 3H), 2.37, 2.25 (2 s, 3H), 1.54, 1.52 (2 s, 6H); MS: 626.3 (M + 1)⁺. |
| 27 /106 | 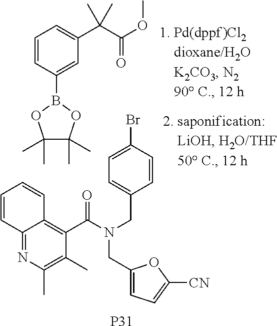 | 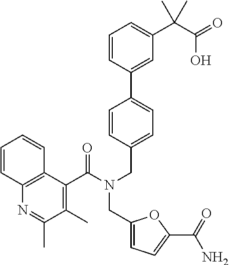 [SMILES: Cc1nc2ccccc2c(C(=O)N(Cc2ccc(-c3cccc(C(C)(C)C(=O)O)c3)cc2)Cc2ccc(C(N)=O)o2)c1C] | |
| 27/ 107 | 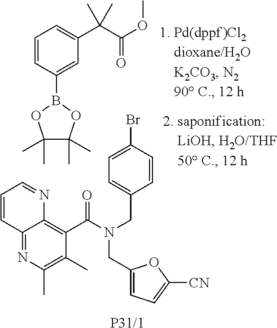 | 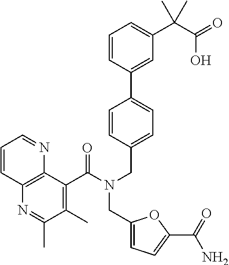 [SMILES: Cc1nc2cccnc2c(C(=O)N(Cc2ccc(-c3cccc(C(C)(C)C(=O)O)c3)cc2)Cc2ccc(C(N)=O)o2)c1C] | |
| 27/ 108 | 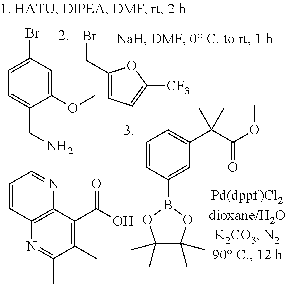 | 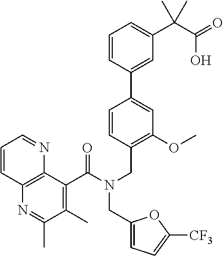 [SMILES: COc1cc(-c2cccc(C(C)(C)C(=O)O)c2)ccc1CN(Cc1ccc(C(F)(F)F)o1)C(=O)c1c(C)c(C)nc2cccnc12] | ¹H-NMR (400 MHz, CD₃OD) δ: 8.97, 8.87 (2 d, J = 4.4 Hz, 1H), 8.38, 8.34 (2 d, J = 8.8 Hz, 1H), 7.84-6.05 (m, 10H), 5.27-4.90 (m, 2H), 4.45-4.28 (m, 2H), 3.98, 3.67 (2 s, 3H), 2.77, 2.69 (2 s, 3H), 2.46, 2.27 (2 s, 3H), 1.65, 1.62 (2 s, 6H); MS: 632.4 (M + 1)⁺. |
| 27/ 109 | 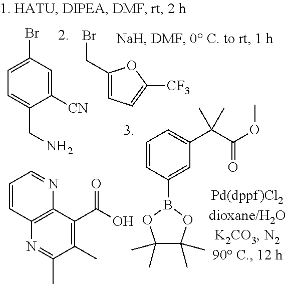 | 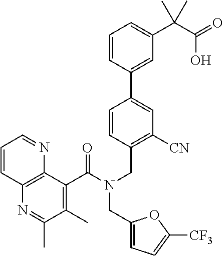 [SMILES: Cc1nc2cccnc2c(C(=O)N(Cc2ccc(C(F)(F)F)o2)Cc2ccc(-c3cccc(C(C)(C)C(=O)O)c3)cc2C#N)c1C] | ¹H-NMR (400 MHz, DMSO-d₆) δ: 9.07 -6.29 (m, 12H), 5.36- 5.24 (m, 1H), 4.86-4.76 (m, 1H), 4.59-4.38 (m, 2H), 2.71, 2.59 (2 s, 3H), 2.39, 2.26 (2 s, 3H), 1.56, 1.53 (2 s, 6H); MS: 627.3 (M + 1)⁺. |
| 27/ 110 | 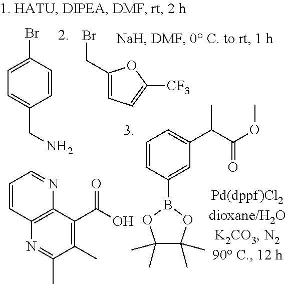 | 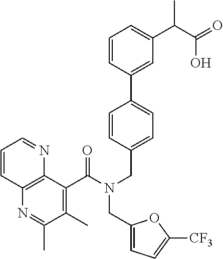 [SMILES: Cc1nc2cccnc2c(C(=O)N(Cc2ccc(-c3cccc(C(C)C(=O)O)c3)cc2)Cc2ccc(C(F)(F)F)o2)c1C] | ¹H-NMR (400 MHz, CD₃OD) δ: 8.99-8.95 (m, 1H), 8.41-8.33 (m, 1H), 7.75-7.31 (m, 8H), 7.06 (d, J = 8.0 Hz, 1H), 7.01-6.78 (m, 1H), 6.71-6.14 (m, 1H), 5.35- 5.13 (m, 1H), 4.92-4.63 (m, 1H), 4.43-4.25 (m, 2H), 3.85-3.77 (m, 1H), 2.78, 2.72 (2 s, 3H), 2.48, 2.38 (2 s, 3H), 1.55-1.50 (m, 3H); MS: 588.3 (M + 1)⁺. |
| 27/ 111 | 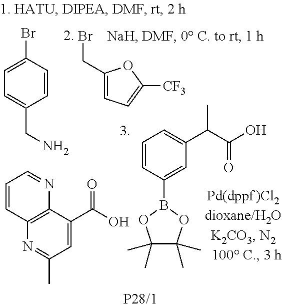 | 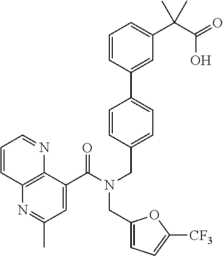 [SMILES: Cc1cc(C(=O)N(Cc2ccc(-c3cccc(C(C)(C)C(=O)O)c3)cc2)Cc2ccc(C(F)(F)F)o2)c2ncccc2n1] | ¹H NMR (400 MHz, DMSO-d₆) δ: 9.03-9.02 (m, 1H), 8.42-8.39 (m, 1H), 7.84-7.81 (m, 1H), 7.67-7.59 (m, 4H), 7.51-6.99 (m, 6H), 6.81-6.31 (m, 1H), 5.01-4.76 (m, 2H), 4.38-4.25 (m, 2H), 2.73, 2.67 (2 s, 3H), 1.54, 1.50 (2 s, 6H); MS: 588.3 (M + 1)⁺. |
| 27/ 112 | 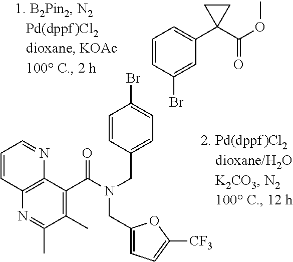 | 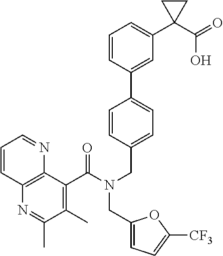 [SMILES: Cc1nc2cccnc2c(C(=O)N(Cc2ccc(-c3cccc(C4(C(=O)O)CC4)c3)cc2)Cc2ccc(C(F)(F)F)o2)c1C] | ¹H-NMR (400 MHz, DMSO-d₆) δ: 9.00, 8.96 (2 d, J = 4.4 Hz, 1H), 8.39-8.34 (m, 1H), 7.77- 6.24 (m, 11H), 5.20-4.13 (m, 4H), 2.69, 2.65 (2 s, 3H), 2.34, 2.29 (2 s, 3H), 1.48-1.43 (m,2H), 1.21- 1.12 (m, 2H); MS: 600.2 (M + 1)⁺. |
| 27/ 113 | 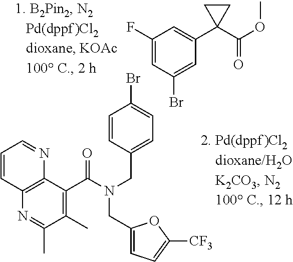 | 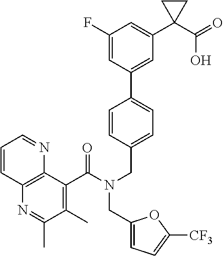 [SMILES: Cc1nc2cccnc2c(C(=O)N(Cc2ccc(-c3cc(F)cc(C4(C(=O)O)CC4)c3)cc2)Cc2ccc(C(F)(F)F)o2)c1C] | ¹H-NMR (400 MHz, DMSO-d₆) δ: 9.01-8.94 (m, 1H), 8.39-8.34 (m, 1H), 7.78-6.25 (m, 10H), 5.20-4.14 (m, 4H), 2.69, 2.65 (2 s, 3H), 2.34, 2.29 (2 s, 3H), 1.50-1.45 (m, 2H), 1.27-1.21 (m, 2H); MS: 618.2 (M + 1)⁺. |
| 27/ 114 | 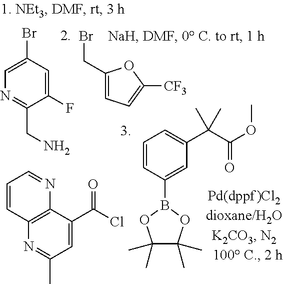 | 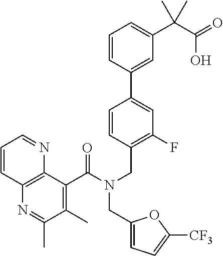 [SMILES: Cc1nc2cccnc2c(C(=O)N(Cc2ccc(C(F)(F)F)o2)Cc2ccc(-c3cccc(C(C)(C)C(=O)O)c3)cc2F)c1C] | ¹H-NMR (400 MHz, CD₃OD) δ: 8.98 (d, J = 4.4 Hz, 0.5H), 8.97 (d, J = 5.6 Hz, 0.5H), 8.39 (d, J = 8.8 Hz, 0.5H), 8.34 (d, J = 8.8 Hz, 0.5H), 7.98- 6.12 (m, 10H), 5.32- 4.30 (m, 4H), 2.78, 2.72 (2 s, 3H), 2.48, 2.36 (2 s, 3H), 1.64, 1.62 (2 s, 6H); MS: 620.2 (M + 1)⁺. |
| 27/ 115 | 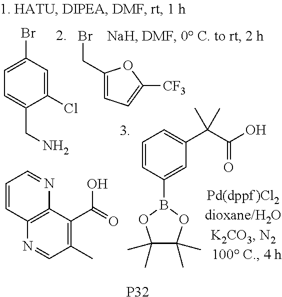 | 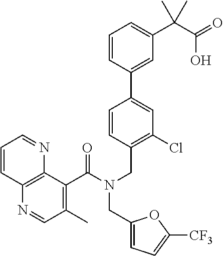 [SMILES: Cc1cnc2cccnc2c1C(=O)N(Cc1ccc(C(F)(F)F)o1)Cc1ccc(-c2cccc(C(C)(C)C(=O)O)c2)cc1Cl] | ¹H-NMR (400 MHz, CD₃OD) δ: 9.13-6.14 (m, 13H), 5.31-4.37 (m, 4H), 2.61, 2.49 (2 s, 3H), 1.64, 1.61 (2 s, 6H); MS: 622.2 (M + 1)⁺. |
| 27/ 116 | 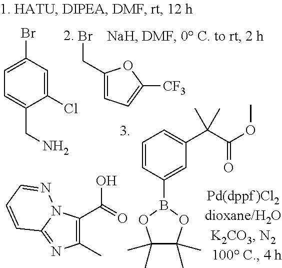 | 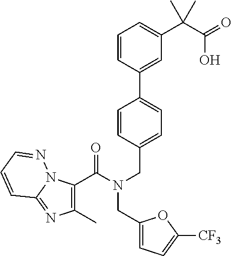 [SMILES: Cc1nc2cccnn2c1C(=O)N(Cc1ccc(-c2cccc(C(C)(C)C(=O)O)c2)cc1)Cc1ccc(C(F)(F)F)o1] | ¹H-NMR (400 MHz, DMSO-d₆) δ: 8.58 (d, J = 14.0 Hz, 1H), 8.14-8.08 (m, 1H), 7.63-6.98 (m, 9H), 6.65 (s, 1H), 6.28 (s, 1H), 4.84 (s, 2H), 4.49 (s, 2H), 2.43, 2.38 (2 s, 3H), 1.55, 1.52 (2 s, 6H); MS: 577.3 (M + 1)⁺. |
| 27/ 117 | 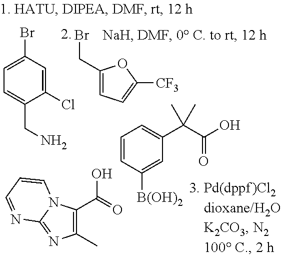 | 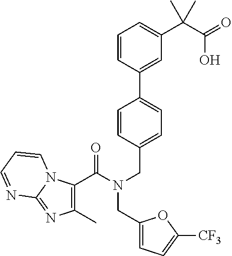 [SMILES: Cc1nc2ncccn2c1C(=O)N(Cc1ccc(-c2cccc(C(C)(C)C(=O)O)c2)cc1)Cc1ccc(C(F)(F)F)o1] | ¹H-NMR (400 MHz, CD₃OD) δ: 8.90-8.78 (m, 2H), 7.60-7.26 (m, 9H), 6.93 (s, 0.5H), 6.81 (s, 0.5H), 6.55 (s, 0.5H), 6.38 (s, 0.5H), 4.97 (s, 2H), 4.84 (s, 2H), 2.69, 2.63 (2 s, 3H), 1.62 (s, 6H); MS: 577.3 (M + 1)⁺. |
| 27/ 118 | 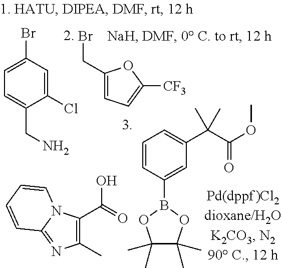 | 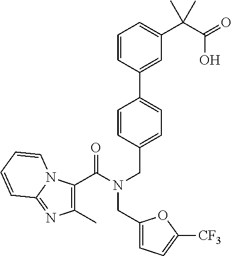 [SMILES: Cc1nc2ccccn2c1C(=O)N(Cc1ccc(-c2cccc(C(C)(C)C(=O)O)c2)cc1)Cc1ccc(C(F)(F)F)o1] | ¹H-NMR (400 MHz, CH₃OD) δ: 8.37 (d, J = 6.8 Hz, 1H), 7.58-7.39 (m, 8H), 7.24 (br s, 2H), 7.04 (t, J = 6.8 Hz, 1H), 6.90 (s, 1H), 6.46 (s, 1H), 4.80 (s, 2H), 4.76 (s, 2H), 2.53 (s, 3H), 1.62 (s, 6H); MS: 576.1 (M + 1)⁺. |
| 27/ 119 | 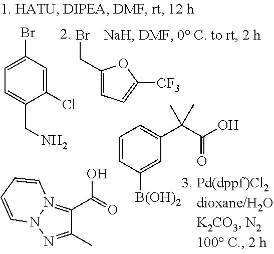 | 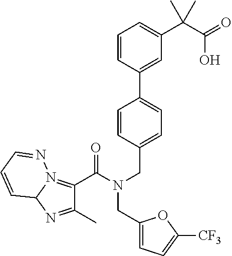 | ¹H-NMR (400 MHz, CD₃OD) δ: 8.89 (s, 1H), 8.67 (s, 1H), 7.62-6.17 (m, 11H), 4.86-4.75 (m, 4H), 2.56, 2.52 (2 s, 3H), 1.62 (s, 6H); MS: 577.3 (M + 1)⁺. |
| 27/ 120 | 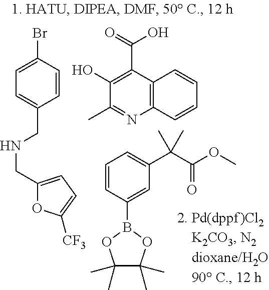 | 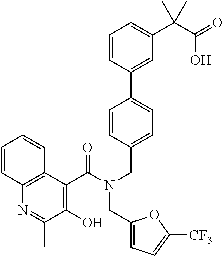 [SMILES: Cc1nc2ccccc2c(C(=O)N(Cc2ccc(-c3cccc(C(C)(C)C(=O)O)c3)cc2)Cc2ccc(C(F)(F)F)o2)c1O] | ¹H-NMR (500 MHz, CD₃OD) δ: 8.07-8.02 (m, 1H), 7.84-7.39 (m, 10H), 7.11 (d, J = 8.5 Hz, 1H), 7.01 (d, J = 2.0 Hz, 0.5H), 6.71 (d, J = 2.0 Hz, 0.5H), 6.59 (d, J = 3.5 Hz, 0.5H), 6.22 (d, J = 3.0 Hz, 0.5H), 5.39- 4.91 (m, 2H), 4.62-4.41 (m, 2H), 2.91, 2.87 (2 s, 3H), 1.64, 1.61 (2 s, 6H); MS: 603.1 (M + 1)⁺. |
| 27/ 121 | 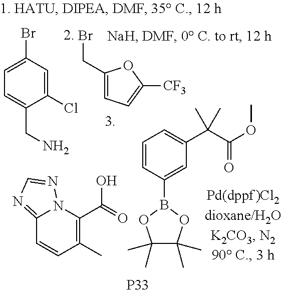 | 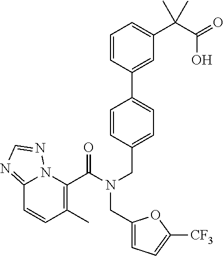 [SMILES: Cc1ccc2ncnn2c1C(=O)N(Cc1ccc(-c2cccc(C(C)(C)C(=O)O)c2)cc1)Cc1ccc(C(F)(F)F)o1] | ¹H-NMR (400 MHz, CD₃OD) δ: 8.47 (d, J = 10.4 Hz, 1H), 7.84- 7.39 (m, 9H), 7.13 (d, J = 8.0 Hz, 1H), 6.99 (d, J = 3.2 Hz, 0.5H), 6.79 (d, J = 3.6 Hz, 0.5H), 6.69 (d, J = 3.2 Hz, 0.5H), 6.19 (d, J = 3.6 Hz, 0.5H), 5.21-5.12 (m, 1H), 4.79-4.74 (m, 1H), 4.53-4.28 (m, 2H), 2.45, 2.36 (2 s, 3H), 1.64, 1.61 (2 s, 6H); MS: 577.3 (M + 1)⁺. |
| 27/ 122 | 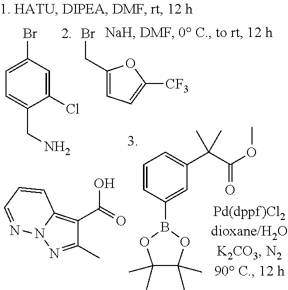 | 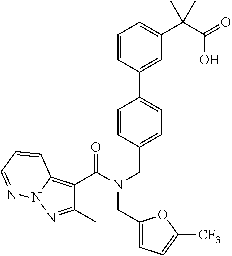 [SMILES: Cc1nn2ncccc2c1C(=O)N(Cc1ccc(-c2cccc(C(C)(C)C(=O)O)c2)cc1)Cc1ccc(C(F)(F)F)o1] | ¹H-NMR (400 MHz, CD₃OD) δ: 8.42-8.40 (m, 1H), 8.06-8.04 (m, 1H), 7.58-7.28 (m, 9H), 6.89 (s, 1H), 6.43 (s, 1H), 4.75 (s, 4H), 2.57 (s, 3H), 1.62 (s, 6H); MS: 577.3 (M + 1)⁺. |
| 27/ 123 | 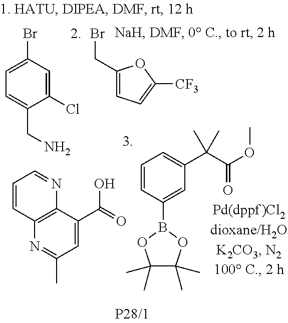 | 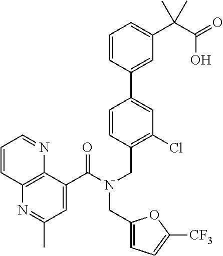 [SMILES: Cc1cc(C(=O)N(Cc2ccc(C(F)(F)F)o2)Cc2ccc(-c3cccc(C(C)(C)C(=O)O)c3)cc2Cl)c2ncccc2n1] | ¹H-NMR (400 MHz, CD₃OD) δ: 9.09-8.97 (m, 1H), 8.45-8.35 (m, 1H), 8.00-7.31 (m, 9H), 6.99 (d, J = 3.0 Hz, 0.5H), 6.81 (d, J = 4.0 Hz, 0.5H), 6.76 (d, J = 3.0 Hz, 0.5H), 6.21 (d, J = 3.5 Hz, 0.5H), 5.21-4.97 (m, 2H), 4.64-4.42 (m, 2H), 2.84, 2.70 (2 s, 3H), 1.64, 1.61 (2 s, 6H); MS: 622.2 (M + 1)⁺. |
| 27/ 124 | 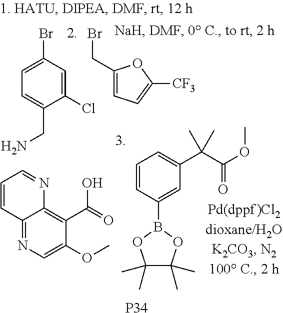 | 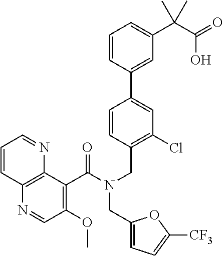 [SMILES: COc1cnc2cccnc2c1C(=O)N(Cc1ccc(C(F)(F)F)o1)Cc1ccc(-c2cccc(C(C)(C)C(=O)O)c2)cc1Cl] | ¹H-NMR (500 MHz, CD₃OD) δ: 9.16-8.93 (m, 2H), 8.50-8.37 (m, 1H), 7.96-7.00 (m, 8H), 7.00 (d, J = 2.0 Hz, 0.5H), 6.79 (d, J = 3.5 Hz, 0.5H), 6.71 (d, J = 3.0 Hz, 0.5H), 6.18 (d, J = 3.5 Hz, 0.5H), 5.21-4.89 (m, 2H), 4.61, 4.45 (2 s, 2H), 4.27, 4.12 (2 s, 3H), 1.65, 1.62 (2 s, 6H); MS: 638.0 (M + 1)⁺. |
| 27/ 125 | 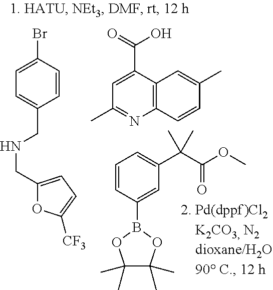 | 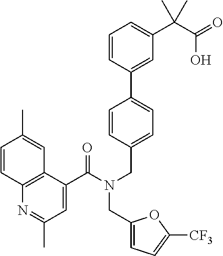 [SMILES: Cc1ccc2nc(C)cc(C(=O)N(Cc3ccc(-c4cccc(C(C)(C)C(=O)O)c4)cc3)Cc3ccc(C(F)(F)F)o3)c2c1] | ¹H-NMR (500 MHz, CD₃OD) δ: 7.94, 7.91 (2 d, J = 9.0 Hz, 1H), 7.67-7.40 (m, 10H), 7.07-7.04 (m, 1.5H), 6.79 (d, J = 2.5 Hz, 0.5H), 6.63 (d, J = 3.5 Hz, 0.5H), 6.26 (d, J = 3.0 Hz, 0.5H), 5.35-4.66 (m, 2H), 4.50-4.32 (m, 2H), 2.76, 2.70 (2 s, 3H), 2.50, 2.48 (2 s, 3H), 1.64, 1.62 (2 s, 6H); MS: 601.3 (M + 1)⁺. |
| 27/ 126 | 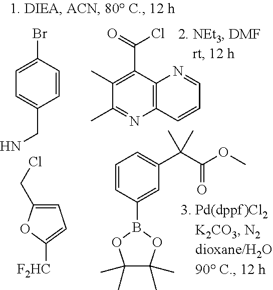 | 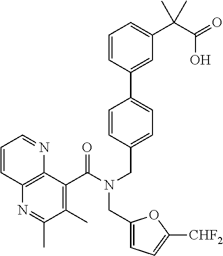 [SMILES: Cc1nc2cccnc2c(C(=O)N(Cc2ccc(-c3cccc(C(C)(C)C(=O)O)c3)cc2)Cc2ccc(C(F)F)o2)c1C] | ¹H-NMR (400 MHz, CD₃OD) δ: 9.08, 9.03 (2 d, J = 3.8 Hz, 1H), 8.44, 8.40 (2 d, J = 8.6 Hz, 1H), 7.86-6.09 (m, 12H), 5.39-4.26 (m, 4H), 2.86, 2.78 (2 s, 3H), 2.53, 2.42 (2 s, 3H), 1.65, 1.61 (2 s, 6H); MS: 582.1 (M − 1)⁻. |
| 27/ 127 | 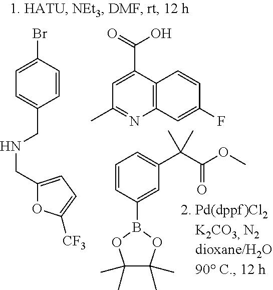 | 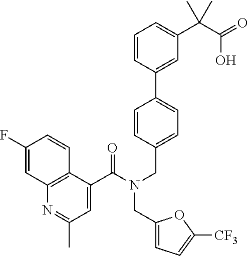 [SMILES: Cc1cc(C(=O)N(Cc2ccc(-c3cccc(C(C)(C)C(=O)O)c3)cc2)Cc2ccc(C(F)(F)F)o2)c2ccc(F)cc2n1] | ¹H-NMR (500 MHz, CD₃OD) δ: 7.92-7.86 (m, 1H), 7.90-7.40 (m, 10H), 7.09 (d, J = 8.5 Hz, 1H), 7.02 (d, J = 2.0 Hz, 0.5H), 6.78 (d, J = 2.5 Hz, 0.5H), 6.60 (d, J = 3.5 Hz, 0.5H), 6.26 (d, J = 3.5 Hz, 0.5H), 5.28-4.68 (m, 2H), 4.49-4.31 (m, 2H), 2.78, 2.71 (2 s, 3H), 1.64, 1.62 (2 s, 6H); MS: 605.3 (M + 1)⁺. |
| 27/ 128 | 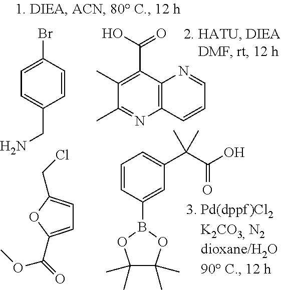 | 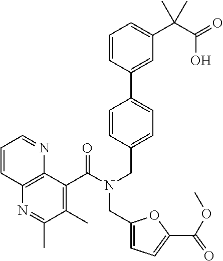 [SMILES: COC(=O)c1ccc(CN(Cc2ccc(-c3cccc(C(C)(C)C(=O)O)c3)cc2)C(=O)c2c(C)c(C)nc3cccnc23)o1] | |
| 27/ 129 | 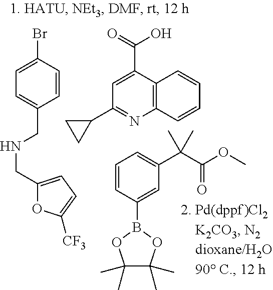 | 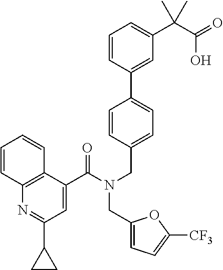 [SMILES: CC(C)(C(=O)O)c1cccc(-c2ccc(CN(Cc3ccc(C(F)(F)F)o3)C(=O)c3cc(C4CC4)nc4ccccc34)cc2)c1] | ¹H-NMR (500 MHz, CD₃OD) δ: 8.01-7.98 (m, 1H), 7.81-7.40 (m, 10H), 7.36, 7.19(2 s, 1H), 7.11 (d, J = 8.5 Hz, 1H), 7.02 (dd, J = 1.0, 3.5 Hz, 0.5H), 6.78 (dd, J = 1.2, 3.3 Hz, 0.5H), 6.61 (d, J = 3.5 Hz, 0.5H), 6.25 (d, J = 2.5 Hz, 0.5H), 5.40-4.34 (m, 4H), 2.36-2.21 (m, 1H), 1.64, 1.62 (2 s, 6H), 1.19-0.91 (m, 4H); MS: 613.1 (M + 1)⁺. |
| 27/ 130 | 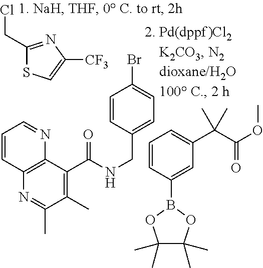 | 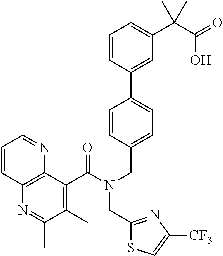 [SMILES: Cc1nc2cccnc2c(C(=O)N(Cc2ccc(-c3cccc(C(C)(C)C(=O)O)c3)cc2)Cc2nc(C(F)(F)F)cs2)c1C] | ¹H-NMR (400 MHz, CD₃OD) δ: 8.85-8.83 (m, 1H), 8.27-7.22 (m, 10H), 7.00 (d, J = 8.4 Hz, 1H), 5.40-4.35 (m, 4H), 2.64, 2.63 (2 s, 3H), 2.35, 2.30 (2 s, 3H), 1.48, 1.44 (2 s, 6H); MS: 619.2 (M + 1)⁺. |
| 27/ 131 | 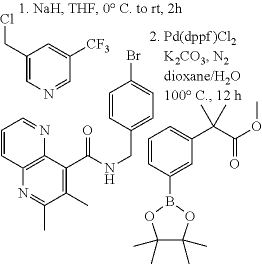 | 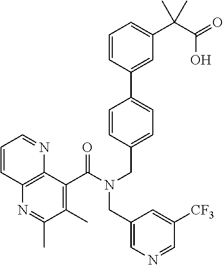 [SMILES: Cc1nc2cccnc2c(C(=O)N(Cc2ccc(-c3cccc(C(C)(C)C(=O)O)c3)cc2)Cc2cncc(C(F)(F)F)c2)c1C] | ¹H-NMR (400 MHz, CD₃OD) δ: 9.05-7.40 (m, 13H), 7.03 (d, J = 8.0 Hz, 1H), 5.64-4.37 (m, 4H), 2.74, 2.74 (2 s, 3H), 2.43, 2.41 (2 s, 3H), 1.64, 1.61 (s, 6H); MS: 613.3 (M + 1)⁺. |
| 27/ 132 | 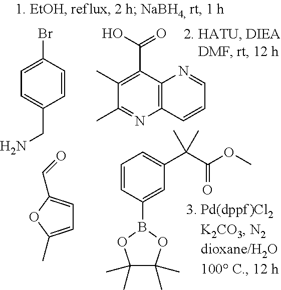 | 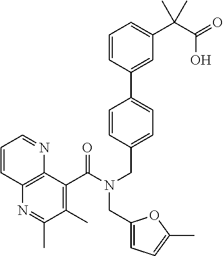 [SMILES: Cc1ccc(CN(Cc2ccc(-c3cccc(C(C)(C)C(=O)O)c3)cc2)C(=O)c2c(C)c(C)nc3cccnc23)o1] | ¹H-NMR (400 MHz, DMSO-d₆) δ: 9.02-8.95 (m, 1H), 8.39-8.32 (m, 1H), 7.78-7.32 (m, 8H), 7.12 (d, J = 8.0 Hz, 1H), 6.36-5.87 (m, 2H), 5.21-4.03 (m, 4H), 2.71, 2.64 (2 s, 3H), 2.35-2.11 (m, 6H), 1.55, 1.51 (2 s, 6H); MS: 548.3 (M + 1)⁺. |
| 27/ 133 | 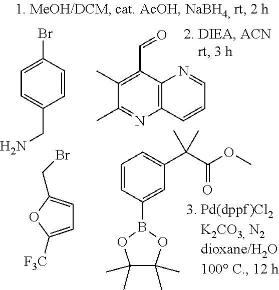 | 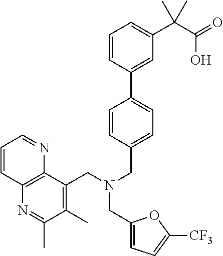 [SMILES: Cc1nc2cccnc2c(CN(Cc2ccc(-c3cccc(C(C)(C)C(=O)O)c3)cc2)Cc2ccc(C(F)(F)F)o2)c1C] | ¹H-NMR (400 MHz, DMSO-d₆) δ: 12.36 (br s, 1H), 8.93 (dd, J = 4.4, 1.6 Hz,1H), 8.23 (dd, J = 8.4, 1.6 Hz, 1H), 7.66 (dd, J = 8.4, 4.4 Hz, 1H), 7.51-7.27 (m, 8H), 7.06 (d, J = 2.0 Hz, 1H), 6.45 (d, J = 3.2 Hz, 1H), 4.47 (s, 2H), 3.71 (s, 2H), 3.61 (s, 2H), 2.63 (s, 3H), 2.47 (s, 3H), 1.52 (s, 6H); MS: 588.3 (M + 1)⁺. |
| 27/ 134 | 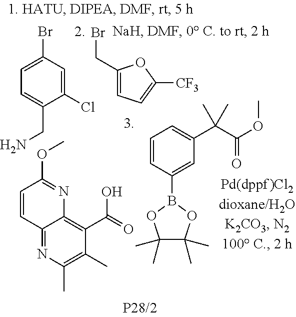 | 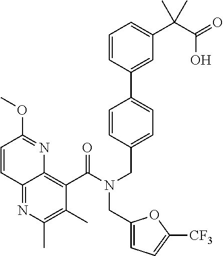 [SMILES: COc1ccc2nc(C)c(C)c(C(=O)N(Cc3ccc(-c4cccc(C(C)(C)C(=O)O)c4)cc3)Cc3ccc(C(F)(F)F)o3)c2n1] | ¹H-NMR (400 MHz, DMSO-d₆) δ: 8.19 (t, J = 9.0 Hz, 1H), 7.61-6.99 (m, 10H), 6.67-6.31 (m, 1H), 5.28-4.29 (m, 4H), 3.82, 3.77 (2 s, 3H), 2.62, 2.58 (2 s, 3H), 2.31, 2.27 (2 s, 3H), 1.54, 1.51 (2 s, 6H); MS: 632.3 (M + 1)⁺. |
| 27/ 135 | 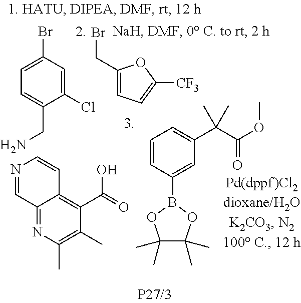 | 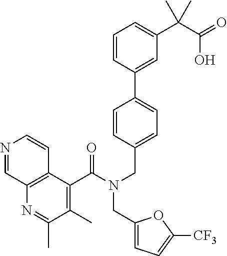 [SMILES: Cc1nc2cnccc2c(C(=O)N(Cc2ccc(-c3cccc(C(C)(C)C(=O)O)c3)cc2)Cc2ccc(C(F)(F)F)o2)c1C] | ¹H-NMR (400 MHz, CD₃OD) δ: 9.29 (d, J = 9.2 Hz, 1H), 8.51, 8.47 (2 d, 5.8 Hz, 1H), 7.67-6.22 (m, 11H), 5.14-4.85 (m, 2H), 4.42-4.32 (m, 2H), 2.81, 2.77 (2 s, 3H), 2.50, 2.43 (2 s, 3H), 1.64, 1.61 (2 s, 6H); MS: 602.2 (M + 1)⁺. |
| 27/ 136 | 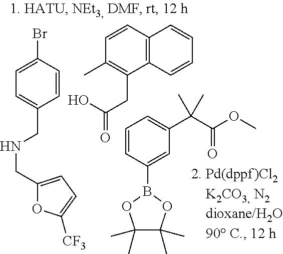 | 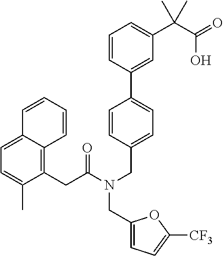 [SMILES: Cc1ccc2ccccc2c1CC(=O)N(Cc1ccc(-c2cccc(C(C)(C)C(=O)O)c2)cc1)Cc1ccc(C(F)(F)F)o1] | |
| 27/ 137 | 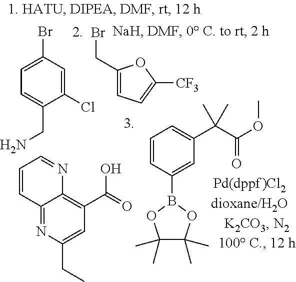 | 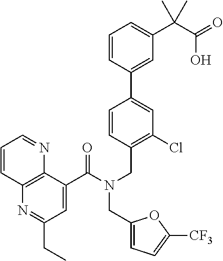 [SMILES: CCc1cc(C(=O)N(Cc2ccc(C(F)(F)F)o2)Cc2ccc(-c3cccc(C(C)(C)C(=O)O)c3)cc2Cl)c2ncccc2n1] | |

### Example 28

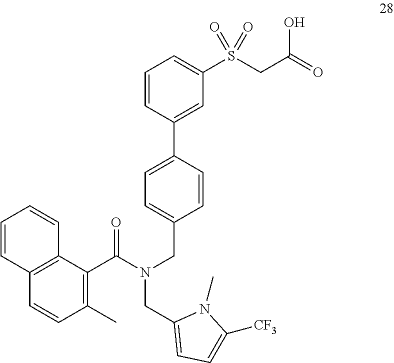 [SMILES: Cc1ccc2ccccc2c1C(=O)N(Cc1ccc(-c2cccc(S(=O)(=O)CC(=O)O)c2)cc1)Cc1ccc(C(F)(F)F)n1C]

### Step 1: N-(4-Bromobenzyl)-2-methyl-N-((1-methyl-5-(trifluoromethyl)-1H-pyrrol-2-yl)methyl)-1-naphthamide (28a)

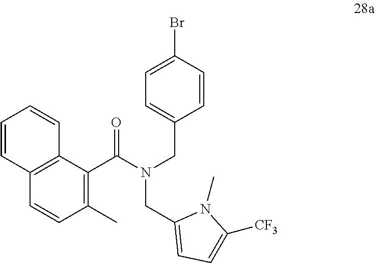 [SMILES: Cc1ccc2ccccc2c1C(=O)N(Cc1ccc(Br)cc1)Cc1ccc(C(F)(F)F)n1C]

To a solution of N-(4-bromobenzyl)-2-methyl-N-((5-(trifluoromethyl)-1H-pyrrol-2-yl)methyl)-1-naphthamide (intermediate from Example 27/3; 120 mg, 0.24 mmol) in DMF (5 mL) was added Cs₂CO₃ (94 mg, 0.29 mmol) and CH₃I (51 mg, 0.36 mmol) at rt. The mixture was stirred overnight at rt, concentrated and purified by prep-TLC (PE:EA=4:1) to give compound 28a as colorless glutinous oil.

### Step 2: 2-((4′-((2-Methyl-N-((1-methyl-5-(trifluoromethyl)-1H-pyrrol-2-yl)methyl)-1-naphthamido)methyl)-[1,1′-biphenyl]-3-yl)sulfonyl)acetic acid (28)

Compound 28a was coupled with boronic ester as described above (Pd₂(dba)₃, PPh₃ and K₃PO₄ in 1,4-dioxane at 95° C.), then saponified with LiOH.H₂O for 2 h and purified by prep-HPLC to obtain compound 28 as a white solid. ¹H-NMR (CDCl₃, 400 MHz) δ: 8.15, 7.98 (2 s, 1H), 7.83-7.20 (m, 12H), 6.77 (d, J=8.4 Hz, 1H), 6.48-6.35 (m, 1H), 6.01-5.93 (m, 1H), 4.96-4.86 (m, 1H), 4.74-4.65 (m, 1H), 4.16-4.05 (m, 4H), 3.74 (s, 2H), 2.80 (s, 1H), 2.35, 2.30 (2 s, 3H); MS: 635.0 (M+H)⁺.

### Example 29

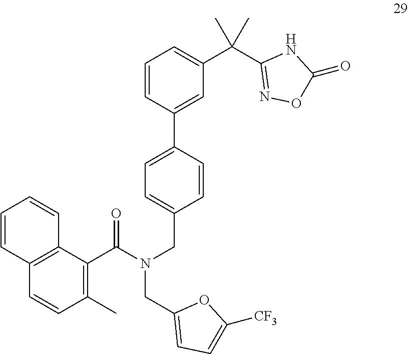 [SMILES: Cc1ccc2ccccc2c1C(=O)N(Cc1ccc(-c2cccc(C(C)(C)c3noc(=O)[nH]3)c2)cc1)Cc1ccc(C(F)(F)F)o1]

### Step 1: N-((3′-(1-Amino-2-methyl-1-oxopropan-2-yl)-[1,1′-biphenyl]-4-yl)methyl)-2-methyl-N-((5-(trifluoromethyl)furan-2-yl)methyl)-1-naphthamide (29a)

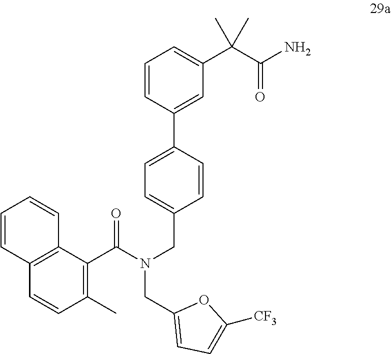 [SMILES: Cc1ccc2ccccc2c1C(=O)N(Cc1ccc(-c2cccc(C(C)(C)C(N)=O)c2)cc1)Cc1ccc(C(F)(F)F)o1]

To a solution of compound 27/26 (200 mg, 0.34 mmol) in DMF (10 mL) was added NH₄Cl (182 mg, 3.4 mmol), HATU (194 mg, 0.51 mmol) and DIPEA (132 mg, 1.02 mmol) and the mixture was stirred at rt for 3 h, diluted with water (100 mL) and extracted with EA (3×50 mL). The combined organic layer was washed with brine (100 mL), dried over Na₂SO₄, filtered, concentrated and purified by FCC (PE:EA=3:1) to give compound 29a as a white solid.

### Step 2: N-(3′-(2-Cyanopropan-2-yl)-[1,1-biphenyl]-4-yl)methyl)-2-methyl-N-((5-(trifluoromethyl)furan-2-yl)methyl)-1-naphthamide (29b)

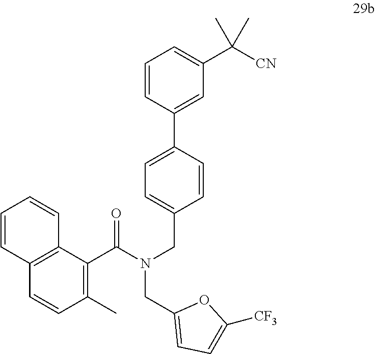 [SMILES: Cc1ccc2ccccc2c1C(=O)N(Cc1ccc(-c2cccc(C(C)(C)C#N)c2)cc1)Cc1ccc(C(F)(F)F)o1]

To a solution of compound 29a (180 mg, 0.31 mmol) in THF (40 mL) were added triethylamine (31 mg, 0.31 mmol) and TFAA (100 mg, 0.46 mmol) under ice-bath cooling. The mixture was stirred at the same temperature for 30 min, diluted with ice water and extracted with EA (2×). The combined organic layer was washed with brine, dried over MgSO₄, filtered, concentrated and purified by FCC (hexane:EA=10:1) to give compound 29b as a white solid.

### Step 3: N-((3′-(1-Amino-1-(hydroxyimino)-2-methylpropan-2-yl)-[1,1′-biphenyl]-4-yl)methyl)-2-methyl-N-((5-(trifluoromethyl)furan-2-yl)methyl)-1-naphthamide (29c)

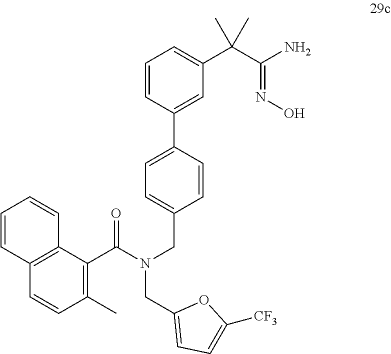 [SMILES: Cc1ccc2ccccc2c1C(=O)N(Cc1ccc(-c2cccc(C(C)(C)/C(N)=N/O)c2)cc1)Cc1ccc(C(F)(F)F)o1]

A suspension of compound 29b (150 mg, 0.26 mmol), hydroxylamine hydrochloride (90 mg, 1.30 mmol) and sodium carbonate (220 mg, 2.6 mmol) in ethanol (20 mL) was heated to reflux for 3 h, cooled, poured into water (30 mL) and extracted with EA (3×20 mL). The combined organic layer was washed with brine (30 mL), dried over Na₂SO₄, filtered and concentrated to give compound 29c as a white solid.

### Step 4: 2-Methyl-N-((3′-(2-(5-oxo-4,5-dihydro-1,2,4-oxadiazol-3-yl)propan-2-yl)-[1,1′-biphenyl]-4-yl)methyl)-N-((5-(trifluoromethyl)furan-2-yl)methy)-1-naphthamide (29)

To a solution of compound 29c (140 mg, 0.23 mmol) in CHCl₃ (10 mL) was added Et₃N (47 mg, 0.46 mmol) and phenyl carbonochloridate (38 mg, 0.23 mmol) at 0° C. The mixture was stirred at rt for 1 h, concentrated, redissolved in toluene (10 mL), refluxed overnight, concentrated and purified by prep-HPLC to give compound 29 as a white solid. ¹H-NMR (500 MHz, CD₃OD) δ: 7.93-7.90 (m, 2H), 7.66-7.34 (m, 11H), 7.05 (d, J=8.0 Hz, 1H), 7.00-6.99 (m, 0.5H), 6.73-6.72 (m, 0.5H), 6.55 (d, J=3.0 Hz, 0.5H), 6.09 (d, J=3.5 Hz, 0.5H), 5.09-4.89 (m, 2H), 4.35-4.29 (m, 2H), 2.48, 2.45 (2 s, 3H), 1.76, 1.72 (2 s, 6H); MS: 626.0 (M+H)⁺.

### Example 30

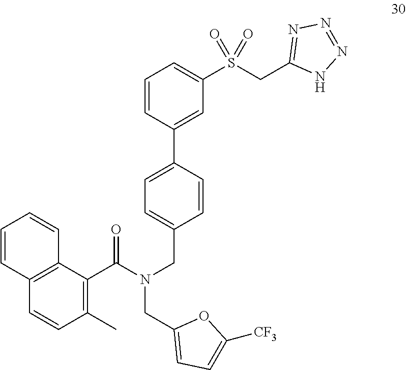 [SMILES: Cc1ccc2ccccc2c1C(=O)N(Cc1ccc(-c2cccc(S(=O)(=O)Cc3nnn[nH]3)c2)cc1)Cc1ccc(C(F)(F)F)o1]

### Step 1: 2-((3-Bromophenyl)thio)acetonitrile (30a)

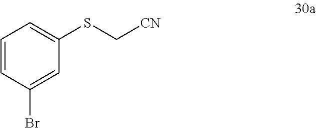 [SMILES: N#CCSc1cccc(Br)c1]

To a solution of 3-bromobenzenethiol (188 mg, 1.0 mmol) in DMF (10 mL) was added K₂CO₃ (414 mg, 3.0 mmol) under N₂ and the mixture was stirred for 10 min. 2-Bromoacetonitrile (143 mg, 1.2 mmol) was added and the mixture was stirred at rt under N₂ for 16 h, diluted with water (100 mL) and extracted with EA (2×20 mL). The combined organic layer was washed with brine (30 mL), dried over Na₂SO₄, filtered, concentrated and purified by FCC (PE:EA=3:1) to give compound 30a as a colorless oil.

### Step 2: 2-((3-Bromophenyl)sulfonyl)acetonitrile (30b)

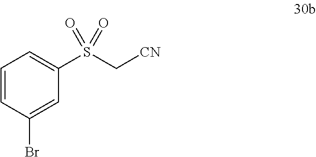 [SMILES: N#CCS(=O)(=O)c1cccc(Br)c1]

To a solution of compound 30a (190 mg, 0.84 mmol) in DCM (10 mL) was added m-CPBA (682 mg, 3.36 mmol, 85%) and the mixture was stirred at rt for 12 h. A sat. solution of Na₂SO₃ (100 mL) was added and the mixture was stirred for 1 h and extracted with DCM (3×30 mL). The combined organic layer was washed with brine (30 mL), dried over Na₂SO₄, filtered, concentrated and purified by FCC (PE:EA=2:1) to give compound 30b as a yellow solid.

### Step 3: 2-((3-(4,4,5,5-Tetramethyl-1,3,2-dioxaborolan-2-yl)phenylsulfonyl)acetonitrile (30c)

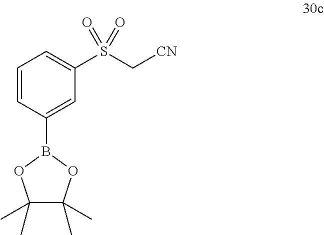 [SMILES: CC1(C)OB(c2cccc(S(=O)(=O)CC#N)c2)OC1(C)C]

To a solution of compound 30b (180 mg, 0.70 mmol) in 1,4-dioxane (10 mL) was added B₂Pin₂ (180 mg, 0.70 mmol), KOAc (137 mg, 1.4 mmol) and Pd(dppf)Cl₂ (20 mg). The mixture was stirred at 90° C. for 3 h under N₂, cooled, diluted with water (100 mL) and extracted with EA (3×50 mL). The combined organic layer was washed with brine (100 mL), dried over Na₂SO₄, filtered, concentrated and purified by FCC (PE:EA=3:1) to give compound 30c as a white solid.

### Step 4: N-((3′-((Cyanomethyl)sulfonyl)-[1,1′-biphenyl]-4-yl)methyl)-2-methyl-N-((5-(trifluoromethyl)furan-2-yl)methyl)-1-naphthamide (30d)

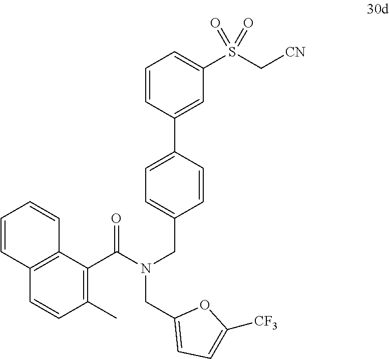 [SMILES: Cc1ccc2ccccc2c1C(=O)N(Cc1ccc(-c2cccc(S(=O)(=O)CC#N)c2)cc1)Cc1ccc(C(F)(F)F)o1]

To a solution of N-(4-bromobenzyl)-2-methyl-N-((5-(trifluoromethyl)furan-2-yl)methyl)-1-naphthamide (245 mg, 0.49 mmol) in 1,4-dioxane (10 mL) and water (1 mL) was added compound 30c (150 mg, 0.49 mmol), KOAc (100 mg, 1.0 mmol) and Pd(dppf)Cl₂ (20 mg) and the mixture was stirred at 90° C. for 3 h under N₂, diluted with water (100 mL) and extracted with EA (3×50 mL). The combined organic layer was washed with brine (100 mL), dried over Na₂SO₄, filtered, concentrated and purified by FCC (PE:EA=3:1) to give compound 30d as a white solid.

### Step 5: N-((3′-(((1H-Tetrazol-5-yl)methyl)sulfonyl)-1,1′-biphenyl-4-yl)methyl)-2-methyl-N-((5-(trifluoromethyl)furan-2-yl)methyl)-1-naphthamide (30)

To a mixture of compound 30d (200 mg, 0.33 mmol) in DMF (5 mL) was added NaN₃ (214 mg, 3.3 mmol) and NH₄Cl (176 mg, 3.3 mmol) and the mixture was stirred at 110° C. overnight, diluted with water (50 mL) and extracted with EA (3×30 mL). The combined organic layer was washed with brine (30 mL), dried over Na₂SO₄, filtered, concentrated and purified by prep-HPLC to give compound 30 as a white solid. ¹H-NMR (500 MHz, CD₃OD) δ: 7.92 (d, J=7.5 Hz, 0.5H), 7.82-7.48 (m, 3.5H), 7.68-7.50 (m, 5H), 7.42-7.31 (m, 4H), 6.95 (d, J=8.0 Hz, 1H), 6.89 (d, J=2.0 Hz, 0.5H), 6.62 (d, J=2.5 Hz, 0.5H), 6.44 (d, J=3.0 Hz, 0.5H), 5.99 (d, J=3.0 Hz, 0.5H), 4.98-4.81 (m, 4H), 4.32-4.16 (m, 2H), 2.36, 2.32 (2 s, 3H); MS: 646.0 (M+H)⁺.

### Example 31

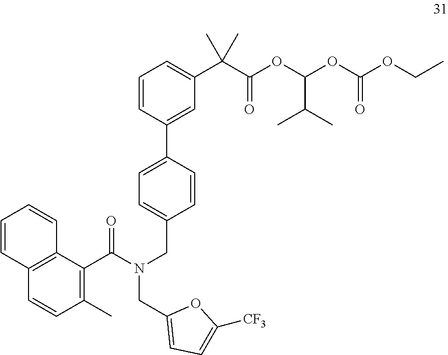 [SMILES: CCOC(=O)OC(OC(=O)C(C)(C)c1cccc(-c2ccc(CN(Cc3ccc(C(F)(F)F)o3)C(=O)c3c(C)ccc4ccccc34)cc2)c1)C(C)C]

### Step 1: 1-Chloro-2-methylpropyl ethyl carbonate (31a)

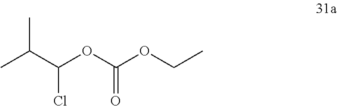

To a solution of EtOH (20 mL) and Et₃N (1.5 g, 15 mmol) was added 1-chloro-2-methylpropyl carbonochloridate (1.7 g, 10 mmol) at 0° C. The mixture was stirred at rt overnight, diluted with water (200 mL) and extracted with EA (3×30 mL). The combined organic layer was washed with brine (30 mL), dried over Na₂SO₄, filtered and concentrated to give compound 31a as a colorless oil.

### Step 2: 1-((Ethoxycarbonyl)oxy)-2-methylpropyl 2-methyl-2-(4′-((2-methyl-N-((5-(trifluoromethyl)furan-2-yl)methyl)-1-naphthamido)methyl)-[1,1′-biphenyl]-3-yl)propanoate (31)

To a mixture of compound 27/26 (150 mg, 0.26 mmol) in EA (5 mL) and DIPEA (139 mg, 1.0 mmol) was added of compound 31a (234 mg, 1.3 mmol) and the mixture was stirred at 70° C. overnight, cooled, diluted with water (40 mL) and extracted with EA (3×20 mL). The combined organic layer was washed with brine (30 mL), dried over Na₂SO₄, filtered, concentrated and purified by prep-HPLC to give compound 31 as a white solid. ¹H-NMR (500 MHz, CD₃COCD₃) δ: 7.92-7.32 (m, 13H), 7.16 (d, J=8.0 Hz, 1H), 7.09 (dd, J=3.5, 1.0 Hz, 0.5H), 6.85 (d, J=2.0 Hz, 0.5H), 6.62 (d, J=3.0 Hz, 0.5H), 6.55 (d, J=4.5 Hz, 0.5H), 6.52 (d, J=5.5 Hz, 0.5H), 6.23 (d, J=3.5 Hz, 0.5H), 5.07-4.90 (m, 2H), 4.38-4.29 (m, 2H), 4.12-4.02 (m, 2H), 2.46, 2.44 (2 s, 3H), 2.09-1.92 (m, 1H), 1.67-1.60 (m, 6H), 1.22-1.14 (m, 3H), 0.89-0.85 (m, 6H); MS: 652.2 (M+Na)⁺.

### Example 32

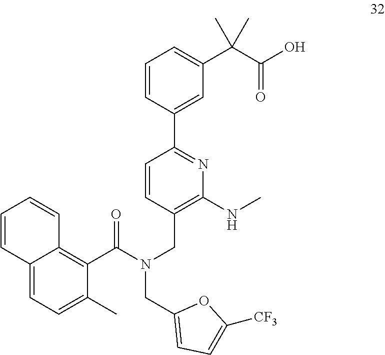 [SMILES: CNc1nc(-c2cccc(C(C)(C)C(=O)O)c2)ccc1CN(Cc1ccc(C(F)(F)F)o1)C(=O)c1c(C)ccc2ccccc12]

### Step 1: Methyl 2-methyl-2-(3-(5-((2-methyl-N-((5-(trifluoromethyl)furan-2-yl)methyl-1-naphthamido)methyl)-6-(methylamino)pyridin-2-yl)phenyl)propanoate (32a)

To a solution of the methyl ester of compound 27/91 (120 mg, 0.20 mmol) in DMF (5 mL) was added NaH (8 mg, 0.2 mmol, 60% in oil) and iodomethane (29 mg, 0.2 mmol) at 0° C. The mixture was stirred at rt for 1 h, diluted with water (50 mL) and extracted with EA (3×30 mL).

The combined organic layer was washed with brine (30 mL), dried over Na₂SO₄, filtered, concentrated and purified by FCC (PE:EA=5:1) to give compound 32a as a white solid.

### Step 2: 2-Methyl-2-(3-(5-((2-methyl-N-((5-(trifluoromethyl)furan-2-yl)methyl)-1-naphthamido)methyl)-6-(methylamino)pyridin-2-yl)phenyl)propanoic acid (32)

To the mixture of compound 32a (38 mg, 60 μmol) in MeOH (5 mL) and THF (2 mL) was added aq. LiOH (1M, 1 mL). The mixture was stirred at rt overnight, neutralized with 1N HCl and extracted with EA (3×). The combined organic layer was washed with brine, dried over Na₂SO₄, filtered, concentrated and purified by prep-HPLC to give compound 32 as a white solid. ¹H-NMR (500 MHz, CD₃OD) δ: 7.96-7.93 (m, 2H), 7.84-7.82 (m, 2H), 7.70-7.53 (m, 6H), 7.46 (d, 7.5 Hz, 1H), 6.99 (d, J=7.5 Hz, 1H), 6.71 (d, J=2.0 Hz, 1H), 6.03 (d, J=3.0 Hz, 1H), 5.15-5.10 (m, 2H), 4.55-4.40 (m, 2H), 3.31 (s, 3H), 2.45, 2.44 (2 s, 3H), 1.67, 1.65 (2 s, 6H); MS: 616.2 (M+H)⁺.

### Example 33

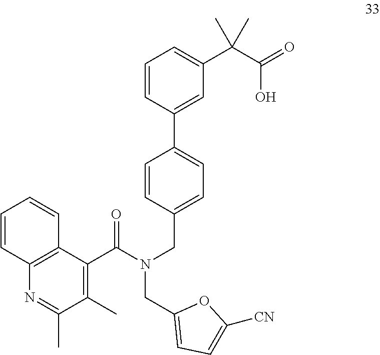 [SMILES: Cc1nc2ccccc2c(C(=O)N(Cc2ccc(-c3cccc(C(C)(C)C(=O)O)c3)cc2)Cc2ccc(C#N)o2)c1C]

### 2-(4-((N-((5-Cyanofuran-2-yl)methyl)-2,3-dimethylquinoline-4-carboxamido)methyl)-[1,1′-biphenyl]-3-yl)-2-methylpropanoic acid (33)

To a solution of compound 27/106 (130 mg, 0.23 mmol) in DCM (15 mL) and pyridine (1 mL) was added POCl₃ (0.5 mL) at 0° C. The mixture was stirred at 0° C. for 30 min, then allowed to reach rt for 1 h, quenched by aq. NaHCO₃ at 0° C., stirred for 15 min, adjusted to pH=3-4 with 2N HCl and extracted with EA (3×20 mL). The combined organic layer was washed with brine, dried over Na₂SO₄, filtered, concentrated and purified by prep-HPLC to give compound 33 as a white solid. ¹H-NMR (400 MHz, DMSO-d6) δ: 7.97-7.94 (m, 1H), 7.71-7.32 (m, 11H), 7.03 (d, J=8.0 Hz, 1H), 6.69 (d, J=3.6 Hz, 0.5H), 6.32 (d, J=3.6 Hz, 0.5H), 5.05-4.75 (m, 2H), 4.37-4.22 (m, 2H), 2.66, 2.64 (2s, 3H), 2.31, 2.28 (2 s, 3H), 1.54, 1.51 (2 s, 6H); MS: 558.3 (M+H)⁺.

### Example 33/1

The following example was synthesized similar as described for Example 33.

| # | building block | structure | analytical data |
|---|---|---|---|
| 33/1 | 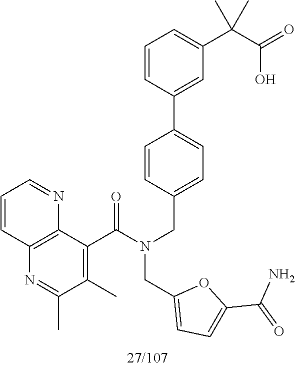 [SMILES: Cc1nc2cccnc2c(C(=O)N(Cc2ccc(-c3cccc(C(C)(C)C(=O)O)c3)cc2)Cc2ccc(C(N)=O)o2)c1C] | 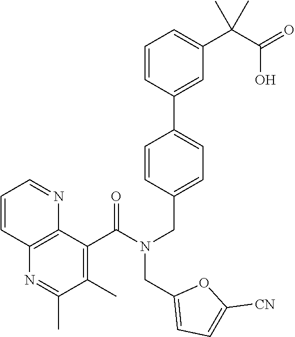 [SMILES: Cc1nc2cccnc2c(C(=O)N(Cc2ccc(-c3cccc(C(C)(C)C(=O)O)c3)cc2)Cc2ccc(C#N)o2)c1C] | ¹H-NMR (400 MHz, DMSO-d₆) δ 8.97 (d, J = 2.0 Hz, 1H), 8.37 (t, J = 7.0 Hz, 1H), 7.77-7.31 (m, 9H), 7.13 (d, J = 8.0 Hz, 1H), 6.86 (d, J = 3.6 Hz, 0.5H), 6.28 (d, J = 3.6 Hz, 0.5H), 5.04-4.68 (m, 2H), 4.36-4.19 (m, 2H), 2.70, 2.66 (2 s, 3H), 2.35, 2.30 (2 s, 3H), 1.55, 1.51 (2 s, 6H); MS: 559.2 (M + H)⁺. |

### Example 34

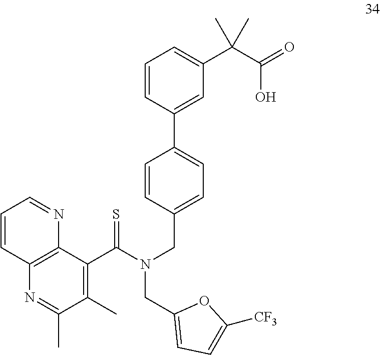 [SMILES: Cc1nc2cccnc2c(C(=S)N(Cc2ccc(-c3cccc(C(C)(C)C(=O)O)c3)cc2)Cc2ccc(C(F)(F)F)o2)c1C]

### Step 1: Methyl 2-(4′-((2,3-dimethyl-N-((5-(trifluoromethyl)furan-2-yl)methyl)-1,5-naphthyridine-4-carbothioamido)methyl)-[1,1′-biphenyl]-3-yl)-2-methylpropanoate (34a)

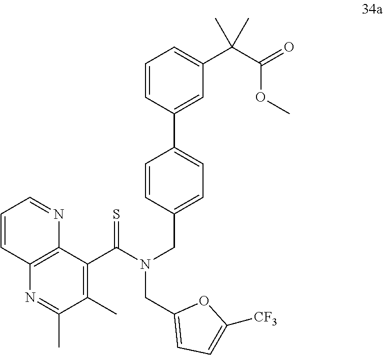 [SMILES: COC(=O)C(C)(C)c1cccc(-c2ccc(CN(Cc3ccc(C(F)(F)F)o3)C(=S)c3c(C)c(C)nc4cccnc34)cc2)c1]

A mixture of the methyl ester of compound 27/93 (280 mg, 0.46 mmol) and Lawesson's Reagent (184 mg, 2.28 mmol) in toluene was stirred at 120° C. for 2 d, cooled to rt, quenched with water and extracted with EA (3×30 mL). The combined organic layer was washed with brine, dried over Na₂SO₄, filtered, concentrated and purified by FCC (PE:EA=1:2) to give compound 34a as a yellow solid.

### Step 2: 2-(4-((2,3-Dimethyl-N-((5-trifluoromethyl)furan-2-yl)methyl-1,5-naphthyridine-4-carbothioamido)methyl)-[1,1′-biphenyl]-3-yl)-2-methylpropanoic acid (34)

To a solution of compound 34a (120 mg, 0.19 mmol) in CH₃OH (2 mL) and THF (2 mL) was added 1N LiOH (5 mL) and the mixture was refluxed overnight, cooled to rt, adjusted to pH=3-4 with 1N HCl and extracted with EA (3×10 mL). The combined organic layer was washed with brine, dried over Na₂SO₄, filtered, concentrated and purified by prep-HPLC to give compound 34 as a white solid. ¹H-NMR (400 MHz, CD₃OD) δ: 8.96, 8.91 (2 d, J=4.4, 1.6 Hz, 1H), 8.36-8.31 (m, 1H), 7.79-7.03 (m, 9.5H), 6.85 (d, J=3.2 Hz, 0.5H), 6.78 (d, J=2.4 Hz, 0.5H), 6.11 (d, J=3.2 Hz, 0.5H), 6.01 (d, J=15.2 Hz, 0.5H), 5.86 (d, J=14.8 Hz, 0.5H), 5.50 (d, J=15.2 Hz, 0.5H), 5.22 (d, J=15.6 Hz, 0.5H), 4.68 (d, J=15.2 Hz, 0.5H), 4.56-4.46 (m, 1.5H), 2.76, 2.70 (2 s, 3H), 2.47, 2.32 (2s, 3H), 1.64, 1.61 (2 s, 6H); MS: 618.4 (M+H)⁺.

### Example 35

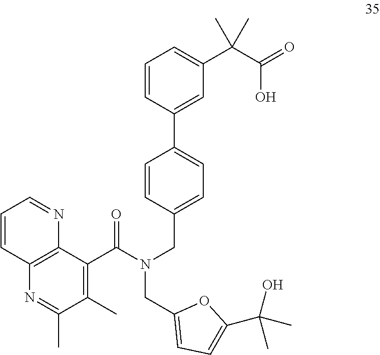 [SMILES: Cc1nc2cccnc2c(C(=O)N(Cc2ccc(-c3cccc(C(C)(C)C(=O)O)c3)cc2)Cc2ccc(C(C)(C)O)o2)c1C]

### 2-(4-((N-((5-(2-Hydroxypropan-2-yl)furan-2-yl)methyl)-2,3-dimethyl-1,5-naphthyridine-4-carboxamido)methyl)-[1,1′-biphenyl]-3-yl)-2-methylpropanoic acid (35)

To a solution of compound 27/128 (300 mg, 0.51 mmol) in THF (20 mL) at 0° C. was added MeMgBr (3M in Et₂O, 5 mL) and the mixture was stirred at 0° C. for 4 h, adjusted to pH=6-7 with 1N HCl and extracted with EA (3×10 mL). The combined organic layer was washed with brine, dried over Na₂SO₄, filtered, concentrated and purified by prep-HPLC to give compound 35 as a white solid. ¹H-NMR (400 MHz, CD₃OD) δ: 8.99-8.91 (m, 1H), 8.37-8.31 (m, 1H), 7.76-7.35 (m, 8H), 6.94 (d, J=8.4 Hz, 1H), 6.41 (d, J=3.2 Hz, 0.5H), 6.26 (d, J=3.2 Hz, 0.5H), 6.05 (d, J=3.2 Hz, 0.5H), 8.82 (d, J=3.2 Hz, 0.5H), 5.42-4.82 (m, 2H), 4.42-4.14 (m, 2H), 2.76, 2.66 (2 s, 3H), 2.47, 2.30 (2 s, 3H), 1.61-1.07 (m, 12H); MS: 592.3 (M+1)⁺.

### Example 36

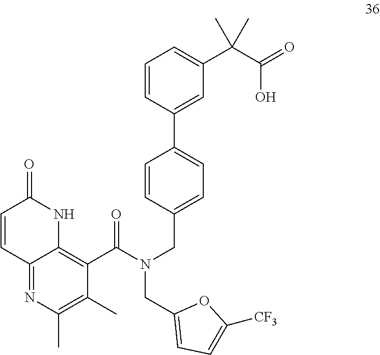 [SMILES: Cc1nc2ccc(=O)[nH]c2c(C(=O)N(Cc2ccc(-c3cccc(C(C)(C)C(=O)O)c3)cc2)Cc2ccc(C(F)(F)F)o2)c1C]

### 2-(4′-((2,3-Dimethyl-6-oxo-N-((5-(trifluoromethyl)furan-2-yl)methyl)-5,6-dihydro-1,5-naphthyridine-4-carboxamido)methyl)-[1,1′-biphenyl]-3-yl)-2-methylpropanoic acid (36)

To a solution of compound 27/134 (50 mg, 80 μmol) in ACN (5 mL) was added TMSCl (13 mg, 0.12 mmol) and NaI (22 mg, 0.12 mmol). The mixture was refluxed overnight, the solvent was removed and the residue was portioned between EA (20 mL) and water (10 mL). The aq. layers were extracted with EA (3×20 mL). The combined organic layers were dried over Na₂SO₄, concentrated, and purified by prep-HPLC to give compound 36 as white solid. ¹H-NMR (400 MHz, CD₃OD) δ: 8.00-7.79 (m, 1H), 7.63 (d, J=8.0 Hz, 1H), 7.54-7.33 (m, 6H), 7.03-6.95 (m, 2H), 6.86-6.26 (m, 2H), 5.79-5.64 (m, 1H), 4.49-4.14 (m, 3H), 2.61 (s, 3H), 2.36, 2.32 (2 s, 3H), 1.64 (s, 6H); MS: 618.3 (M+1)⁺.

If one were to follow the procedures described above using appropriate building blocks, the following compounds can be prepared:

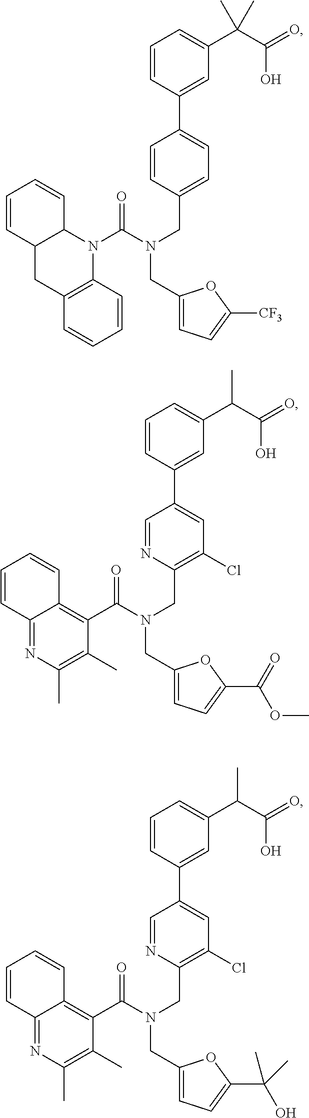

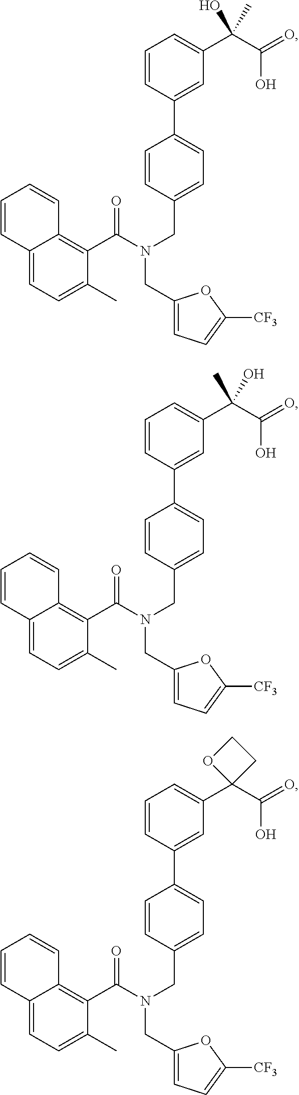

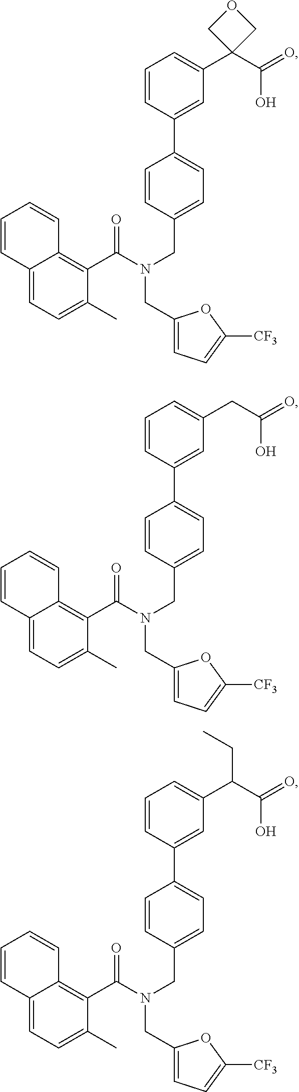

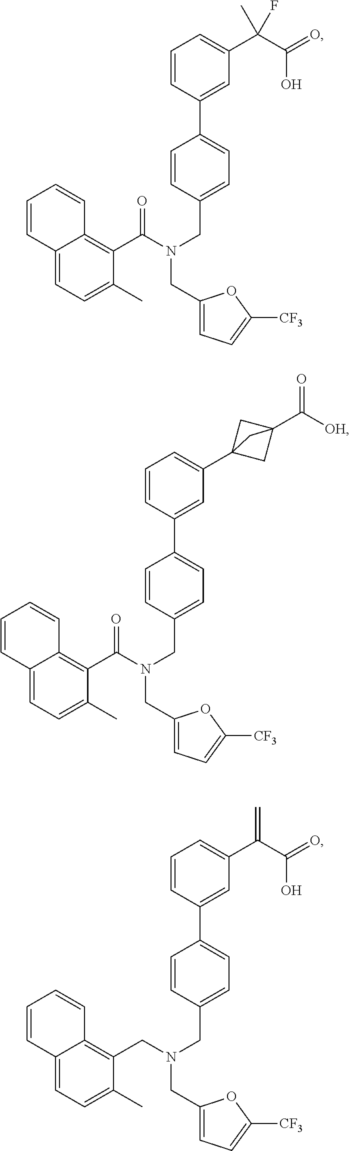 [SMILES: Cc1ccc2ccccc2c1C(=O)N(Cc1ccc(-c2cccc(C(C)(F)C(=O)O)c2)cc1)Cc1ccc(C(F)(F)F)o1 ; Cc1ccc2ccccc2c1C(=O)N(Cc1ccc(-c2cccc(C34CC(C(=O)O)(C3)C4)c2)cc1)Cc1ccc(C(F)(F)F)o1 ; C=C(C(=O)O)c1cccc(-c2ccc(CN(Cc3ccc(C(F)(F)F)o3)Cc3c(C)ccc4ccccc34)cc2)c1]

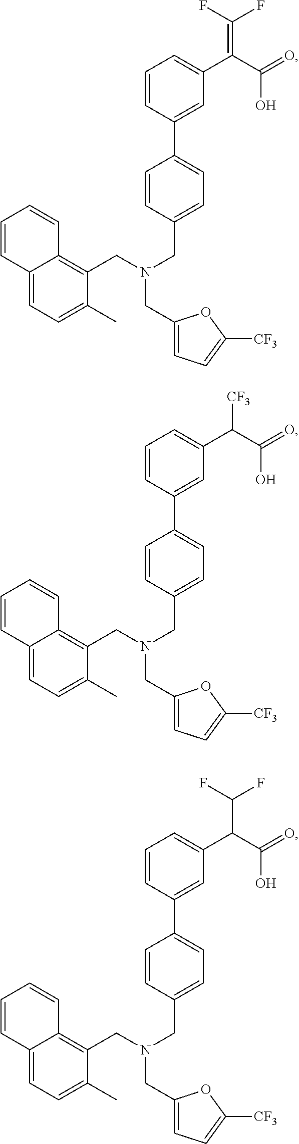

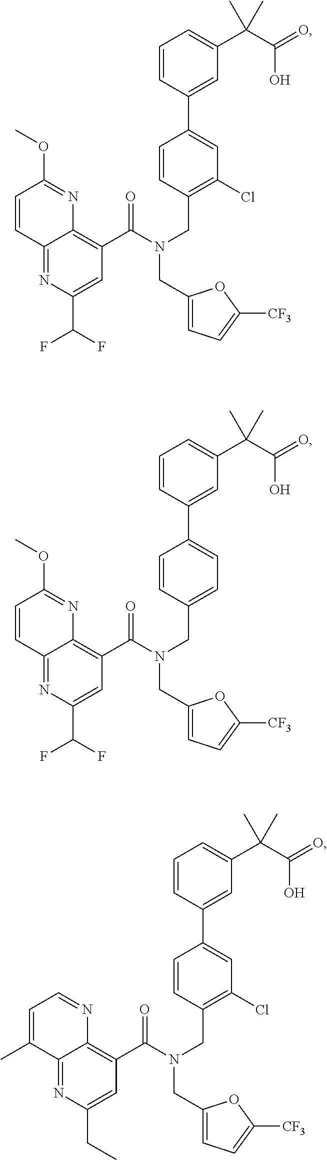 [SMILES: COc1ccc2nc(C(F)F)cc(C(=O)N(Cc3ccc(C(F)(F)F)o3)Cc3ccc(-c4cccc(C(C)(C)C(=O)O)c4)cc3Cl)c2n1 ; COc1ccc2nc(C(F)F)cc(C(=O)N(Cc3ccc(-c4cccc(C(C)(C)C(=O)O)c4)cc3)Cc3ccc(C(F)(F)F)o3)c2n1 ; CCc1cc(C(=O)N(Cc2ccc(C(F)(F)F)o2)Cc2ccc(-c3cccc(C(C)(C)C(=O)O)c3)cc2Cl)c2nccc(C)c2n1]

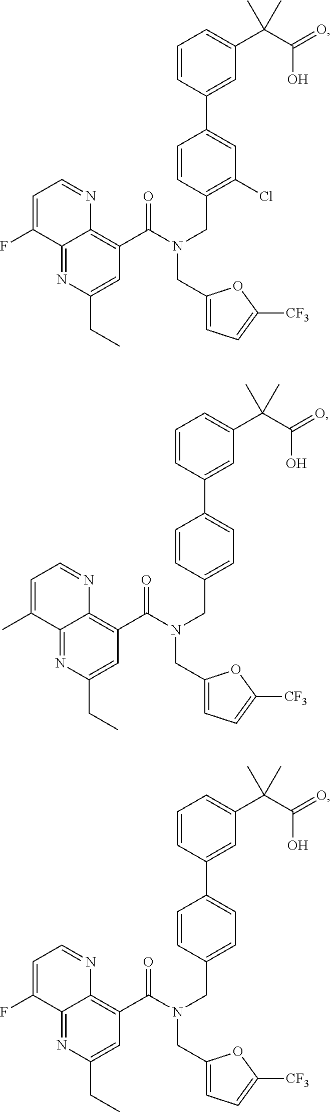

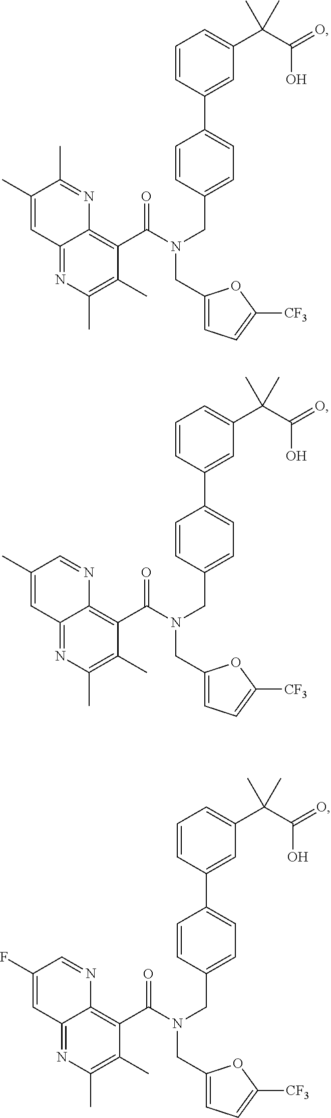

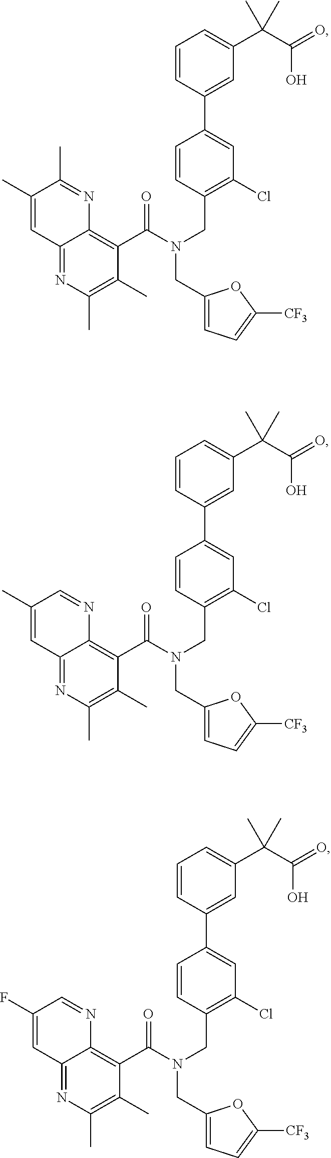 [SMILES: Cc1cc2nc(C)c(C)c(C(=O)N(Cc3ccc(C(F)(F)F)o3)Cc3ccc(-c4cccc(C(C)(C)C(=O)O)c4)cc3Cl)c2nc1C ; Cc1cnc2c(C(=O)N(Cc3ccc(C(F)(F)F)o3)Cc3ccc(-c4cccc(C(C)(C)C(=O)O)c4)cc3Cl)c(C)c(C)nc2c1 ; Cc1nc2cc(F)cnc2c(C(=O)N(Cc2ccc(C(F)(F)F)o2)Cc2ccc(-c3cccc(C(C)(C)C(=O)O)c3)cc2Cl)c1C]

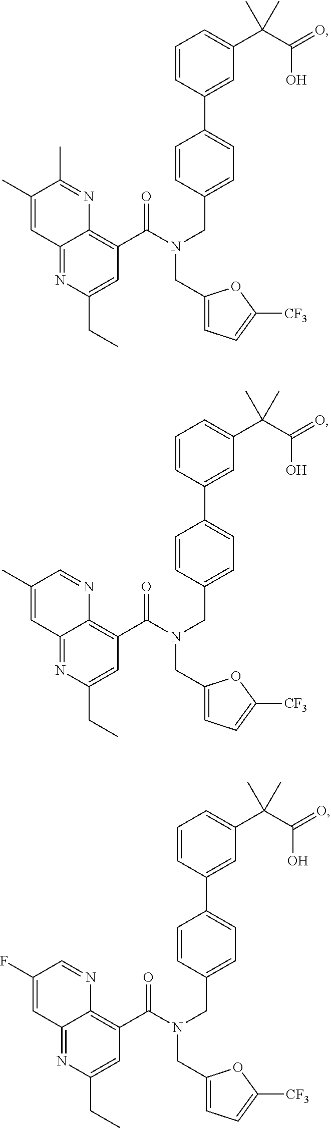 [SMILES: CCc1cc(C(=O)N(Cc2ccc(-c3cccc(C(C)(C)C(=O)O)c3)cc2)Cc2ccc(C(F)(F)F)o2)c2nc(C)c(C)cc2n1 ; CCc1cc(C(=O)N(Cc2ccc(-c3cccc(C(C)(C)C(=O)O)c3)cc2)Cc2ccc(C(F)(F)F)o2)c2ncc(C)cc2n1 ; CCc1cc(C(=O)N(Cc2ccc(-c3cccc(C(C)(C)C(=O)O)c3)cc2)Cc2ccc(C(F)(F)F)o2)c2ncc(F)cc2n1]

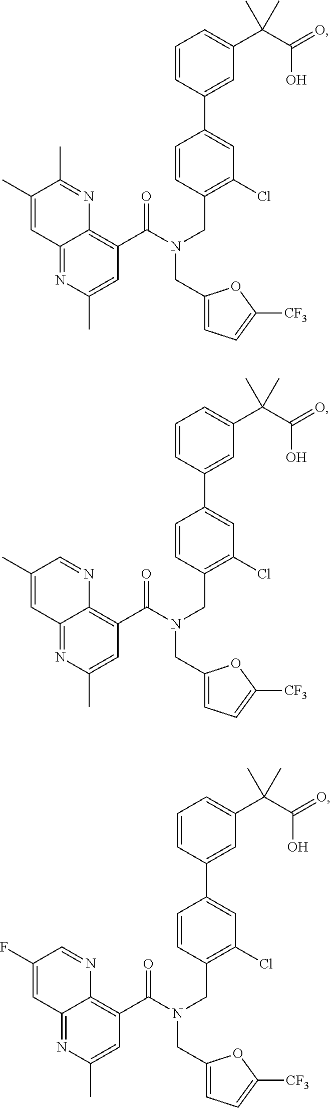

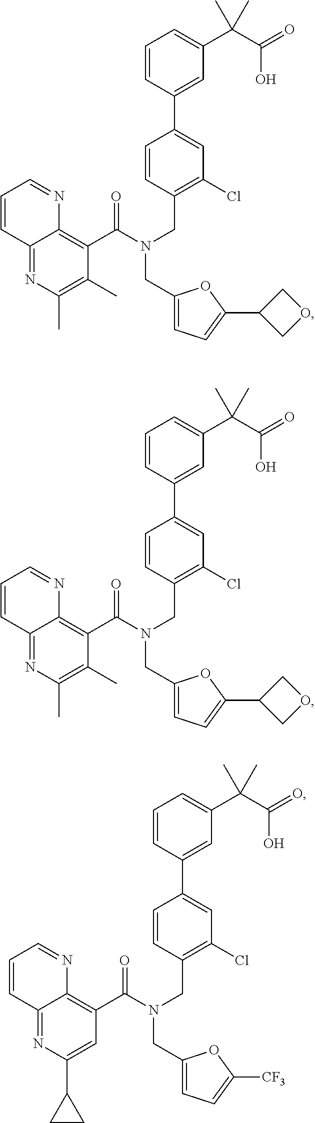

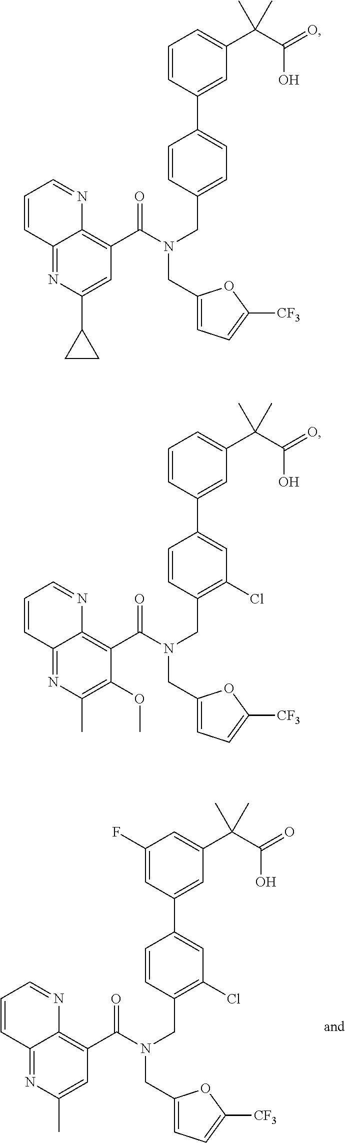 [SMILES: CC(C)(C(=O)O)c1cccc(-c2ccc(CN(Cc3ccc(C(F)(F)F)o3)C(=O)c3cc(C4CC4)nc4cccnc34)cc2)c1 ; COc1c(C)nc2cccnc2c1C(=O)N(Cc1ccc(C(F)(F)F)o1)Cc1ccc(-c2cccc(C(C)(C)C(=O)O)c2)cc1Cl ; Cc1cc(C(=O)N(Cc2ccc(C(F)(F)F)o2)Cc2ccc(-c3cc(F)cc(C(C)(C)C(=O)O)c3)cc2Cl)c2ncccc2n1]

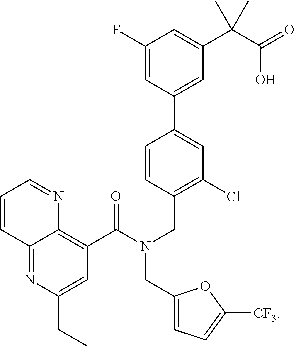 [SMILES: CCc1cc(C(=O)N(Cc2ccc(C(F)(F)F)o2)Cc2ccc(-c3cc(F)cc(C(C)(C)C(=O)O)c3)cc2Cl)c2ncccc2n1]

### Compound Stock Solutions

The tested compounds were usually dissolved, tested and stored as 20 mM stock solutions in DMSO. Since sulfonyl acetic acid derivatives tend to decarboxylate under these conditions, these stock solutions were prepared, tested and stored as 20 mM DMSO stock solutions containing 100 mM trifluoroacetic acid (5 equivalents). Sulfonyl acetic acid derivatives are shelf stable as solid at rt for long time as reported by Griesbrecht et al. (Synlett 2010:374) or Faucher et al. (J. Med. Chem. 2004; 47:18).

### TR-FRETβ Activity Assay

Recombinant GST-LXRβ ligand-binding domain (LBD; amino acids 156-461; NP009052; SEQ ID NO:4) was expressed in E. coli and purified via gluthatione-sepharose affinity chromatography. N-terminally biotinylated NCoA3 coactivator peptide (SEQ ID NO:7) was chemically synthesized (Eurogentec). Assays were done in 384 well format (final assay volume of 25 μL/well) in a Tris/HCl buffer (pH 6.8) containing KCl, bovine serum albumin, Triton-X-100 and 1 μM 24(S)-25-epoxycholesterol as LXR-prestimulating agonist. Assay buffer was provided and test articles (potential LXR inverse agonists) were titrated to yield final assay concentrations of 50 μM, 16.7 μM, 5.6 μM, 1.9 μM, 0.6 μM, 0.2 μM, 0.07 μM, 0.02 μM, 0.007 μM, 0.002 μM with one vehicle control. Finally, a detection mix was added containing anti GST-Tb cryptate (CisBio; 610SAXLB) and Streptavidin-XL665 (CisBio; 610SAXLB) as fluorescent donor and acceptor, respectively, as well as the coactivator peptide and LXRβ-LBD protein (SEQ ID NO:4). The reaction was mixed thoroughly, equilibrated for 1 h at 4° C. and vicinity of LXRβ and coactivator peptide was detected by measurement of fluorescence in a VictorX4 multiplate reader (PerkinElmer Life Science) using 340 nm as excitation and 615 and 665 nm as emission wavelengths. Assays were performed in triplicates.

### Final Assay Concentrations of Components:

240 mM KCl, 1 μg/μL BSA, 0.002% Triton-X-100, 125 pg/μL anti GST-Tb cryptate, 2.5 ng/μL Streptavidin-XL665, coactivator peptide (400 nM), LXRP protein (530 μg/mL, i.e. 76 nM).

### LXR Gal4 Reporter Transient Transfection Assays

LXRα and LXRβ activity status was determined via detection of interaction with coactivator and corepressor proteins in mammalian two-hybrid experiments (M2H). For this, via transient transfection the full length (FL) proteins of LXRα (amino acids 1-447; NP005684; SEQ ID NO:1) or LXRβ-(amino acids 1-461; NP009052; SEQ ID NO:2) or the ligand-binding domains (LBD) of LXRα (amino acids 155-447 SEQ ID NO:3) or LXR (amino acids 156-461; SEQ ID NO:4) were expressed from pCMV-AD (Stratagene) as fusions to the transcriptional activation domain of NFkB. As cofactors, domains of either the steroid receptor coactivator 1 (SRC1; amino acids 552-887; SEQ ID NO:5) or of the corepressor NCoR (amino acids 1906-2312; NP006302; SEQ ID NO:6) were expressed as fusions to the DNA binding domain of the yeast transcription factor GAL4 (from pCMV-BD; Stratagene). Interaction was monitored via activation of a coexpressed Firefly Luciferase Reporter gene under control of a promoter containing repetitive GAL4 response elements (vector pFRLuc; Stratagene). Transfection efficiency was controlled via cotransfection of constitutively active pRL-CMV Renilla reniformis luciferase reporter (Promega). HEK293 cells were grown in minimum essential medium (MEM) with 2 mM L-glutamine and Earle's balanced salt solution supplemented with 8.3% fetal bovine serum, 0.1 mM non-essential amino acids, 1 mM sodium pyruvate, at 37° C. in 5% CO₂. 3.5×10⁴ cells/well were plated in 96-well cell culture plates in growth medium supplemented with 8.3% fetal bovine serum for 16-20 h to—90% confluency. For transfection, medium was taken off and LXR and cofactor expressing plasmids as well as the reporter plasmids are added in 30 μL OPTIMEM/well including polyethylene-imine (PEI) as vehicle. Typical amounts of plasmids transfected/well: pCMV-AD-LXR (5 ng), pCMV-BD-cofactor (5 ng), pFR-Luc (100 ng), pRL-CMV (0.5 ng). Compound stocks were prepared in DMSO, prediluted in MEM to a total volume of 120 μL, and added 4 h after addition of the transfection mixture (final vehicle concentration not exceeding 0.2%). Cells were incubated for additional 16 h, lysed for 10 min in 1×Passive Lysis Buffer (Promega) and Firefly and Renilla luciferase activities were measured sequentially in the same cell extract using buffers containing D-luciferine and coelenterazine, respectively. Measurements of luminescence were done in a BMG-luminometer.

| Materials | Company | Cat.No. |
|---|---|---|
| HEK293 cells | DSMZ | ACC305 |
| MEM | Sigma-Aldrich | M2279 |
| OPTIMEM | LifeTechnologies | 11058-021 |
| FCS | Sigma-Aldrich | F7542 |
| Glutamax | Invitrogen | 35050038 |
| Pen/Strep | Sigma Aldrich | P4333 |
| Sodium Pyruvate | Sigma Aldrich | S8636 |
| Non Essential Amino Acids | Sigma Aldrich | M7145 |
| Trypsin | Sigma-Aldrich | T3924 |
| PBS | Sigma Aldrich | D8537 |
| PEI | Sigma Aldrich | 40.872-7 |
| Passive Lysis Buffer (5x) | Promega | E1941 |
| D-Luciferine | PJK | 260150 |
| Coelentrazine | PJK | 260350 |

TABLE 1

| Ex. # | FRETβ | LBD-M2H Gal4α | LBD-M2H Gal4β | FL-M2H Gal4α | FL-M2H Gal4β |
|---|---|---|---|---|---|
| 1 | B | B | C | | |
| 2 | B | | | B | C |
| 2/1 | A | | | — | — |
| 4 | B | C | C | | |
| 5 | C | | | C | C |
| 5/1 | C | | | C | C |
| 5/2 | D | | | C | D |
| 5/3 | D | | | D | D |
| 5/4 | C | | | B | B |
| 7 | D | D | D | | |
| 7/1 | B | | | C | D |
| 7/2 | B | | | C | C |
| 7/3 | | | | — | B |
| 7/3 | B* | | | B | C |
| 7/5 | C | | | C | C |
| 7/6 | B | | | C | C |
| 7/7 | B | | | B | C |
| 7/8 | A | | | — | B |
| 7/9 | B | B | D | | |
| 7/10 | C | | | B | C |
| 7/11 | | | | — | B |
| 7/12 | B | | | C | C |
| 7/13 | B | | | B | B |
| 7/14 | B | | | B | C |
| 7/15 | B | C | D | | |
| 9 | B | C | C | | |
| 9/1 | | | | — | B |
| 10 | D | | | C | C |
| 10/1 | C | C | D | D | D |
| 10/2 | B | C | D | | |
| 10/3 | A | C | C | | |
| 10/4 | C | D | D | | |
| 10/5 | D | | | D | D |
| 10/6 | D | | | D | D |
| 12 | B | | | — | — |
| 12/1 | C | | | C | C |
| 13 | C | B | D | | |
| 14 | B | B | D | | |
| 14/1 | B | C | D | | |
| 14/2 | B | C | D | | |
| 14/3 | C | D | D | | |
| 15 | B | | | C | C |
| 15/1 | B | | | B | C |
| 15/2 | B | | | — | B |
| 15/3 | B | B | C | | |
| 15/4 | A | | | — | C |
| 16 | — | | | — | B |
| 17 | A | | | B | C |
| 18 | — | | | — | C |
| 20 | B | | | — | C |
| 20/1 | C | | | B | C |
| 22 | A | | | B | C |
| 22/1 | B | — | C | | |
| 22/2 | B | — | C | | |
| 22/3 | — | | | B | C |
| 22/4 | C | B | D | | |
| 22/5 | C | | | C | D |
| 22/6 | B | | | — | B |
| 22/7 | B | | | C | C |
| 22/8 | B | D | D | | |
| 22/9 | B | | | C | D |
| 22/10 | B | | | B | C |
| 22/11 | C | D | D | | |
| 22/12 | C | C | D | | |
| 22/13 | B | | | C | C |
| 24 | D | D | D | D | D |
| 24/1 | D | D | D | | |
| 24/2 | B | C | D | | |
| 24/3 | C | D | D | | |
| 24/4 | C | D | D | | |
| 24/5 | D* | D | D | | |
| 24/6 | C | D | D | | |
| 25 | A | — | C | | |
| 25/1 | B* | C | D | | |
| 25/2 | — | C | D | | |
| 26/1 | B | C | D | | |
| 26/2 | B | C | D | | |
| 26/3 | B | — | D | | |
| 26/7 | A | | | B | C |
| 26/8 | B | | | C | C |
| 27 | A | | | — | — |
| 27/1 | B | | | C | D |
| 27/2 | B | | | B | B |
| 27/3 | B | | | B | B |
| 27/4 | A | | | C | C |
| 27/5 | C | | | D | D |
| 27/6 | D | | | D | D |
| 27/7 | D | | | D | D |
| 27/8 | B | | | C | C |
| 27/9 | C | | | D | D |
| 27/10 | C | | | D | D |
| 27/11 | B | | | D | D |
| 27/12 | D | | | D | D |
| 27/13 | B | | | C | D |
| 27/14 | C | | | B | C |
| 27/15 | C | | | D | D |
| 27/16 | C | | | D | D |
| 27/17 | C | | | D | D |
| 27/18 | C | | | D | D |
| 27/19 | C | | | D | D |
| 27/20 | C | | | D | D |
| 27/21 | C | | | D | D |
| 27/22 | C | | | C | D |
| 27/23 | C | | | D | D |
| 27/24 | B | | | C | D |
| 27/25 | B | | | C | D |
| 27/26 | D | | | D | D |
| 27/27 | C | | | D | D |
| 27/28 | D | | | D | D |
| 27/29 | — | | | B | B |
| 27/30 | B | | | C | D |
| 27/31 | D | | | D | D |
| 27/32 | D | | | D | D |
| 27/33 | C | | | D | D |
| 27/34 | B | | | B | C |
| 27/35 | B | | | B | C |
| 27/36 | C | | | D | D |
| 27/37 | C | | | C | D |
| 27/38 | D | | | C | D |
| 27/39 | D | | | C | D |
| 27/40 | A | | | — | B |
| 27/41 | B | | | B | B |
| 27/42 | C | | | B | C |
| 27/43 | B | | | D | D |
| 27/44 | C | | | D | D |
| 27/45 | D | | | D | D |
| 27/46 | D | | | D | D |
| 27/47 | D | | | D | D |
| 27/48 | C | | | D | D |
| 27/49 | C | | | D | D |
| 27/50 | C | | | D | D |
| 27/51 | B* | | | C | C |
| 27/52 | C | | | D | D |
| 27/53 | D | | | D | D |
| 27/54 | C | | | D | D |
| 27/55 | C | | | D | D |
| 27/56 | B* | C | D | | |
| 27/57 | A | | | — | — |
| 27/58 | B | | | C | C |
| 27/59 | C | | | C | C |
| 27/60 | B | | | C | C |
| 27/61 | B | | | C | C |
| 27/62 | B | | | B | C |
| 27/63 | C | | | | |
| 27/64 | C | | | C | D |
| 27/65 | C | | | D | D |
| 27/66 | C | | | D | D |
| 27/67 | D | D | D | | |
| 27/68 | D | D | D | | |
| 27/69 | C | D | D | | |
| 27/70 | C | C | D | | |
| 27/71 | C | | | D | D |
| 27/72 | C | | | D | |
| 27/73 | C | | | D | D |
| 27/74 | C | | | C | D |
| 27/75 | C | | | D | D |
| 27/76 | C | | | D | D |
| 27/77 | B | | | D | D |
| 27/78 | D | | | D | D |
| 27/79 | C | | | D | D |
| 27/80 | C | | | C | C |
| 27/81 | C | | | D | D |
| 27/82 | B | | | C | C |
| 27/83 | D | | | D | D |
| 27/84 | C | | | D | D |
| 27/85 | B | | | C | C |
| 27/86 | D | | | D | D |
| 27/87 | C | | | D | D |
| 27/88 | C | | | D | D |
| 27/89 | B | | | C | C |
| 27/90 | C | | | D | D |
| 27/91 | B | | | C | D |
| 27/92 | C | | | C | D |
| 27/93 | C | | | D | D |
| 27/94 | C | | | D | D |
| 27/95 | D | | | D | D |
| 27/96 | — | | | D | D |
| 27/97 | C* | | | D | D |
| 27/98 | C | | | C | C |
| 27/99 | B | | | B | B |
| 27/100 | A | | | B | B |
| 27/101 | A | | | B | C |
| 27/102 | C | | | D | D |
| 27/103 | D | | | D | D |
| 27/104 | C | | | D | D |
| 27/105 | C | | | D | D |
| 27/108 | C | | | D | D |
| 27/109 | B | | | C | C |
| 27/110 | C | | | D | D |
| 27/111 | B | | | C | D |
| 27/112 | C | | | D | D |
| 27/113 | C | | | D | D |
| 27/114 | C | | | D | D |
| 27/115 | C | | | D | D |
| 27/116 | B | | | C | C |
| 27/117 | B | | | B | B |
| 27/118 | C | | | C | C |
| 27/119 | B | | | C | C |
| 27/120 | B | | | C | C |
| 27/121 | D | | | D | D |
| 27/122 | B | | | C | C |
| 27/123 | C | | | D | D |
| 27/124 | D | | | D | D |
| 27/125 | C | | | D | D |
| 27/126 | C | | | D | C |
| 27/127 | B | | | C | C |
| 27/129 | C | | | C | D |
| 27/130 | C | | | D | D |
| 27/131 | C | | | C | C |
| 27/132 | B | | | C | D |
| 27/133 | C* | | | D | D |
| 27/134 | — | | | D | D |
| 27/135 | C | | | D | D |
| 28 | A | | | C | B |
| 29 | C | | | D | D |
| 30 | C | | | C | C |
| 31 | B | | | D | D |
| 32 | A | | | C | C |
| 33 | D | | | D | D |
| 33/1 | C | | | D | D |
| 33 | B | | | D | D |
| 35 | A | | | C | B |
| 36 | B | | | B | B |

Ranges (EC₅₀):
—: no activity measured;
A: >10 μM, B: 1 μM to <10 μM, C: 100 nM to <1 μM, D: <100 nM; inverse agonist behavior observed, if not otherwise stated by asterix (*); italic numbers indicate that efficacy (compared to GW2033) is below 40%.

### Pharmacokinetics

The pharmacokinetics of the compounds was assessed in mice after single dosing and oral administrations. Blood and liver exposure was measured via LC-MS.

The study design was as follows:

Animals: C57/bl6/J (Janvier) males
Diet: standard rodent chow
Dose: 20 mg/kg
Animal handling: animals were withdrawn from food at least 12 h before administration
Design: single dose oral administration, n=3 animals per group
Sacrifice: at stated time point (4, 12 or 24 h) after administration
Bioanalytics: LC-MS of liver and blood samples

TABLE 2
Study results:

| | time | blood/ | liver | liver/ |
|---|---|---|---|---|
| | point | plasma | expo- | blood |
| Example # | (h) | exposure | sure | ratio, |
| GSK2033 (neutral | 4 | below | below | — |
| comparative | | LLOQ | LLOQ | |
| example) | | (14.4 ng/mL) | (9.6 ng/mL) | |
| SR9238 (comparative | 4 | below | below | — |
| example with ester | | LLOQ | LLOQ | |
| moiety) | | | | |
| 1 | 4 | 0.83 μM | 42 μM | 51 |
| 1 | 12 | 0.06 μM | 3.2 μM | 54 |
| 4 | 12 | blow | 3.45 μM | — |
| | | LLOQ | | |
| 5/3 | 4 | 0.08 μM | 0.61 μM | 7.6 |
| 6 | 4 | 0.20 μM | 9.08 μM | 45 |
| 7/1 | 4 | 0.21 μM | 18 μM | 86 |
| 7/7 | 4 | 0.01 μM | 0.42 μM | 44 |
| 9 | 4 | 0.18 μM | 12.7 μM | 72 |
| 9 | 24 | 0.00 μM | 0.10 μM | 25 |
| 10 | 12 | 0.57 μM | 1.5 μM | 2.7 |
| 10/5 | 4 | 1.06 μM | 47.9 μM | 45 |
| 12/2 | 12 | 0.34 μM | 0.83 μM | 2.4 |
| 20/1 | 4 | 1.0 μM | 64 μM | 64 |
| 22/8 | 4 | 1.3 μM | 23 μM | 19 |
| 22/8 | 12 | 0.15 μM | 4.1 μM | 27 |
| 22/11 | 4 | 0.57 μM | 2.75 μM | 4.8 |
| 24 | 4 | 0.96 μM | 10.3 μM | 11 |
| 24 | 12 | 0.21 μM | 1.2 μM | 5.7 |
| 24 | 24 | 0.04 μM | 0.13 μM | 2.9 |
| 24/1 | 4 | 2.25 μM | 18 μM | 8 |
| 24/3 | 4 | 1.22 μM | 11.8 μM | 9.7 |
| 26/8 | 4 | 0.01 μM | 1.41 μM | 178 |
| 27/10 | 12 | 0.01 μM | 1.3 μM | 129 |
| 27/12 | 12 | 3.99 μM | 43.7 μM | 11 |
| 27/23 | 4 | 0.15 μM | 2.9 μM | 19 |
| 27/26 | 4 | 16 μM | 89 μM | 5.5 |
| 27/26 | 12 | 6.4 μM | 21 μM | 3.3 |
| 27/26 | 24 | 0.75 μM | 2.7 μM | 3.6 |
| 27/28 | 4 | 0.05 μM | 38.8 μM | 844 |
| 27/43 | 12 | 0.03 μM | 1.3 μM | 49 |
| 27/67 | 4 | 4.46 μM | 12.1 μM | 2.7 |
| 27/78 | 4 | 0.35 μM | 40.9 μM | 116 |

We confirmed that neutral sulfonamide GSK2033 and SR9238 are not orally bioavailable. Surprisingly we found, that when an acid moiety or acidic bioisostere is installed at another area of the molecule, i.e. instead or near the methylsulfone moiety of GSK2033/SR9238, these acidic compounds maintained to be potent on LXR and in addition are now orally bioavailable. The target tissue liver was effectively reached by compounds of the present invention and a systemic exposure, which is not desired, could be minimized.

In addition, the compounds of the present invention are more hepatotropic due to the acid moiety or acidic bioisosteric moiety (indicated by liver/blood ratios of 11 to 125).

### Short Term HFD Mouse Model:

The in vivo transcriptional regulation of several LXR target genes by LXR modulators was assessed in mice.

For this, C57BL/6J were purchased from Elevage Janvier (Rennes, France) at the age of 8 weeks. After an acclimation period of two weeks, animals were prefed on a high fat diet (HFD) (Ssniff Spezialdiäten GmbH, Germany, Surwit EF D12330 mod, Cat. No. E15771-34), with 60 kcal % from fat plus 1% (w/w) extra cholesterol (Sigma-Aldrich, St. Louis, Mo.) for 5 days. Animals were maintained on this diet during treatment with LXR modulators. The test compounds were formulated in 0.5% hydroxypropylmethylcellulose (HPMC) and administered in three doses (from 1.5 to 20 mg/kg each) by oral gavage according to the following schedule: on day one, animals received treatment in the morning and the evening (ca. 17:00), on day two animals received the final treatment in the morning after a 4 h fast and were sacrificed 4 h thereafter. Animal work was conducted according to the national guidelines for animal care in Germany.

Upon termination, liver was collected, dipped in ice cold PBS for 30 seconds and cut into appropriate pieces. Pieces were snap frozen in liquid nitrogen and stored at −80° C. For the clinical chemistry analysis from plasma, alanine aminotransferase (ALT, IU/mL), cholesterol (CHOL, mg/dL) and triglycerides (TG, mg/dL) were determined using a fully-automated bench top analyzer (Respons®910, DiaSys Greiner GmbH, Flacht, Germany) with system kits provided by the manufacturer.

Analysis of Gene Expression in Liver Tissue.

To obtain total RNA from frozen liver tissue, samples (25 mg liver tissue) were first homogenized with RLA buffer (4M guanidin thiocyanate, 10 mM Tris, 0.97% w:v β-mercapto-ethanol). RNA was prepared using a SV 96 total RNA Isolation system (Promega, Madison, Wis., USA) following the manufacturer's instructions. cDNAs were synthesized from 0.8-1 μg of total RNA using All-in-One cDNA Supermix reverse transcriptase (Absource Diagnostics, Munich, Germany).

Quantitative PCR was performed and analyzed using Prime time Gene expression master mix (Integrated DNA Technologies, Coralville, Iowa, USA) and a 384-format ABI 7900HT Sequence Detection System (Applied Biosystems, Foster City, USA). The expression of the following genes was analysed: Stearoyl-CoA desaturase1 (Scd1), fatty acid synthase (Fas) and sterol regulatory element-binding protein1 (Srebp1). Specific primer and probe sequences (commercially available) are listed in Table 2. qPCR was conducted at 95° C. for 3 min, followed by 40 cycles of 95° C. for 15 s and 60° C. for 30 s. All samples were run in duplicates from the same RT-reaction. Gene expression was expressed in arbitrary units and normalized relative to the mRNA of the housekeeping gene TATA box binding protein (Tbp) using the comparative Ct method.

TABLE 3
Primers used for quantitative PCR.

| | Forward | Reverse | Sequence |
|---|---|---|---|
| Gene | Primer | Primer | Probe |
| Fasn | CCCCTCTGTTA | TTGTGGAAGTGC | CAGGCTCAGGGTG |
| | ATTGGCTCC | AGGTTAGG | TCCCATGTT |
| | (SEQ ID | (SEQ ID | (SEQ ID |
| | NO: 8) | NO: 9) | NO: 10) |
| Scd1 | CTGACCTGAAA | AGAAGGTGCTAA | TGTTTACAAAAGT |
| | GCCGAGAAG | CGAACAGG | CTCGCCCCAGCA |
| | (SEQ ID | (SEQ ID | (SEQ ID |
| | NO: 11) | NO: 12) | NO: 13) |
| Srebp1c | CCATCGACTAC | GCCCTCCATAGA | TCTCCTGCTTGAG |
| | ATCCGCTTC | CACATCTG | CTTCTGGTTGC |
| | (SEQ ID | (SEQ ID | (SEQ ID |
| | NO: 14) | NO: 15) | NO: 16) |
| Tbp | CACCAATGACT | CAAGTTTACAGC | ACTCCTGCCACAC |
| | CCTATGACCC | CAAGATTCACG | CAGCCTC |
| | (SEQ ID | (SEQ ID | (SEQ ID |
| | NO: 17) | NO: 18) | NO: 19) |

TABLE 4
Study results

| Exam- | dose | | | |
|---|---|---|---|---|
| ple | [mg/ | plasma expo- | liver expo- | liver/plasma |
| # | kg] | sure, 4 h [nM] | sure, 4 h [nM] | ratio, 4 h |
| 9 | 20 | 134 | 18200 | 135 |
| 10/5 | 10 | 3160 | 24900 | 7.9 |
| 22/8 | 20 | 51 | 2820 | 55.7 |
| 24 | 5 | 893 | 2600 | 2.9 |
| 24 | 20 | 3520 | 8930 | 2.5 |
| 27/7 | 20 | 281 | 14800 | 52.5 |
| 27/10 | 3 | 47 | 9930 | 211 |
| 27/10 | 10 | 1440 | 43300 | 30.0 |
| 27/17 | 10 | 2920 | 6800 | 2.3 |
| 27/26 | 1.5 | 1040 | 6730 | 6.5 |
| 27/26 | 20 | 15300 | 44600 | 2.9 |
| 27/28 | 1.5 | 7 | 4300 | 600 |
| 27/28 | 20 | 8 | 13800 | 1790 |
| 27/36 | 10 | 3020 | 80200 | 26.6 |
| 27/38 | 20 | 2370 | 37500 | 15.8 |
| 27/43 | 20 | 1360 | 44300 | 32.5 |
| 27/45 | 10 | 871 | 320000 | 367 |
| 27/47 | 20 | 1070 | 38400 | 36.0 |
| 27/66 | 10 | 399 | 75300 | 189 |
| 27/72 | 10 | 1440 | 2020 | 1.4 |
| 27/76 | 10 | 2310 | 37900 | 16.4 |
| 27/78 | 10 | 300 | 18400 | 61.3 |
| 27/79 | 10 | 931 | 36500 | 39.2 |
| 27/81 | 10 | 849 | 43200 | 50.8 |
| 27/93 | 10 | 2100 | 155000 | 73.7 |
| | | Fasn | Srebp1c | Scd1 |
| Exam- | | suppression | suppression | suppression |
| ple | | compared to | compared to | compared to |
| # | | vehicle | vehicle | vehicle |
| 9 | 20 | 0.50 | 0.80 | 0.91 |
| 10/5 | 10 | 0.23 | 0.16 | 0.18 |
| 22/8 | 20 | 1.29 | 1.25 | 1.81 |
| 24 | 5 | 0.47 | 0.50 | 0.39 |
| 24 | 20 | 0.21 | 0.29 | 0.29 |
| 27/7 | 20 | 0.79 | 0.92 | 0.27 |
| 27/10 | 3 | 0.71 | 0.71 | 0.67 |
| 27/10 | 10 | 0.37 | 0.18 | 0.14 |
| 27/17 | 10 | 0.44 | 0.57 | 0.26 |
| 27/26 | 1.5 | 0.33 | 0.58 | 0.12 |
| 27/26 | 20 | 0.11 | 0.05 | 0.11 |
| 27/28 | 1.5 | 1.94 | 1.52 | 0.73 |
| 27/28 | 20 | 1.37 | 0.49 | 0.61 |
| 27/36 | 10 | 0.70 | 0.59 | 0.26 |
| 27/38 | 20 | 0.32 | 0.52 | 0.20 |
| 27/43 | 20 | 0.43 | 0.17 | 0.16 |
| 27/45 | 10 | 0.16 | 0.08 | 0.16 |
| 27/47 | 20 | 0.43 | 0.15 | 0.12 |
| 27/66 | 10 | 0.38 | 0.30 | 0.18 |
| 27/72 | 10 | 0.39 | 0.46 | 0.39 |
| 27/76 | 10 | 0.73 | 0.36 | 0.28 |
| 27/78 | 10 | 0.69 | 0.66 | 0.28 |
| 27/79 | 10 | 0.58 | 0.35 | 0.21 |
| 27/81 | 10 | 0.66 | 0.34 | 0.27 |
| 27/93 | 10 | 0.21 | 0.10 | 0.19 |

Multiple oral dosing of compounds from the present invention in mice lead to a high liver exposure with a favourable liver to plasma ratio. Hepatic LXR target genes were effectively suppressed. These genes are related to hepatic de-novo lipogenesis. A suppression of these genes will reduce liver fat (liver triglycerides).

### Comparative Examples

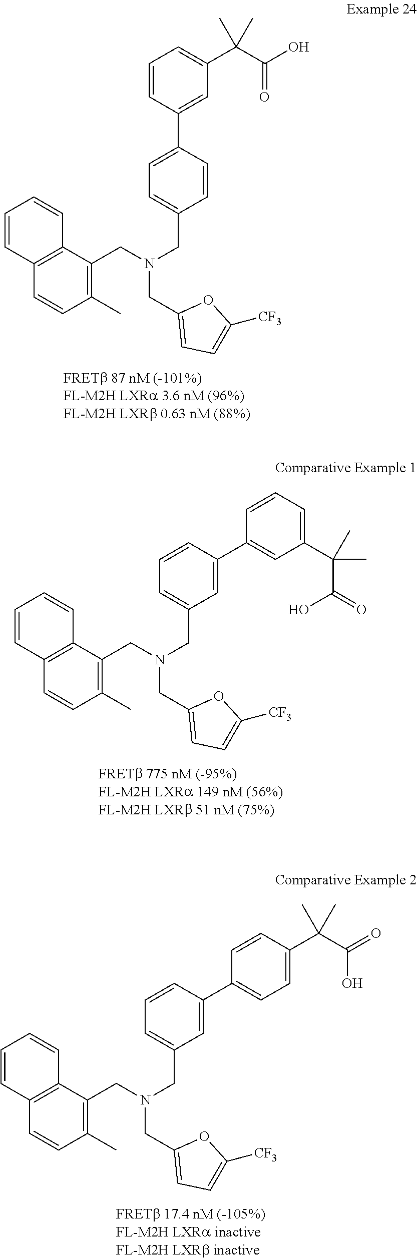 [SMILES: Cc1ccc2ccccc2c1CN(Cc1ccc(-c2cccc(C(C)(C)C(=O)O)c2)cc1)Cc1ccc(C(F)(F)F)o1 ; Cc1ccc2ccccc2c1CN(Cc1cccc(-c2cccc(C(C)(C)C(=O)O)c2)c1)Cc1ccc(C(F)(F)F)o1 ; Cc1ccc2ccccc2c1CN(Cc1cccc(-c2ccc(C(C)(C)C(=O)O)cc2)c1)Cc1ccc(C(F)(F)F)o1]

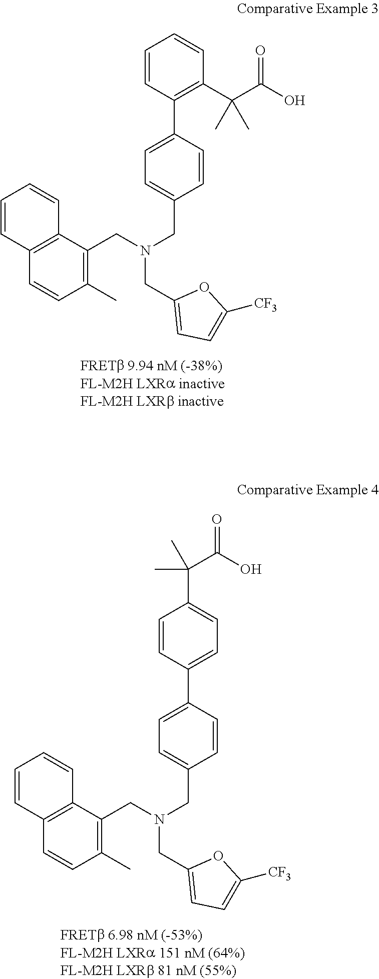 [SMILES: Cc1ccc2ccccc2c1CN(Cc1ccc(-c2ccccc2C(C)(C)C(=O)O)cc1)Cc1ccc(C(F)(F)F)o1 ; Cc1ccc2ccccc2c1CN(Cc1ccc(-c2ccc(C(C)(C)C(=O)O)cc2)cc1)Cc1ccc(C(F)(F)F)o1]

The Comparative Examples illustrate that the 1,4-connected biphenyls with a meta-substituent containing the acidic moiety (or bioisoster thereof) are preferred.

1. A compound represented by Formula (I) 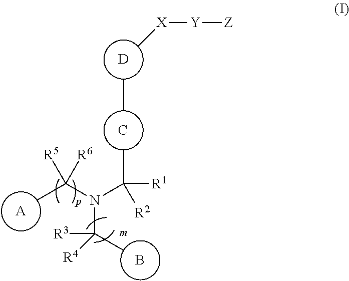
an enantiomer, diastereomer, tautomer, N-oxide, solvate, prodrug and pharmaceutically acceptable salt thereof, wherein
R¹, R² are independently selected from H and C₁₋₄-alkyl,
wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;
or R¹ and R² together are a 3- to 6-membered cycloalkyl or a 3- to 6-membered heterocycloalkyl containing 1 to 4 heteroatoms independently selected from N, O and S,
wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl, O-halo-C₁₋₄-alkyl;
or R¹ and an adjacent residue from ring C form a 5- to 8-membered saturated or partially unsaturated cycloalkyl or a 5- to 8-membered saturated or partially unsaturated heterocycloalkyl containing 1 to 4 heteroatoms independently selected from N, O and S,
wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
R³, R⁴ are independently selected from H and C₁₋₄-alkyl,
wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;
or R³ and R⁴ together are a 3- to 6-membered cycloalkyl or a 3- to 6-membered heterocycloalkyl containing 1 to 4 heteroatoms independently selected from N, O and S,
wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
or R³ and an adjacent residue from ring B form a 5- to 8-membered partially unsaturated cycloalkyl or a 5- to 8-membered partially unsaturated heterocycloalkyl containing 1 to 4 heteroatoms independently selected from N, O and S,
wherein the cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
R⁵, R⁶ are independently selected from H and C₁₋₄-alkyl,
wherein alkyl is unsubstituted or substituted with 1 to 3 substituents independently selected from halogen, CN, OH, oxo, C₁₋₄-alkyl, halo-C₁₋₄-alkyl, O—C₁₋₄-alkyl and O-halo-C₁₋₄-alkyl;
or R⁵ and R⁶ together are oxo, thioxo, a 3- to 6-membered cycloalkyl or a 3- to 6-membered heterocycloalkyl containing 1 to 4 heteroatoms independently selected from N, O and S,
wherein cycloalkyl and heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl;
or R⁵ and an adjacent residue from ring A form a 5- to 8-membered saturated or partially unsaturated cycloalkyl or a 5- to 8-membered saturated or partially unsaturated heterocycloalkyl containing 1 to 4 heteroatoms independently selected from N, O and S,
wherein the cycloalkyl or the heterocycloalkyl is unsubstituted or substituted with 1 to 4 substituents independently selected from halogen, CN, OH, oxo, C₁₋₄-alkyl, halo-C₁₋₄-alkyl, O—C₁₋₄-alkyl and O-halo-C₁₋₄-alkyl; 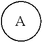
is selected from the group consisting of 4- to 10-membered cycloalkyl, 4- to 10-membered heterocycloalkyl containing 1 to 4 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N,
wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; 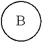
is selected from the group consisting of 6- or 10-membered aryl and 5- to 10-membered heteroaryl containing 1 to 4 heteroatoms independently selected from N, O and S,
wherein the 6-membered aryl and 5- or 6-membered heteroaryl are substituted with 1 to 4 substituents independently selected from the group consisting of halogen, CN, NO₂, OXO, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
and wherein the 10-membered aryl or 7- to 10-membered heteroaryl are unsubstituted or substituted with 1 to 4 substituents independently selected from the group consisting of halogen, CN, NO₂, OXO, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-S(O)₂NR⁶¹R⁶², C₀₋₆-alkylene-NR⁶¹S(O)₂NR⁶¹R⁶², C₀₋₆-alkylene-CO₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and
wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; 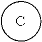
is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 4 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹—CO₂R⁷¹, C₀₋₆-alkylene-NR⁷¹R⁷²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; wherein the residue —CR¹R²— on ring C is linked at least with one 1,4-orientation regarding the connection towards ring D; 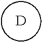
is selected from the group consisting of 6-membered aryl and 5- to 6-membered heteroaryl containing 1 to 4 heteroatoms independently selected from N, O and S,
wherein aryl and heteroaryl are unsubstituted or substituted with 1 to 4 substituents independently selected from the group consisting of halogen, CN, NO₂, oxo, C₁₋₄-alkyl, C₀₋₆-alkylene-OR⁸¹, C₀₋₆-alkylene-(3- to 6-membered cycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R and C₀₋₆-alkylene-NR⁸¹R⁸²,
wherein alkyl, alkylene and cycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N,
wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl;
wherein the residue X—Y—Z on ring D is linked in 1,3-orientation regarding the connection towards ring C;
X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹—;
Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S,
wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂;
Z is selected from —CO₂H, —CONH—CN, —CONHOH, —CONHOR⁹⁰, —CONR⁹⁰OH, —CONHS(═O)₂R⁹⁰, —NR⁹¹CONHS(═O)₂R⁹⁰, —CONHS(═O)₂NR⁹¹R⁹², —SO₃H, —S(═O)₂NHCOR⁹⁰, —NHS(═O)₂R⁹⁰, —NR⁹¹S(═O)₂NHCOR⁹⁰, —S(═O)₂NHR⁹⁰, —P(═O)(OH)₂, —P(═O)(NR⁹¹R⁹²)OH, 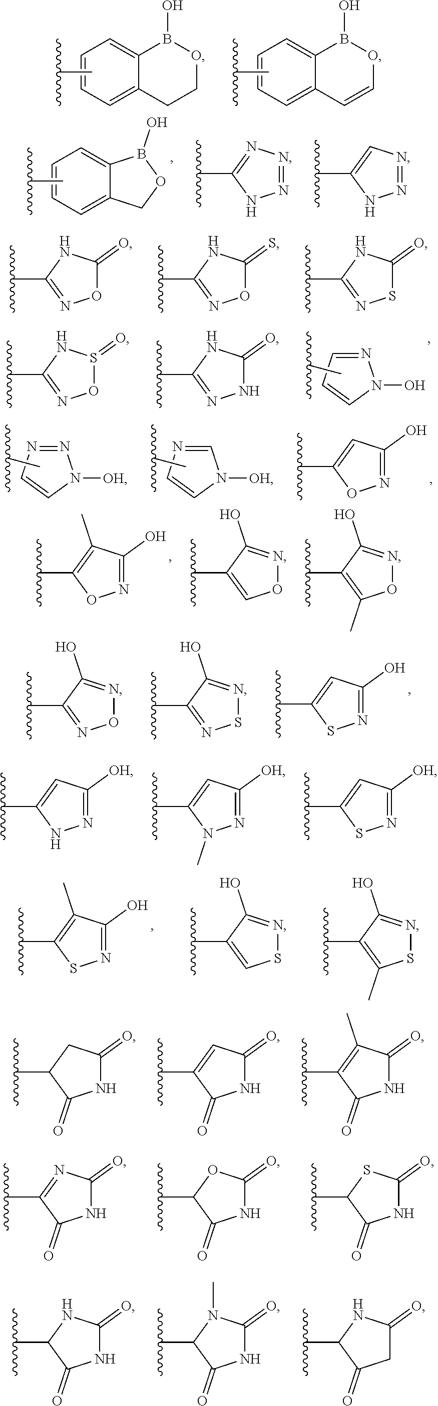 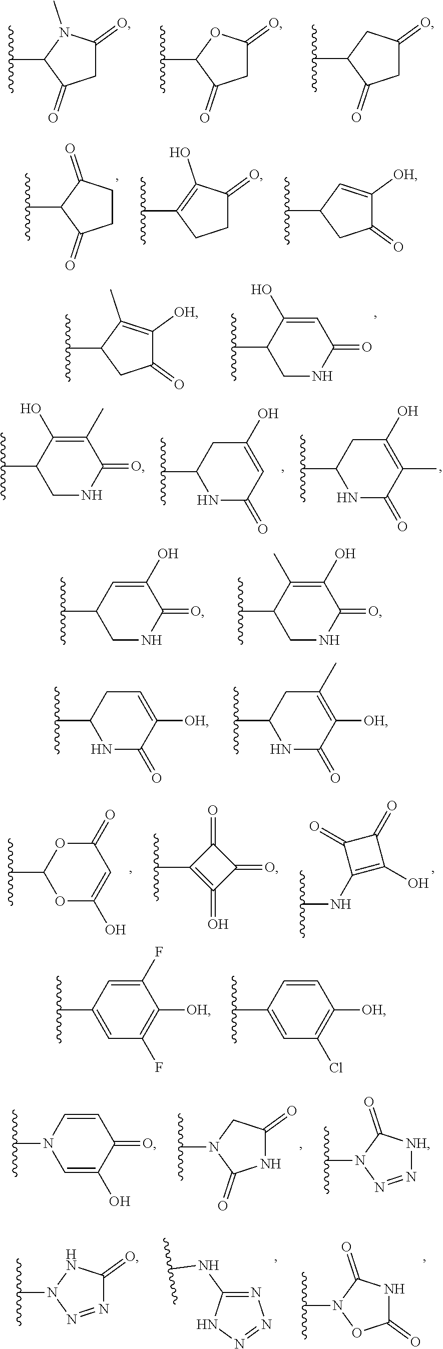 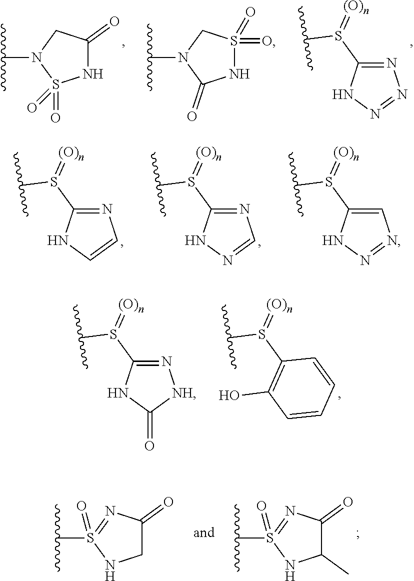
R¹¹ is selected from H, CN, NO₂, C₁₋₄-alkyl, C(═O)—C₁₋₄-alkyl, C(═O)—O—C₁₋₄-alkyl, halo C₁₋₄-alkyl, C(═O)-halo-C₁₋₄-alkyl and C(═O)—O-halo-C₁₋₄-alkyl;
R⁵¹, R⁵², R⁶¹, R⁶², R⁷¹, R⁷², R⁸¹, R⁸² are independently selected from H and C₁₋₄-alkyl,
wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
or R⁵¹ and R⁵², R⁶¹ and R⁶², R⁷¹ and R⁷², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
R⁹⁰ is independently selected from C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
R⁹¹, R⁹² are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, SO₃H, O—C₁₋₄-alkyl and O-halo-C₁₋₄-alkyl;
or R⁹¹ and R⁹² when taken together with the nitrogen to which they are attached complete a 3- to 6-membered ring containing carbon atoms and optionally containing 1 or 2 heteroatoms selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl and O-halo-C₁₋₄-alkyl;
n is selected from 0 to 2;
m and p is independently selected from 1 and 2.

2. The compound according to claim 1 wherein
R¹, R², R³ and R⁴ are independently selected from H or Me;
R⁵ and R⁶ are independently selected from H or Me or R⁵ and R⁶ together are oxo;
m and p is 1.

3. The compound according to claim 1 wherein 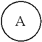
is selected from the group consisting of 6- to 14-membered aryl and 5- to 14-membered heteroaryl containing 1 to 4 heteroatoms independently selected from N, O and S,
wherein aryl and heteroaryl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵—CO₂R⁵¹ and C₀₋₆-alkylene-NR⁵¹R⁵²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N,
wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl; or 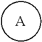
is selected from the group consisting of 4- to 10-membered cycloalkyl and 4- to 10-membered heterocycloalkyl containing 1 to 4 heteroatoms independently selected from N, O and S,
wherein cycloalkyl and heterocycloalkyl are unsubstituted or substituted with 1 to 6 substituents independently selected from the group consisting of halogen, CN, NO₂, oxo, C₁₋₄-alkyl, C₀₋₆-alkylene-OR⁵¹, C₀₋₆-alkylene-(3- to 6-membered-cycloalkyl), C₀₋₆-alkylene-(3- to 6-membered-heterocycloalkyl), C₀₋₆-alkylene-S(O)ₙR⁵¹, C₀₋₆-alkylene-NR⁵¹S(O)₂R⁵¹, C₀₋₆-alkylene-S(O)₂NR⁵¹R⁵², C₀₋₆-alkylene-NR⁵¹S(O)₂NR⁵¹R⁵², C₀₋₆-alkylene-CO₂R⁵¹, C₀₋₆-alkylene-O—COR⁵¹, C₀₋₆-alkylene-CONR⁵¹R⁵², C₀₋₆-alkylene-NR⁵¹—COR⁵¹, C₀₋₆-alkylene-NR⁵¹—CONR⁵¹R⁵², C₀₋₆-alkylene-O—CONR⁵¹R⁵², C₀₋₆-alkylene-NR⁵¹—CO₂R⁵¹ and C₀₋₆-alkylene-NR⁵¹R⁵²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
and wherein two adjacent substituents on the cycloalkyl or heterocycloalkyl moiety form a 5- to 6-membered unsaturated cycle optionally containing 1 to 3 heteroatoms independently selected from O, S or N, wherein this additional cycle is unsubstituted or substituted with 1 to 4 substituents independently selected from halogen, CN, oxo, OH, C₁₋₄-alkyl, halo-C₁₋₄-alkyl, O—C₁₋₄-alkyl and O-halo-C₁₋₄-alkyl.

4. The compound according to claim 1 wherein 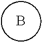
is selected from the group consisting of 6-membered aryl and 5- to 6-membered heteroaryl containing 1 to 4 heteroatoms independently selected from N, O and S,
wherein the 6-membered aryl and 5- or 6-membered heteroaryl are substituted with 1 to 4 substituents independently selected from the group consisting of halogen, CN, NO₂, oxo, C₁₋₄-alkyl, C₀₋₆-alkylene-OR⁶¹, C₀₋₆-alkylene-(3- to 6-membered cycloalkyl), C₀₋₆-alkyl-(3- to 6-membered heterocycloalkyl), C₀₋₆-alkylene-S(O)ₙR⁶¹, C₀₋₆-alkylene-NR⁶¹S(O)₂R⁶¹, C₀₋₆-alkylene-O—COR⁶¹, C₀₋₆-alkylene-CONR⁶¹R⁶², C₀₋₆-alkylene-NR⁶¹—COR⁶¹, C₀₋₆-alkylene-NR⁶¹—CONR⁶¹R⁶², C₀₋₆-alkylene-O—CONR⁶¹R⁶², C₀₋₆-alkylene-NR⁶¹—CO₂R⁶¹ and C₀₋₆-alkylene-NR⁶¹R⁶²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl.

5. The compound according to claim 1 wherein 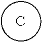
is selected from the group consisting of 6-membered aryl and 5- to 6-membered heteroaryl containing 1 to 4 heteroatoms independently selected from N, O and S,
wherein aryl and heteroaryl are unsubstituted or substituted with 1 to 4 substituents independently selected from the group consisting of halogen, CN, NO₂, oxo, C₁₋₄-alkyl, C₀₋₆-alkylene-OR⁷¹, C₀₋₆-alkylene-(3- to 6-membered cycloalkyl), C₀₋₆-alkylene-(3- to 6-membered heterocycloalkyl), C₀₋₆-alkylene-S(O)ₙR⁷¹, C₀₋₆-alkylene-NR⁷¹S(O)₂R⁷¹, C₀₋₆-alkylene-S(O)₂NR⁷¹R⁷², C₀₋₆-alkylene-NR⁷¹S(O)₂NR⁷¹R⁷², C₀₋₆-alkylene-CO₂R⁷¹, C₀₋₆-alkylene-O—COR⁷¹, C₀₋₆-alkylene-CONR⁷¹R⁷², C₀₋₆-alkylene-NR⁷¹—COR⁷¹, C₀₋₆-alkylene-NR⁷¹—CONR⁷¹R⁷², C₀₋₆-alkylene-O—CONR⁷¹R⁷², C₀₋₆-alkylene-NR⁷¹CO₂R⁷¹, C₀₋₆-alkylene-NR⁷¹R⁷²,
wherein alkyl, alkylene, cycloalkyl and heterocycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
wherein the residue —CR¹R²— on ring C is linked at least with one 1,4-orientation regarding the connection towards ring D.

6. The compound according to claim 1 wherein 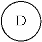
is selected from the group consisting of 6-membered aryl and 5- to 6-membered heteroaryl containing 1 to 4 heteroatoms independently selected from N, O and S,
wherein aryl and heteroaryl are unsubstituted or substituted with 1 to 4 substituents independently selected from the group consisting of halogen, CN, NO₂, oxo, C₁₋₄-alkyl, C₀₋₆-alkylene-OR⁸¹, C₀₋₆-alkylene-(3- to 6-membered cycloalkyl), C₀₋₆-alkylene-S(O)ₙR⁸¹, C₀₋₆-alkylene-NR⁸¹S(O)₂R⁸¹, C₀₋₆-alkylene-S(O)₂NR⁸¹R⁸², C₀₋₆-alkylene-NR⁸¹S(O)₂NR⁸¹R⁸², C₀₋₆-alkylene-CO₂R⁸¹, C₀₋₆-alkylene-O—COR⁸¹, C₀₋₆-alkylene-CONR⁸¹R⁸², C₀₋₆-alkylene-NR⁸¹—COR⁸¹, C₀₋₆-alkylene-NR⁸¹—CONR⁸¹R⁸², C₀₋₆-alkylene-O—CONR⁸¹R⁸², C₀₋₆-alkylene-NR⁸¹—CO₂R⁸¹ and C₀₋₆-alkylene-NR⁸¹R⁸²,
wherein alkyl, alkylene and cycloalkyl is unsubstituted or substituted with 1 to 6 substituents independently selected from halogen, CN, oxo, hydroxy, C₁₋₄-alkyl, halo-C₁₋₄-alkyl, O—C₁₋₄-alkyl and O-halo-C₁₋₄-alkyl;
wherein the residue X—Y—Z on ring D is linked in 1,3-orientation regarding the connection towards ring C.

7. The compound according to claim 1 wherein
X is selected from a bond, C₀₋₆-alkylene-S(═O)ₙ—, C₀₋₆-alkylene-S(═NR¹¹)(═O)—, C₀₋₆-alkylene-S(═NR¹¹)—, C₀₋₆-alkylene-O—, C₀₋₆-alkylene-NR⁹¹—, C₀₋₆-alkylene-S(═O)₂NR⁹¹—, C₀₋₆-alkylene-S(═NR¹¹)(═O)—NR⁹¹— and C₀₋₆-alkylene-S(═NR¹¹)—NR⁹¹—;
Y is selected from C₁₋₆-alkylene, C₂₋₆-alkenylene, C₂₋₆-alkinylene, 3- to 8-membered cycloalkylene, 3- to 8-membered heterocycloalkylene containing 1 to 4 heteroatoms independently selected from N, O and S,
wherein alkylene, alkenylene, alkinylene, cycloalkylene or heterocycloalkylene is unsubstituted or substituted with 1 to 6 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, O—C₁₋₄-alkyl, O-halo-C₁₋₄-alkyl, NH₂, NH(C₁₋₄-alkyl), N(C₁₋₄-alkyl)₂, NH(halo-C₁₋₄-alkyl) and N(halo-C₁₋₄-alkyl)₂;
Z is selected from —CO₂H, —CONHO—C₁₋₄-alkyl, —CON(C₁₋₄-alkyl)OH, —CONHOH, —CONHSO₂—C₁₋₄-alkyl, —CONHSO₂—N(C₁₋₄-alkyl)₂, 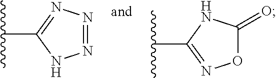 [SMILES: *c1nnn[nH]1 ; *c1noc(=O)[nH]1]
or a prodrug and pharmaceutically acceptable salt thereof.

8. The compound according to claim 1 wherein
X is selected from a bond, O and S(═O)₂;
Y is selected from C₁₋₃-alkylene, 3- to 6-membered cycloalkylene and 3- to 6-membered heterocycloalkylene containing 1 to 4 heteroatoms independently selected from N, O and S,
wherein alkylene, cycloalkylene or heterocycloalkylene is unsubstituted or substituted with 1 to 2 substituents independently selected from fluoro, CN, C₁₋₄-alkyl, halo-C₁₋₄-alkyl, OH, NH₂, oxo, O—C₁₋₄-alkyl and O-halo-C₁₋₄-alkyl; and
Z is selected from —CO₂H, —CONHO—C₁₋₄-alkyl, —CON(C₁₋₄-alkyl)OH, —CONHOH, —CONHSO₂—C₁₋₄-alkyl, —CONHSO₂—N(C₁₋₄-alkyl)₂, 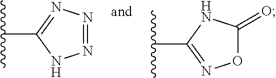 [SMILES: *c1nnn[nH]1 ; *c1noc(=O)[nH]1]
or a prodrug and pharmaceutically acceptable salt thereof.

9. The compound according to claim 1 wherein 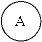
is selected from 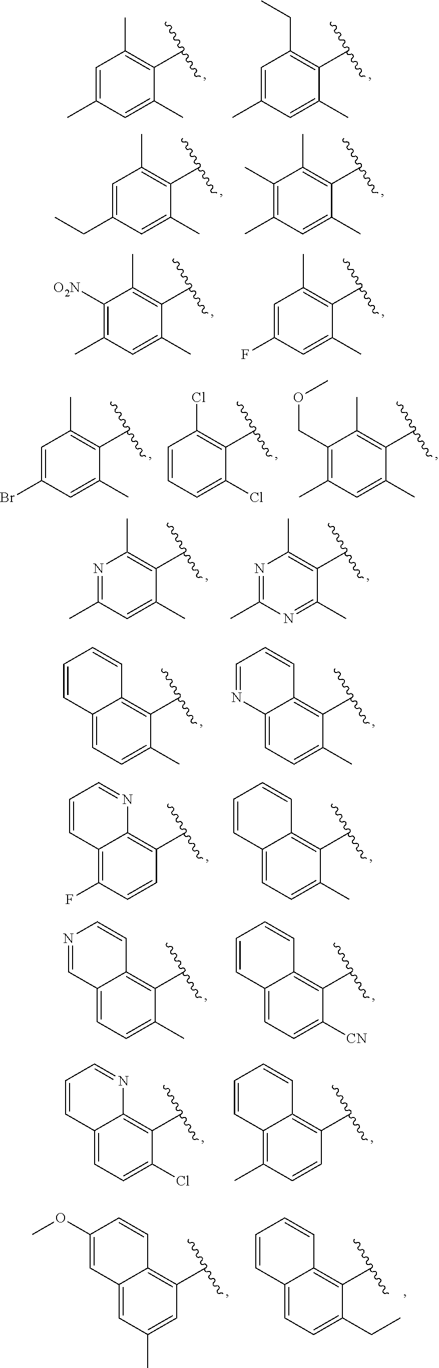 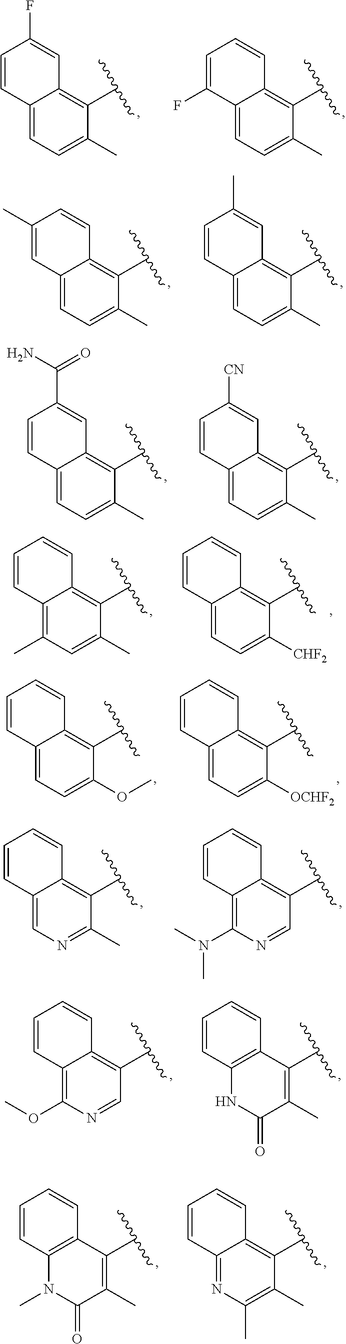 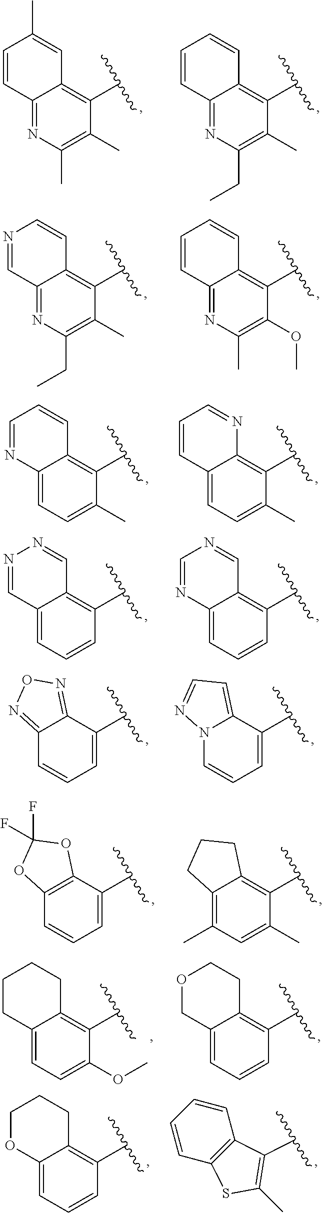 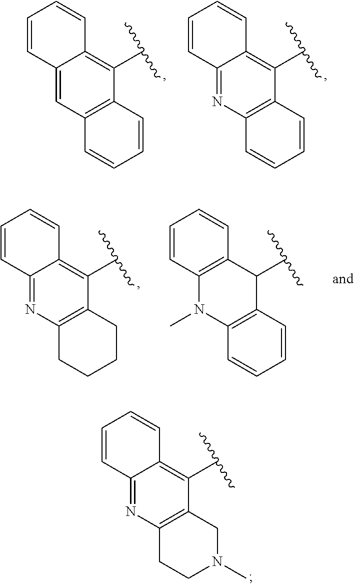 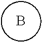
is selected from 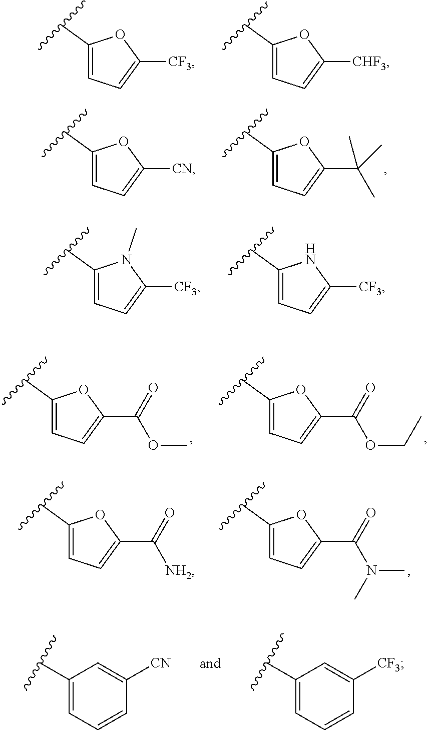 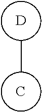
is selected from 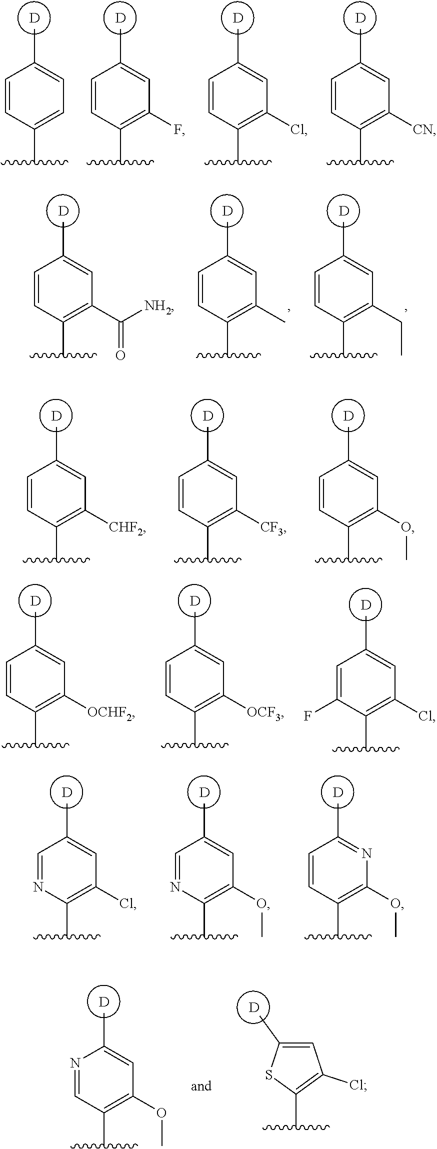 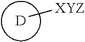
is selected from 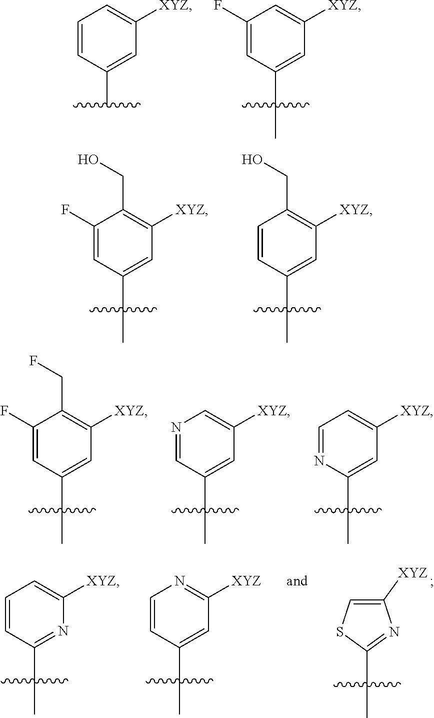
XYZ is selected from 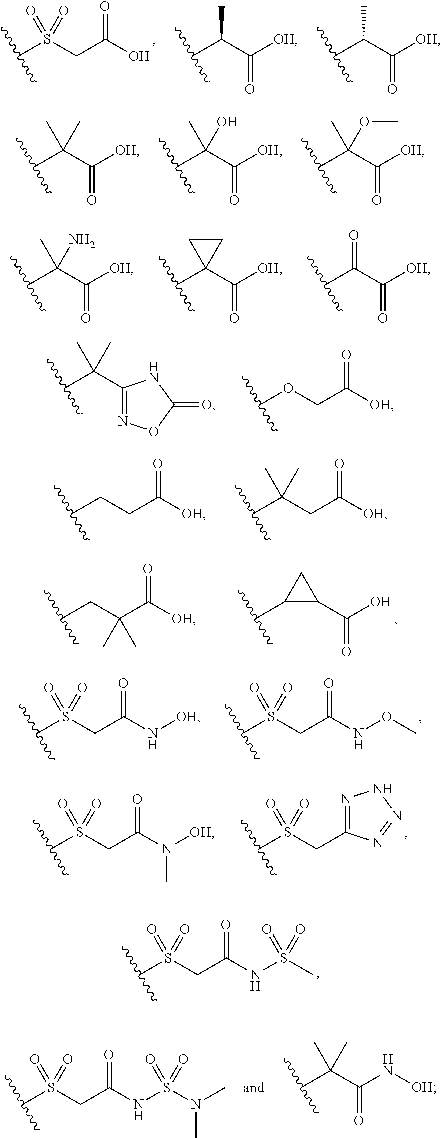
R¹, R², R³ and R⁴ are independently selected from H and Me;
R⁵ and R⁶ are independently selected from H and Me or R⁵ and R⁶ together are oxo;
m and p is 1.

10. The compound according to claim 1 wherein 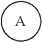
is selected from 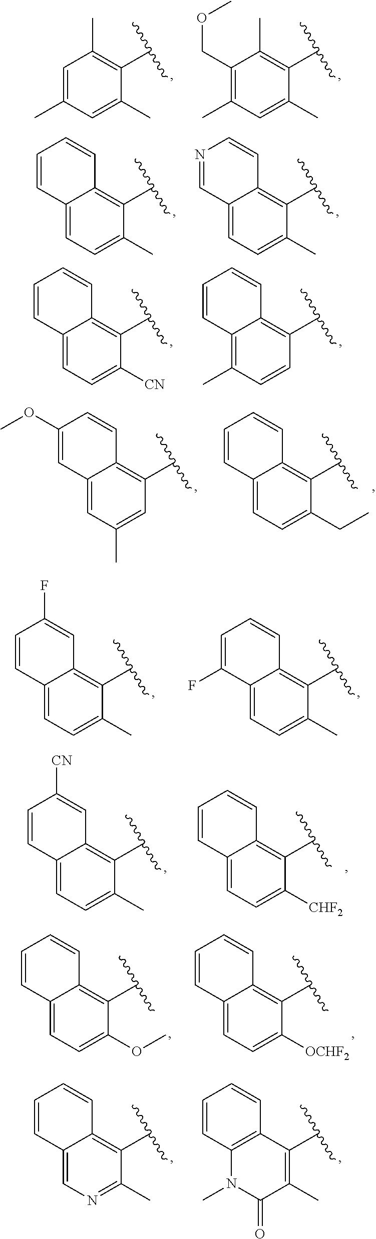 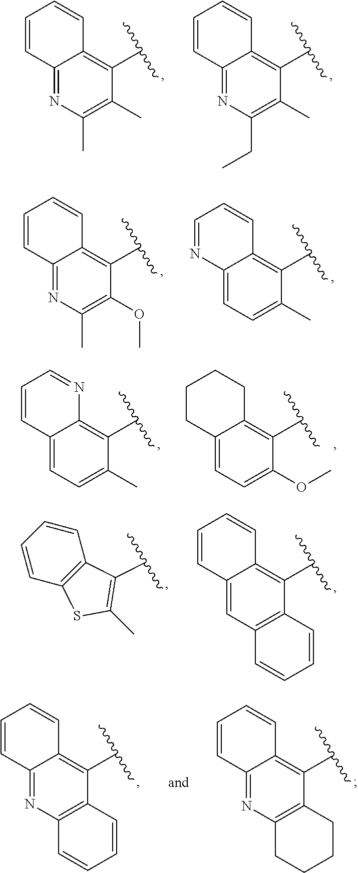 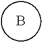
is selected from 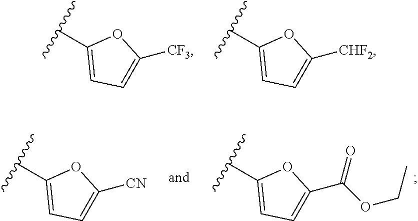 [SMILES: *c1ccc(C(F)(F)F)o1 ; *c1ccc(C(F)F)o1 ; *c1ccc(C#N)o1 ; *c1ccc(C(=O)OCC)o1] 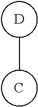
is selected from 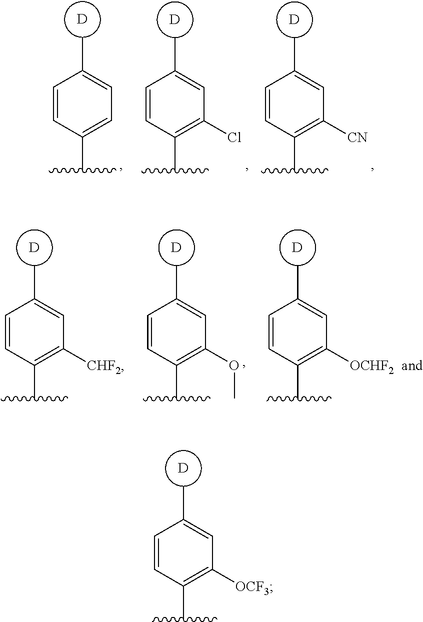 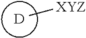
is selected from 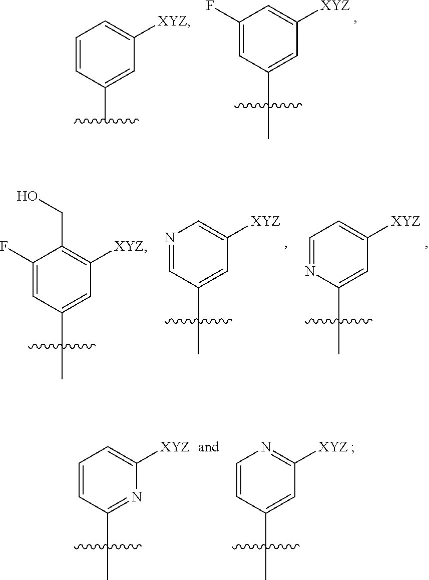
XYZ is selected from 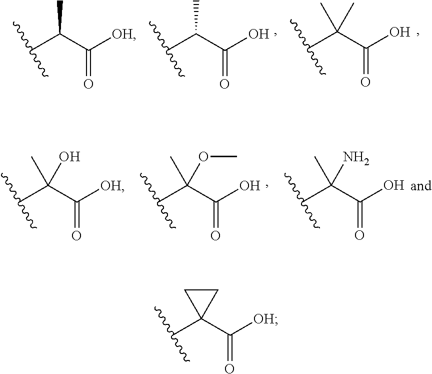
R¹, R², R³ and R⁴ are H;
R⁵ and R⁶ are independently H or R⁵ and R⁶ together are oxo;
m and p is 1.

11. The compound according to claim 1 wherein 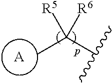
is selected from 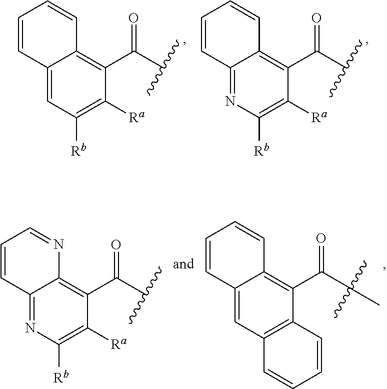
wherein R^{a} and R^{b} is independently selected from H, Cl, CN, Me, Et, cyclopropyl, CHF₂, CF₃, OH, OMe, OCHF₂ and OCF₃; and 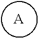
may be further substituted with 1 to 3 additional substituents independently selected from F, Cl, Br, CN, OH, Me, Et, CHF₂, CF₃, OMe, OEt, OCHF₂ and OCF₃; 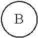
is selected from 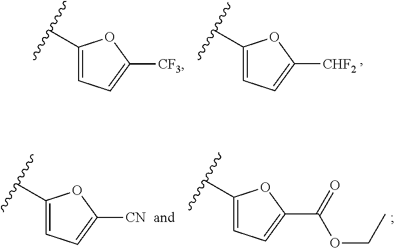 [SMILES: *c1ccc(C(F)(F)F)o1 ; *c1ccc(C(F)F)o1 ; "," ; *c1ccc(C#N)o1 ; *c1ccc(C(=O)OCC)o1] 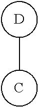
is selected from 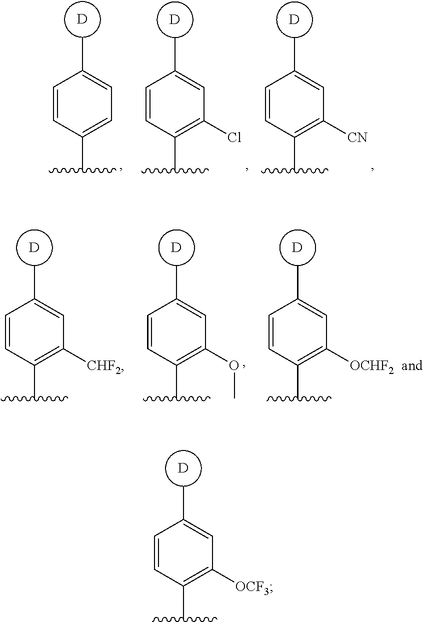
is selected from 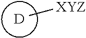
is selected from 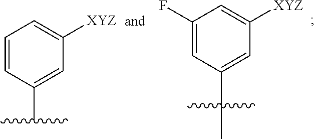
XYZ is selected from 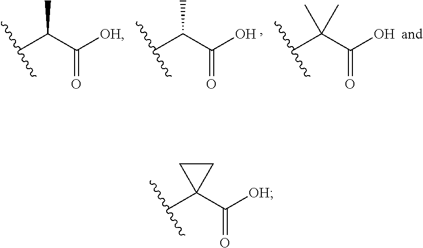
R¹, R², R³ and R⁴ are H; and
m is 1.

12. The compound according to claim 1 selected from 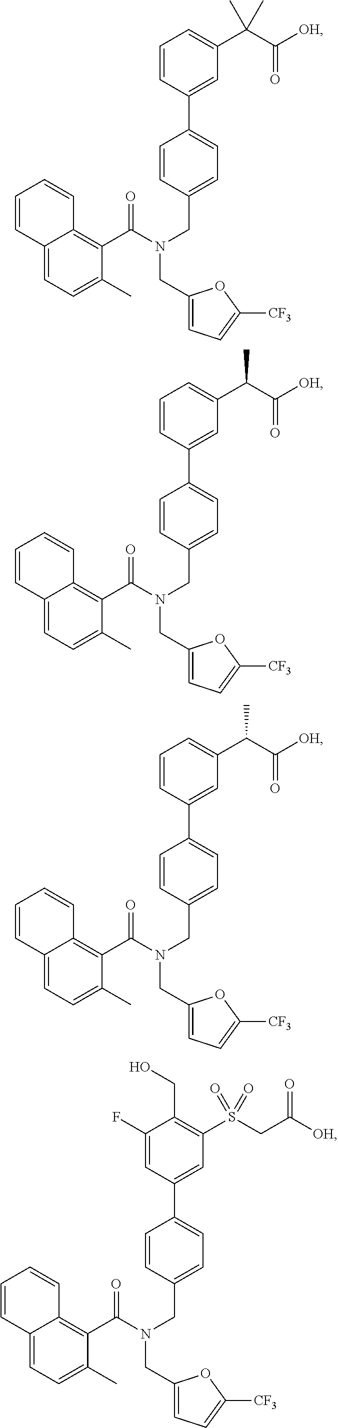 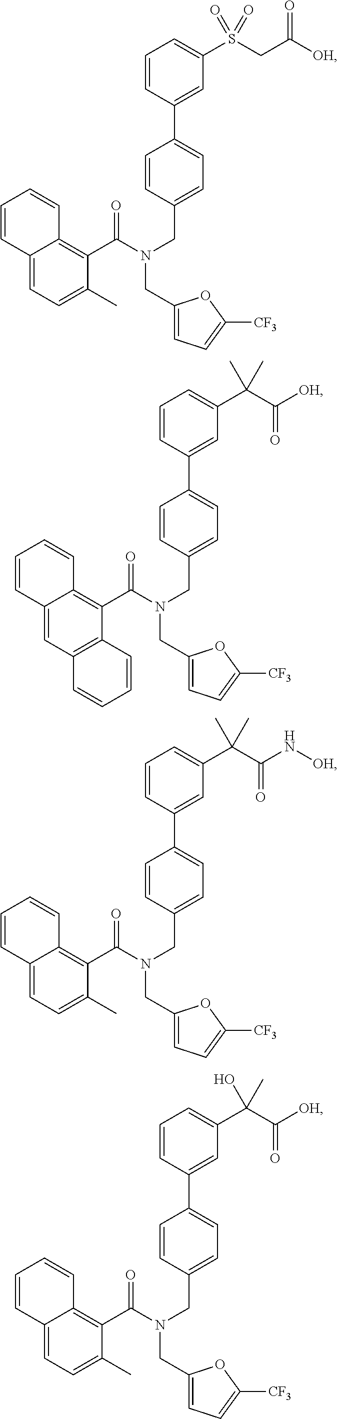 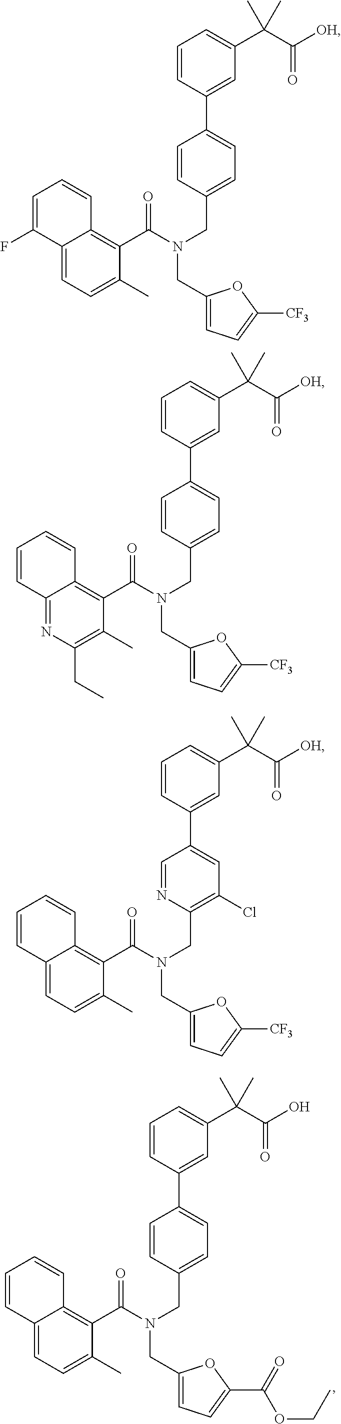 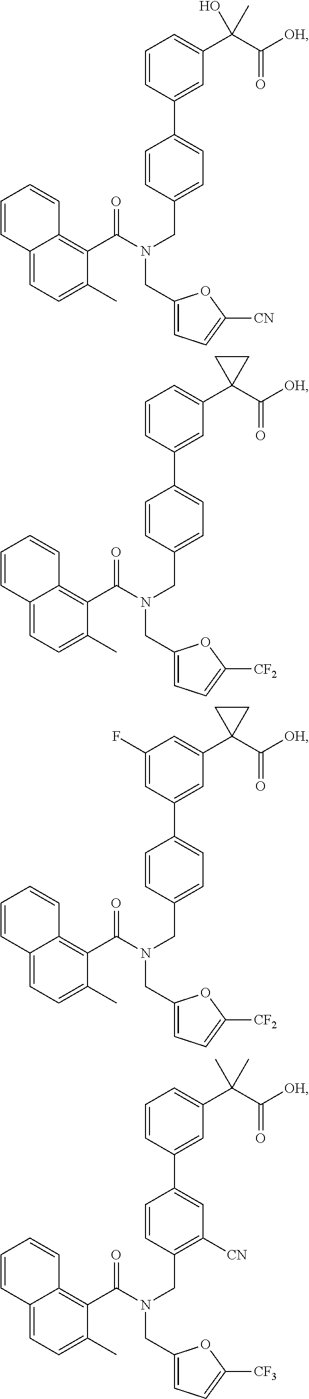 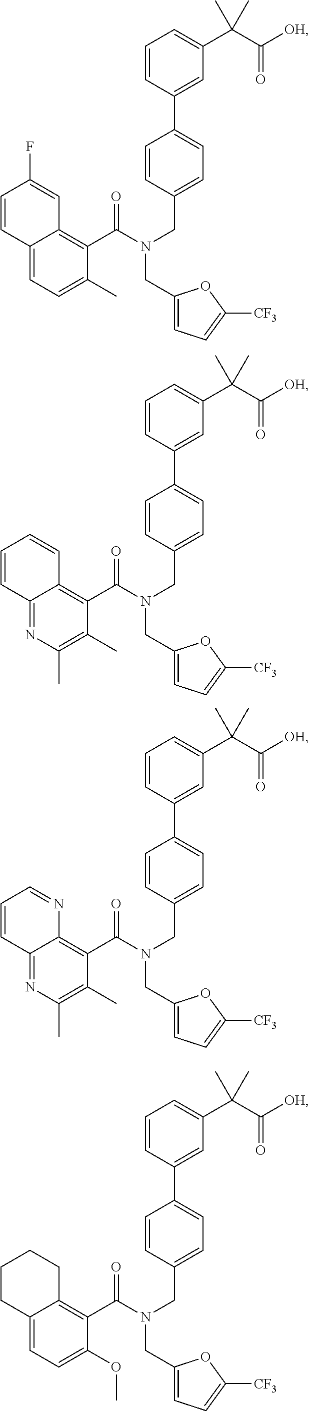 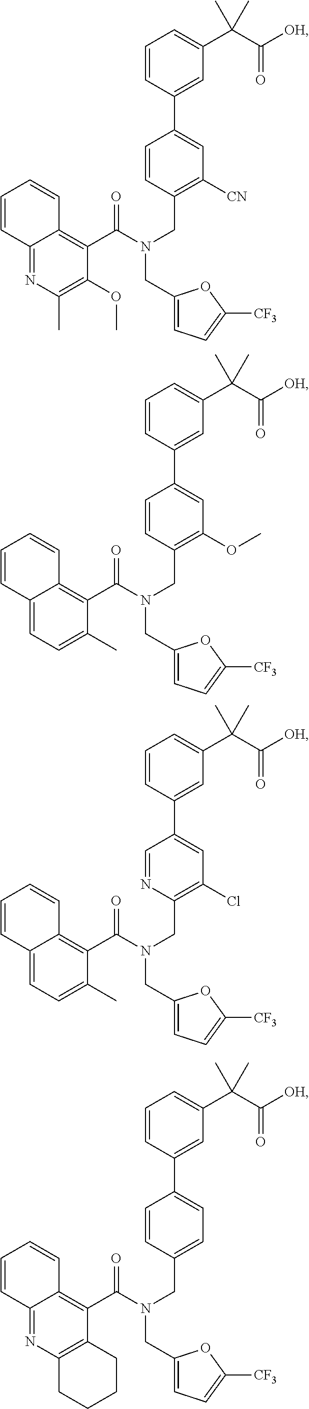 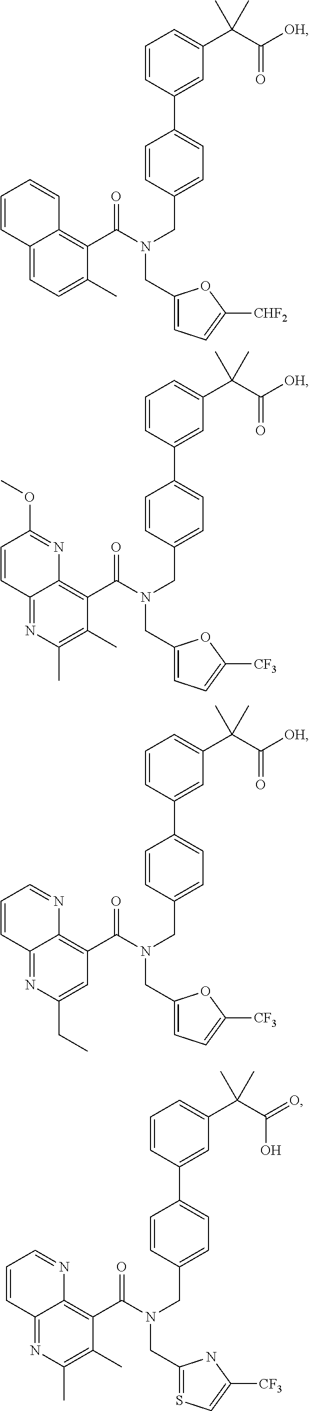 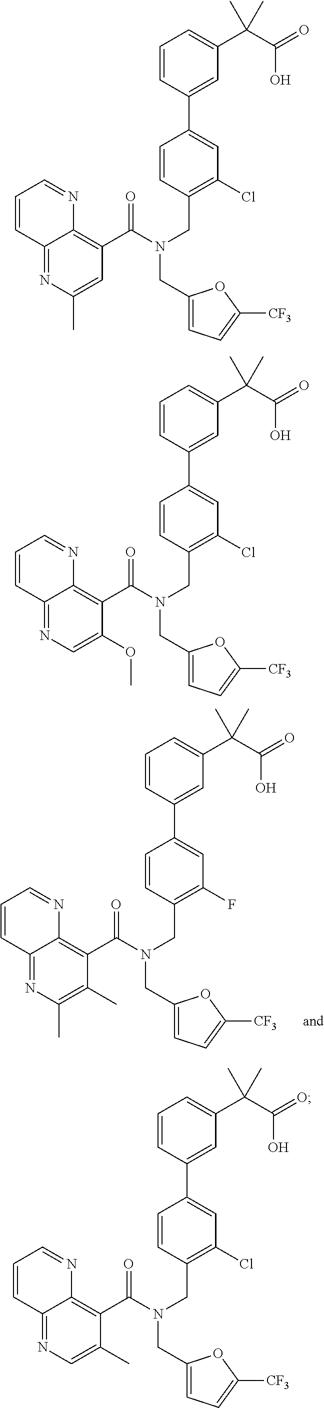
an enantiomer, diastereomer, tautomer, N-oxide, solvate, prodrug and pharmaceutically acceptable salt thereof.

13. (canceled)

14. A method for the prophylaxis and/or treatment of diseases mediated by LXRs, comprising administering a therapeutically effective amount of a compound of claim 1 to a subject in need thereof.

15. The method according to claim 14 wherein the disease is selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

16. A pharmaceutical composition comprising a compound according to claim 1 and a pharmaceutically acceptable carrier or excipient.


SEQUENCE LISTING (19 sequences)

SEQ ID NO: 1 - DNA, 1344 bp, Artificial Sequence
  DNA-Sequence LXR alpha-full length
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagtgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccagggcttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtagagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 2 - DNA, 1386 bp, Artificial Sequence
  DNA-Sequence LXR beta-full length
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacaacagcagcaggagtcacagtcacagtcgcagtcacctgtggggccgcagggcagcagcagctcagcctctgggcctggggcttcccctggtggatctgaggcaggcagccagggctccggggaaggcgagggcgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 3 - DNA, 882 bp, Artificial Sequence
  DNA-Sequence LXR alpha-LBD
cttcgcaaatgccgtcaggctggcatgcgggaggagtgtgtcctgtcagaagaacagatccgcctgaagaaactgaagcggcaagaggaggaacaggctcatgccacatccttgccccccagggcttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtagagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 4 - DNA, 1011 bp, Artificial Sequence
  DNA-sequence LXR beta-LBD
gagcagtgcgtcctttctgaagaacagatccggaagaagaagattcggaaacaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctctgcttttatttaa

SEQ ID NO: 5 - DNA, 1011 bp, Artificial Sequence
  DNA-sequence SRC1-fragment
gttggcttctctgccagttctccagtcctcaggcagatgagctcacagaattcacctagcagattaaatatacaaccagcaaaagctgagtccaaagataacaaagagattgcctcaattttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagaacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgaccaaacccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctaaatagattacctgagctggaattggaagcaattgataaccaatttggacaaccaggaacaggcgattag

SEQ ID NO: 6 - DNA, 1225 bp, Artificial Sequence
  DNA-sequence NCoR-fragment
gataaagggcctcctccaaaatccagatatgaggaagagctaaggaccagagggaagactaccattactgcagctaacttcatagacgtgatcatcacccggcaaattgcctcggacaaggatgcgagggaacgtggctctcaaagttcagactcttctagtagcttatcttctcacaggtatgaaacacctagcgatgctattgaggtgataagtcctgccagctcacctgcgccaccccaggagaaactgcagacctatcagccagaggttgttaaggcaaatcaagcggaaaatgatcctaccagacaatatgaaggaccattacatcactatcgaccacagcaggaatcaccatctccccaacaacagctgcccccttcttcacaggcagagggaatggggcaagtgcccaggacccatcggctgatcacacttgctgatcacatctgtcaaattatcacacaagattttgctagaaatcaagtttcctcgcagactccccagcagcctcctacttctacattccagaactcaccttctgctttggtatctacacctgtgaggactaaaacatcaaaccgttacagcccagaatcccaggctcagtctgtccatcatcaaagaccaggttcaagggtctctacagaaaatcttgtggacaaatccaggggaagtaggcctggaaaatccccagagaggagtcacgtctcttcggagccctacgagcccatctccccaccccaggttccggttgtgcatgagaaacaggacagcttgctgctcttgtctcagaggggcgcagagcctgcagagcagaggaatgatgcccgctcaccagggagtataagctacttgccttcattcttcaccaagcttgaaaatacatcacccatggttaaatcaaagaagcaggagatttttcgtaagttgaactcctctggtggaggtgactctgatatggcagctgctcagccaggaactgagatctttaatctgccagcagttactacgtcaggctcagttagctctagaggccattcttttgctgatcctgccagtaatcttgggctggaagacattatcaggaaggctctcatgggaagctttgatgacaaagttgaggatcatggagttgtcatgtcccagcctatgggagtagtgcctggtactgccaacacctcagttgtgaccagtggtgagacacgaagagaggaaggggtga

SEQ ID NO: 7 - protein, 25 aa, Artificial Sequence
  Peptide
ENQRGPLESKGHKKLLQLLTCSSDD

SEQ ID NO: 8 - DNA, 20 bp, Artificial Sequence
  Forward primer
cccctctgttaattggctcc

SEQ ID NO: 9 - DNA, 20 bp, Artificial Sequence
  Reverse primer
ttgtggaagtgcaggttagg

SEQ ID NO: 10 - DNA, 22 bp, Artificial Sequence
  Sequence probe
caggctcagggtgtcccatgtt

SEQ ID NO: 11 - DNA, 20 bp, Artificial Sequence
  Forward primer
ctgacctgaaagccgagaag

SEQ ID NO: 12 - DNA, 20 bp, Artificial Sequence
  Reverse primer
agaaggtgctaacgaacagg

SEQ ID NO: 13 - DNA, 25 bp, Artificial Sequence
  Sequence probe
tgtttacaaaagtctcgccccagca

SEQ ID NO: 14 - DNA, 20 bp, Artificial Sequence
  Forward primer
ccatcgactacatccgcttc

SEQ ID NO: 15 - DNA, 20 bp, Artificial Sequence
  Reverse primer
gccctccatagacacatctg

SEQ ID NO: 16 - DNA, 24 bp, Artificial Sequence
  Sequence probe
tctcctgcttgagcttctggttgc

SEQ ID NO: 17 - DNA, 21 bp, Artificial Sequence
  Forward primer
caccaatgactcctatgaccc

SEQ ID NO: 18 - DNA, 23 bp, Artificial Sequence
  Reverse primer
caagtttacagccaagattcacg

SEQ ID NO: 19 - DNA, 20 bp, Artificial Sequence
  Sequence probe
actcctgccacaccagcctc